(19) United States
(12) Patent Application Publication
(10) Pub. No.: US 2024/0352458 A1
(43) Pub. Date: Oct. 24, 2024

(54) DOUBLE STRANDED NUCLEIC ACID COMPOUNDS INHIBITING ZPI

(71) Applicant: E-Therapeutics PLC, London (GB)

(72) Inventors: Amy MCCARTHY, London (GB); Graham CRAGGS, London (GB); James LONGDEN, London (GB); Ines DE SANTIAGO, London (GB); Duncan BROWN, London (GB); Ahmad Ali MORTAZAVI, London (GB); Viviana MANNELLA, London (GB); Muthusamy JAYARAMAN, London (GB); Alexandre DEBACKER, London (GB); Adrian James MOGG, London (GB)

(21) Appl. No.: 18/624,912

(22) Filed: Apr. 2, 2024

Related U.S. Application Data
(63) Continuation of application No. PCT/EP2023/070899, filed on Jul. 27, 2023.
(60) Provisional application No. 63/369,631, filed on Jul. 27, 2022.

(30) Foreign Application Priority Data
Feb. 6, 2023 (EP) 23155105.2

(51) Int. Cl. C12N 15/113; A61P 7/04

(52) U.S. Cl. CPC C12N 15/113; A61P 7/04; C12N 2310/14; C12N 2310/315; C12N 2310/351

(57) ABSTRACT

The present invention provides novel nucleic acid compound suitable for therapeutic use. Additionally, the present invention provides methods of making these compounds, as well as methods of using such compounds for the treatment of various diseases and conditions.

### CROSS REFERENCE TO RELATED APPLICATIONS

This application is Continuation of International Application No. PCT/EP2023/070899, filed internationally on Jul. 27, 2023, which claims the priority benefit of European Application No. 23155105.2, filed on Feb. 6, 2023, and U.S. Provisional Patent Application No. 63/369,631, filed on Jul. 27, 2022, the contents of each of which are incorporated herein by reference in their entirety.

### REFERENCE TO AN ELECTRONIC SEQUENCE LISTING

The contents of the electronic sequence listing (228792000901SUBSEQLIST.xml; Size: 6,295,702 bytes; and Date of Creation: Apr. 12, 2024) is herein incorporated by reference in its entirety.

### FIELD OF THE INVENTION

The present invention provides novel nucleic acid compounds, suitable for therapeutic use. Additionally, the present invention provides methods of making these compounds, as well as methods of using such compounds for the treatment of various diseases and conditions.

### BACKGROUND OF THE INVENTION

Nucleic acid compounds have important therapeutic applications in medicine. Nucleic acids can be used to silence genes that are responsible for a particular disease. Gene-silencing prevents formation of a protein by inhibiting translation. Importantly, gene-silencing agents are a promising alternative to traditional small, organic compounds that inhibit the function of the protein linked to the disease. siRNA, antisense RNA, and micro-RNA are oligonucleotides/oligonucleosides that prevent the formation of proteins by gene-silencing.

A number of modified siRNA compounds in particular have been developed in the last two decades for diagnostic and therapeutic purposes, including siRNA/RNAi therapeutic agents for the treatment of various diseases including central-nervous-system diseases, inflammatory diseases, metabolic disorders, oncology, infectious diseases, and ocular diseases.

The present invention relates to nucleic acid compounds, for use in the treatment and/or prevention of disease.

### BRIEF SUMMARY OF THE INVENTION

According to a first aspect of the present invention, there is provided a nucleic acid for inhibiting expression of ZPI, comprising a duplex region that comprises a first strand and a second strand that is at least partially complementary to the first strand, wherein said first strand is: (i) at least partially complementary to a portion of RNA transcribed from the ZPI gene, and (ii) comprises at least 17 contiguous nucleosides differing by 0 or 1 nucleosides from any one of the first strand sequences as listed in Table 2.

According to a second aspect of the present invention, there is provided a nucleic acid for inhibiting expression of ZPI, comprising a duplex region that comprises a first strand and a second strand that is at least partially complementary to the first strand, wherein said first strand is: (i) at least partially complementary to a portion of RNA transcribed from the ZPI gene, and (ii) comprises at least 17 contiguous nucleosides differing by 0 or 1 nucleosides from any one of the first strand modified sequences as listed in Table 3.

A nucleic acid as described herein, wherein the first strand comprises nucleosides 2-18 of any one of the sequences according to the above first and second aspects of the present invention.

A nucleic acid according to the above first aspect of the present invention, wherein the second strand comprises a nucleoside sequence of at least 17 contiguous nucleosides differing by 0 or 1 nucleosides from any one of the second strand sequences as listed in Table 2, and wherein the second strand has a region of at least 85% complementarity over the 17 contiguous nucleosides to the first strand.

A nucleic acid according to the above first aspect of the present invention, wherein the second strand comprises a nucleoside sequence of at least 17 contiguous nucleosides differing by 0 or 1 nucleosides from any one of the second strand sequences as listed in Table 2, and wherein the duplex region comprises at least 14, 15, 16 or 17 complementary base pairs.

A nucleic acid according to the above second aspect of the present invention, wherein the second strand comprises a nucleoside sequence of at least 17 contiguous nucleosides differing by 0 or 1 nucleosides from any one of the second strand modified sequences as listed in Table 4, and wherein the second strand has a region of at least 85% complementarity over the 17 contiguous nucleosides to the first strand.

A nucleic acid according to the above second aspect of the present invention, wherein the second strand comprises a nucleoside sequence of at least 17 contiguous nucleosides differing by 0 or 1 nucleosides from any one of the second strand modified sequences as listed in Table 4, and wherein the duplex region comprises at least 14, 15, 16 or 17 complementary base pairs.

A nucleic acid according to the above first aspect of the present invention, wherein the first strand comprises any one of the first strand sequences as listed in Table 2.

A nucleic acid according to the above second aspect of the present invention, wherein the first strand comprises any one of the first strand modified sequences as listed in Table 3.

A nucleic acid according to the above first aspect of the present invention, wherein the second strand comprises any one of the second strand sequences as listed in Table 2.

A nucleic acid according to the above second aspect of the present invention, wherein the second strand comprises any one of the second strand modified sequences as listed in Table 4.

A nucleic acid according to the above first aspect of the present invention, wherein the first strand comprises any one of the following sequences: SEQ ID NO: 126, SEQ ID NO: 127, SEQ ID NO: 128, SEQ ID NO: 129, SEQ ID NO: 131, SEQ ID NO: 132, SEQ ID NO: 138, SEQ ID NO: 139, SEQ ID NO: 144, SEQ ID NO: 145, SEQ ID NO: 147, SEQ ID NO: 148, SEQ ID NO: 149, SEQ ID NO: 226, SEQ ID NO: 227, SEQ ID NO: 228, SEQ ID NO: 229, SEQ ID NO: 231, SEQ ID NO: 232, SEQ ID NO: 238, SEQ ID NO: 239.

A nucleic acid according to the above second aspect of the present invention, wherein the first strand comprises any one of the following sequences: SEQ ID NO: 366, SEQ ID NO: 367, SEQ ID NO: 368, SEQ ID NO: 369, SEQ ID NO: 371, SEQ ID NO: 372, SEQ ID NO: 378, SEQ ID NO: 379, SEQ ID NO: 384, SEQ ID NO: 385, SEQ ID NO: 387, SEQ ID NO: 388, SEQ ID NO: 389, SEQ ID NO: 466, SEQ ID NO: 467, SEQ ID NO: 468, SEQ ID NO: 469, SEQ ID NO: 471, SEQ ID NO: 472, SEQ ID NO: 478, SEQ ID NO: 479, SEQ ID NO: 498, SEQ ID NO: 518, SEQ ID NO: 538, SEQ ID NO: 546, SEQ ID NO: 547, SEQ ID NO: 548, SEQ ID NO: 549, SEQ ID NO: 551, SEQ ID NO: 552, SEQ ID NO: 558, SEQ ID NO: 559.

A nucleic acid according to the above first aspect of the present invention, wherein the second strand comprises any one of the following sequences: SEQ ID NO: 246, SEQ ID NO: 247, SEQ ID NO: 248, SEQ ID NO: 249, SEQ ID NO: 251, SEQ ID NO: 252, SEQ ID NO: 258, SEQ ID NO: 259, SEQ ID NO: 264, SEQ ID NO: 265, SEQ ID NO: 267, SEQ ID NO: 268, SEQ ID NO: 269, SEQ ID NO: 346, SEQ ID NO: 347, SEQ ID NO: 348, SEQ ID NO: 349, SEQ ID NO: 351, SEQ ID NO: 352, SEQ ID NO: 358, SEQ ID NO: 359.

A nucleic acid according to the above second aspect of the present invention, wherein the second strand comprises any one of the following sequences: SEQ ID NO: 566, SEQ ID NO: 567, SEQ ID NO: 568, SEQ ID NO: 569, SEQ ID NO: 571, SEQ ID NO: 572, SEQ ID NO: 578, SEQ ID NO: 579, SEQ ID NO: 584, SEQ ID NO: 585, SEQ ID NO: 587, SEQ ID NO: 588, SEQ ID NO: 589, SEQ ID NO: 666, SEQ ID NO: 667, SEQ ID NO: 668, SEQ ID NO: 669, SEQ ID NO: 671, SEQ ID NO: 672, SEQ ID NO: 678, SEQ ID NO: 679, SEQ ID NO: 698, SEQ ID NO: 718, SEQ ID NO: 738, SEQ ID NO: 746, SEQ ID NO: 747, SEQ ID NO: 748, SEQ ID NO: 749, SEQ ID NO: 751, SEQ ID NO: 752, SEQ ID NO: 758, SEQ ID NO: 759.

A nucleic acid comprising first and second strands that comprise, consist of, or consist essentially of a nucleoside sequence differing by 0 or 1 nucleosides from any one of the following first and second sequences:

| Unmodified first strand | Unmodified second strand |
|---|---|
| SEQ ID NO: 126 | SEQ ID NO: 246 |
| SEQ ID NO: 127 | SEQ ID NO: 247 |
| SEQ ID NO: 128 | SEQ ID NO: 248 |
| SEQ ID NO: 129 | SEQ ID NO: 249 |
| SEQ ID NO: 131 | SEQ ID NO: 251 |
| SEQ ID NO: 132 | SEQ ID NO: 252 |
| SEQ ID NO: 138 | SEQ ID NO: 258 |
| SEQ ID NO: 139 | SEQ ID NO: 259 |
| SEQ ID NO: 144 | SEQ ID NO: 264 |
| SEQ ID NO: 145 | SEQ ID NO: 265 |
| SEQ ID NO: 147 | SEQ ID NO: 267 |
| SEQ ID NO: 148 | SEQ ID NO: 268 |
| SEQ ID NO: 149 | SEQ ID NO: 269 |
| SEQ ID NO: 226 | SEQ ID NO: 346 |
| SEQ ID NO: 227 | SEQ ID NO: 347 |
| SEQ ID NO: 228 | SEQ ID NO: 348 |
| SEQ ID NO: 229 | SEQ ID NO: 349 |
| SEQ ID NO: 231 | SEQ ID NO: 351 |
| SEQ ID NO: 232 | SEQ ID NO: 352 |
| SEQ ID NO: 238 | SEQ ID NO: 358 |
| SEQ ID NO: 239 | SEQ ID NO: 359 |
| SEQ ID NO: 138 | SEQ ID NO: 258 |
| SEQ ID NO: 138 | SEQ ID NO: 258 |
| SEQ ID NO: 138 | SEQ ID NO: 258 |
| SEQ ID NO: 126 | SEQ ID NO: 246 |
| SEQ ID NO: 127 | SEQ ID NO: 247 |
| SEQ ID NO: 128 | SEQ ID NO: 248 |
| SEQ ID NO: 129 | SEQ ID NO: 249 |
| SEQ ID NO: 131 | SEQ ID NO: 251 |
| SEQ ID NO: 132 | SEQ ID NO: 252 |
| SEQ ID NO: 138 | SEQ ID NO: 258 |
| SEQ ID NO: 139 | SEQ ID NO: 259 |

A nucleic acid comprising first and second strands that comprise, consist of, or consist essentially of a nucleoside sequence differing by 0 or 1 nucleosides from any one of the following first and second sequences:

| Modified first strand | Modified second strand |
|---|---|
| SEQ ID NO: 366 | SEQ ID NO: 566 |
| SEQ ID NO: 367 | SEQ ID NO: 567 |
| SEQ ID NO: 368 | SEQ ID NO: 568 |
| SEQ ID NO: 369 | SEQ ID NO: 569 |
| SEQ ID NO: 371 | SEQ ID NO: 571 |
| SEQ ID NO: 372 | SEQ ID NO: 572 |
| SEQ ID NO: 378 | SEQ ID NO: 578 |
| SEQ ID NO: 379 | SEQ ID NO: 579 |
| SEQ ID NO: 384 | SEQ ID NO: 584 |
| SEQ ID NO: 385 | SEQ ID NO: 585 |
| SEQ ID NO: 387 | SEQ ID NO: 587 |
| SEQ ID NO: 388 | SEQ ID NO: 588 |
| SEQ ID NO: 389 | SEQ ID NO: 589 |
| SEQ ID NO: 466 | SEQ ID NO: 666 |
| SEQ ID NO: 467 | SEQ ID NO: 667 |
| SEQ ID NO: 468 | SEQ ID NO: 668 |
| SEQ ID NO: 469 | SEQ ID NO: 669 |
| SEQ ID NO: 471 | SEQ ID NO: 671 |
| SEQ ID NO: 472 | SEQ ID NO: 672 |
| SEQ ID NO: 478 | SEQ ID NO: 678 |
| SEQ ID NO: 479 | SEQ ID NO: 679 |
| SEQ ID NO: 498 | SEQ ID NO: 698 |
| SEQ ID NO: 518 | SEQ ID NO: 718 |
| SEQ ID NO: 538 | SEQ ID NO: 738 |
| SEQ ID NO: 546 | SEQ ID NO: 746 |
| SEQ ID NO: 547 | SEQ ID NO: 747 |
| SEQ ID NO: 548 | SEQ ID NO: 748 |
| SEQ ID NO: 549 | SEQ ID NO: 749 |
| SEQ ID NO: 551 | SEQ ID NO: 751 |
| SEQ ID NO: 552 | SEQ ID NO: 752 |
| SEQ ID NO: 558 | SEQ ID NO: 758 |
| SEQ ID NO: 559 | SEQ ID NO: 759 |

A nucleic acid comprising first and second strands that comprise, consist of, or consist essentially of a nucleoside sequence differing by 0 or 1 nucleosides from any one of the following first and second sequences:

| Unmodified first strand | Unmodified second strand |
|---|---|
| SEQ ID NO: 128 | SEQ ID NO: 248 |
| SEQ ID NO: 144 | SEQ ID NO: 264 |
| SEQ ID NO: 148 | SEQ ID NO: 268 |
| SEQ ID NO: 149 | SEQ ID NO: 269 |
| SEQ ID NO: 138 | SEQ ID NO: 258 |

A nucleic acid comprising first and second strands that comprise, consist of, or consist essentially of a nucleoside sequence differing by 0 or 1 nucleosides from any one of the following first and second sequences:

| Modified first strand | Modified second strand |
|---|---|
| SEQ ID NO: 368 | SEQ ID NO: 568 |
| SEQ ID NO: 384 | SEQ ID NO: 584 |
| SEQ ID NO: 388 | SEQ ID NO: 588 |
| SEQ ID NO: 389 | SEQ ID NO: 589 |
| SEQ ID NO: 538 | SEQ ID NO: 738 |

A nucleic acid comprising first and second strands that comprise, consist of, or consist essentially of a nucleoside sequence differing by 0 or 1 nucleosides from any one of the following first and second sequences:

| Unmodified first strand | Unmodified second strand |
|---|---|
| SEQ ID NO: 148 | SEQ ID NO: 268 |
| SEQ ID NO: 145 | SEQ ID NO: 265 |
| SEQ ID NO: 144 | SEQ ID NO: 264 |
| SEQ ID NO: 165 | SEQ ID NO: 285 |
| SEQ ID NO: 202 | SEQ ID NO: 322 |

A nucleic acid comprising first and second strands that comprise, consist of, or consist essentially of a nucleoside sequence differing by 0 or 1 nucleosides from any one of the following first and second sequences:

| Modified first strand | Modified second strand |
|---|---|
| SEQ ID NO: 388 | SEQ ID NO: 588 |
| SEQ ID NO: 385 | SEQ ID NO: 585 |
| SEQ ID NO: 384 | SEQ ID NO: 584 |
| SEQ ID NO: 405 | SEQ ID NO: 605 |
| SEQ ID NO: 442 | SEQ ID NO: 642 |

A nucleic acid comprising first and second strands that comprise, consist of, or consist essentially of a nucleoside sequence differing by 0 or 1 nucleosides from any one of the following first and second sequences:

| Modified first strand | Modified second strand |
|---|---|
| SEQ ID NO: 762 | SEQ ID NO: 772 |
| SEQ ID NO: 763 | SEQ ID NO: 773 |
| SEQ ID NO: 764 | SEQ ID NO: 774 |
| SEQ ID NO: 765 | SEQ ID NO: 775 |
| SEQ ID NO: 766 | SEQ ID NO: 776 |
| SEQ ID NO: 767 | SEQ ID NO: 777 |
| SEQ ID NO: 768 | SEQ ID NO: 778 |
| SEQ ID NO: 769 | SEQ ID NO: 779 |
| SEQ ID NO: 770 | SEQ ID NO: 780 |
| SEQ ID NO: 771 | SEQ ID NO: 781 |
| SEQ ID NO: 782 | SEQ ID NO: 773 |
| SEQ ID NO: 783 | SEQ ID NO: 775 |
| SEQ ID NO: 784 | SEQ ID NO: 777 |
| SEQ ID NO: 785 | SEQ ID NO: 779 |
| SEQ ID NO: 786 | SEQ ID NO: 781 |

A nucleic acid comprising first and second strands that comprise, consist of, or consist essentially of a nucleoside sequence differing by 0 or 1 nucleosides from any one of the following first and second sequences:

| Modified first strand | Modified second strand |
|---|---|
| SEQ ID NO: 385 | SEQ ID NO: 585 |
| SEQ ID NO: 388 | SEQ ID NO: 588 |
| SEQ ID NO: 764 | SEQ ID NO: 774 |
| SEQ ID NO: 765 | SEQ ID NO: 775 |
| SEQ ID NO: 766 | SEQ ID NO: 776 |
| SEQ ID NO: 767 | SEQ ID NO: 777 |

A nucleic acid comprising first and second strands that comprise, consist of, or consist essentially of a nucleoside sequence differing by 0 or 1 nucleosides from any one of the following first and second sequences:

| Unmodified first strand | Unmodified second strand |
|---|---|
| SEQ ID NO: 145 | SEQ ID NO: 265 |
| SEQ ID NO: 148 | SEQ ID NO: 268 |

A nucleic acid comprising first and second strands that comprise, consist of, or consist essentially of a nucleoside sequence differing by 0 or 1 nucleosides from any one of the following first and second sequences:

| Modified first strand | Modified second strand |
|---|---|
| SEQ ID NO: 385 | SEQ ID NO: 585 |
| SEQ ID NO: 764 | SEQ ID NO: 774 |
| SEQ ID NO: 765 | SEQ ID NO: 775 |
| SEQ ID NO: 783 | SEQ ID NO: 775 |
| SEQ ID NO: 801 | SEQ ID NO: 819 |
| SEQ ID NO: 802 | SEQ ID NO: 819 |
| SEQ ID NO: 803 | SEQ ID NO: 819 |
| SEQ ID NO: 804 | SEQ ID NO: 819 |
| SEQ ID NO: 805 | SEQ ID NO: 819 |
| SEQ ID NO: 806 | SEQ ID NO: 819 |
| SEQ ID NO: 807 | SEQ ID NO: 819 |
| SEQ ID NO: 808 | SEQ ID NO: 819 |
| SEQ ID NO: 809 | SEQ ID NO: 819 |

A nucleic acid comprising first and second strands that comprise, consist of, or consist essentially of a nucleoside sequence differing by 0 or 1 nucleosides from any one of the following first and second sequences:

| Modified first strand | Modified second strand |
|---|---|
| SEQ ID NO: 388 | SEQ ID NO: 588 |
| SEQ ID NO: 766 | SEQ ID NO: 776 |
| SEQ ID NO: 767 | SEQ ID NO: 777 |
| SEQ ID NO: 810 | SEQ ID NO: 820 |
| SEQ ID NO: 811 | SEQ ID NO: 820 |
| SEQ ID NO: 812 | SEQ ID NO: 820 |
| SEQ ID NO: 813 | SEQ ID NO: 820 |
| SEQ ID NO: 814 | SEQ ID NO: 820 |
| SEQ ID NO: 815 | SEQ ID NO: 820 |
| SEQ ID NO: 816 | SEQ ID NO: 820 |
| SEQ ID NO: 817 | SEQ ID NO: 820 |
| SEQ ID NO: 818 | SEQ ID NO: 820 |

A nucleic acid comprising first and second strands that comprise, consist of, or consist essentially of a nucleoside sequence differing by 0 or 1 nucleosides from any one of the following first and second sequences:

| Modified first strand | Modified second strand |
|---|---|
| SEQ ID NO: 764 | SEQ ID NO: 774 |
| SEQ ID NO: 766 | SEQ ID NO: 776 |

A conjugate for inhibiting expression of ZPI target gene in a cell, said conjugate comprising a nucleic acid as disclosed herein and one or more ligand moieties.

A pharmaceutical composition comprising a nucleic acid as disclosed herein, in combination with a pharmaceutically acceptable excipient or carrier.

A nucleic acid or pharmaceutical composition, for use in therapy.

A nucleic acid or pharmaceutical composition, for use in prevention or treatment of a disease related to a disorder of haemostasis, such as a disease related to a disorder of haemostasis, such as haemophilia.

### BRIEF DESCRIPTION OF THE DRAWINGS

FIG. 1: Linker and ligand portions of constructs suitable for use according to the present invention including tether 1a. While FIG. 1 depicts the linker to be conjugated to an oligonucleotide, it is to be understood that the present invention also encompasses conjugates of the same linker with an oligonucleoside as disclosed herein.

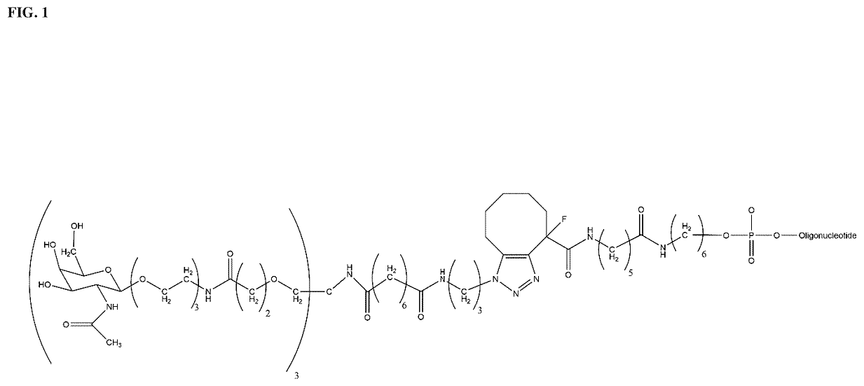

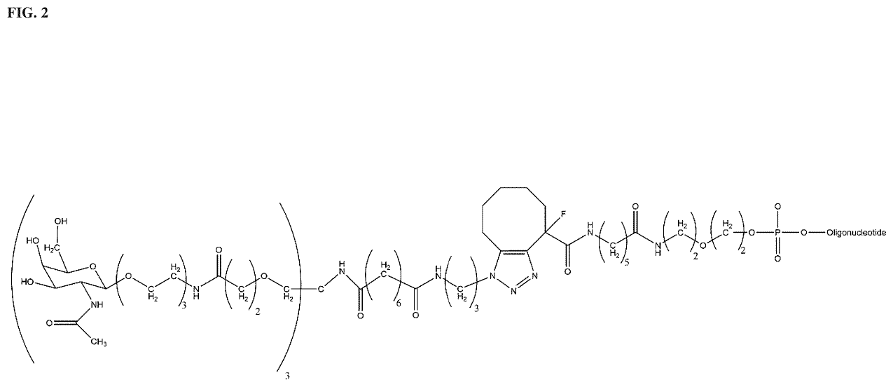

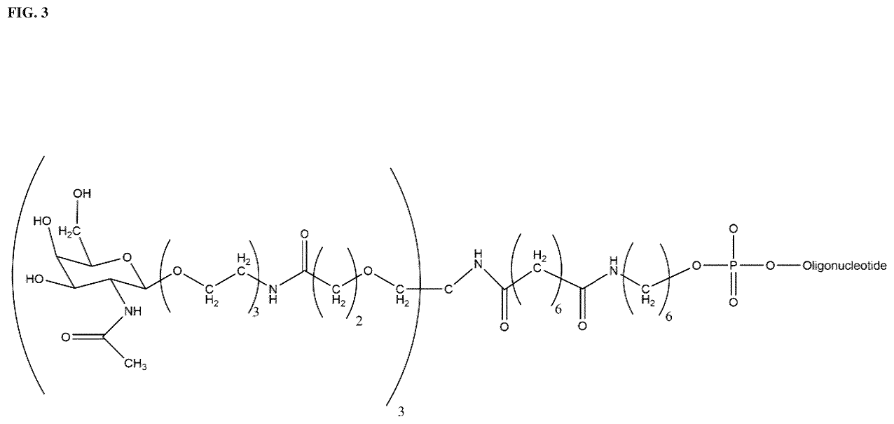

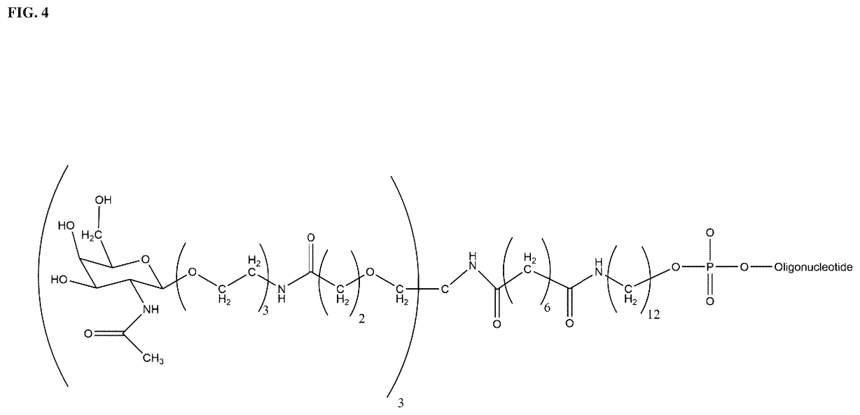

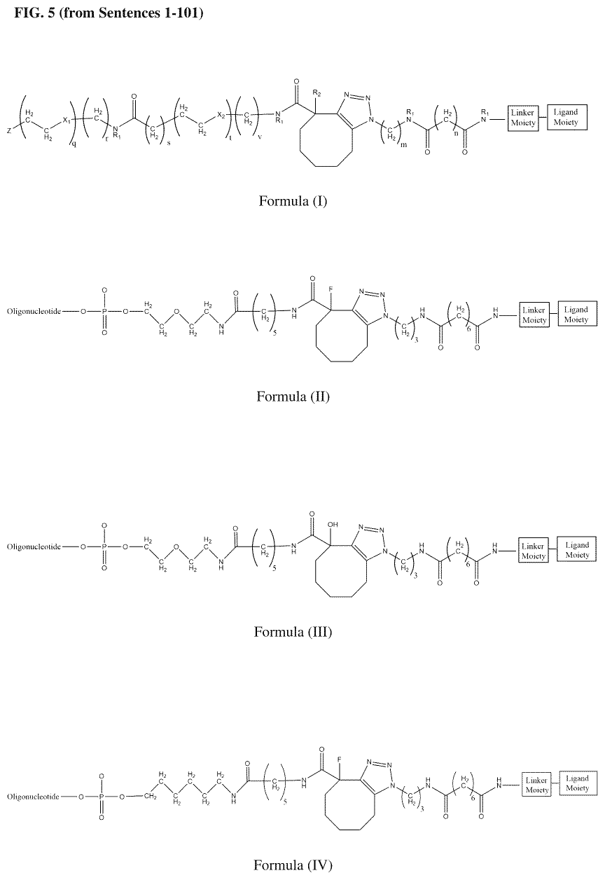

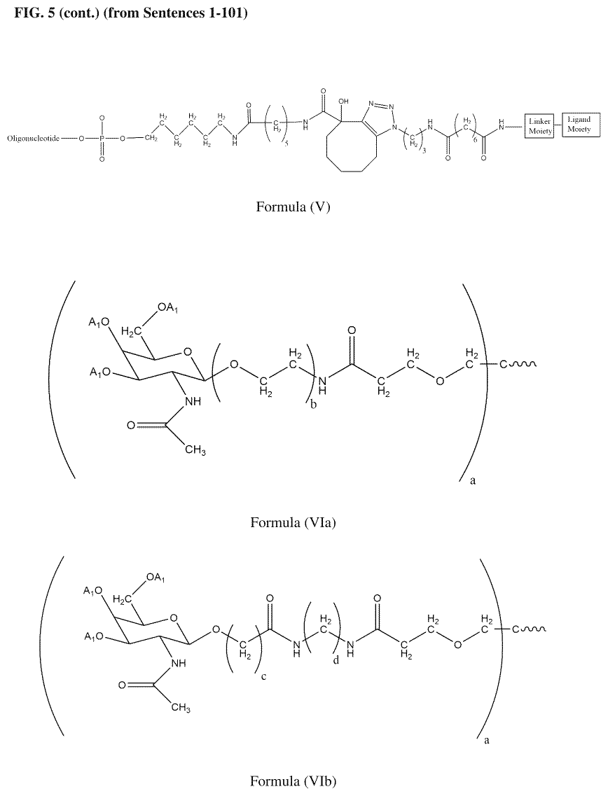

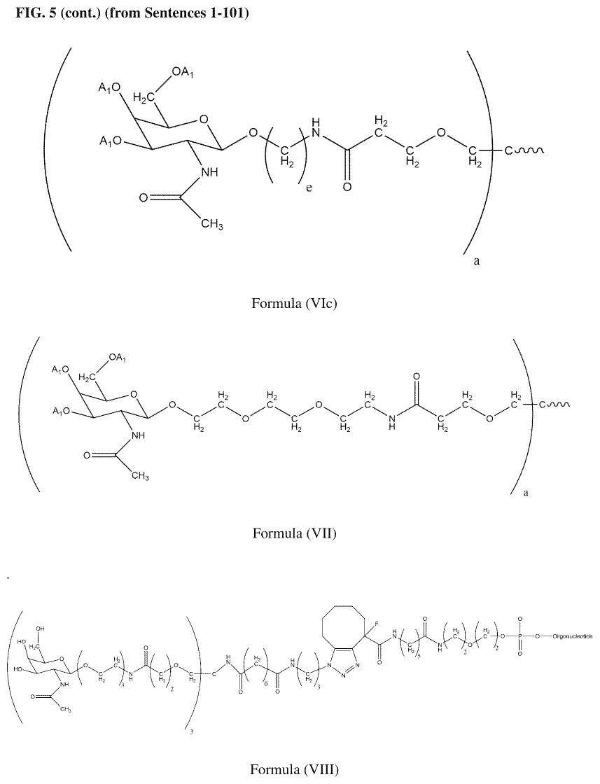

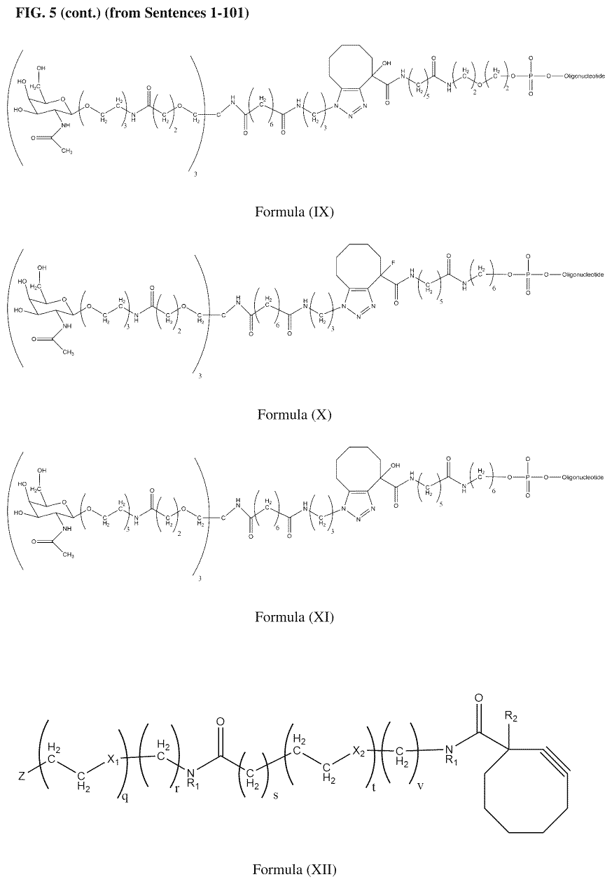

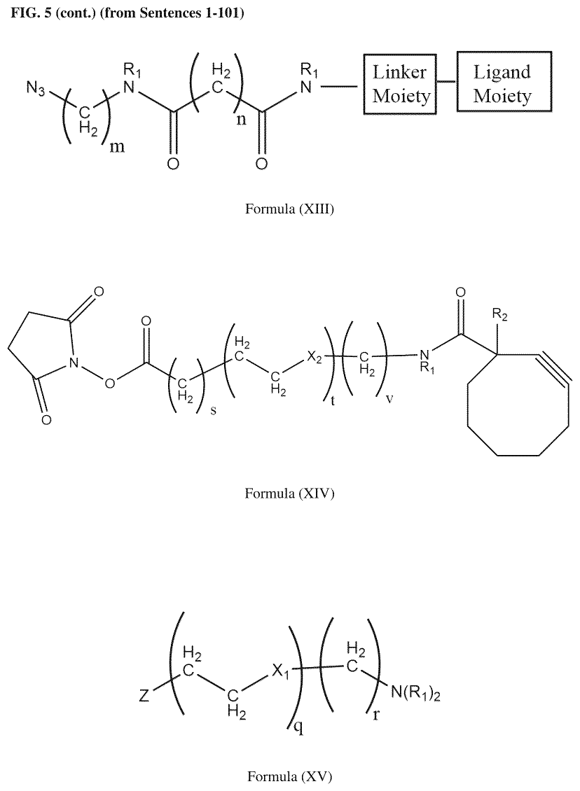

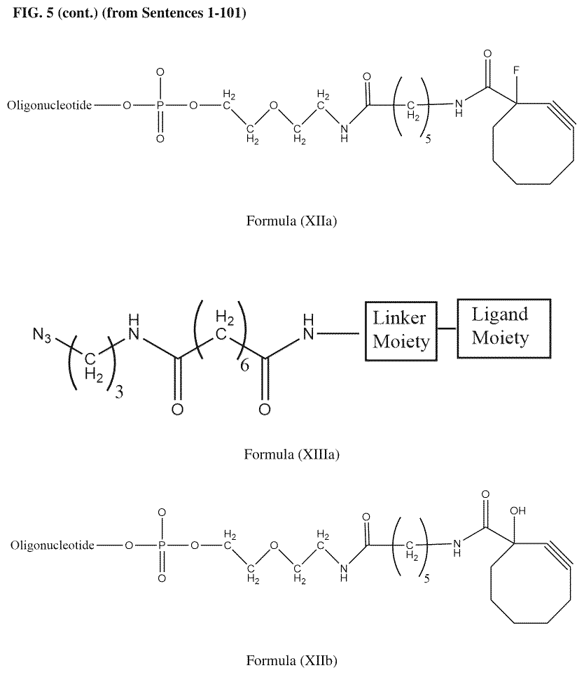

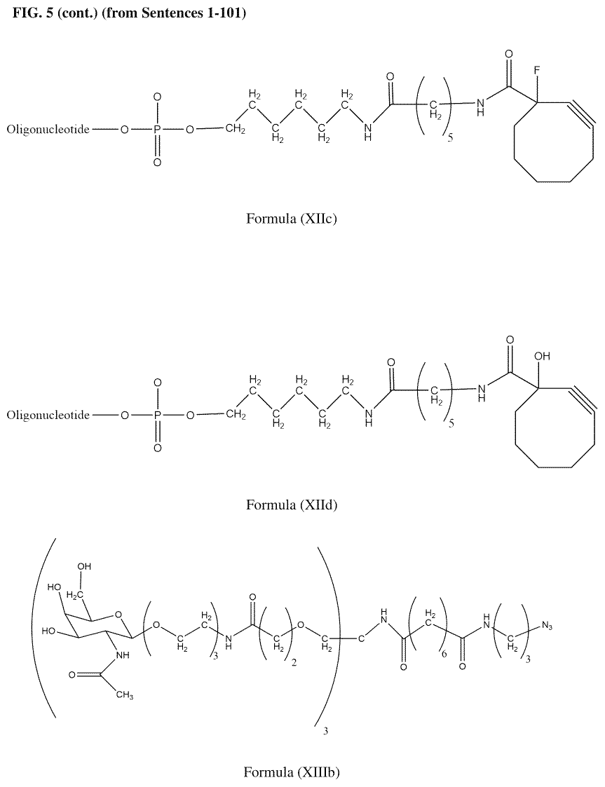

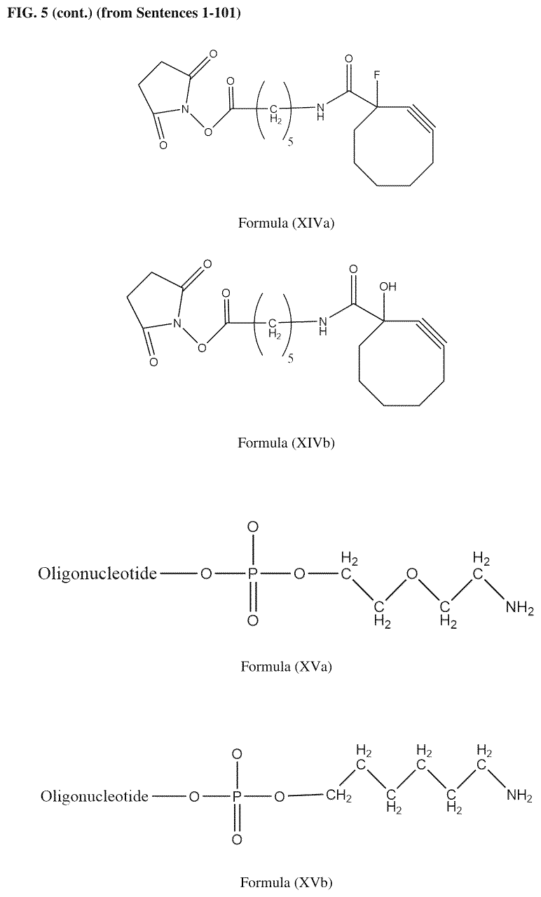

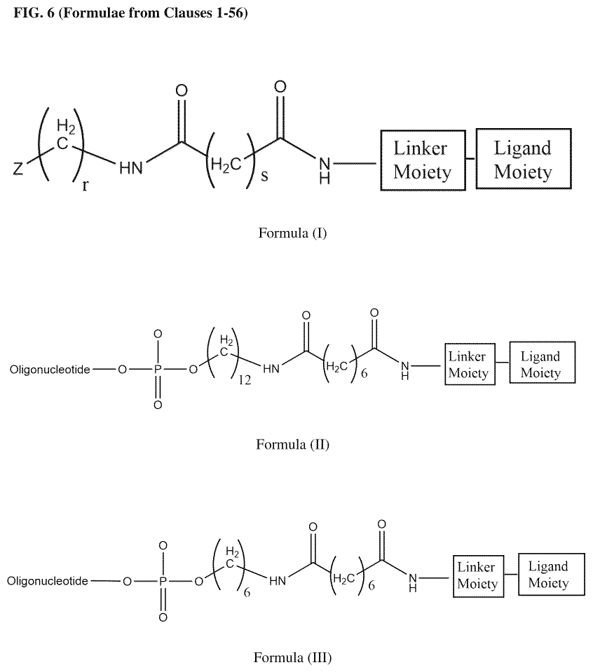

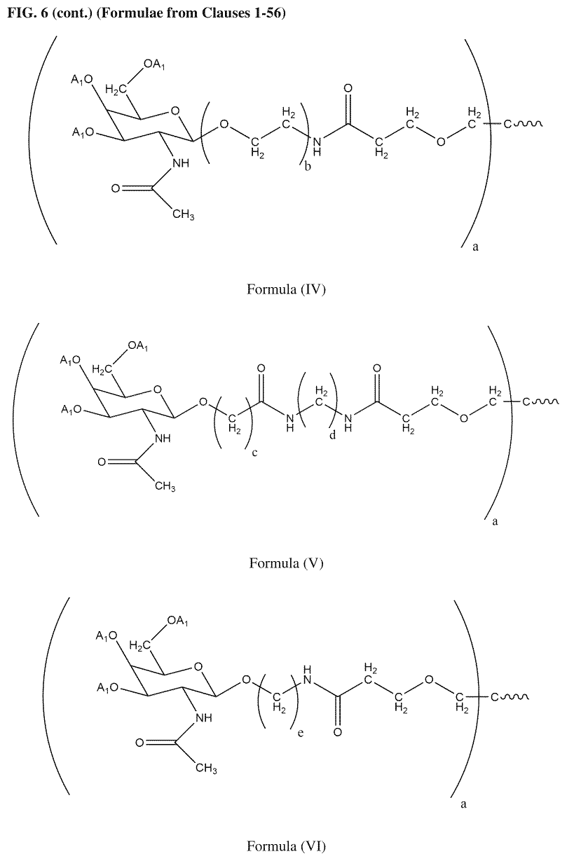

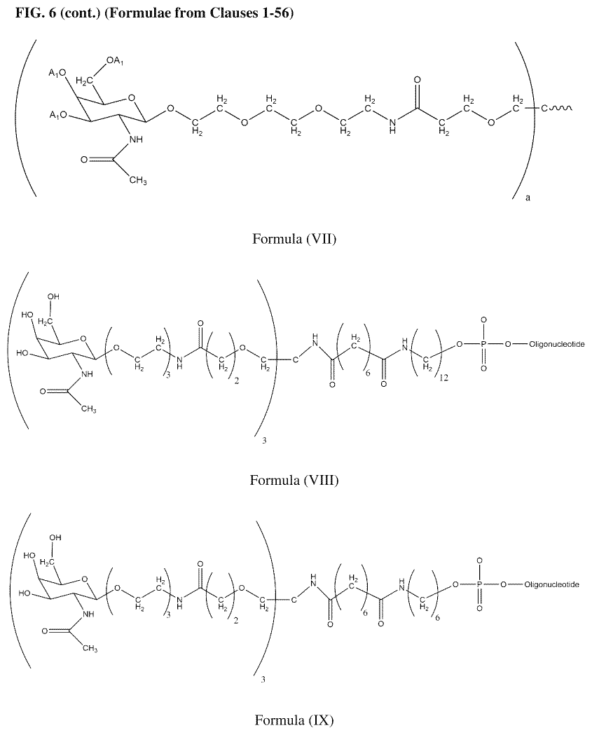

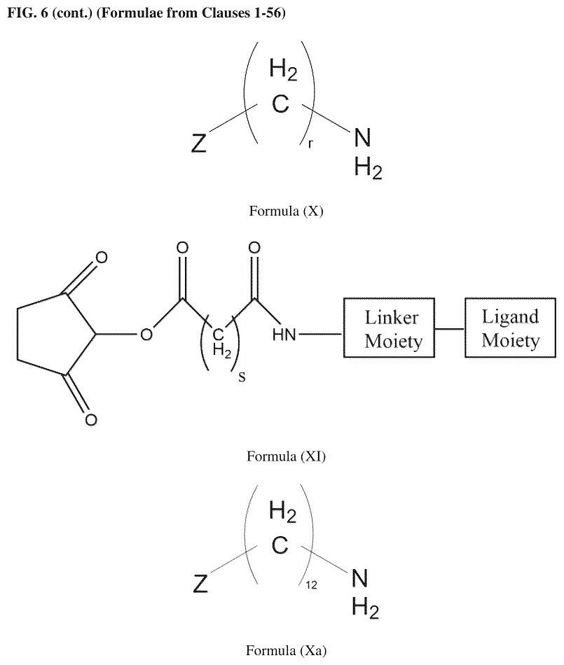

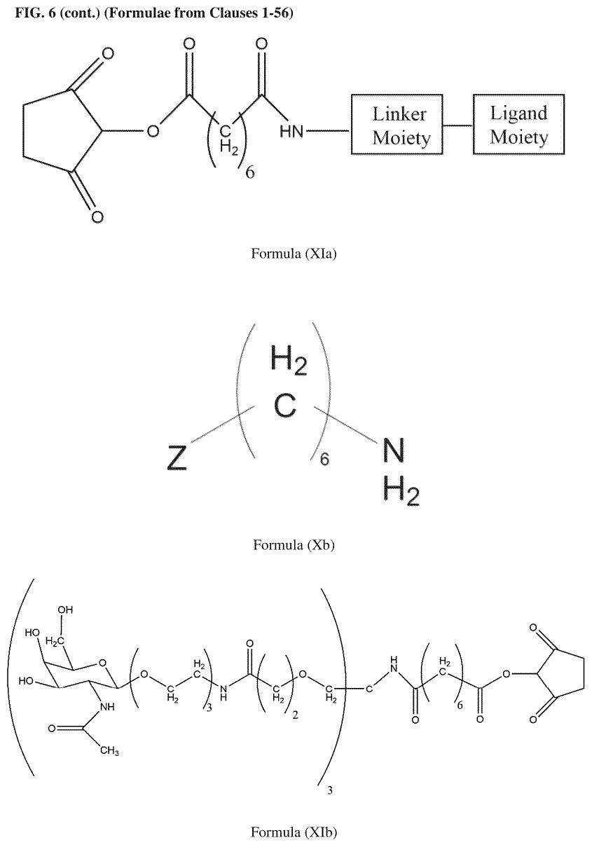

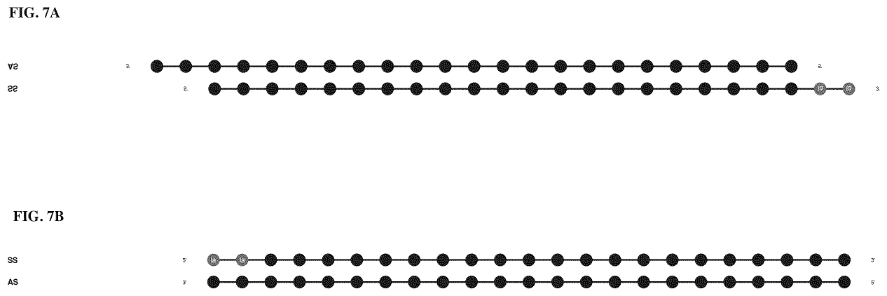

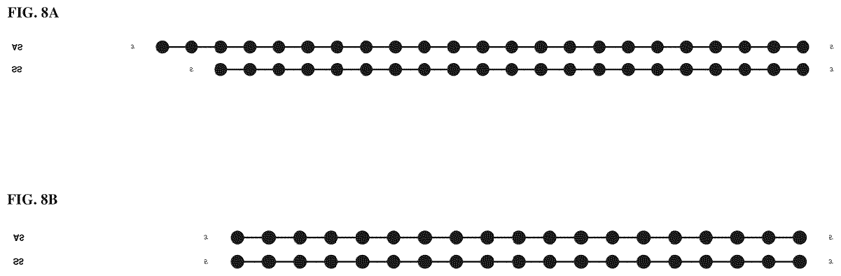

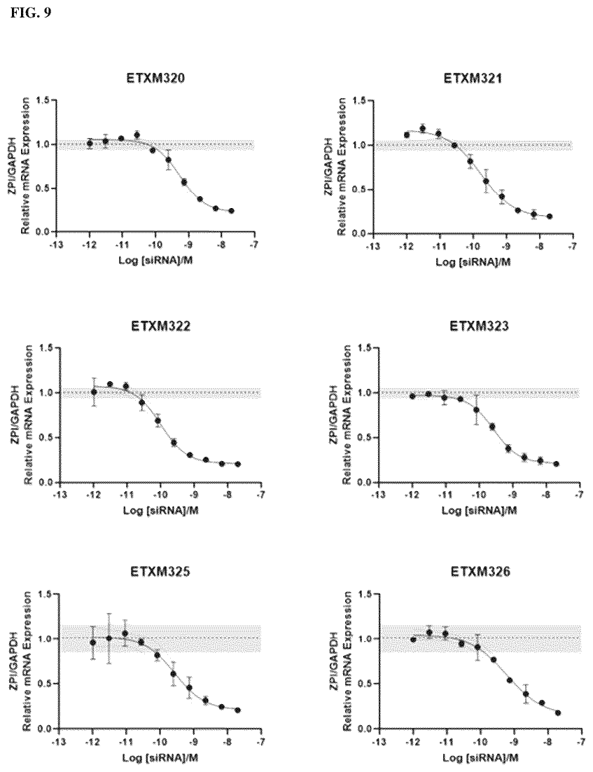

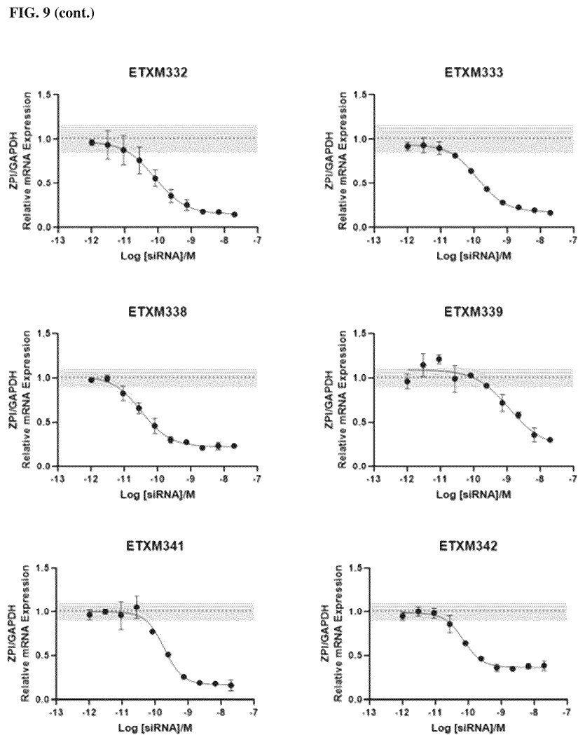

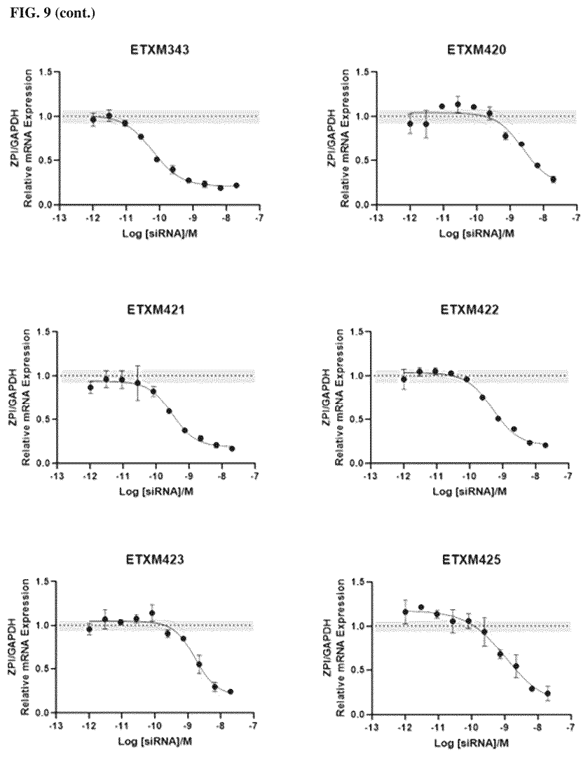

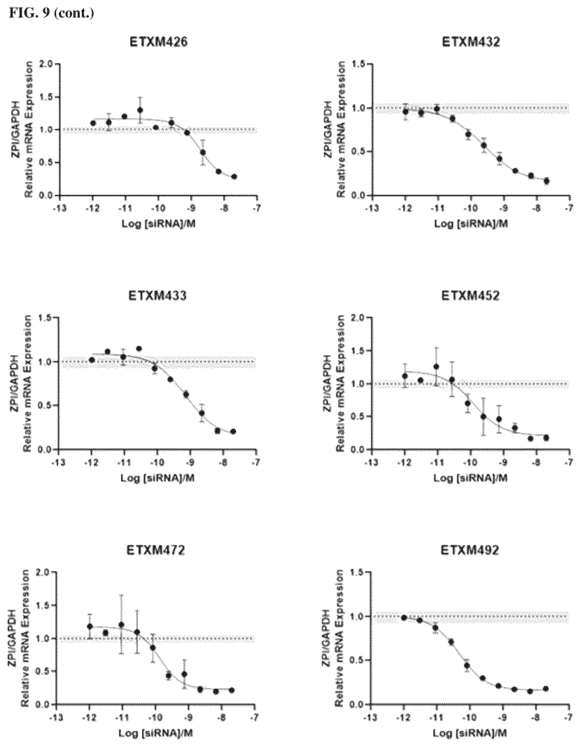

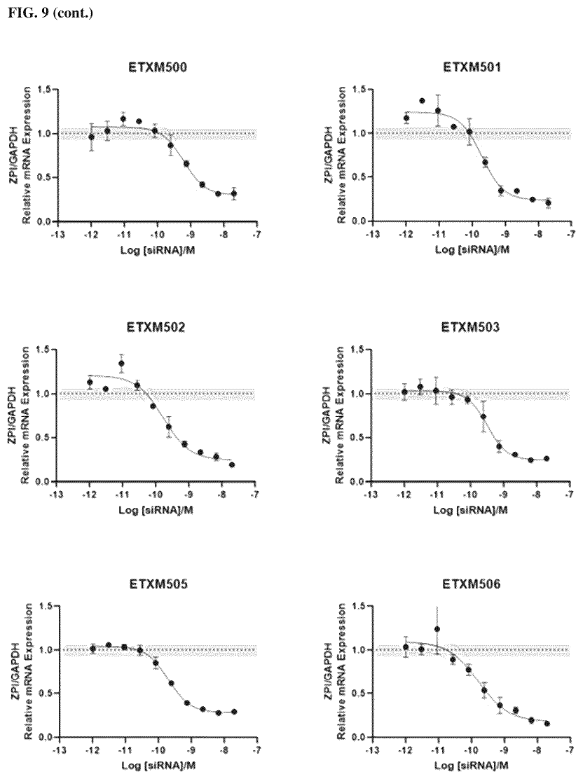

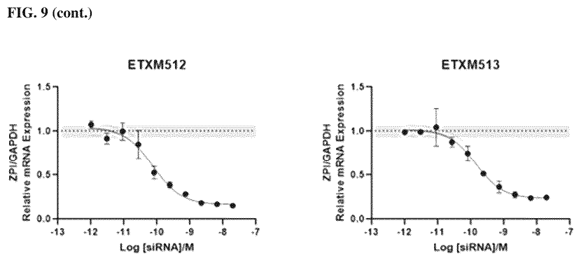

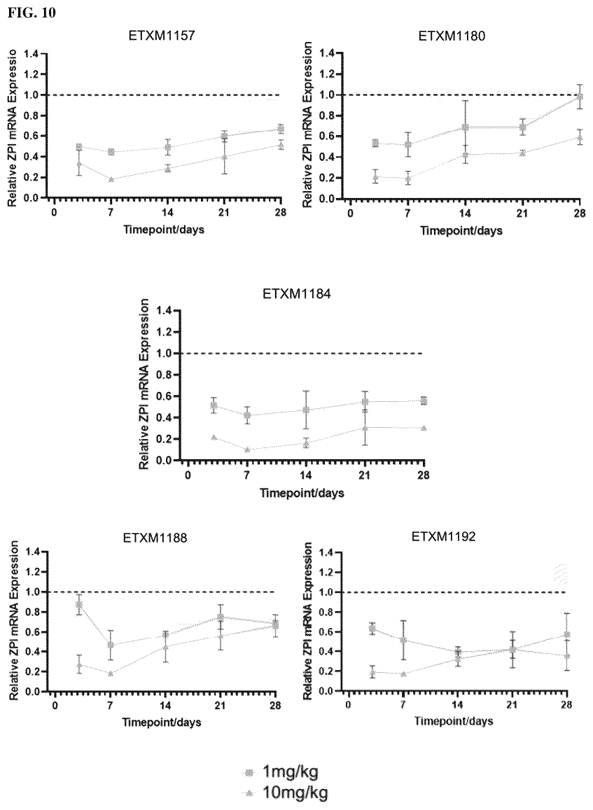

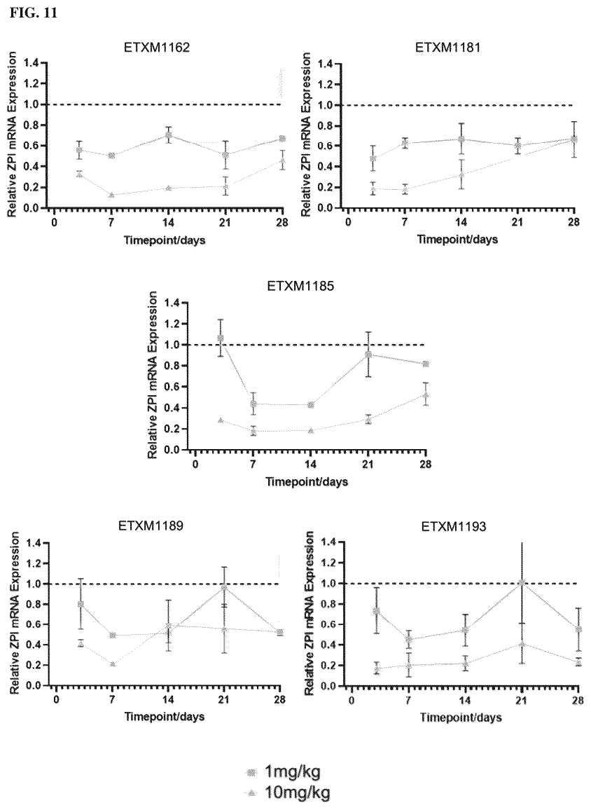

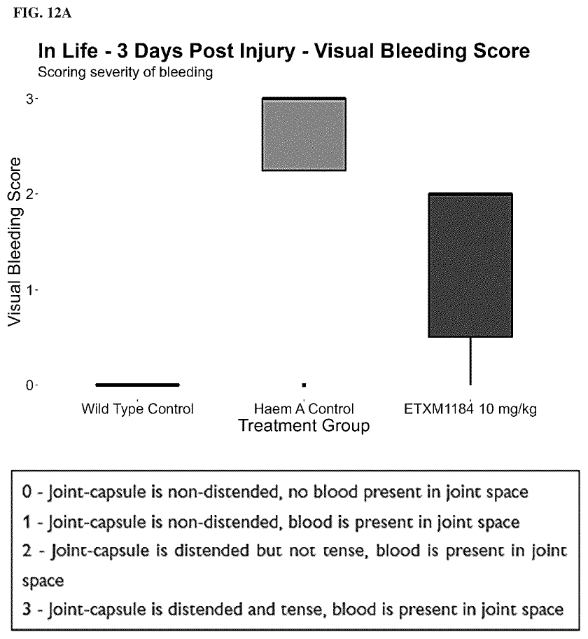

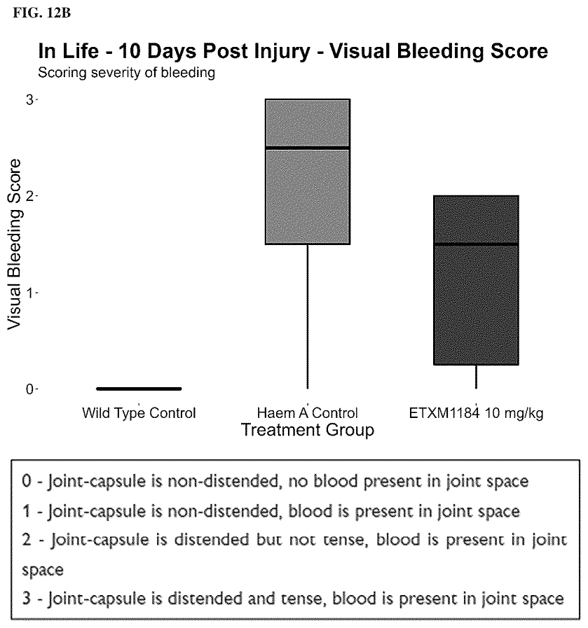

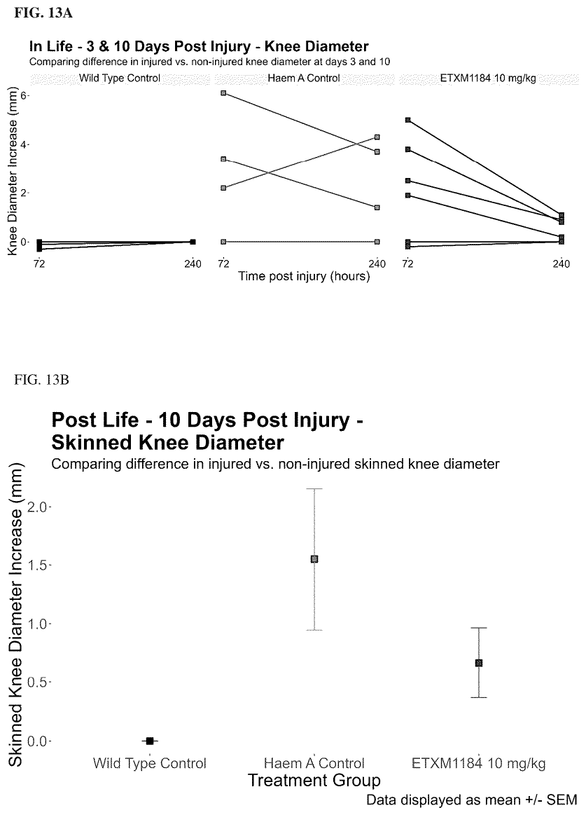

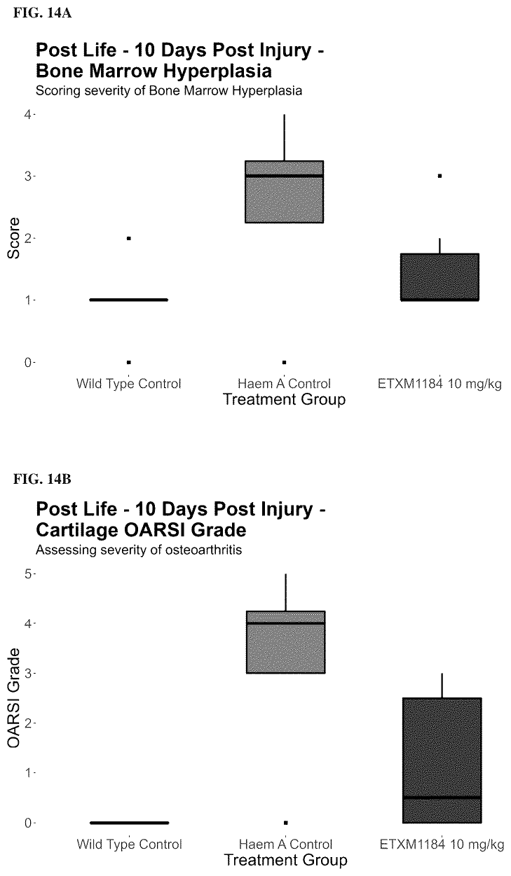

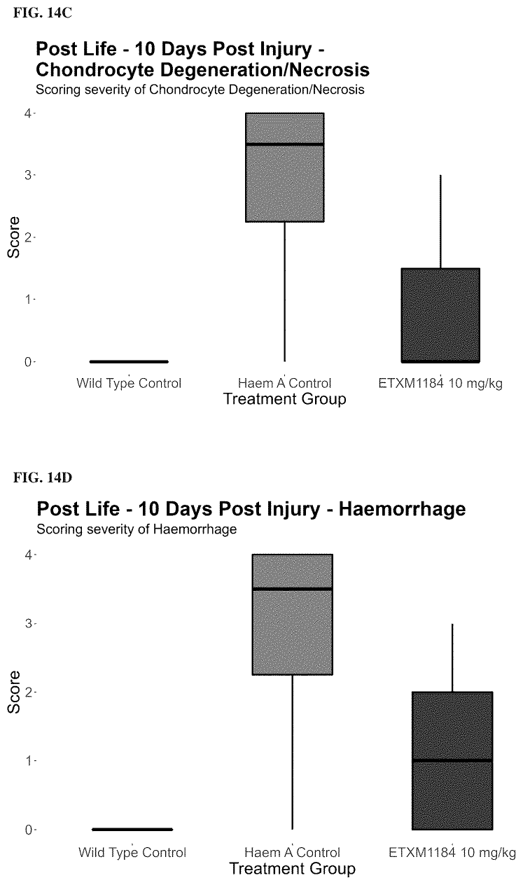

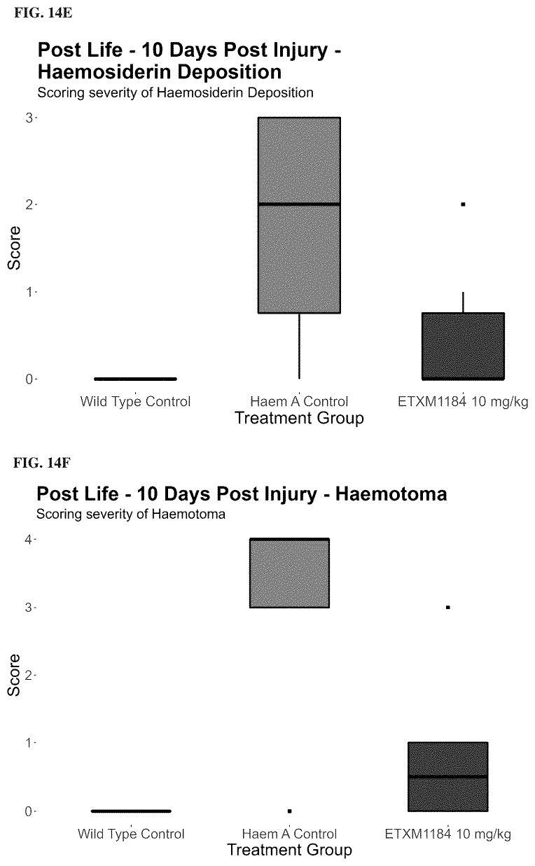

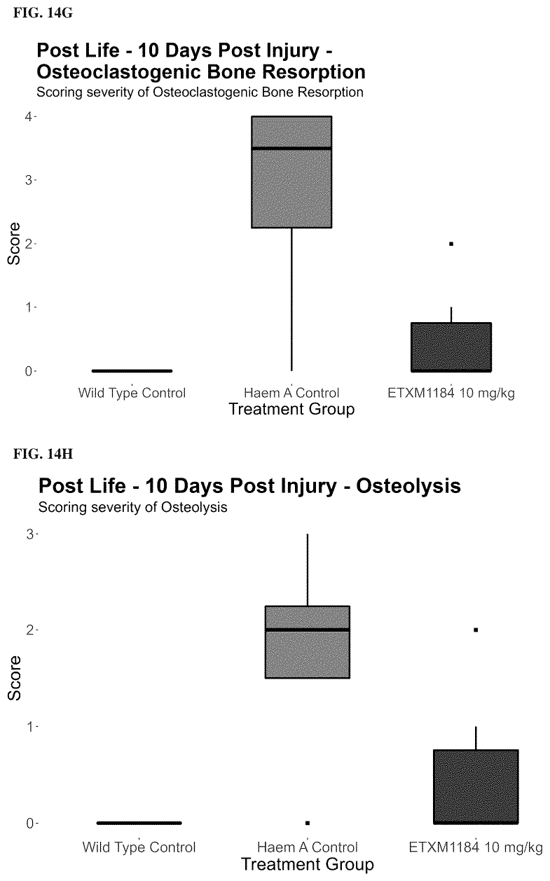

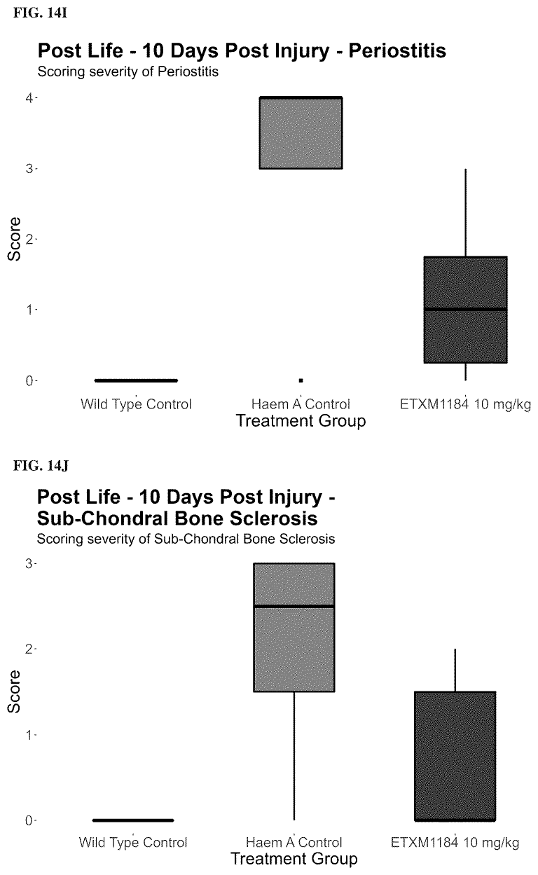

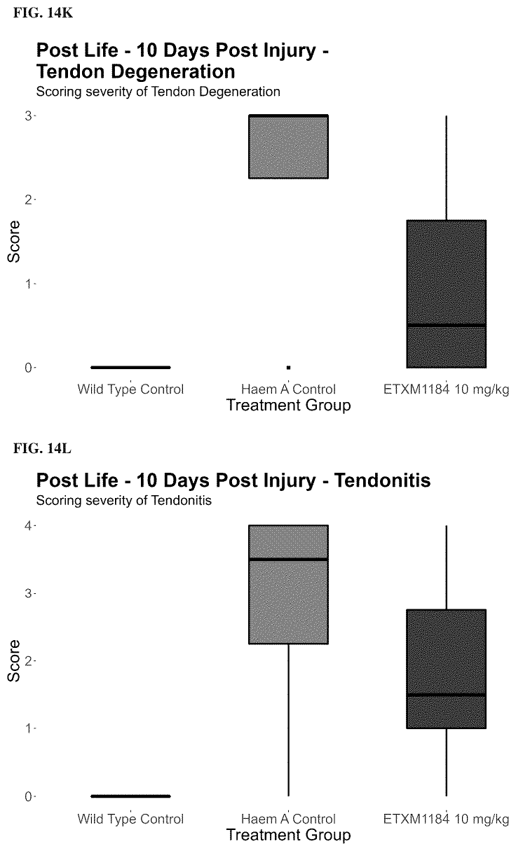

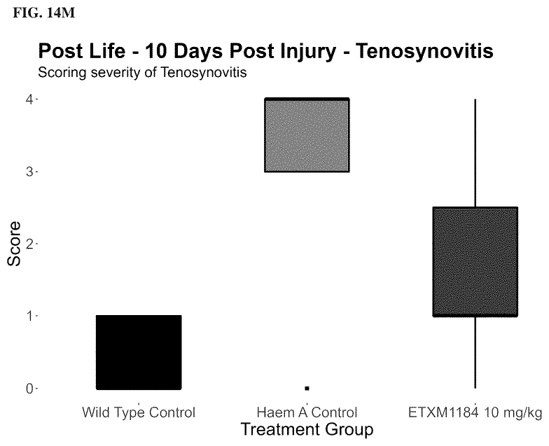

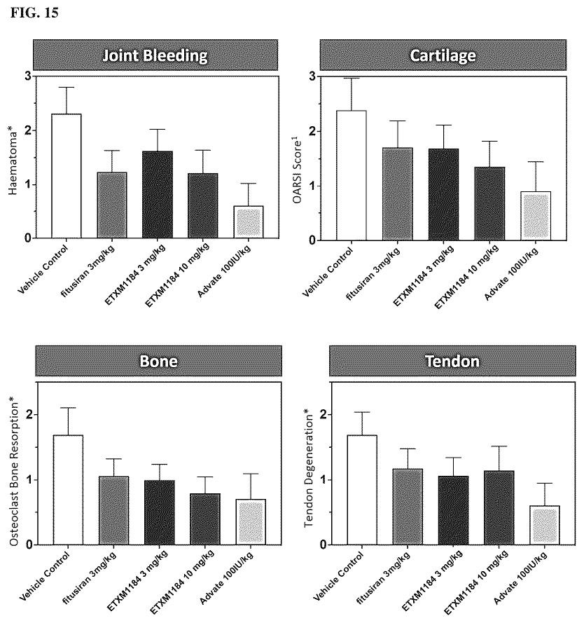

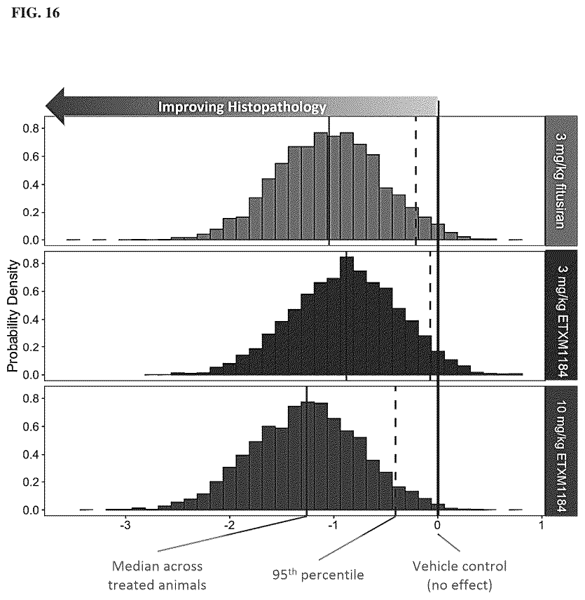

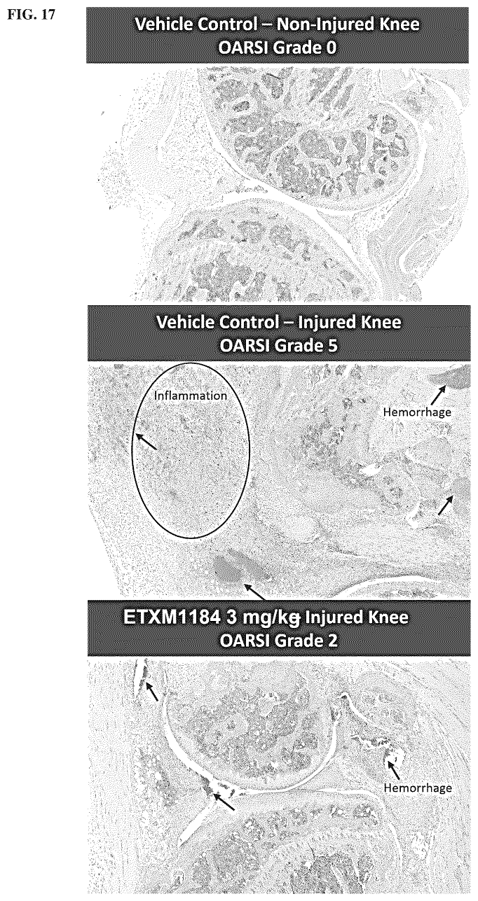

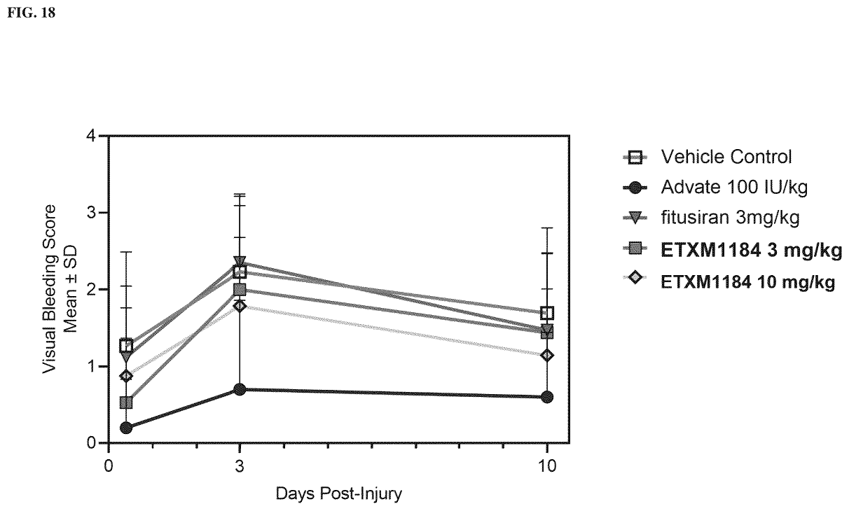

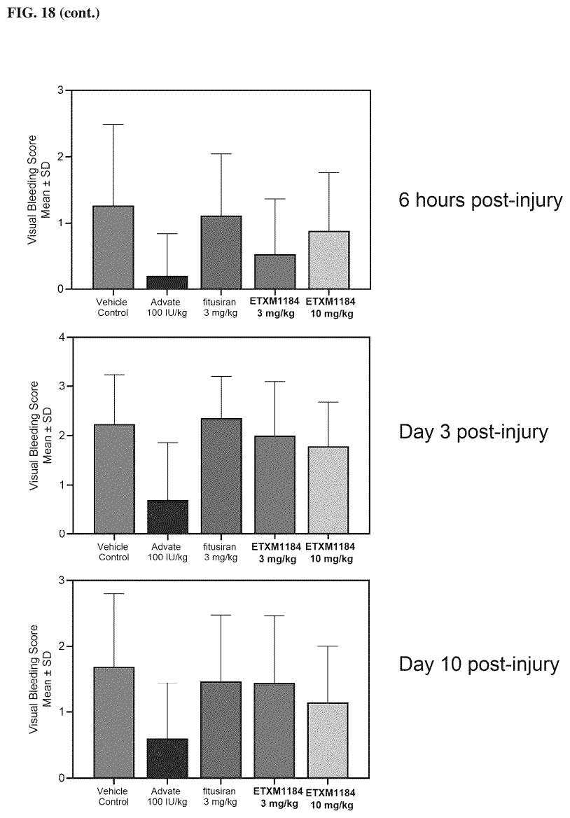

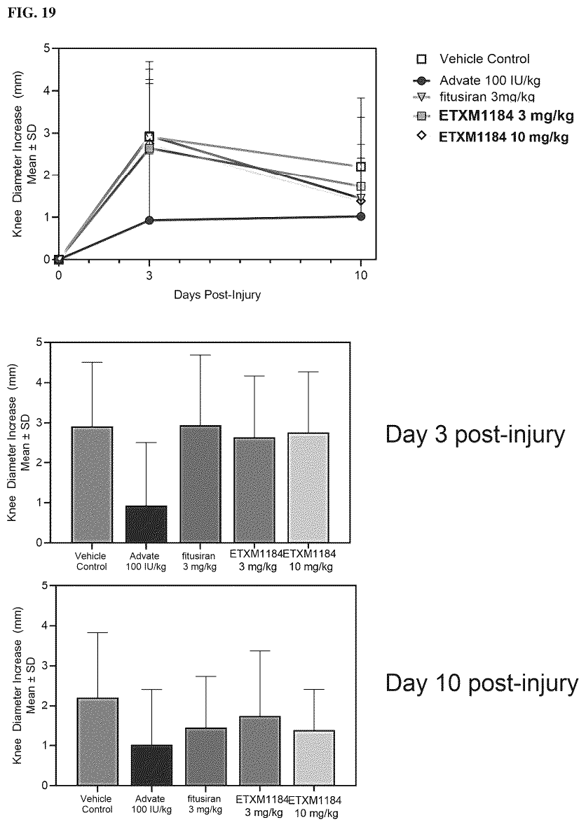

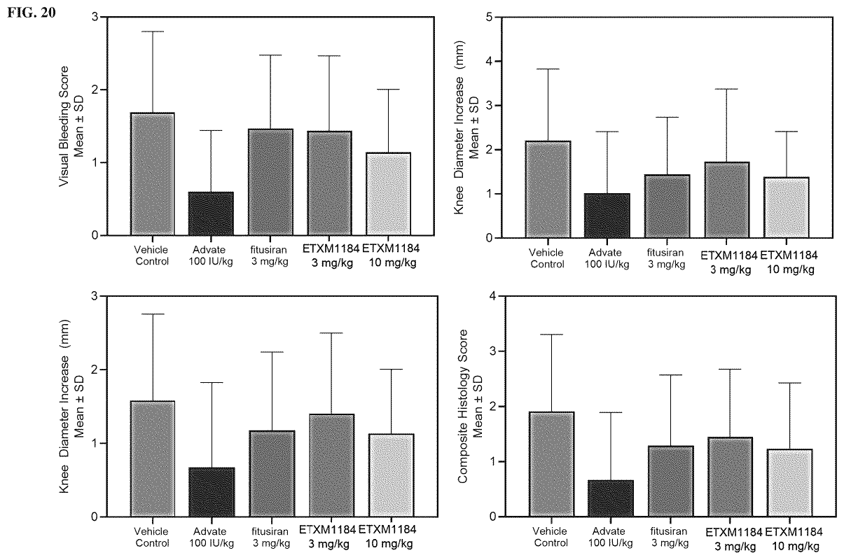

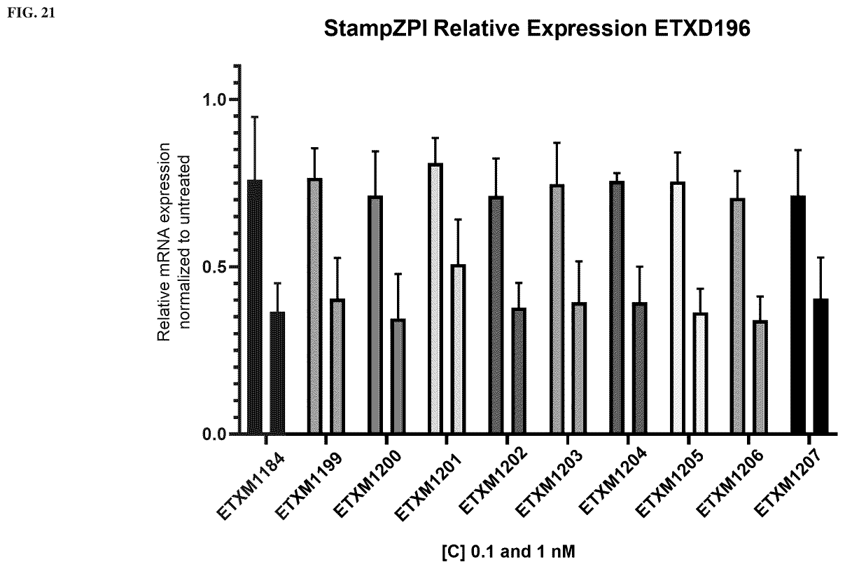

It should also be understood that while FIG. 1 depicts as a product molecules based on the linker and ligand portions as specifically depicted in FIG. 1 attached to an oligonucleoside moiety as also depicted herein, this product may alternatively further comprise, or consist essentially of, molecules wherein the linker and ligand portions are essentially as depicted in FIG. 1 attached to an oligonucleoside moiety but having the F substituent as shown in FIG. 1 on the cyclo-octyl ring replaced by a substituent occurring as a result of hydrolytic displacement, such as an OH substituent. In this way, (a) tether 1a constructs can consist essentially of molecules having linker and ligand portions specifically as depicted in FIG. 1, with a F substituent on the cyclo-octyl ring; or (b) tether 1a constructs can consist essentially of molecules having linker and ligand portions essentially as depicted in FIG. 1 but having the F substituent as shown in FIG. 1 on the cyclo-octyl ring replaced by a substituent occurring as a result of hydrolytic displacement, such as an OH substituent, or (c) tether 1a constructs can comprise a mixture of molecules as defined in (a) and/or (b).

FIG. 2: Linker and ligand portions of constructs suitable for use according to the present invention including tether 1b. While FIG. 2 depicts the linker to be conjugated to an oligonucleotide, it is to be understood that the present invention also encompasses conjugates of the same linker with an oligonucleoside as disclosed herein.

The comments made in relation to FIG. 1 and the possible replacement of the F substituent as shown in FIG. 1 on the cyclo-octyl ring replaced by a substituent occurring as a result of hydrolytic displacement, such as an OH substituent, apply equally to tether 1b constructs. In this way, (a) tether 1b constructs can consist essentially of molecules having linker and ligand portions specifically as depicted in FIG. 2, with a F substituent on the cyclo-octyl ring: or (b) tether 1b constructs can consist essentially of molecules having linker and ligand portions essentially as depicted in FIG. 2 but having the F substituent as shown in FIG. 2 on the cyclo-octyl ring replaced by a substituent occurring as a result of hydrolytic displacement, such as an OH substituent, or (c) tether 1b constructs can comprise a mixture of molecules as defined in (a) and/or (b).

FIG. 3: Linker and ligand portions of constructs suitable for use according to the present invention including tether 2a. While FIG. 3 depicts the linker to be conjugated to an oligonucleotide, it is to be understood that the present invention also encompasses conjugates of the same linker with an oligonucleoside as disclosed herein.

FIG. 4: Linker and ligand portions of constructs suitable for use according to the present invention including tether 2b. While FIG. 4 depicts the linker to be conjugated to an oligonucleotide, it is to be understood that the present invention also encompasses conjugates of the same linker with an oligonucleoside as disclosed herein.

FIG. 5: Formulae described in Sentences 1-101 disclosed herein.

FIG. 6: Formulae described in Clauses 1-56 disclosed herein.

FIG. 7A-7B: Inverted abasic constructs that can be used with nucleic acid sequences according to the present invention as described herein. For FIG. 7A, a galnac linker is attached to the 5′ end region of the sense strand in use (not depicted in FIG. 7A). For FIG. 7B, a galnac linker is attached to the 3′ end region of the sense strand in use (not depicted in FIG. 7B).

iaia as shown at the 3′ end region of the sense strand in FIG. 7A represents (i) two abasic nucleosides provided as the penultimate and terminal nucleosides at the 3′ end region of the sense strand, (ii) wherein a 3′-3′ reversed linkage is provided between the antepenultimate nucleoside (namely at position 21 of the sense strand, wherein position 1 is the terminal 5′ nucleoside of the sense strand) and the adjacent penultimate abasic residue of the sense strand, and (iii) the linkage between the terminal and penultimate abasic nucleosides is 5′-3′ when reading towards the 3′ end region comprising the terminal and penultimate abasic nucleosides.

iaia as shown at the 5′ end region of the sense strand in FIG. 7B represents (i) two abasic nucleosides provided as the penultimate and terminal nucleosides at the 5′ end region of the sense strand, (ii) wherein a 5′-5′ reversed linkage is provided between the antepenultimate nucleoside (namely at position 1 of the sense strand, not including the iaia motif at the 5′ end region of the sense strand in the nucleoside position numbering on the sense strand) and the adjacent penultimate abasic residue of the sense strand, and (iii) the linkage between the terminal and penultimate abasic nucleosides is 3′-5′ when reading towards the 5′ end region comprising the terminal and penultimate abasic nucleosides.

FIG. 8A-8B: Duplex constructs according to Table 5.

FIG. 9: Results of dose-response experiments for inhibition of ZPI mRNA expression in human Huh7 cells. Points represent mean relative expression of ZPI mRNA compared to untreated wells after treatment with siRNA construct at the indicated concentrations on the x-axis. Error bars represent standard deviation of the mean. Dotted curves represent 95% confidence intervals. Dotted lines and shaded areas represent the mean relative expression +/−standard deviation from untreated wells on the same plate.

FIG. 10: Change in liver ZPI mRNA expression over time following subcutaneous delivery of GalNAc conjugated siRNAs in C57BL/6 mice. ETXM1180, 1188, and 1192 were all murinised for this assay as described in Example 11. Data are mean+/−standard deviation, n=3 mice per timepoint.

FIG. 11: Change in liver ZPI mRNA expression over time following subcutaneous delivery of GalNAc conjugated siRNAs in C57BL/6 mice. ETXM1181, 1189 and 1193 were all murinised for this assay as described in Example 11. Data are mean+/−standard deviation, n=3 mice per timepoint.

FIG. 12A-12B: Visual bleeding score of mice in three different treatment groups (wild type control group. Haem A mice that received a vehicle (0.9% saline), and Haem A mice that received the GalNAc-siRNA construct ETXM1184) 3 days (FIG. 12A) and 10 days (FIG. 12B) post injury. Definition of the bleeding scores is provided below.

FIG. 13A-13B: FIG. 13A shows a comparison of knee diameters at day 3 and 10 post injury of mice in three different treatment groups (wild-type control group. Haem A mice receiving vehicle (0.9% saline), and Haem A mice receiving the GalNAc-siRNA construct ETXM1184). FIG. 13B shows a comparison of skinned knee diameter at day 10 post injury of mice in the same three treatment groups.

FIG. 14A-14M: Comparison of the severity of bone marrow hyperplasia (FIG. 14A), osteoarthritis (FIG. 14B), chondrocyte degeneration/necrosis (FIG. 14C), haemorrhage (FIG. 14D), haemosiderin deposition (FIG. 14E), haematoma (FIG. 14F), osteoclastogenic bone resorption (FIG. 14G), osteolysis (FIG. 14H), periostitis (FIG. 14I), sub-chondral bone sclerosis (FIG. 14J), tendon degeneration (FIG. 14K), tendonitis (FIG. 14L) and tenosynovitis (FIG. 14M) in mice in three different treatment groups (wild-type control group, Haem A mice receiving vehicle (0.9% saline), and Haem A mice receiving the GalNAc-siRNA construct ETXM1184).

FIG. 15: Joint Protection: Several Endpoints Document Dose-Responsive Effect. Prophylactic administration of ETXM1184 shows dose-dependent protection in key tissue readouts at 10 days post-injury. ETXM1184 shows efficacy in the same range as clinical comparators: FVIII replacement therapy as gold-standard for emergency treatments (Advate) and siRNA-based rebalancing agent for prophylaxis that demonstrated good bleed protection in late-stage clinical development (fitusiran). * Scale: ( )=Normal: 1=Minimal: 2=Moderate: 3=Marked: 4=Severe. [1] Glasson et al., Osteoarthritis Cartilage. 2010 October; 18 Suppl 3:S17-23. doi: 10.1016/j.joca.2010.05.025. PMID: 20864019.

FIG. 16: Composite haemarthrosis histopathology score quantifies: Tendonitis, Tendon degeneration, Tenosynovitis, Periostitis, Osteolysis, Osteoclastic bone resorption, Haemorrhage, Haematoma, Haemosiderin deposition, Chondrocyte necrosis, Cartilage OARSI Grade, Subchondral bone sclerosis and Bone marrow hyperplasia. ETX-148 shows significant dose-responsive effect (Bayesian linear model fitted to composite score). Median reduction of composite score compared to control: −1.25 for the ETXM1184 10 mg/kg group (significance level equivalent to p<0.01): −0.91 for the ETXM1184 3 mg/kg group (significance level equivalent to p<0.05). Comparator fitusiran shows median reduction of −1.04 for the 3 mg/kg group (significance level equivalent to p<0.05).

FIG. 17: Prophylactic administration of ETXM1184 improves haemarthrosis joint pathology in haemophilia A mice. Administration of 3 mg/kg ETXM1184 resulted in improved hemarthrosis knee joint pathology, reduced inflammation, and resulted in smaller areas of haemorrhage.

FIG. 18: Prophylactic administration of ETXM1184 reduces post-injury bleeding in hemophilia A mice (in-life visual bleeding score (VBS)). A bleeding event was introduced into the knee joint of Hemophilia A mice 8 days after siRNA administration. Bleeding was monitored for 10 days post-injury and terminal histological analysis was conducted. Prophylactic administration of a single 10 mg/kg dose of ETXM1184 effectively reduced visual bleeding score (VBS) comparably to Factor VIII replacement (Advate) by 10 days post-injury.

FIG. 19: Prophylactic administration of ETXM1184 reduces post-injury bleeding into the knee joint of hemophilia A mice (in-life measurement of injured knee diameter compared to non-injured knee diameter). A bleeding event was introduced into the knee joint of Hemophilia A mice 8 days after siRNA administration. Bleeding was monitored for 10 days post-injury and terminal histological analysis conducted. Prophylactic ETXM1184 administered as a single 10 mg/kg dose effectively reduced blood accumulation in knee joint comparably to Factor VIII replacement (Advate) by 10 days post-injury.

FIG. 20: Prophylactic administration of ETXM1184 reduces hemarthrosis in a Hemophilia A mouse model (terminal measurements taken 18 days post-siRNA dosing and 10) days post-injury). Prophylactic ETXM1184 administered as a single 10 mg/kg dose effectively reduced joint bleeding and characteristics of hemophilic arthropathy comparably to Factor VIII replacement (Advate) by 10 days post-injury.

FIG. 21: Inhibition of ZPI expression by ETXM1184 (ETXS1036 & ETXS1035), ETXM1199 (ETXS2398 & ETXS2397), ETXM1200 (ETXS2400 & ETXS2397), ETXM1201 (ETXS2402 & ETXS2397), ETXM1202 (ETXS2404 & ETCS2397), ETXM1203 (ETXS2406 & ETXS2397), ETXM 1204 (ETXS2408 & ETXS2397), ETXM 1205 (ETXS2410 & ETXS2397), ETXM1206 (ETXS2412 & ETXS2397) and ETXM1207 (ETXS2414 & ETXS2397).

### DEFINITIONS

The “first strand”, also called the antisense strand or guide strand herein and which can be used interchangeably herein, refers to the nucleic acid strand, e.g. the strand of an siRNA, e.g. a dsiRNA, which includes a region that is substantially complementary to a target sequence, e.g. to an mRNA. As used herein, the term “region of complementarity” refers to the region on the antisense strand that is substantially complementary to a sequence, for example a target sequence. Where the region of complementarity is not fully complementary to the target sequence, the mismatches can typically be in the internal or terminal regions of the molecule. In some embodiments, a double stranded nucleic acid e.g. an siRNA agent of the invention includes a nucleoside mismatch in the antisense strand.

The “second strand” (also called the sense strand or passenger strand herein, and which can be used interchangeably herein), refers to the strand of a nucleic acid e.g. siRNA that includes a region that is substantially complementary to a region of the antisense strand as that term is defined herein.

In the context of molecule comprising a nucleic acid provided with a ligand moiety, optionally also with a linker moiety, the nucleic acid of the invention may be referred to as an oligonucleoside or an oligonucleoside moiety.

Oligonucleotides are short nucleic acid polymers. Whilst oligonucleotides contain phosphodiester bonds between the nucleoside component thereof (base plus sugar), the present invention is not limited to oligonucleotides always joined by such a phosphodiester bond between adjacent nucleosides, and other oligomers of nucleosides joined by bonds which are bonds other than a phosphodiester bond are contemplated. For example, a bond between nucleosides may be a phosphorothioate bond. Therefore, the term “oligonucleoside” as used herein covers both oligonucleotides and other oligomers of nucleosides. An oligonucleoside which is a nucleic acid having at least a portion which is an oligonucleotide is preferred according to the present invention. An oligonucleoside having one or more, or a majority of, phosphodiester backbone bonds between nucleosides is also preferred according to the present invention. An oligonucleoside having one or more, or a majority of, phosphodiester backbone bonds between nucleosides, and also having one or more phosphorothioate backbone bonds between nucleosides (typically in a terminal region of the first and/or second strands) is also preferred according to the present invention.

It is preferred herein that the nucleic acid according to the invention is a double stranded oligonucleoside comprising one or more phosphorothioate backbone bonds between nucleosides. Accordingly, in all instances in which the present application refers to an oligonucleotide, particularly in the chemical structures disclosed herein, the oligonucleotide may equally be an oligonucleoside as defined herein.

In some embodiments, a double stranded nucleic acid e.g. siRNA agent of the invention includes a nucleoside mismatch in the sense strand. In some embodiments, the nucleoside mismatch is, for example, within 5, 4, 3, 2, or 1 nucleosides from the 3′-end of the nucleic acid e.g. siRNA.

In another embodiment, the nucleoside mismatch is, for example, in the 3′-terminal nucleoside of the nucleic acid e.g. siRNA.

A “target sequence” (which may also be called a target RNA or a target mRNA) refers to a contiguous portion of the nucleoside sequence of an mRNA molecule formed during the transcription of a gene, including mRNA that is a product of RNA processing of a primary transcription product.

The target sequence may be from about 10-35 nucleosides in length, e.g., about 15-30 nucleosides in length. For example, the target sequence can be from about 15-30 nucleosides, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleosides in length. Ranges and lengths intermediate to the above recited ranges and lengths are also contemplated to be part of the invention.

The term “ribonucleoside” or “nucleoside” can also refer to a modified nucleoside, as further detailed below.

A nucleic acid can be a DNA or an RNA, and can comprise modified nucleosides. RNA is a preferred nucleic acid.

The terms “iRNA”, “siRNA”, “RNAi agent,” and “iRNA agent,” “RNA interference agent” as used interchangeably herein, refer to an agent that contains RNA, and which mediates the targeted cleavage of an RNA transcript via an RNA-induced silencing complex (RISC) pathway. siRNA directs the sequence-specific degradation of mRNA through RNA interference (RNAi).

A double stranded RNA is referred to herein as a “double stranded siRNA (dsiRNA) agent”, “double stranded siRNA (dsiRNA) molecule”, “double stranded RNA (dsRNA) agent”, “double stranded RNA (dsRNA) molecule”, “dsiRNA agent”, “dsiRNA molecule”, or “dsiRNA”, which refers to a complex of ribonucleic acid molecules, having a duplex structure comprising two anti-parallel and substantially complementary nucleic acid strands, referred to as having “sense” and “antisense” orientations with respect to a target RNA.

The majority of nucleosides of each strand of the nucleic acid, e.g. a dsiRNA molecule, are preferably ribonucleosides, but in that case each or both strands can also include one or more non-ribonucleosides, e.g., a deoxyribonucleoside or a modified nucleoside. In addition, as used in this specification, an “siRNA” may include ribonucleosides with chemical modifications.

The term “modified nucleoside” refers to a nucleoside having, independently, a modified sugar moiety, a modified internucleoside linkage, or modified nucleobase, or any combination thereof. Thus, the term modified nucleoside encompasses substitutions, additions or removal of, e.g., a functional group or atom, to internucleoside linkages, sugar moieties, or nucleobases. Any such modifications, as used in an siRNA type molecule, are encompassed by “iRNA” or “RNAi agent” or “siRNA” or “siRNA agent” for the purposes of this specification and claims.

The two strands forming the duplex structure may be different portions of one larger molecule, or they may be separate molecules e.g. RNA molecules.

The term “nucleoside overhang” refers to at least one unpaired nucleoside that extends from the duplex structure of a nucleic acid according to the present invention. A nucleic acid according to the present invention can comprise an overhang of at least one nucleoside; alternatively the overhang can comprise at least two nucleosides, at least three nucleosides, at least four nucleosides, at least five nucleosides or more. A nucleoside overhang can comprise or consist of a nucleoside/nucleoside analog, including a deoxynucleoside. The overhang(s) can be on the sense strand, the antisense strand, or any combination thereof. Furthermore, the nucleoside(s) of an overhang can be present on the 5′-end, 3′-end, or both ends of either an antisense or sense strand.

In certain embodiments, the antisense strand has a 1-10 nucleoside, e.g., 0-3, 1-3, 2-4, 2-5, 4-10, 5-10, 1, 2, 3, 4, 5, 6, 7, 8, 9, or 10 nucleoside, overhang at the 3′-end or the 5′-end.

“Blunt” or “blunt end” means that there are no unpaired nucleosides at that end of the double stranded nucleic acid, i.e., no nucleoside overhang. The nucleic acids of the invention include those with no nucleoside overhang at one end or with no nucleoside overhangs at either end.

Unless otherwise indicated, the term “complementary,” when used to describe a first nucleoside sequence in relation to a second nucleoside sequence, refers to the ability of an oligonucleoside comprising the first nucleoside sequence to hybridize and form a duplex structure under certain conditions with an oligonucleoside comprising the second nucleoside sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing (see, e.g., “Molecular Cloning: A Laboratory Manual, Sambrook, et al. (1989) Cold Spring Harbor Laboratory Press).

Complementary sequences within nucleic acid e.g. a dsiRNA, as described herein, include base-pairing of the oligonucleoside comprising a first nucleoside sequence to an oligonucleoside comprising a second nucleoside sequence over the entire length of one or both nucleoside sequences. Such sequences can be referred to as “fully complementary” with respect to each other herein. However, where a first sequence is referred to as “substantially complementary” or “partially complementary.” with respect to a second sequence herein, the two sequences can be fully complementary, or they can form one or more mismatched base pairs, such as 2, 4, or 5 mismatched base pairs, but preferably not more than 5, while retaining the ability to hybridize under the conditions most relevant to their ultimate application, e.g., inhibition of gene expression via a RISC pathway. Overhangs shall not be regarded as mismatches with regard to the determination of complementarity. For example, a nucleic acid e.g. dsiRNA comprising one oligonucleoside 17 nucleosides in length and another oligonucleoside 19 nucleosides in length, wherein the longer oligonucleoside comprises a sequence of 17 nucleosides that is fully complementary to the shorter oligonucleoside, can yet be referred to as “fully complementary”.

“Complementary” sequences, as used herein, can also include, or be formed entirely from, non-Watson-Crick base pairs or base pairs formed from non-natural and modified nucleosides, in so far as the above requirements with respect to their ability to hybridize are fulfilled. Such non-Watson-Crick base pairs include, but are not limited to, G: U Wobble or Hoogstein base pairing.

The terms “complementary,” “fully complementary” and “substantially/partially complementary” herein can be used with respect to the base matching between the sense strand and the antisense strand of a nucleic acid eg dsiRNA, or between the antisense strand of a double stranded nucleic acid e.g. siRNA agent and a target sequence.

Within the present invention, the second strand of the nucleic acid according to the invention, in particular a dsiRNA for inhibiting ZPI, is at least partially complementary to the first strand of said nucleic acid. In certain embodiments, a first and second strand of a nucleic acid according to the invention are partially complementary if they form a duplex region having a length of at least 17 base pairs and comprising not more than 1, 2, 3, 4, or 5 mismatched base pairs.

In certain embodiments, a first and second strand of the nucleic acid according to the invention are partially complementary if they form a duplex region having a length of 19 base pairs and comprising not more than 1, 2, 3, 4, or 5 mismatched base pairs. In certain embodiments, a first and second strand of the nucleic acid according to the invention are partially complementary if they form a duplex region having a length of 21 base pairs comprising not more than 1, 2, 3, 4, or 5 mismatched base pairs.

Alternatively, a first and second strand of the nucleic acid according to the invention are partially complementary if they form a duplex region having a length of at least 17 base pairs, wherein at least 14, 15, 16 or 17 of said base pairs are complementary base pairs, in particular Watson-Crick base pairs.

In certain embodiments, a first and second strand of the nucleic acid according to the invention are partially complementary if they form a duplex region having a length of 19 base pairs, wherein at least 14, 15, 16, 17, 18 or all 19 base pairs are complementary base pairs, in particular Watson-Crick base pairs. In certain embodiments, a first and second strand of the nucleic acid according to the invention are partially complementary if they form a duplex region having a length of 21 base pairs, wherein at least 16, 17, 18, 19, 20 or all 21 base pairs are complementary base pairs, in particular Watson-Crick base pairs.

As used herein, a nucleic acid that is “substantially complementary” or “partially complementary” to at least part of a messenger RNA (mRNA) refers to a nucleic acid that is substantially or partially complementary to a contiguous portion of the mRNA of interest (e.g., an mRNA encoding a gene). In certain embodiments, the contiguous portion of the mRNA is a sequence as listed in Table 1, i.e., any one of SEQ ID NOs: 2-121. For example, a nucleic acid is complementary to at least a part of an mRNA of a gene of interest if the sequence is substantially or partially complementary to a non-interrupted portion of an mRNA encoding that gene.

Accordingly, in some preferred embodiments, the antisense oligonucleosides as disclosed herein are fully complementary to the target gene sequence.

In other embodiments, the antisense oligonucleosides disclosed herein are substantially or partially complementary to a target RNA sequence and comprise a contiguous nucleoside sequence which is at least about 80% complementary over its entire length to the equivalent region of the target RNA sequence, such as at least about 85%, 86%, 87%, 88%, 89%, about 90%, 91%, 92%, 93%, 94%, 95%, 96%, 97%, 98%, or 99% complementary or 100% complementary.

In certain embodiments, the first (antisense) strand of a nucleic acid according to the invention is partially or fully complementary to a contiguous portion of RNA transcribed from the ZPI gene. In certain embodiments, the first strand of the nucleic acid according to the invention is partially or fully complementary to a contiguous portion of at least 17 nucleosides of the ZPI mRNA. In certain embodiments, the first strand of the nucleic acid according to the invention is partially or fully complementary to a contiguous portion of 17, 18, 19, 20, 21, 22 or 23 nucleosides of the ZPI mRNA. In certain embodiments, the first strand of the nucleic acid according to the invention is partially or fully complementary to a contiguous portion of 17, 18, 19, 20, 21, 22 or 23 nucleosides of any one of the sequences as listed in Table 1, i.e., any one of SEQ ID NOs: 2-121.

In certain embodiments, the first (antisense) strand of the nucleic acid according to the invention is partially complementary to a contiguous portion of the ZPI mRNA if it comprises a contiguous nucleoside sequence of at least 17 nucleosides, wherein at least 14, 15, 16 or 17 nucleosides of said contiguous nucleoside sequence are complementary to a contiguous portion of the ZPI mRNA. In certain embodiments, the first strand of the nucleic acid according to the invention comprises a contiguous nucleoside sequence of at least 17 nucleosides, wherein at least 14, 15, 16 or 17 nucleosides of said contiguous nucleoside sequence are complementary to a contiguous portion of any one of the sequences listed in Table 1, i.e., any one of SEQ ID NOs: 2-121. In certain embodiments, the first strand of the nucleic acid according to the invention comprises a contiguous nucleoside sequence of 19 nucleosides, wherein at least 14, 15, 16, 17, 18 or all 19 nucleosides of said contiguous nucleoside sequence are complementary to a contiguous portion of any one of the sequences listed in Table 1, i.e., any one of SEQ ID NOs: 2-121. In certain embodiments, the first strand of the nucleic acid according to the invention comprises a contiguous nucleoside sequence of 23 nucleosides, wherein at least 18, 19, 20, 21, 22 or all 23 nucleosides of said contiguous nucleoside sequence are complementary to a contiguous portion of any one of the sequences listed in Table 1, i.e., any one of SEQ ID NOs: 2-101.

In some embodiments, a nucleic acid e.g. an siRNA of the invention includes a sense strand that is substantially or partially complementary to an antisense oligonucleoside which, in turn, is complementary to a target gene sequence and comprises a contiguous nucleoside sequence. The nucleoside sequence of the sense strand is typically at least about 80% complementary over its entire length to the equivalent region of the nucleoside sequence of the antisense strand, such as about 85%, 86%, 87%, 88%, 89%, 90%, 91%, 92%, 93%, 94%, 95%, 96%, 97%, 98%, or 99% complementary, or 100% complementary.

In some embodiments, a nucleic acid e.g. an siRNA of the invention includes an antisense strand that is substantially or partially complementary to the target sequence and comprises a contiguous nucleoside sequence which is at least 80% complementary over its entire length to the target sequence such as about 85%, 86%, 87%, 88%, 89%, 90%, 91%, 92%, 93%, 94%, 95%, 96%, 97%, 98%, or 99% complementary, or 100% complementary.

As used herein, a “subject” is an animal, such as a mammal, including a primate (such as a human, a non-human primate, e.g., a monkey, and a chimpanzee), or a non-primate or a bird that expresses the target gene, either endogenously or heterologously, when the target gene sequence has sufficient complementarity to the nucleic acid e.g. siRNA agent to promote target knockdown. In certain preferred embodiments, the subject is a human.

The terms “treating” or “treatment” refer to a beneficial or desired result including, but not limited to, alleviation or amelioration of one or more symptoms associated with gene expression. “Treatment” can also mean prolonging survival as compared to expected survival in the absence of treatment. Treatment can include prevention of development of co-morbidities, e.g., reduced liver damage in a subject with a hepatic infection.

“Therapeutically effective amount,” as used herein, is intended to include the amount of a nucleic acid e.g. an siRNA that, when administered to a patient for treating a subject having disease, is sufficient to effect treatment of the disease (e.g., by diminishing, ameliorating or maintaining the existing disease or one or more symptoms of disease or its related comorbidities).

The phrase “pharmaceutically acceptable” is employed herein to refer to compounds, materials, compositions, or dosage forms which are suitable for use in contact with the tissues of human subjects and animal subjects without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition, or vehicle, such as a liquid or solid filler, diluent, excipient, manufacturing aid or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated.

Where a value or range of values of a parameter are recited, it is intended that values and ranges intermediate to the recited values are also intended to be part of this invention.

The articles “a” and “an” are used herein to refer to one or to more than one (i.e. to at least one) of the grammatical object of the article.

The term “including” is used herein to mean, and is used interchangeably with, the phrase “including but not limited to”.

The term “or” is used herein to mean, and is used interchangeably with, the term “and/or,” unless context clearly indicates otherwise. For example, “sense strand or antisense strand” is understood as “sense strand or antisense strand or sense strand and antisense strand.”

The term “about” is used herein to mean within the typical ranges of tolerances in the art. For example, “about” can be understood as about 2 standard deviations from the mean. In certain embodiments, about means +10%. In certain embodiments, about means +5%. When about is present before a series of numbers or a range, it is understood that “about” can modify each of the numbers in the series or range.

The term “at least” prior to a number or series of numbers is understood to include the number adjacent to the term “at least”, and all subsequent numbers or integers that could logically be included, as clear from context. For example, the number of nucleosides in a nucleic acid molecule must be an integer. For example, “at least 18 nucleosides of a 21 nucleoside nucleic acid molecule” means that 18, 19, 20, or 21 nucleosides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. For example, a duplex with an overhang of “no more than 2 nucleosides” has a 2, 1, or 0 nucleoside overhang. When “no more than” is present before a series of numbers or a range, it is understood that “no more than” can modify each of the numbers in the series or range.

The terminal region of a strand is the last 5 nucleosides from the 5′ or the 3′ end.

Various embodiments of the invention can be combined as determined appropriate by one of skill in the art.

### Abasic Nucleosides

In certain embodiments, there are 1, e.g. 2, e.g. 3, e.g. 4 or more abasic nucleosides present in nucleic acids according to the present invention. Abasic nucleosides are modified nucleosides because they lack the base normally seen at position 1 of the sugar moiety. Typically, there will be a hydrogen at position 1 of the sugar moiety of the abasic nucleosides present in a nucleic acid according to the present invention.

The abasic nucleosides are in the terminal region of the second strand, preferably located within the terminal 5 nucleosides of the end of the strand. The terminal region may be the terminal 5 nucleosides, which includes abasic nucleosides.

The second strand may comprise, as preferred features (which are all specifically contemplated in combination unless mutually exclusive):
2, or more than 2, abasic nucleosides in a terminal region of the second strand; and/or
2, or more than 2, abasic nucleosides in either the 5′ or 3′ terminal region of the second strand; and/or
2, or more than 2, abasic nucleosides in either the 5′ or 3′ terminal region of the second strand, wherein the abasic nucleosides are present in an overhang as herein described; and/or
2, or more than 2, consecutive abasic nucleosides in a terminal region of the second strand, wherein preferably one such abasic nucleoside is a terminal nucleoside; and/or
2, or more than 2, consecutive abasic nucleosides in either the 5′ or 3′ terminal region of the second strand, wherein preferably one such abasic nucleoside is a terminal nucleoside in either the 5′ or 3′ terminal region of the second strand; and/or
a reversed internucleoside linkage connects at least one abasic nucleoside to an adjacent basic nucleoside in a terminal region of the second strand; and/or
a reversed internucleoside linkage connects at least one abasic nucleoside to an adjacent basic nucleoside in either the 5′ or 3′ terminal region of the second strand; and/or
an abasic nucleoside as the penultimate nucleoside which is connected via the reversed linkage to the nucleoside which is not the terminal nucleoside (called the antepenultimate nucleoside herein); and/or
abasic nucleosides as the 2 terminal nucleosides connected via a 5′-3′ linkage when reading the strand in the direction towards the terminus comprising the terminal nucleosides;
abasic nucleosides as the 2 terminal nucleosides connected via a 3′-5′ linkage when reading the strand in the direction towards the terminus comprising the terminal nucleosides;
abasic nucleosides as the terminal 2 positions, wherein the penultimate nucleoside is connected via the reversed linkage to the antepenultimate nucleoside, and wherein the reversed linkage is a 5-5′ reversed linkage or a 3′-3′ reversed linkage;
abasic nucleosides as the terminal 2 positions, wherein the penultimate nucleoside is connected via the reversed linkage to the antepenultimate nucleoside, and wherein either
(1) the reversed linkage is a 5-5′ reversed linkage and the linkage between the terminal and penultimate abasic nucleosides is 3′5′ when reading towards the terminus comprising the terminal and penultimate abasic nucleosides: or
(2) the reversed linkage is a 3-3′ reversed linkage and the linkage between the terminal and penultimate abasic nucleosides is 5′3′ when reading towards the terminus comprising the terminal and penultimate abasic nucleosides.

Preferably there is an abasic nucleoside at the terminus of the second strand.

Preferably there are 2 or at least 2 abasic nucleosides in the terminal region of the second strand, preferably at the terminal and penultimate positions.

Preferably 2 or more abasic nucleosides are consecutive, for example all abasic nucleosides may be consecutive. For example, the terminal 1 or terminal 2 or terminal 3 or terminal 4 nucleosides may be abasic nucleosides.

An abasic nucleoside may also be linked to an adjacent nucleoside through a 5′-3′ phosphodiester linkage or reversed linkage unless there is only 1 abasic nucleoside at the terminus, in which case it will have a reversed linkage to the adjacent nucleoside.

A reversed linkage (which may also be referred to as an inverted linkage, which is also seen in the art), comprises either a 5′-5′, a 3′3′, a 3′-2′ or a 2′-3′ phosphodiester linkage between the adjacent sugar moieties of the nucleosides.

Abasic nucleosides which are not terminal will have 2 phosphodiester bonds, one with each adjacent nucleoside, and these may be a reversed linkage or may be a 5′-3 phosphodiester bond or may be one of each.

A preferred embodiment comprises 2 abasic nucleosides at the terminal and penultimate positions of the second strand, and wherein the reversed internucleoside linkage is located between the penultimate (abasic) nucleoside and the antepenultimate nucleoside.

Preferably there are 2 abasic nucleosides at the terminal and penultimate positions of the second strand and the penultimate nucleoside is linked to the antepenultimate nucleoside through a reversed internucleoside linkage and is linked to the terminal nucleoside through a 5′-3′ or 3′-5′ phosphodiester linkage (reading in the direction of the terminus of the molecule).

Preferably a nucleic acid according to the present invention comprises one or more abasic nucleosides, optionally wherein the one or more abasic nucleosides are in a terminal region of the second strand, and/or wherein at least one abasic nucleoside is linked to an adjacent basic nucleoside through a reversed internucleoside linkage.

Typically the second strand comprises 2 consecutive abasic nucleosides in the 5′ terminal region of the second strand, wherein one such abasic nucleoside is a terminal nucleoside at the 5′ terminal region of the second strand and the other abasic nucleoside is a penultimate nucleoside at the 5′ terminal region of the second strand, wherein: (a) said penultimate abasic nucleoside is connected to an adjacent first basic nucleoside in an adjacent 5′ near terminal region through a reversed internucleoside linkage; and (b) the reversed linkage is a 5-5′ reversed linkage; and (c) the linkage between the terminal and penultimate abasic nucleosides is 3′5′ when reading towards the terminus comprising the terminal and penultimate abasic nucleosides. More typically, (i) the first strand and the second strand each has a length of 23 nucleosides: (ii) two phosphorothioate internucleoside linkages are respectively between three consecutive positions in said 5′ near terminal region of the second strand, wherein a first phosphorothioate internucleoside linkage is present between said adjacent first basic nucleoside of (a) and an adjacent second basic nucleoside in said 5′ near terminal region of the second strand, and a second phosphorothioate internucleoside linkage is present between said adjacent second basic nucleoside and an adjacent third basic nucleoside in said 5′ near terminal region of the second strand: (iii) two phosphorothioate internucleoside linkages are respectively between three consecutive positions in both 5′ and 3′ terminal regions of the first strand. whereby a terminal nucleoside respectively at each of the 5′ and 3′ terminal regions of said first strand is each attached to a respective 5′ and 3′ adjacent penultimate nucleoside by a phosphorothioate internucleoside linkage, and each first 5′ and 3′ penultimate nucleoside is attached to a respective 5′ and 3′ adjacent antepenultimate nucleoside by a phosphorothioate internucleoside linkage; and (iv) the second strand of the nucleic acid is conjugated directly or indirectly to one or more ligand moieties at the 3′ terminal region of the second strand.

Alternatively the second strand comprises 2 consecutive abasic nucleosides preferably in an overhang in the 3′ terminal region of the second strand, wherein one such abasic nucleoside is a terminal nucleoside at the 3′ terminal region of the second strand and the other abasic nucleoside is a penultimate nucleoside at the 3′ terminal region of the second strand, wherein: (a) said penultimate abasic nucleoside is connected to an adjacent first basic nucleoside in an adjacent 3′ near terminal region through a reversed internucleoside linkage; and (b) the reversed linkage is a 3-3′ reversed linkage; and (c) the linkage between the terminal and penultimate abasic nucleosides is 5′-3′ when reading towards the terminus comprising the terminal and penultimate abasic nucleosides. More typically. (i) the first strand and the second strand each has a length of 23 nucleosides: (ii) two phosphorothioate internucleoside linkages are respectively between three consecutive positions in said 3′ near terminal region of the second strand, wherein a first phosphorothioate internucleoside linkage is present between said adjacent first basic nucleoside of (a) and an adjacent second basic nucleoside in said 3′ near terminal region of the second strand, and a second phosphorothioate internucleoside linkage is present between said adjacent second basic nucleoside and an adjacent third basic nucleoside in said 3′ near terminal region of the second strand: (iii) two phosphorothioate internucleoside linkages are respectively between three consecutive positions in both 5′ and 3′ terminal regions of the first strand, whereby a terminal nucleoside respectively at each of the 5′ and 3′ terminal regions of said first strand is each attached to a respective 5′ and 3′ adjacent penultimate nucleoside by a phosphorothioate internucleoside linkage, and each first 5′ and 3′ penultimate nucleoside is attached to a respective 5′ and 3′ adjacent antepenultimate nucleoside by a phosphorothioate internucleoside linkage; and (iv) the second strand of the nucleic acid is conjugated directly or indirectly to one or more ligand moieties at the 5′ terminal region of the second strand.

Examples of the structures are as follows (where the specific RNA nucleosides shown are not limiting and could be any RNA nucleoside):
A A 3′-3′ reversed bond (and also showing the 5′-3 direction of the last phosphodiester bond between the two abasic molecules reading towards the terminus of the molecule)

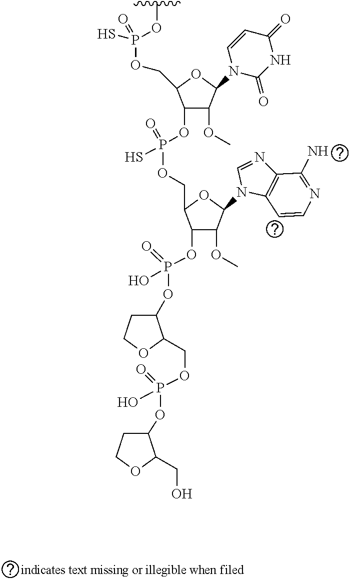 [SMILES: *OP(=O)(S)OCC1O[C@@H](n2ccc(=O)[nH]c2=O)C(OC)C1OP(=O)(S)OCC1O[C@@H](n2cnc3c(N)nccc32)C(OC)C1OP(=O)(O)OC1CCOC1COP(=O)(O)OC1CCOC1CO]

B Illustrating a 5′-5′ reversed bond (and also showing the 3′-5′ direction of the last phosphodiester bond between the two abasic molecules reading towards the terminus of the molecule)

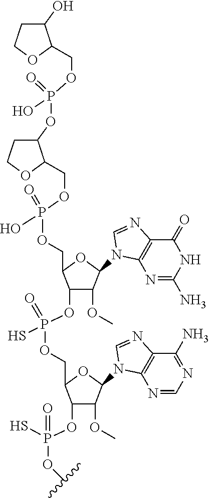 [SMILES: *OP(=O)(S)OC1C(COP(=O)(S)OC2C(COP(=O)(O)OCC3OCCC3OP(=O)(O)OCC3OCCC3O)O[C@@H](n3cnc4c(=O)[nH]c(N)nc43)C2OC)O[C@@H](n2cnc3c(N)ncnc32)C1OC]

[symbol]

The abasic nucleoside or abasic nucleosides present in the nucleic acid are provided in the presence of a reversed internucleoside linkage or linkages, namely a 5′-5′ or a 3′-3′ reversed internucleoside linkage. A reversed linkage occurs as a result of a change of orientation of an adjacent nucleoside sugar, such that the sugar will have a 3′-5′ orientation as opposed to the conventional 5′-3′ orientation (with reference to the numbering of ring atoms on the nucleoside sugars). The abasic nucleoside or nucleosides as present in the nucleic acids of the invention preferably include such inverted nucleoside sugars.

In the case of a terminal nucleoside having an inverted orientation, then this will result in an “inverted” end configuration for the overall nucleic acid. Whilst certain structures drawn and referenced herein are represented using conventional 5′-3′ direction (with reference to the numbering of ring atoms on the nucleoside sugars), it will be appreciated that the presence of a terminal nucleoside having a change of orientation and a proximal 3′-3′ reversed linkage, will result in a nucleic acid having an overall 5′-5′ end structure (i.e. the conventional 3′ end nucleoside becomes a 5′ end nucleoside). Alternatively, it will be appreciated that the presence of a terminal nucleoside having a change of orientation and a proximal 5′-5′ reversed linkage will result in a nucleic acid with an overall 3′-3′ end structure.

The proximal 3′-3′ or 5′-5′ reversed linkage as herein described, may comprise the reversed linkage being directly adjacent/attached to a terminal nucleoside having an inverted orientation, such as a single terminal nucleoside having an inverted orientation. Alternatively, the proximal 3′-3′ or 5′-5′ reversed linkage as herein described, may comprise the reversed linkage being adjacent 2, or more than 2, nucleosides having an inverted orientation, such as 2, or more than 2, terminal region nucleosides having an inverted orientation, such as the terminal and penultimate nucleosides. In this way, the reversed linkage may be attached to a penultimate nucleoside having an inverted orientation. While a skilled addressee will appreciate that inverted orientations as described above can result in nucleic acid molecules having overall 3′-3′ or 5′-5′ end structures as described herein, it will also be appreciated that with the presence of one or more additional reversed linkages and/or nucleosides having an inverted orientation, then the overall nucleic acid may have 3′-5′ end structures corresponding to the conventionally positioned 5′/3′ ends.

In one aspect the nucleic acid may have a 3′-3′ reversed linkage, and the terminal sugar moiety may comprise a 5′ OH rather than a 5′ phosphate group at the 5′ position of that terminal sugar.

A skilled person would therefore clearly understand that 5′-5′, 3′-3′ and 3′-5′ (reading in the direction of that terminus) end variants of the more conventional 5′-3′ structures (with reference to the numbering of ring atoms on the end nucleoside sugars) drawn herein are included in the scope of the disclosure, where a reversed linkage or linkages is/are present.

In the situation of, e.g., a reversed internucleoside linkage and/or one or more nucleosides having an inverted orientation creating an inverted end, and where the relative position of a linkage (e.g., to a linker) or the location of an internal feature (such as a modified nucleoside) is defined relative to the 5′ or 3′ end of the nucleic acid, then the 5′ or 3′ end is the conventional 5′ or 3′ end which would have existed had a reversed linkage not been in place, and wherein the conventional 5′ or 3′ end is determined by consideration of the directionality of the majority of the internal nucleoside linkages and/or nucleoside orientation within the nucleic acid. It is possible to tell from these internal bonds and/or nucleoside orientation which ends of the nucleic acid would constitute the conventional 5′ and 3′ ends (with reference to the numbering of ring atoms on the end nucleoside sugars) of the molecule absent the reversed linkage.

For example, in the structure shown below there are abasic residues in the first 2 positions located at the 5′ end. Where the terminal nucleoside has an inverted orientation then the 5′ end indicated in the diagram below, which is the conventional 5′ end, can in fact comprise a 3′ OH in view of the inverted nucleoside at the terminal position. Nevertheless the majority of the molecule will comprise conventional internucleoside linkages that run from the 3′ OH of the sugar to the 5′ phosphate of the next sugar, when reading in the standard 5′ [PO4] to 3′ [OH] direction of a nucleic acid molecule (with reference to the numbering of ring atoms on the nucleoside sugars), which can be used to determine the conventional 5′ and 3′ ends that would be found absent the inverted end configuration.

### 5′ A-A-Me-Me-Me-Me-Me-Me-F-Me-F—F—F-Me-Me-Me-Me-Me-Me-Me-Me-Me-Me 3′

In some embodiments, the second (sense) strand of the nucleic acid according to the invention comprises 2 consecutive abasic nucleosides in the 5′ terminal region as shown in the following 5′ terminal motif:

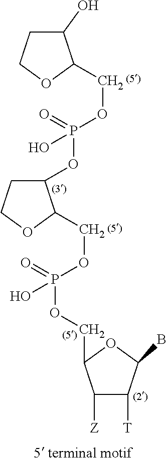

wherein:
B represents a nucleoside base,
T represent H, OH or a 2′ ribose modification,
Z represents the remaining nucleosides of said second strand.

In some embodiments, the second (sense) strand of the nucleic acid according to the invention comprises 2 consecutive abasic nucleosides in the 5′ terminal region as shown in the following 5′ terminal motif:

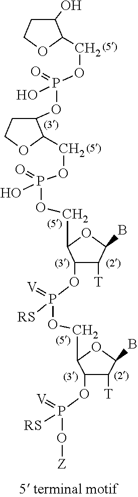

wherein:
B represents a nucleoside base,
T represents H, OH or a 2′ ribose modification (preferably a 2′ ribose modification, more
preferably a 2′Me or 2′F ribose modification),
V represents O or S (preferably O),
R represents H or C1-4 alkyl (preferably H),
Z represents the remaining nucleosides of said second strand,
more preferably the following 5′ terminal motif:

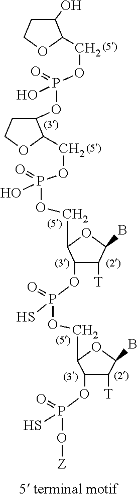

[symbol] [0165] wherein:
B represents a nucleoside base,
T represents a 2′ ribose modification (preferably a 2′Me or 2′F ribose modification),
Z represents the remaining nucleosides of said second strand.

The reversed bond is preferably located at the end of the nucleic acid eg RNA which is distal to a ligand moiety, such as a GalNAc containing portion, of the molecule.

GalNAc-siRNA constructs with a 5′-GalNAc on the sense strand can have a reversed linkage on the opposite end of the sense strand.

GalNAc-siRNA constructs with a 3′-GalNAc on the sense strand can have a reversed linkage on the opposite end of the sense strand.

In a preferred embodiment, the second (sense) strand of the nucleic acid according to the invention comprises 2 consecutive abasic nucleosides in the 5′ terminal region as shown in the following 5′ terminal motif:

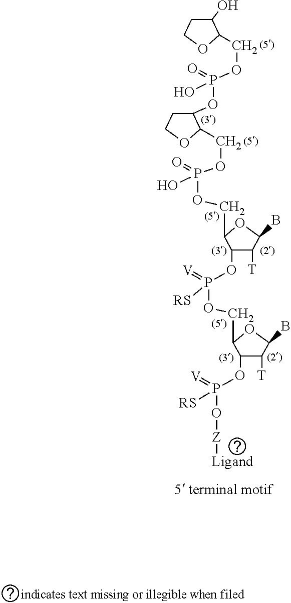

[symbol] [0174] wherein:
B represents a nucleoside base,
T represent H, OH or a 2′ ribose modification (preferably a 2′ ribose modification, more preferably a 2′Me or 2′F ribose modification),
V represent O or S (preferably O),
R represent H or C1-4 alkyl (preferably H),
Z comprises 11 to 26 contiguous nucleosides, preferably 15 to 21 contiguous nucleosides, and more preferably 19 contiguous nucleosides,
more preferably the following 5′ terminal motif:

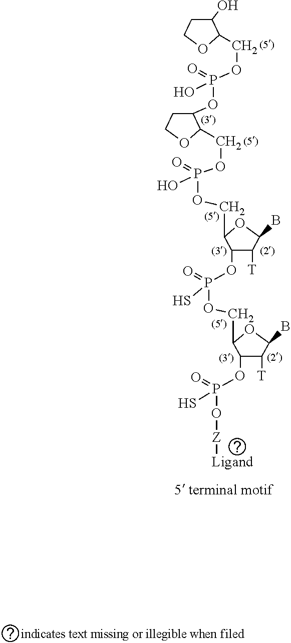

[symbol] [0182] wherein:
B represents a nucleoside base,
T represents a 2′ ribose modification (preferably a 2′Me or 2′F ribose modification).
Z comprises 19 contiguous nucleosides.

### Nucleic Acid Lengths

In one aspect the i) the first strand of the nucleic acid has a length in the range of 17 to 30 nucleosides, preferably 19 to 25 nucleosides, more preferably 19 or 23 nucleosides; and/or ii) the second strand of the nucleic acid has a length in the range of 17 to 30 nucleosides, preferably 19 to 25 nucleosides, more preferably 19 or 21 nucleosides.

Typically the duplex region of the nucleic acid is between 17 and 30 nucleosides in length, more preferably is 19 or 21 nucleosides in length. Similarly, the region of complementarity between the first strand and the portion of RNA transcribed from the ZPI gene is between 17 and 30 nucleosides in length.

### Nucleic Acid Modifications

In certain embodiments, the nucleic acid e.g. an RNA of the invention e.g., a dsiRNA, does not comprise further modifications, e.g., chemical modifications or conjugations known in the art and described herein.

In other preferred embodiments, the nucleic acid e.g. RNA of the invention, e.g., a dsiRNA, is further chemically modified to enhance stability or other beneficial characteristics.

In certain embodiments of the invention, substantially all of the nucleosides are modified.

The nucleic acids featured in the invention can be synthesized or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference.

Modifications include, for example, end modifications, e.g., 5′-end modifications (phosphorylation, conjugation, inverted linkages) or 3′-end modifications (conjugation, DNA nucleosides within an RNA, or RNA nucleosides within a DNA, inverted linkages, etc.); base modifications, e.g., replacement with stabilizing bases, destabilizing bases, or bases that base pair with an expanded repertoire of partners, conjugated bases: sugar modifications (e.g., at the 2′-position or 4′-position) or replacement of the sugar: or backbone modifications, including modification or replacement of the phosphodiester linkages.

Specific examples of nucleic acids such as siRNA compounds useful in the embodiments described herein include, but are not limited to RNAs containing modified backbones or no natural internucleoside linkages. Nucleic acids such as RNAs having modified backbones include, among others, those that do not have a phosphorus atom in the backbone. For the purposes of this specification, and as sometimes referenced in the art, modified nucleic acids e.g. RNAs that do not have a phosphorus atom in their internucleoside backbone can also be considered to be oligonucleosides. In some embodiments, a modified nucleic acid e.g. an siRNA will have a phosphorus atom in its internucleoside backbone.

Modified nucleic acid e.g. RNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′-linked analogs of these, and those having inverted polarity wherein the adjacent pairs of nucleoside units are linked 5′-3′ or 5′-2′. Various salts, mixed salts and free acid forms are also included.

Modified nucleic acids e.g. RNAs can also contain one or more substituted sugar moieties. The nucleic acids e.g. siRNAs, e.g., dsiRNAs, featured herein can include one of the following at the 2′-position: OH; F; O-, S-, or N-alkyl; O-, S-, or N-alkenyl: O-, S- or N-alkynyl; or O-alkyl-O-alkyl, wherein the alkyl, alkenyl and alkynyl can be substituted or unsubstituted. 2′ O-methyl and 2′-F are preferred modifications.

In certain preferred embodiments, the nucleic acid comprises at least one modified nucleoside.

The nucleic acid of the invention may comprise one or more modified nucleosides on the first strand and/or the second strand.

In some embodiments, substantially all of the nucleosides of the sense strand and all of the nucleosides of the antisense strand comprise a modification.

In some embodiments, all of the nucleosides of the sense strand and substantially all of the nucleosides of the antisense strand comprise a modification.

In some embodiments, all of the nucleosides of the sense strand and all of the nucleosides of the antisense strand comprise a modification.

In one embodiment, at least one of the modified nucleosides is selected from the group consisting of a deoxy-nucleoside, a 3′-terminal deoxy-thymine (dT) nucleoside, a 2′-O-methyl modified nucleoside (also called herein 2′-Me, where Me is a methoxy), a 2′-fluoro modified nucleoside, a 2′-deoxy-modified nucleoside, a locked nucleoside, an unlocked nucleoside, a conformationally restricted nucleoside, a constrained ethyl nucleoside, an abasic nucleoside, a 2′-amino-modified nucleoside, a 2′-O-allyl-modified nucleoside, 2′-O-alkyl-modified nucleoside, 2′-hydroxyl-modified nucleoside, a 2′-methoxyethyl modified nucleoside, a 2′-O-alkyl-modified nucleoside, a morpholino nucleoside, a phosphoramidate, a non-natural base comprising nucleoside, a tetrahydropyran modified nucleoside, a 1,5-anhydrohexitol modified nucleoside, a cyclohexenyl modified nucleoside, a nucleoside comprising a phosphorothioate group, a nucleoside comprising a methylphosphonate group, a nucleoside comprising a 5′-phosphate, and a nucleoside comprising a 5′-phosphate mimic. In another embodiment, the modified nucleosides comprise a short sequence of 3′-terminal deoxy-thymine nucleosides (dT).

Modifications on the nucleosides may preferably be selected from the group including, but not limited to, LNA, HNA, CeNA, 2′-methoxyethyl, 2′-O-alkyl, 2′-O-allyl, 2′-C-allyl, 2′-fluoro, 2′-deoxy, 2′-hydroxyl, and combinations thereof. In another embodiment, the modifications on the nucleosides are 2-O-methyl (“2′-Me”) or 2′-fluoro modifications.

One preferred modification is a modification at the 2′-OH group of the ribose sugar, optionally selected from 2′-Me or 2′-F modifications.

Preferred nucleic acid comprise one or more nucleosides on the first strand and/or the second strand which are modified, to form modified nucleosides, as follows:

A nucleic acid wherein the modification is a modification at the 2′-OH group of the ribose sugar, optionally selected from 2′-Me or 2′-F modifications.

A nucleic acid wherein the first strand comprises a 2′-F modification at any of position 2, position 6, position 14, or any combination thereof, counting from position 1 of said first strand.

A nucleic acid wherein the second strand comprises a 2′-F modification at any of position 7, position 9, position 11, or any combination thereof, counting from position 1 of said second strand.

A nucleic acid wherein the first and second strand each comprise 2′-Me and 2′-F modifications.

A nucleic which comprises at least one thermally destabilizing modification, suitably at one or more of positions 1 to 9 of the first strand counting from position 1 of the first strand, and/or at one or more of positions on the second strand aligned with positions 1 to 9 of the first strand, wherein the destabilizing modification is selected from a modified unlocked nucleic acid (UNA) and a glycol nucleic acid (GNA), preferably a glycol nucleic acid, more preferably an (S)-glycol nucleic acid.

A nucleic acid which comprises at least one thermally destabilizing modification at position 7 of the first strand, counting from position 1 of the first strand.

A nucleic acid which is an siRNA oligonucleoside, wherein the siRNA oligonucleoside comprises 3 or more 2′-F modifications at positions 6 to 12 of the second strand, such as 4, 5, 6 or 7 2′-F modifications at positions 6 to 12 of the second strand, counting from position 1 of said second strand.

A nucleic acid which is an siRNA oligonucleoside, wherein said second strand comprises at least 3, such as 4, 5 or 6, 2′-Me modifications at positions 1 to 6 of the second strand, counting from position 1 of said second strand.

A nucleic acid which is an siRNA oligonucleoside, wherein said first strand comprises at least 5 2′-Me consecutive modifications at the 3′ terminal region, preferably including the terminal nucleoside at the 3′ terminal region, or at least within 1 or 2 nucleosides from the terminal nucleoside at the 3′ terminal region.

A nucleic acid which is an siRNA oligonucleoside, wherein said first strand comprises 7 2′-Me consecutive modifications at the 3′ terminal region, preferably including the terminal nucleoside at the 3′ terminal region.

A nucleic acid which is an siRNA oligonucleoside, wherein each of the first and second strands comprises an alternating modification pattern, preferably a fully alternating modification pattern along the entire length of each of the first and second strands, wherein the nucleosides of the first strand are modified by (i) 2′Me modifications on the odd numbered nucleosides counting from position 1 of the first strand, and (ii) 2′F modifications on the even numbered nucleosides counting from position 1 of the first strand, and nucleosides of the second strand are modified by (i) 2′F modifications on the odd numbered nucleosides counting from position 1 of the second strand, and (ii) 2′Me modifications on the even numbered nucleosides counting from position 1 of the second strand. Typically such fully alternating modification patterns are present in a blunt ended oligonucleoside, wherein each of the first and second strands are 19 nucleosides in length.

Position 1 of the first or the second strand is the nucleoside which is the closest to the end of the nucleic acid (ignoring any abasic nucleosides) and that is joined to an adjacent nucleoside (at Position 2) via a 3′ to 5′ internal bond, with reference to the bonds between the sugar moieties of the backbone, and reading in a direction away from that end of the molecule.

It can therefore be seen that “position 1 of the sense strand” is the 5′ most nucleoside (not including abasic nucleosides) at the conventional 5′ end of the sense strand. Typically, the nucleoside at this position 1 of the sense strand will be equivalent to the 5′ nucleoside of the selected target nucleic acid sequence, and more generally the sense strand will have equivalent nucleosides to those of the target nucleic acid sequence starting from this position 1 of the sense strand, whilst also allowing for acceptable mismatches between the sequences.

As used herein, “position 1 of the antisense strand” is the 5′ most nucleoside (not including abasic nucleosides) at the conventional 5′ end of the antisense strand. As hereinbefore described, there will be a region of complementarity between the sense and antisense strands, and in this way the antisense strand will also have a region of complementarity to the target nucleic acid sequence as referred to above.

In certain embodiments, the nucleic acid e.g. siRNA agent further comprises at least one phosphorothioate or methylphosphonate internucleoside linkage. For example the phosphorothioate or methylphosphonate internucleoside linkage can be at the 3′-terminus or in the terminal region of one strand, i.e., the sense strand or the antisense strand: or at the ends of both strands, the sense strand and the antisense strand.

In certain embodiments, the phosphorothioate or methylphosphonate internucleoside linkage is at the 5′terminus or in the terminal region of one strand, i.e., the sense strand or the antisense strand: or at the ends of both strands, the sense strand and the antisense strand.

In certain embodiments, a phosphorothioate or a methylphosphonate internucleoside linkage is at both the 5′- and 3′-terminus or in the terminal region of one strand, i.e., the sense strand or the antisense strand: or at the ends of both strands, the sense strand and the antisense strand.

Any nucleic acid may comprise one or more phosphorothioate (PS) modifications within the nucleic acid, such as at least two PS internucleoside bonds at the ends of a strand.

At least one of the oligoribonucleotide strands preferably comprises at least two consecutive phosphorothioate modifications in the last 3 nucleosides of the oligonucleoside.

The invention therefore also relates to: A nucleic acid disclosed herein which comprises phosphorothioate internucleoside linkages respectively between at least two or three consecutive positions, such as in a 5′ and/or 3′ terminal region and/or near terminal region of the second strand, whereby said near terminal region is preferably adjacent said terminal region wherein said one or more abasic nucleosides of said second strand is/are located.

A nucleic acid disclosed herein which comprises phosphorothioate internucleoside linkages respectively between at least two or three consecutive positions in a 5′ and/or 3′ terminal region of the first strand, whereby preferably the terminal position at the 5′ and/or 3′ terminal region of said first strand is attached to its adjacent position by a phosphorothioate internucleoside linkage.

The nucleic acid strand may be an RNA comprising a phosphorothioate internucleoside linkage between the three nucleosides contiguous with 2 terminally located abasic nucleosides.

A preferred nucleic acid is a double stranded RNA comprising 2 adjacent abasic nucleosides at the 5′ terminus of the second strand and a ligand moiety comprising one or more GalNAc ligand moieties at the opposite 3′ end of the second strand. Further preferred, the same nucleic acid may also comprise a phosphorothioate bond between nucleotides at positions 3-4 and 4-5 of the second strand, reading from the position 1 of the second strand. Further preferred, the same nucleic acid may also comprise a 2′ F modification at positions 7, 9 and 11 of the second strand.

Preferred modifications are as follows.

A nucleic acid wherein modified nucleosides of said second strand comprise a modification pattern according to any one of the following (5′-3′):
Me-Me-Me-Me-Me-Me-F—F—F—F—F-Me-Me-Me-Me-Me-Me-Me-F-Me-Me, or
Me-Me-Me-Me-Me-F—F-Me-F—F—F—F-Me-Me-Me-Me-Me-Me-Me-Me-Me, or
Me-Me-Me-Me-Me-Me-F-Me-F—F—F—F-Me-Me-Me-Me-Me-Me-Me-Me-Me, or
Me-Me-Me-Me-Me-Me-Me-Me-F—F—F-Me-Me-Me-Me-Me-Me-Me-Me-Me-Me, or
Me-Me-Me-Me-Me-Me-F-Me-F—F—F-Me-Me-Me-Me-Me-Me-Me-Me-Me-Me.

A nucleic acid wherein modified nucleosides of said second strand comprise a modification pattern according to any one of the following (5′-3′):
Me(s)Me(s)Me-Me-Me-Me-F—F—F—F—F-Me-Me-Me-Me-Me-Me-Me-F-Me-Me, or
Me(s)Me(s)Me-Me-Me-F—F-Me-F—F—F—F-Me-Me-Me-Me-Me-Me-Me-Me-Me, or
Me(s)Me(s)Me-Me-Me-Me-F-Me-F—F—F—F-Me-Me-Me-Me-Me-Me-Me-Me-Me, or
Me(s)Me(s)Me-Me-Me-Me-Me-Me-F—F—F-Me-Me-Me-Me-Me-Me-Me-Me-Me-Me, or
Me(s)Me(s)Me-Me-Me-Me-F-Me-F—F—F-Me-Me-Me-Me-Me-Me-Me-Me-Me-Me, or
Me-Me-Me-Me-Me-Me-F—F—F—F—F-Me-Me-Me-Me-Me-Me-Me-F(s)Me(s)Me, or
Me-Me-Me-Me-Me-F—F-Me-F—F—F—F-Me-Me-Me-Me-Me-Me-Me(s)Me(s)Me, or
Me-Me-Me-Me-Me-Me-F-Me-F—F—F—F-Me-Me-Me-Me-Me-Me-Me(s)Me(s)Me, or
Me-Me-Me-Me-Me-Me-Me-Me-F—F—F-Me-Me-Me-Me-Me-Me-Me-Me(s)Me(s)Me, or
Me-Me-Me-Me-Me-Me-F-Me-F—F—F-Me-Me-Me-Me-Me-Me-Me-Me(s)Me(s)Me,
wherein(s) is a phosphorothioate internucleoside linkage.

A nucleic acid wherein modified nucleosides of said second strand comprise a modification pattern according to any one of the following (5′-3′):
ia-ia-Me-Me-Me-Me-Me-Me-F—F—F—F—F-Me-Me-Me-Me-Me-Me-Me-F-Me-Me, or
ia-ia-Me-Me-Me-Me-Me-F—F-Me-F—F—F—F-Me-Me-Me-Me-Me-Me-Me-Me-Me, or
ia-ia-Me-Me-Me-Me-Me-Me-F-Me-F—F—F—F-Me-Me-Me-Me-Me-Me-Me-Me-Me, or
ia-ia-Me-Me-Me-Me-Me-Me-Me-Me-F—F—F-Me-Me-Me-Me-Me-Me-Me-Me-Me-Me, or
ia-ia-Me-Me-Me-Me-Me-Me-F-Me-F—F—F-Me-Me-Me-Me-Me-Me-Me-Me-Me-Me, or
Me-Me-Me-Me-Me-Me-F—F—F—F—F-Me-Me-Me-Me-Me-Me-Me-F-Me-Me-ia-ia, or
Me-Me-Me-Me-Me-F—F-Me-F—F—F—F-Me-Me-Me-Me-Me-Me-Me-Me-Me-ia-ia, or
Me-Me-Me-Me-Me-Me-F-Me-F—F—F—F-Me-Me-Me-Me-Me-Me-Me-Me-Me-ia-ia, or
Me-Me-Me-Me-Me-Me-Me-Me-F—F—F-Me-Me-Me-Me-Me-Me-Me-Me-Me-Me-ia-ia, or
Me-Me-Me-Me-Me-Me-F-Me-F—F—F-Me-Me-Me-Me-Me-Me-Me-Me-Me-Me-ia-ia,
wherein ia represents an inverted abasic nucleoside, and when the inverted abasic nucleosides as represented by ia-ia are present at the 3′ terminus of the second strand, said inverted abasic nucleosides are present in a 2 nucleoside overhang.

A nucleic acid wherein modified nucleosides of said second strand comprise a modification pattern according to any one of the following (5′-3′):
ia-ia-Me(s)Me(s)Me-Me-Me-Me-F—F—F—F—F-Me-Me-Me-Me-Me-Me-Me-F-Me-Me, or
ia-ia-Me(s)Me(s)Me-Me-Me-F—F-Me-F—F—F—F-Me-Me-Me-Me-Me-Me-Me-Me-Me, or
ia-ia-Me(s)Me(s)Me-Me-Me-Me-F-Me-F—F—F—F-Me-Me-Me-Me-Me-Me-Me-Me-Me, or
ia-ia-Me(s)Me(s)Me-Me-Me-Me-Me-Me-F—F—F-Me-Me-Me-Me-Me-Me-Me-Me-Me-Me, or
ia-ia-Me(s)Me(s)Me-Me-Me-Me-F-Me-F—F—F-Me-Me-Me-Me-Me-Me-Me-Me-Me-Me, or
Me-Me-Me-Me-Me-Me-F—F—F—F—F-Me-Me-Me-Me-Me-Me-Me-F(s)Me(s)Me-ia-ia, or
Me-Me-Me-Me-Me-F—F-Me-F—F—F—F-Me-Me-Me-Me-Me-Me-Me(s)Me(s)Me-ia-ia, or
Me-Me-Me-Me-Me-Me-F-Me-F—F—F—F-Me-Me-Me-Me-Me-Me-Me(s)Me(s)Me-ia-ia, or
Me-Me-Me-Me-Me-Me-Me-Me-F—F—F-Me-Me-Me-Me-Me-Me-Me-Me(s)Me(s)Me-ia-ia, or
Me-Me-Me-Me-Me-Me-F-Me-F—F—F-Me-Me-Me-Me-Me-Me-Me-Me(s)Me(s)Me-ia-ia,
wherein:
(s) is a phosphorothioate internucleoside linkage, ia represents an inverted abasic nucleoside, and when the inverted abasic nucleosides as represented by ia-ia are present at the 3′ terminus of the second strand, said inverted abasic nucleosides are present in a 2 nucleoside overhang.

A nucleic acid wherein modified nucleosides comprise any one of the following modification patterns:
Modification pattern 1: Second strand (5′-3′): Me-Me-Me-Me-Me-Me-F—F—F—F—F-Me-Me-Me-Me-Me-Me-Me-F-Me-Me, First strand (5′-3′): Me-F-Me-F-Me-F-Me-F—F-Me-Me-Me-Me-F-Me-F-Me-Me-Me-Me-Me-Me-Me
Or Modification pattern 2: Second strand (5′-3′): Me-Me-Me-Me-Me-F—F-Me-F—F—F—F-Me-Me-Me-Me-Me-Me-Me-Me-Me, First strand (5′-3′): Me-F-Me-F-Me-F-Me-F—F-Me-Me-Me-Me-F-Me-F-Me-Me-Me-Me-Me-Me-Me
Or Modification pattern 3: Second strand (5′-3′): Me-Me-Me-Me-Me-Me-F-Me-F—F—F—F-Me-Me-Me-Me-Me-Me-Me-Me-Me, First strand (5′-3′): Me-F-Me-F-Me-F-Me-F—F-Me-Me-Me-Me-F-Me-F-Me-Me-Me-Me-Me-Me-Me
Or Modification pattern 4: Second strand (5′-3′): Me-Me-Me-Me-Me-Me-F-Me-F—F—F—F-Me-Me-Me-Me-Me-Me-Me-Me-Me, First strand (5′-3′): Me-F-Me-F-Me-F-Me-Me-Me-Me-Me-Me-Me-F-Me-F-Me-Me-Me-Me-Me-Me-Me
Or Modification pattern 5: Second strand (5′-3′): Me-Me-Me-Me-Me-Me-Me-Me-F—F—F-Me-Me-Me-Me-Me-Me-Me-Me-Me-Me, First strand (5′-3′): Me-F-Me-Me-Me-F-Me-Me-F-Me-Me-Me-Me-F-Me-F-Me-Me-Me-Me-Me-Me-Me
Or Modification pattern 6: Second strand (5′-3′): Me-Me-Me-Me-Me-Me-F-Me-F—F—F-Me-Me-Me-Me-Me-Me-Me-Me-Me-Me, First strand (5′-3′): Me-F-Me-Me-Me-F-Me-Me-F-Me-Me-Me-Me-F-Me-F-Me-Me-Me-Me-Me-Me-Me.

A nucleic acid wherein modified nucleosides comprise any one of the following modification patterns:
Modification pattern 1: Second strand (5′-3′): Me(s)Me(s)Me-Me-Me-Me-F—F—F—F—F-Me-Me-Me-Me-Me-Me-Me-F-Me-Me, First strand (5′-3′): Me(s)F(s)Me-F-Me-F-Me-F—F-Me-Me-Me-Me-F-Me-F-Me-Me-Me-Me-Me(s)Me(s)Me
Or Modification pattern 2: Second strand (5′-3′): Me(s)Me(s)Me-Me-Me-F—F-Me-F—F—F—F-Me-Me-Me-Me-Me-Me-Me-Me-Me, First strand (5′-3′): Me(s)F(s)Me-F-Me-F-Me-F—F-Me-Me-Me-Me-F-Me-F-Me-Me-Me-Me-Me(s)Me(s)Me
Or Modification pattern 3: Second strand (5′-3′): Me(s)Me(s)Me-Me-Me-Me-F-Me-F—F—F—F-Me-Me-Me-Me-Me-Me-Me-Me-Me, First strand (5′-3′): Me(s)F(s)Me-F-Me-F-Me-F—F-Me-Me-Me-Me-F-Me-F-Me-Me-Me-Me-Me(s)Me(s)Me
Or Modification pattern 4: Second strand (5′-3′): Me(s)Me(s)Me-Me-Me-Me-F-Me-F—F—F—F-Me-Me-Me-Me-Me-Me-Me-Me-Me, First strand (5′-3′): Me(s)F(s)Me-F-Me-F-Me-Me-Me-Me-Me-Me-Me-F-Me-F-Me-Me-Me-Me-Me(s)Me(s)Me
Or Modification pattern 5: Second strand (5′-3′): Me(s)Me(s)Me-Me-Me-Me-Me-Me-F—F—F-Me-Me-Me-Me-Me-Me-Me-Me-Me-Me, First strand (5′-3′): Me(s)F(s)Me-Me-Me-F-Me-Me-F-Me-Me-Me-Me-F-Me-F-Me-Me-Me-Me-Me(s)Me(s)Me
Or Modification pattern 6: Second strand (5′-3′): Me(s)Me(s)Me-Me-Me-Me-F-Me-F—F—F-Me-Me-Me-Me-Me-Me-Me-Me-Me-Me, First strand (5′-3′): Me(s)F(s)Me-Me-Me-F-Me-Me-F-Me-Me-Me-Me-F-Me-F-Me-Me-Me-Me-Me(s)Me(s)Me
wherein(s) is a phosphorothioate internucleoside linkage.

A nucleic acid wherein modified nucleosides comprise any one of the following modification patterns:
Modification pattern 1: Second strand (5′-3′): Me-Me-Me-Me-Me-Me-F—F—F—F—F-Me-Me-Me-Me-Me-Me-Me-F(s)Me(s)Me, First strand (5′-3′): Me(s)F(s)Me-F-Me-F-Me-F—F-Me-Me-Me-Me-F-Me-F-Me-Me-Me-Me-Me(s)Me(s)Me
Or Modification pattern 2: Second strand (5′-3′): Me-Me-Me-Me-Me-F—F-Me-F—F—F—F-Me-Me-Me-Me-Me-Me-Me(s)Me(s)Me, First strand (5′-3′): Me(s)F(s)Me-F-Me-F-Me-F—F-Me-Me-Me-Me-F-Me-F-Me-Me-Me-Me-Me(s)Me(s)Me
Or Modification pattern 3: Second strand (5′-3′): Me-Me-Me-Me-Me-Me-F-Me-F—F—F—F-Me-Me-Me-Me-Me-Me-Me(s)Me(s)Me, First strand (5′-3′): Me(s)F(s)Me-F-Me-F-Me-F—F-Me-Me-Me-Me-F-Me-F-Me-Me-Me-Me-Me(s)Me(s)Me
Or Modification pattern 4: Second strand (5′-3′): Me-Me-Me-Me-Me-Me-F-Me-F—F—F—F-Me-Me-Me-Me-Me-Me-Me(s)Me(s)Me, First strand (5′-3′): Me(s)F(s)Me-F-Me-F-Me-Me-Me-Me-Me-Me-Me-F-Me-F-Me-Me-Me-Me-Me(s)Me(s)Me
Or Modification pattern 5: Second strand (5′-3′): Me-Me-Me-Me-Me-Me-Me-Me-F—F—F-Me-Me-Me-Me-Me-Me-Me-Me(s)Me(s)Me, First strand (5′-3′): Me(s)F(s)Me-Me-Me-F-Me-Me-F-Me-Me-Me-Me-F-Me-F-Me-Me-Me-Me-Me(s)Me(s)Me
Or Modification pattern 6: Second strand (5′-3′): Me-Me-Me-Me-Me-Me-F-Me-F—F—F-Me-Me-Me-Me-Me-Me-Me-Me(s)Me(s)Me, First strand (5′-3′): Me(s)F(s)Me-Me-Me-F-Me-Me-F-Me-Me-Me-Me-F-Me-F-Me-Me-Me-Me-Me(s)Me(s)Me
wherein(s) is a phosphorothioate internucleoside linkage.

A nucleic acid wherein modified nucleosides comprise any one of the following modification patterns:
Modification pattern 1: Second strand (5′-3′): ia-ia-Me-Me-Me-Me-Me-Me-F—F—F—F—F-Me-Me-Me-Me-Me-Me-Me-F-Me-Me, First strand (5′-3′): Me-F-Me-F-Me-F-Me-F—F-Me-Me-Me-Me-F-Me-F-Me-Me-Me-Me-Me-Me-Me
Or Modification pattern 2: Second strand (5′-3′): ia-ia-Me-Me-Me-Me-Me-F—F-Me-F—F—F—F-Me-Me-Me-Me-Me-Me-Me-Me-Me, First strand (5′-3′): Me-F-Me-F-Me-F-Me-F—F-Me-Me-Me-Me-F-Me-F-Me-Me-Me-Me-Me-Me-Me
Or Modification pattern 3: Second strand (5′-3′): ia-ia-Me-Me-Me-Me-Me-Me-F-Me-F—F—F—F-Me-Me-Me-Me-Me-Me-Me-Me-Me, First strand (5′-3′): Me-F-Me-F-Me-F-Me-F—F-Me-Me-Me-Me-F-Me-F-Me-Me-Me-Me-Me-Me-Me
Or Modification pattern 4: Second strand (5′-3′): ia-ia-Me-Me-Me-Me-Me-Me-F-Me-F—F—F—F-Me-Me-Me-Me-Me-Me-Me-Me-Me, First strand (5′-3′): Me-F-Me-F-Me-F-Me-Me-Me-Me-Me-Me-Me-F-Me-F-Me-Me-Me-Me-Me-Me-Me
Or Modification pattern 5: Second strand (5′-3′): ia-ia-Me-Me-Me-Me-Me-Me-Me-Me-F—F—F-Me-Me-Me-Me-Me-Me-Me-Me-Me-Me, First strand (5′-3′): Me-F-Me-Me-Me-F-Me-Me-F-Me-Me-Me-Me-F-Me-F-Me-Me-Me-Me-Me-Me-Me
Or Modification pattern 6: Second strand (5′-3′): ia-ia-Me-Me-Me-Me-Me-Me-F-Me-F—F—F-Me-Me-Me-Me-Me-Me-Me-Me-Me-Me, First strand (5′-3′): Me-F-Me-Me-Me-F-Me-Me-F-Me-Me-Me-Me-F-Me-F-Me-Me-Me-Me-Me-Me-Me,
wherein ia represents an inverted abasic nucleoside.

A nucleic acid wherein modified nucleosides comprise any one of the following modification patterns:
Modification pattern 1: Second strand (5′-3′): Me-Me-Me-Me-Me-Me-F—F—F—F—F-Me-Me-Me-Me-Me-Me-Me-F-Me-Me-ia-ia, First strand (5′-3′): Me-F-Me-F-Me-F-Me-F—F-Me-Me-Me-Me-F-Me-F-Me-Me-Me-Me-Me-Me-Me
Or Modification pattern 2: Second strand (5′-3′): Me-Me-Me-Me-Me-F—F-Me-F—F—F—F-Me-Me-Me-Me-Me-Me-Me-Me-Me-ia-ia, First strand (5′-3′): Me-F-Me-F-Me-F-Me-F—F-Me-Me-Me-Me-F-Me-F-Me-Me-Me-Me-Me-Me-Me
Or Modification pattern 3: Second strand (5′-3′): Me-Me-Me-Me-Me-Me-F-Me-F—F—F—F-Me-Me-Me-Me-Me-Me-Me-Me-Me-ia-ia, First strand (5′-3′): Me-F-Me-F-Me-F-Me-F—F-Me-Me-Me-Me-F-Me-F-Me-Me-Me-Me-Me-Me-Me
Or Modification pattern 4: Second strand (5′-3′): Me-Me-Me-Me-Me-Me-F-Me-F—F—F—F-Me-Me-Me-Me-Me-Me-Me-Me-Me-ia-ia, First strand (5′-3′): Me-F-Me-F-Me-F-Me-Me-Me-Me-Me-Me-Me-F-Me-F-Me-Me-Me-Me-Me-Me-Me
Or Modification pattern 5: Second strand (5′-3′): Me-Me-Me-Me-Me-Me-Me-Me-F—F—F-Me-Me-Me-Me-Me-Me-Me-Me-Me-Me-ia-ia, First strand (5′-3′): Me-F-Me-Me-Me-F-Me-Me-F-Me-Me-Me-Me-F-Me-F-Me-Me-Me-Me-Me-Me-Me
Or Modification pattern 6: Second strand (5′-3′): Me-Me-Me-Me-Me-Me-F-Me-F—F—F-Me-Me-Me-Me-Me-Me-Me-Me-Me-Me-ia-ia, First strand (5′-3′): Me-F-Me-Me-Me-F-Me-Me-F-Me-Me-Me-Me-F-Me-F-Me-Me-Me-Me-Me-Me-Me,
wherein ia represents an inverted abasic nucleoside, and when the inverted abasic nucleosides as represented by ia-ia are present at the 3′ terminus of the second strand, said inverted abasic nucleosides are present in a 2 nucleoside overhang.

A nucleic acid wherein modified nucleosides comprise any one of the following modification patterns:
Modification pattern 1: Second strand (5′-3′): ia-ia-Me(s)Me(s)Me-Me-Me-Me-F—F—F—F—F-Me-Me-Me-Me-Me-Me-Me-F-Me-Me, First strand (5′-3′): Me(s)F(s)Me-F-Me-F-Me-F—F-Me-Me-Me-Me-F-Me-F-Me-Me-Me-Me-Me(s)Me(s)Me
Or Modification pattern 2: Second strand (5′-3′): ia-ia-Me(s)Me(s)Me-Me-Me-F—F-Me-F—F—F—F-Me-Me-Me-Me-Me-Me-Me-Me-Me, First strand (5′-3′): Me(s)F(s)Me-F-Me-F-Me-F—F-Me-Me-Me-Me-F-Me-F-Me-Me-Me-Me-Me(s)Me(s)Me
Or Modification pattern 3: Second strand (5′-3′): ia-ia-Me(s)Me(s)Me-Me-Me-Me-F-Me-F—F—F—F-Me-Me-Me-Me-Me-Me-Me-Me-Me, First strand (5′-3′): Me(s)F(s)Me-F-Me-F-Me-F—F-Me-Me-Me-Me-F-Me-F-Me-Me-Me-Me-Me(s)Me(s)Me
Or Modification pattern 4: Second strand (5′-3′): ia-ia-Me(s)Me(s)Me-Me-Me-Me-F-Me-F—F—F—F-Me-Me-Me-Me-Me-Me-Me-Me-Me, First strand (5′-3′): Me(s)F(s)Me-F-Me-F-Me-Me-Me-Me-Me-Me-Me-F-Me-F-Me-Me-Me-Me-Me(s)Me(s)Me
Or Modification pattern 5: Second strand (5′-3′): ia-ia-Me(s)Me(s)Me-Me-Me-Me-Me-Me-F—F—F-Me-Me-Me-Me-Me-Me-Me-Me-Me-Me, First strand (5′-3′): Me(s)F(s)Me-Me-Me-F-Me-Me-F-Me-Me-Me-Me-F-Me-F-Me-Me-Me-Me-Me(s)Me(s)Me
Or Modification pattern 6: Second strand (5′-3′): ia-ia-Me(s)Me(s)Me-Me-Me-Me-F-Me-F—F—F-Me-Me-Me-Me-Me-Me-Me-Me-Me-Me, First strand (5′-3′): Me(s)F(s)Me-Me-Me-F-Me-Me-F-Me-Me-Me-Me-F-Me-F-Me-Me-Me-Me-Me(s)Me(s)Me
wherein:
(s) is a phosphorothioate internucleoside linkage, ia represents an inverted abasic nucleoside.

A nucleic acid wherein modified nucleosides comprise any one of the following modification patterns:
Modification pattern 1: Second strand (5′-3′): Me-Me-Me-Me-Me-Me-F—F—F—F—F-Me-Me-Me-Me-Me-Me-Me-F(s)Me(s)Me-ia-ia, First strand (5′-3′): Me(s)F(s)Me-F-Me-F-Me-F—F-Me-Me-Me-Me-F-Me-F-Me-Me-Me-Me-Me(s)Me(s)Me
Or Modification pattern 2: Second strand (5′-3′): Me-Me-Me-Me-Me-F—F-Me-F—F—F—F-Me-Me-Me-Me-Me-Me-Me(s)Me(s)Me-ia-ia, First strand (5′-3′): Me(s)F(s)Me-F-Me-F-Me-F—F-Me-Me-Me-Me-F-Me-F-Me-Me-Me-Me-Me(s)Me(s)Me
Or Modification pattern 3: Second strand (5′-3′): Me-Me-Me-Me-Me-Me-F-Me-F—F—F—F-Me-Me-Me-Me-Me-Me-Me(s)Me(s)Me-ia-ia, First strand (5′-3′): Me(s)F(s)Me-F-Me-F-Me-F—F-Me-Me-Me-Me-F-Me-F-Me-Me-Me-Me-Me(s)Me(s)Me
Or Modification pattern 4: Second strand (5′-3′): Me-Me-Me-Me-Me-Me-F-Me-F—F—F—F-Me-Me-Me-Me-Me-Me-Me(s)Me(s)Me-ia-ia, First strand (5′-3′): Me(s)F(s)Me-F-Me-F-Me-Me-Me-Me-Me-Me-Me-F-Me-F-Me-Me-Me-Me-Me(s)Me(s)Me
Or Modification pattern 5: Second strand (5′-3′): Me-Me-Me-Me-Me-Me-Me-Me-F—F—F-Me-Me-Me-Me-Me-Me-Me-Me(s)Me(s)Me-ia-ia, First strand (5′-3′): Me(s)F(s)Me-Me-Me-F-Me-Me-F-Me-Me-Me-Me-F-Me-F-Me-Me-Me-Me-Me(s)Me(s)Me
Or Modification pattern 6: Second strand (5′-3′): Me-Me-Me-Me-Me-Me-F-Me-F—F—F-Me-Me-Me-Me-Me-Me-Me-Me(s)Me(s)Me-ia-ia, First strand (5′-3′): Me(s)F(s)Me-Me-Me-F-Me-Me-F-Me-Me-Me-Me-F-Me-F-Me-Me-Me-Me-Me(s)Me(s)Me
wherein: (s) is a phosphorothioate internucleoside linkage, ia represents an inverted abasic nucleoside, and when the inverted abasic nucleosides as represented by ia-ia are present at the 3′ terminus of the second strand, said inverted abasic nucleosides are present in a 2 nucleoside overhang.

Particularly preferred is a nucleic acid wherein the modified nucleosides comprise the following modification pattern:
Modification pattern 4: Second strand (5′-3′): ia-ia-Me(s)Me(s)Me-Me-Me-Me-F-Me-F—F—F—F-Me-Me-Me-Me-Me-Me-Me-Me-Me, First strand (5′-3′): Me(s)F(s)Me-F-Me-F-Me-Me-Me-Me-Me-Me-Me-F-Me-F-Me-Me-Me-Me-Me(s)Me(s)Me
wherein: (s) is a phosphorothioate internucleoside linkage, ia represents an inverted abasic nucleoside.

A nucleic acid wherein the first strand comprises a 2′ sugar modification pattern wherein said modifications are selected at least from 2′Me and 2′F sugar modifications, provided that the overall number of 2′F sugar modifications in the first strand does not consist of four, or six, 2′F modifications.

A nucleic acid wherein the first strand comprises a 2′ sugar modification pattern wherein said modifications are selected at least from 2′Me and 2′F sugar modifications, wherein the overall number of 2′F sugar modifications in the first strand consists of three, five or seven 2′F modifications.

A nucleic acid wherein the first strand comprises a 2′ sugar modification pattern wherein said modifications are selected at least from 2′Me and 2′F sugar modifications, wherein the overall number of 2′F sugar modifications in the first strand consists of three 2′F modifications.

A nucleic acid wherein the first strand comprises a 2′ sugar modification pattern wherein said modifications are selected at least from 2′Me and 2′F sugar modifications, wherein the overall number of 2′F sugar modifications in the first strand consists of five 2′F modifications.

A nucleic acid wherein the first strand comprises a 2′ sugar modification pattern as follows (5′-3′):
Me-F-Me-X₂-Me-F-(Me)₇-(F-Me)₂-X₃-Me-X₄-(Me)₃
wherein X₂, X₃ and X₄ are selected from 2′Me and 2′F sugar modifications, provided that for X₂, X₃ and X₄ at least one is a 2′F sugar modification, and the other two sugar modifications are 2′Me sugar modifications.

A nucleic acid wherein the first strand comprises a 2′ sugar modification pattern as follows (5′-3′):
Me-F-Me-X₂-Me-F-(Me)₇-(F-Me)₂-X₃-Me-X₄-(Me)₃
wherein X₂ is a 2′F sugar modification, and X₃ and X₄ are 2′Me sugar modifications.

A nucleic acid wherein the first strand comprises a 2′ sugar modification pattern as follows (5′-3′):
Me-F-Me-X₂-Me-F-(Me)₇-(F-Me)₂-X₃-Me-X₄-(Me)₃
wherein X₃ is a 2′F sugar modification, and X₂ and X₄ are 2′Me sugar modifications.

A nucleic acid wherein the first strand comprises a 2′ sugar modification pattern as follows (5′-3′):
Me-F-Me-X₂-Me-F-(Me)₇-(F-Me)₂-X₃-Me-X₄-(Me)₃
wherein X₄ is a 2′F sugar modification, and X₂ and X₃ are 2′Me sugar modifications.

A nucleic acid wherein the first strand comprises a 2′ sugar modification pattern wherein said modifications are selected at least from 2′Me and 2′F sugar modifications, wherein the overall number of 2′F sugar modifications in the first strand consists of seven 2′F modifications.

A nucleic acid wherein the first strand comprises a 2′ sugar modification pattern as follows (5′-3′):
Me-F-Me-X₂-Me-F-Me-(F)₂-(Me)₄-(F-Me)₂-X₃-Me-X₄-(Me)₃
wherein X₂, X₃ and X₄ are selected from 2′Me and 2′F sugar modifications, provided that for X₂, X₃ and X₄ at least one is a 2′F sugar modification, and the other two sugar modifications are 2′Me sugar modifications.

A nucleic acid wherein the first strand comprises a 2′ sugar modification pattern as follows (5′-3′):
Me-F-Me-X₂-Me-F-Me-(F)₂-(Me)₄-(F-Me)₂-X₃-Me-X₄-(Me)₃
wherein X₂ is a 2′F sugar modification, and X₃ and X₄ are 2′Me sugar modifications.

A nucleic acid wherein the first strand comprises a 2′ sugar modification pattern as follows (5′-3′):
Me-F-Me-X₂-Me-F-Me-(F)₂-(Me)₄-(F-Me)₂-X₃-Me-X₄-(Me)₃
wherein X₃ is a 2′F sugar modification, and X₂ and X₄ are 2′Me sugar modifications.

A nucleic acid wherein the first strand comprises a 2′ sugar modification pattern as follows (5′-3′):
Me-F-Me-X₂-Me-F-Me-(F)₂-(Me)₄-(F-Me)₂-X₃-Me-X₄-(Me)₃
wherein X₄ is a 2′F sugar modification, and X₂ and X₃ are 2′Me sugar modifications.

A nucleic acid wherein the first strand comprises a 2′ sugar modification pattern as follows (5′-3′):
Me-F-(Me)₃-X₁-(Me)₇-F-Me-F-(Me)₇
wherein X₁ is a thermally destabilising modification.

A nucleic acid wherein the first strand comprises a 2′ sugar modification pattern as follows (5′-3′):
Me-F-(Me)₃-X₁-Me-(F)₂-(Me)₄-F-Me-F-(Me)₇
wherein X₁ is a thermally destabilising modification.

A nucleic acid wherein the second strand comprises a 2′ sugar modification pattern as follows (5′-3′):
(Me)₈-(F)₃-(Me)₁₀.

A nucleic acid wherein the second strand comprises a 2′ sugar modification pattern as follows (5′-3′):
(Me)₈-(F)₃-(Me)₁₀, and
wherein the first strand comprises a 2′ sugar modification pattern wherein said modifications are selected at least from 2′Me and 2′F sugar modifications, provided that the overall number of 2′F sugar modifications in the first strand does not consist of four, or six, 2′F modifications.

A nucleic acid wherein the second strand comprises a 2′ sugar modification pattern as follows (5′-3′):
(Me)₈-(F)₃-(Me)₁₀, and
   wherein the first strand comprises a 2′ sugar modification pattern wherein said modifications are selected at least from 2′Me and 2′F sugar modifications, wherein the overall number of 2′F sugar modifications in the first strand consists of three, five or seven 2′F modifications.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar modification pattern as follows (5′-3′):
(Me)₈-(F)₃-(Me)₁₀, and
   wherein nucleosides of said first strand comprise a 2′ sugar modification pattern as follows (5′-3′):
Me-F-(Me)₃-X₁-(Me)₇-F-Me-F-(Me)₇, wherein X₁ is a thermally destabilising modification.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar modification pattern as follows (5′-3′):
(Me)₈-(F)₃-(Me)₁₀, and
wherein nucleosides of said first strand comprise a 2′ sugar modification pattern as follows (5′-3′):
(Me-F)₃-(Me)₇-F-Me-F-(Me)₇.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar modification pattern as follows (5′-3′):
(Me)₈-(F)₃-(Me)₁₀, and
wherein nucleosides of said first strand comprise a 2′ sugar modification pattern as follows (5′-3′):
Me-F-(Me)₃-F-(Me)₇-(F-Me)₂-F-(Me)₅.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar modification pattern as follows (5′-3′):
(Me)₈-(F)₃-(Me)₁₀, and
   wherein nucleosides of said first strand comprise a 2′ sugar modification pattern as follows (5′-3′):
Me-F-(Me)₃-F-(Me)₇-F-Me-F-(Me)₃-F-(Me)₃.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar modification pattern as follows (5′-3′):
(Me)₈-(F)₃-(Me)₁₀, and
wherein nucleosides of said first strand comprise a 2′ sugar modification pattern as follows (5′-3′):
Me-F-(Me)₃-X₁-Me-(F)₂-(Me)₄-F-Me-F-(Me)₇, wherein X₁ is a thermally destabilising modification.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar modification pattern as follows (5′-3′):
(Me)₈-(F)₃-(Me)₁₀, and
wherein nucleosides of said first strand comprise a 2′ sugar modification pattern as follows (5′-3′):
(Me-F)₃-Me-(F)₂-(Me)₄-(F-Me)₂-(Me)₆.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar modification pattern as follows (5′-3′):
(Me)₈-(F)₃-(Me)₁₀, and
wherein nucleosides of said first strand comprise a 2′ sugar modification pattern as follows (5′-3′):
Me-F-(Me)₃-F-Me-(F)₂-(Me)₄-(F-Me)₂-F-(Me)₅.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar modification pattern as follows (5′-3′):
(Me)₈-(F)₃-(Me)₁₀, and
wherein nucleosides of said first strand comprise a 2′ sugar modification pattern as follows (5′-3′):
Me-F-(Me)₃-F-Me-(F)₂-(Me)₄-(F-Me)₂-(Me)₂-F-(Me)₃.

A nucleic acid wherein the second strand comprises a 2′ sugar, and abasic modification pattern as follows (5′-3′):
ia-ia-(Me)₈-(F)₃-(Me)₁₀
wherein ia represents an inverted abasic nucleoside.

A nucleic acid wherein the second strand comprises a 2′ sugar, and abasic modification pattern as follows (5′-3′):
ia-ia-(Me)₈-(F)₃-(Me)₁₀, wherein ia represents an inverted abasic nucleoside; and
wherein the first strand comprises a 2′ sugar modification pattern wherein said modifications are selected at least from 2′Me and 2′F sugar modifications, provided that the overall number of 2′F sugar modifications in the first strand does not consist of four, or six, 2′F modifications.

A nucleic acid wherein the second strand comprises a 2′ sugar, and abasic modification pattern as follows (5′-3′):
ia-ia-(Me)₈-(F)₃-(Me)₁₀, wherein ia represents an inverted abasic nucleoside; and
wherein the first strand comprises a 2′ sugar modification pattern wherein said modifications are selected at least from 2′Me and 2′F sugar modifications, wherein the overall number of 2′F sugar modifications in the first strand consists of three, five or seven 2′F modifications.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):
ia-ia-(Me)₈-(F)₃-(Me)₁₀, wherein ia represents an inverted abasic nucleoside; and
wherein nucleosides of said first strand comprise a 2′ sugar modification pattern as follows (5′-3′):
Me-F-(Me)₃-X₁-(Me)₇-F-Me-F-(Me)₇, wherein X₁ is a thermally destabilising modification.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):
ia-ia-(Me)₈-(F)₃-(Me)₁₀, wherein ia represents an inverted abasic nucleoside; and
wherein nucleosides of said first strand comprise a 2′ sugar modification pattern as follows (5′-3′):
(Me-F)₃-(Me)₇-F-Me-F-(Me)₇.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):
ia-ia-(Me)₈-(F)₃-(Me)₁₀, wherein ia represents an inverted abasic nucleoside; and
wherein nucleosides of said first strand comprise a 2′ sugar modification pattern as follows (5′-3′):
Me-F-(Me)₃-F-(Me)₇-(F-Me)₂-F-(Me)₅.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):
ia-ia-(Me)₈-(F)₃-(Me)₁₀, wherein ia represents an inverted abasic nucleoside; and
wherein nucleosides of said first strand comprise a 2′ sugar modification pattern as follows (5′-3′):
Me-F-(Me)₃-F-(Me)₇-F-Me-F-(Me)₃-F-(Me)₃.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):
ia-ia-(Me)₈-(F)₃-(Me)₁₀, wherein ia represents an inverted abasic nucleoside; and
wherein nucleosides of said first strand comprise a 2′ sugar modification pattern as follows (5′-3′):
Me-F-(Me)₃-X₁-Me-(F)₂-(Me)₄-F-Me-F-(Me)₇,
wherein X₁ is a thermally destabilising modification.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):
ia-ia-(Me)₈-(F)₃-(Me)₁₀, wherein ia represents an inverted abasic nucleoside; and
wherein nucleosides of said first strand comprise a 2′ sugar modification pattern as follows (5′-3′):
(Me-F)₃-Me-(F)₂-(Me)₄-(F-Me)₂-(Me)₆.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):
ia-ia-(Me)₈-(F)₃-(Me)₁₀, wherein ia represents an inverted abasic nucleoside; and
wherein nucleosides of said first strand comprise a 2′ sugar modification pattern as follows (5′-3′):
Me-F-(Me)₃-F-Me-(F)₂-(Me)₄-(F-Me)₂-F-(Me)₅.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):
ia-ia-(Me)₈-(F)₃-(Me)₁₀, wherein ia represents an inverted abasic nucleoside; and
wherein nucleosides of said first strand comprise a 2′ sugar modification pattern as follows (5′-3′):
Me-F-(Me)₃-F-Me-(F)₂-(Me)₄-(F-Me)₂-(Me)₂-F-(Me)₃.

A nucleic acid wherein the second strand comprises a 2′ sugar modification pattern as follows (5′-3′):
ia-ia-Me(s)Me(s)(Me)₆-(F)₃-(Me)₁₀.
wherein ia represents an inverted abasic nucleoside, and(s) represents a phosphorothioate linkage.

A nucleic acid wherein the second strand comprises a 2′ sugar modification pattern as follows (5′-3′):
ia-ia-Me(s)Me(s)(Me)₆-(F)₃-(Me)₁₀, wherein ia represents an inverted abasic nucleoside, and(s) represents a phosphorothioate linkage; and
wherein the first strand comprises a 2′ sugar modification pattern wherein said modifications are selected at least from 2′Me and 2′F sugar modifications, provided that the overall number of 2′F sugar modifications in the first strand does not consist of four, or six, 2′F modifications.

A nucleic acid wherein the second strand comprises a 2′ sugar modification pattern as follows (5′-3′):
ia-ia-Me(s)Me(s)(Me)₆-(F)₃-(Me)₁₀, wherein ia represents an inverted abasic nucleoside, and(s) represents a phosphorothioate linkage; and
wherein the first strand comprises a 2′ sugar modification pattern wherein said modifications are selected at least from 2′Me and 2′F sugar modifications, wherein the overall number of 2′F sugar modifications in the first strand consists of three, five or seven 2′F modifications.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):
ia-ia-Me(s)Me(s)(Me)₆-(F)₃-(Me)₁₀, wherein ia represents an inverted abasic nucleoside, and(s) represents a phosphorothioate linkage, and
wherein nucleosides of said first strand comprise a 2′ sugar modification pattern as follows (5′-3′):
Me(s)F(s)(Me)₃-X₁-(Me)₇-F-Me-F-(Me)₅(s)Me(s)Me, wherein X₁ is a thermally destabilising modification.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):
ia-ia-Me(s)Me(s)(Me)₆-(F)₃-(Me)₁₀, wherein ia represents an inverted abasic nucleoside, and(s) represents a phosphorothioate linkage, and
wherein nucleosides of said first strand comprise a 2′ sugar modification pattern as follows (5′-3′):
Me(s)F(s)Me-F-Me-F-(Me)₇-F-Me-F-(Me)₅(s)Me(s)Me.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):
ia-ia-Me(s)Me(s)(Me)₆-(F)₃-(Me)₁₀, wherein ia represents an inverted abasic nucleoside, and(s) represents a phosphorothioate linkage, and
wherein nucleosides of said first strand comprise a 2′ sugar modification pattern as follows (5′-3′):
Me(s)F(s)(Me)₃-F-(Me)₇-(F-Me)₂-F-(Me)₃ (s)Me(s)Me.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):
ia-ia-Me(s)Me(s)(Me)₆-(F)₃-(Me)₁₀, wherein ia represents an inverted abasic nucleoside, and(s) represents a phosphorothioate linkage, and
wherein nucleosides of said first strand comprise a 2′ sugar modification pattern as follows (5′-3′):
Me(s)F(s)(Me)₃-F-(Me)₇-F-Me-F-(Me)₃-F-Me(s)Me(s)Me.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):
ia-ia-Me(s)Me(s)(Me)₆-(F)₃-(Me)₁₀, wherein ia represents an inverted abasic nucleoside, and(s) represents a phosphorothioate linkage, and
wherein nucleosides of said first strand comprise a 2′ sugar modification pattern as follows (5′-3′):
Me(s)F(s)(Me)₃-X₁-Me-(F)₂-(Me)₄-F-Me-F-(Me) s(s)Me(s)Me, wherein X₁ is a thermally destabilising modification.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):
ia-ia-Me(s)Me(s)(Me)₆-(F)₆-(Me)₁₀, wherein ia represents an inverted abasic nucleoside, and(s) represents a phosphorothioate linkage, and
wherein nucleosides of said first strand comprise a 2′ sugar modification pattern as follows (5′-3′):
Me(s)F(s)Me-F-Me-F-Me-(F)₂-(Me)₄-(F-Me)₂-(Me)₄(s)Me(s)Me.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):
ia-ia-Me(s)Me(s)(Me)₆-(F)₃-(Me)₁₀, wherein ia represents an inverted abasic nucleoside, and(s) represents a phosphorothioate linkage, and
wherein nucleosides of said first strand comprise a 2′ sugar modification pattern as follows (5′-3′):
Me(s)F(s)(Me)₃-F-Me-(F)₂-(Me)₄-(F-Me)₂-F-(Me)₃ (s)Me(s)Me.

A nucleic acid comprising a first strand that is at least partially complementary to a portion of RNA transcribed from the target gene, and a second strand that is at least partially complementary to the first strand, wherein said first and second strands form a duplex region of at least 17 nucleosides in length, and wherein nucleosides of said second strand comprise a 2′ sugar, and abasic modification pattern as follows (5′-3′):
ia-ia-Me(s)Me(s)(Me)₆-(F)₃-(Me)₁₀, wherein ia represents an inverted abasic nucleoside, and(s) represents a phosphorothioate linkage, and
wherein nucleosides of said first strand comprise a 2′ sugar modification pattern as follows (5′-3′):
Me(s)F(s)(Me)₃-F-Me-(F)₂-(Me)₄-(F-Me)₂-(Me)₂-F-Me(s)Me(s)Me.

Preferred modifications are as follows:

Modification pattern 1:
Second strand (5′-3′): ia-ia-Me-Me-Me-Me-Me-Me-Me-Me-F—F—F-Me-Me-Me-Me-Me-Me-Me-Me-Me-Me,
First strand (5′-3′): Me-F-Me-Me-Me-X₁-Me-Me-Me-Me-Me-Me-Me-F-Me-F-Me-Me-Me-Me-Me-Me-Me, wherein X₁ is a thermally destabilising modification:

Or Modification pattern 2:
Second strand (5′-3′): ia-ia-Me-Me-Me-Me-Me-Me-Me-Me-F—F—F-Me-Me-Me-Me-Me-Me-Me-Me-Me-Me,
First strand (5′-3′): Me-F-Me-F-Me-F-Me-Me-Me-Me-Me-Me-Me-F-Me-F-Me-Me-Me-Me-Me-Me-Me.

Or Modification pattern 3:
Second strand (5′-3′): ia-ia-Me-Me-Me-Me-Me-Me-Me-Me-F—F—F-Me-Me-Me-Me-Me-Me-Me-Me-Me-Me,
First strand (5′-3′): Me-F-Me-Me-Me-F-Me-Me-Me-Me-Me-Me-Me-F-Me-F-Me-F-Me-Me-Me-Me-Me:

Or Modification pattern 4:
Second strand (5′-3′): ia-ia-Me-Me-Me-Me-Me-Me-Me-Me-F—F—F-Me-Me-Me-Me-Me-Me-Me-Me-Me-Me,
First strand (5′-3′): Me-F-Me-Me-Me-F-Me-Me-Me-Me-Me-Me-Me-F-Me-F-Me-Me-Me-F-Me-Me-Me:

Or Modification pattern 5:
Second strand (5′-3′): ia-ia-Me-Me-Me-Me-Me-Me-Me-Me-F—F—F-Me-Me-Me-Me-Me-Me-Me-Me-Me-Me,
First strand (5′-3′): Me-F-Me-Me-Me-X₁-Me-F—F-Me-Me-Me-Me-F-Me-F-Me-Me-Me-Me-Me-Me-Me, wherein X₁ is a thermally destabilising modification;

Or Modification pattern 6:
Second strand (5′-3′): ia-ia-Me-Me-Me-Me-Me-Me-Me-Me-F—F—F-Me-Me-Me-Me-Me-Me-Me-Me-Me-Me,
First strand (5′-3′): Me-F-Me-F-Me-F-Me-F—F-Me-Me-Me-Me-F-Me-F-Me-Me-Me-Me-Me-Me-Me:

Or Modification pattern 7:
Second strand (5′-3′): ia-ia-Me-Me-Me-Me-Me-Me-Me-Me-F—F—F-Me-Me-Me-Me-Me-Me-Me-Me-Me-Me,
First strand (5′-3′): Me-F-Me-Me-Me-F-Me-F—F-Me-Me-Me-Me-F-Me-F-Me-F-Me-Me-Me-Me-Me;

Or Modification pattern 8:
Second strand (5′-3′): ia-ia-Me-Me-Me-Me-Me-Me-Me-Me-F—F—F-Me-Me-Me-Me-Me-Me-Me-Me-Me-Me,
First strand (5′-3′): Me-F-Me-Me-Me-F-Me-F—F-Me-Me-Me-Me-F-Me-F-Me-Me-Me-F-Me-Me-Me
wherein ia represents an inverted abasic nucleoside.

Further preferred modifications are as follows:

Modification pattern 1:
Second strand (5′-3′): ia-ia-Me(s)Me(s)Me-Me-Me-Me-Me-Me-F—F—F-Me-Me-Me-Me-Me-Me-Me-Me-Me-Me,
First strand (5′-3′): Me(s)F(s)Me-Me-Me-X₁-Me-Me-Me-Me-Me-Me-Me-F-Me-F-Me-Me-Me-Me-Me(s)Me(s)Me, wherein X₁ is a thermally destabilising modification;

Or Modification pattern 2:
Second strand (5′-3′): ia-ia-Me(s)Me(s)Me-Me-Me-Me-Me-Me-F—F—F-Me-Me-Me-Me-Me-Me-Me-Me-Me-Me,
First strand (5′-3′): Me(s)F(s)Me-F-Me-F-Me-Me-Me-Me-Me-Me-Me-F-Me-F-Me-Me-Me-Me-Me(s)Me(s)Me;

Or Modification pattern 3:
Second strand (5′-3′): ia-ia-Me(s)Me(s)Me-Me-Me-Me-Me-Me-F—F—F-Me-Me-Me-Me-Me-Me-Me-Me-Me-Me,
First strand (5′-3′): Me(s)F(s)Me-Me-Me-F-Me-Me-Me-Me-Me-Me-Me-F-Me-F-Me-F-Me-Me-Me(s)Me(s)Me:

Or Modification pattern 4:
Second strand (5′-3′): ia-ia-Me(s)Me(s)Me-Me-Me-Me-Me-Me-F—F—F-Me-Me-Me-Me-Me-Me-Me-Me-Me-Me,
First strand (5′-3′): Me(s)F(s)Me-Me-Me-F-Me-Me-Me-Me-Me-Me-Me-F-Me-F-Me-Me-Me-F-Me(s)Me(s)Me:

Or Modification pattern 5:
Second strand (5′-3′): ia-ia-Me(s)Me(s)Me-Me-Me-Me-Me-Me-F—F—F-Me-Me-Me-Me-Me-Me-Me-Me-Me-Me,
First strand (5′-3′): Me(s)F(s)Me-Me-Me-X₁-Me-F—F-Me-Me-Me-Me-F-Me-F-Me-Me-Me-Me-Me(s)Me(s)Me, wherein X₁ is a thermally destabilising modification;

Or Modification pattern 6:
Second strand (5′-3′): ia-ia-Me(s)Me(s)Me-Me-Me-Me-Me-Me-F—F—F-Me-Me-Me-Me-Me-Me-Me-Me-Me-Me,
First strand (5′-3′): Me(s)F(s)Me-F-Me-F-Me-F—F-Me-Me-Me-Me-F-Me-F-Me-Me-Me-Me-Me(s)Me(s)Me:

Or Modification pattern 7:
Second strand (5′-3′): ia-ia-Me(s)Me(s)Me-Me-Me-Me-Me-Me-F—F—F-Me-Me-Me-Me-Me-Me-Me-Me-Me-Me,
First strand (5′-3′): Me(s)F(s)Me-Me-Me-F-Me-F—F-Me-Me-Me-Me-F-Me-F-Me-F-Me-Me-Me(s)Me(s)Me:

Or Modification pattern 8:
Second strand (5′-3′): ia-ia-Me(s)Me(s)Me-Me-Me-Me-Me-Me-F—F—F-Me-Me-Me-Me-Me-Me-Me-Me-Me-Me,
First strand (5′-3′): Me(s)F(s)Me-Me-Me-F-Me-F—F-Me-Me-Me-Me-F-Me-F-Me-Me-Me-F-Me(s)Me(s)Me:
wherein(s) is a phosphorothioate internucleoside linkage and ia represents an inverted abasic nucleoside.

### Conjugation

Another modification of the nucleic acid e.g. RNA e.g. an siRNA of the invention involves linking the nucleic acid e.g. the siRNA to one or more ligand moieties e.g. to enhance the activity, cellular distribution, or cellular uptake of the nucleic acid e.g. siRNA e.g. into a cell.

In some embodiments, the ligand moiety described can be attached to a nucleic acid, e.g., an siRNA oligonucleoside, via a linker that can be cleavable or non-cleavable. The term “linker” or “linking group” means an organic moiety that connects two parts of a compound, e.g., covalently attaches two parts of a compound.

The ligand can be attached to the 3′ or 5′ end of the sense strand.

The ligand is preferably conjugated to 3′ end of the sense strand of the nucleic acid e.g. an siRNA agent.

The invention therefore relates in a further aspect to a conjugate for inhibiting expression of a target gene in a cell, said conjugate comprising a nucleic acid portion and one or more ligand moieties, said nucleic acid portion comprising a nucleic acid as disclosed herein.

In one aspect the second strand of the nucleic acid is conjugated directly or indirectly (e.g. via a linker) to the one or more ligand moiety(s), wherein said ligand moiety is typically present at a terminal region of the second strand, preferably at the 3′ terminal region thereof.

In certain embodiments, the ligand moiety comprises a GalNAc or GalNAc derivative attached to the nucleic acid e.g. dsiRNA through a linker.

Therefore the invention relates to a conjugate wherein the ligand moiety comprises
i) one or more GalNAc ligands; and/or
ii) one or more GalNAc ligand derivatives; and/or
iii) one or more GalNAc ligands conjugated to said nucleic acid through a linker.

Said GalNAc ligand may be conjugated directly or indirectly to the 5′ or 3′ terminal region of the second strand of the nucleic acid, preferably at the 3′ terminal region thereof.

GalNAc ligands are well known in the art and described in, inter alia, EP3775207A1.

In some embodiments, the GalNAc ligand is comprised in any one of the linkers shown in FIGS. 1-4 or FIG. 5 (Formula XI), wherein the “oligonucleotide” may be any nucleic acid disclosed herein. Accordingly, the “oligonucleotide” may comprise other bonds than a phosphodiester bond, such as one or more phosphorothioate bonds. Preferably, the nucleic acid according to the invention is a double stranded oligonucleoside as defined herein and the linker is conjugated to the second strand, more preferably to the 3′ terminal region of the second strand, via a phosphodiester bond.

In some embodiments, the GalNAc ligand is comprised in the linker shown in FIG. 3, wherein the “oligonucleotide” may be any nucleic acid disclosed herein. Accordingly, the “oligonucleotide” may comprise other bonds than a phosphodiester bond, such as one or more phosphorothioate bonds. Preferably, the nucleic acid according to the invention is a double stranded oligonucleoside as defined herein and the linker is conjugated to the second strand, more preferably to the 3′ terminal region of the second strand, via a phosphodiester bond.

In some embodiments, the GalNAc ligand is comprised in the linker shown in FIG. 5 (Formula XI), wherein the “oligonucleotide” may be any nucleic acid disclosed herein. Accordingly, the “oligonucleotide” may comprise other bonds than a phosphodiester bond, such as one or more phosphorothioate bonds. Preferably, the nucleic acid according to the invention is a double stranded oligonucleoside as defined herein and the linker is conjugated to the second strand, more preferably to the 3′ terminal region of the second strand, via a phosphodiester bond.

In some embodiments, the GalNAc ligand is comprised in any one of the linkers shown in FIGS. 1-4 or FIG. 5 (Formula XI), wherein the “oligonucleotide” represents a nucleic acid according to the invention, wherein the nucleic acid according to the invention comprises a modified or unmodified second strand comprising or consisting of SEQ ID NO: 265 or SEQ ID NO: 268, preferably wherein the linker is conjugated to the 3′ terminal region of the second strand, i.e., to the 3′ terminal region of SEQ ID NO: 265 or SEQ ID NO: 268, via a phosphodiester bond.

In some embodiments, the GalNAc ligand is comprised in the linker shown in FIG. 3, wherein the “oligonucleotide” represents a nucleic acid according to the invention, wherein the nucleic acid according to the invention comprises a modified or unmodified second strand comprising or consisting of SEQ ID NO: 265 or SEQ ID NO: 268, preferably wherein the linker is conjugated to the 3′ terminal region of the second strand, i.e., to the 3′ terminal region of SEQ ID NO: 265 or SEQ ID NO: 268, via a phosphodiester bond.

In some embodiments, the GalNAc ligand is comprised in the linker shown in FIG. 5 (Formula XI), wherein the “oligonucleotide” represents a nucleic acid according to the invention, wherein the nucleic acid according to the invention comprises a modified or unmodified second strand comprising or consisting of SEQ ID NO: 265 or SEQ ID NO: 268, preferably wherein the linker is conjugated to the 3′ terminal region of the second strand, i.e., to the 3′ terminal region of SEQ ID NO: 265 or SEQ ID NO: 268, via a phosphodiester bond.

In some embodiments, the GalNAc ligand is comprised in any one of the linkers shown in FIGS. 1-4 or FIG. 5 (Formula XI), wherein the “oligonucleotide” represents a nucleic acid according to the invention, wherein the nucleic acid according to the invention comprises a modified second strand comprising or consisting of SEQ ID NO: 774 or SEQ ID NO: 776, preferably wherein the linker is conjugated to the 3′ terminal region of the second strand, i.e., to the 3′ terminal region of SEQ ID NO: 774 or SEQ ID NO: 776, via a phosphodiester bond.

In some embodiments, the GalNAc ligand is comprised in the linker shown in FIG. 3, wherein the “oligonucleotide” represents a nucleic acid according to the invention, wherein the nucleic acid according to the invention comprises a modified second strand comprising or consisting of SEQ ID NO: 774 or SEQ ID NO: 776, preferably wherein the linker is conjugated to the 3′ terminal region of the second strand, i.e., to the 3′ terminal region of SEQ ID NO: 774 or SEQ ID NO: 776, via a phosphodiester bond.

In some embodiments, the GalNAc ligand is comprised in the linker shown in FIG. 5 (Formula XI), wherein the “oligonucleotide” represents a nucleic acid according to the invention, wherein the nucleic acid according to the invention comprises a modified second strand comprising or consisting of SEQ ID NO: 774 or SEQ ID NO: 776, preferably wherein the linker is conjugated to the 3′ terminal region of the second strand, i.e., to the 3′ terminal region of SEQ ID NO: 774 or SEQ ID NO: 776, via a phosphodiester bond.

In some embodiments, the GalNAc ligand is comprised in any one of the linkers shown in FIGS. 1-4 or FIG. 5 (Formula XI), wherein the “oligonucleotide” represents a nucleic acid according to the invention, wherein the nucleic acid according to the invention comprises a modified first strand comprising or consisting of SEQ ID NO: 764 and a modified second strand comprising or consisting of SEQ ID NO: 774, preferably wherein the linker is conjugated to the 3′ terminal region of the second strand, i.e., to the 3′ terminal region of SEQ ID NO: 774, via a phosphodiester bond.

In some embodiments, the GalNAc ligand is comprised in the linker shown in FIG. 3, wherein the “oligonucleotide” represents a nucleic acid according to the invention, wherein the nucleic acid according to the invention comprises a modified first strand comprising or consisting of SEQ ID NO: 764 and a modified second strand comprising or consisting of SEQ ID NO: 774, preferably wherein the linker is conjugated to the 3′ terminal region of the second strand, i.e., to the 3′ terminal region of SEQ ID NO: 774, via a phosphodiester bond.

In some embodiments, the GalNAc ligand is comprised in the linker shown in FIG. 5 (Formula XI), wherein the “oligonucleotide” represents a nucleic acid according to the invention, wherein the nucleic acid according to the invention comprises a modified first strand comprising or consisting of SEQ ID NO: 764 and a modified second strand comprising or consisting of SEQ ID NO: 774, preferably wherein the linker is conjugated to the 3′ terminal region of the second strand, i.e., to the 3′ terminal region of SEQ ID NO: 774, via a phosphodiester bond.

In some embodiments, the GalNAc ligand is comprised in any one of the linkers shown in FIGS. 1-4 or FIG. 5 (Formula XI), wherein the “oligonucleotide” represents a nucleic acid according to the invention, wherein the nucleic acid according to the invention comprises a modified first strand comprising or consisting of SEQ ID NO: 766 and a modified second strand comprising or consisting of SEQ ID NO: 776, preferably wherein the linker is conjugated to the 3′ terminal region of the second strand, i.e., to the 3′ terminal region of SEQ ID NO: 776, via a phosphodiester bond.

In some embodiments, the GalNAc ligand is comprised in the linker shown in FIG. 3, wherein the “oligonucleotide” represents a nucleic acid according to the invention, wherein the nucleic acid according to the invention comprises a modified first strand comprising or consisting of SEQ ID NO: 766 and a modified second strand comprising or consisting of SEQ ID NO: 776, preferably wherein the linker is conjugated to the 3′ terminal region of the second strand, i.e., to the 3′ terminal region of SEQ ID NO: 776, via a phosphodiester bond.

In some embodiments, the GalNAc ligand is comprised in the linker shown in FIG. 5 (Formula XI), wherein the “oligonucleotide” represents a nucleic acid according to the invention, wherein the nucleic acid according to the invention comprises a modified first strand comprising or consisting of SEQ ID NO: 766 and a modified second strand comprising or consisting of SEQ ID NO: 776, preferably wherein the linker is conjugated to the 3′ terminal region of the second strand, i.e., to the 3′ terminal region of SEQ ID NO: 776, via a phosphodiester bond.

In some embodiments, the GalNAc ligand is comprised in the linker shown in FIG. 3, wherein the “oligonucleotide” represents a nucleic acid according to the invention, wherein the nucleic acid according to the invention comprises a modified first strand comprising or consisting of SEQ ID NO: 764 and a modified second strand comprising or consisting of SEQ ID NO: 774, wherein the second strand has the following structure:

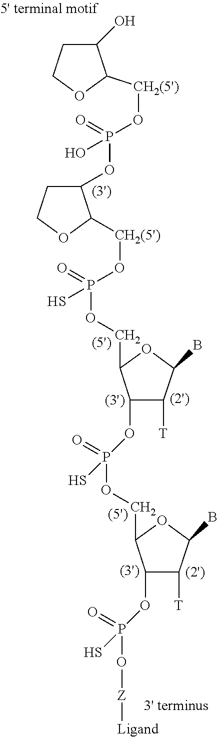

wherein:
T represents a 2′Me ribose modification,
B represents the nucleoside bases of the first two basic nucleosides in the 5′ terminal region of SEQ ID NO: 774, and
Z represents the remaining 19 contiguous basic nucleosides of SEQ ID NO: 774.

In some embodiments, the GalNAc ligand is comprised in the linker shown in FIG. 5 (Formula XI), wherein the “oligonucleotide” represents a nucleic acid according to the invention, wherein the nucleic acid according to the invention comprises a modified first strand comprising or consisting of SEQ ID NO: 764 and a modified second strand comprising or consisting of SEQ ID NO: 774, wherein the second strand has the following structure:

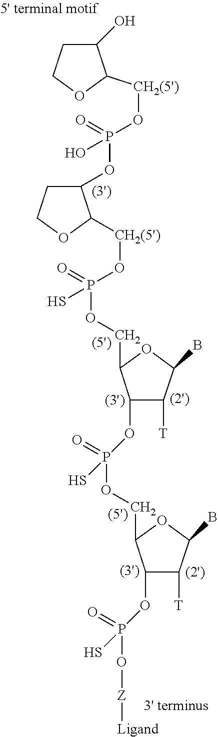

wherein:
T represents a 2′Me ribose modification,
B represents the nucleoside bases of the first two basic nucleosides in the 5′ terminal region of SEQ ID NO: 774, and
Z represents the remaining 19 contiguous basic nucleosides of SEQ ID NO: 774.

In some embodiments, the GalNAc ligand is comprised in the linker shown in FIG. 3, wherein the “oligonucleotide” represents a nucleic acid according to the invention, wherein the nucleic acid according to the invention comprises a modified first strand comprising or consisting of SEQ ID NO: 766 and a modified second strand comprising or consisting of SEQ ID NO: 776, wherein the second strand has the following structure:

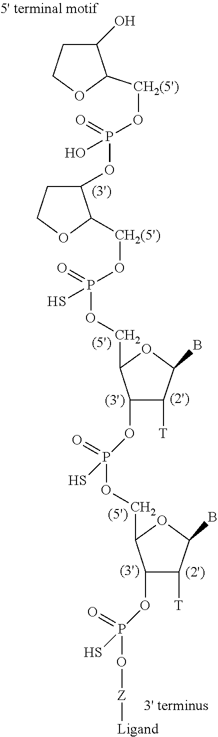

wherein:
T represents a 2′Me ribose modification,
B represents the nucleoside bases of the first two basic nucleosides in the 5′ terminal region of SEQ ID NO: 776, and
Z represents the remaining 19 contiguous basic nucleosides of SEQ ID NO: 776.

In some embodiments, the GalNAc ligand is comprised in the linker shown in FIG. 5 (Formula XI), wherein the “oligonucleotide” represents a nucleic acid according to the invention, wherein the nucleic acid according to the invention comprises a modified first strand comprising or consisting of SEQ ID NO: 766 and a modified second strand comprising or consisting of SEQ ID NO: 776, wherein the second strand has the following structure:

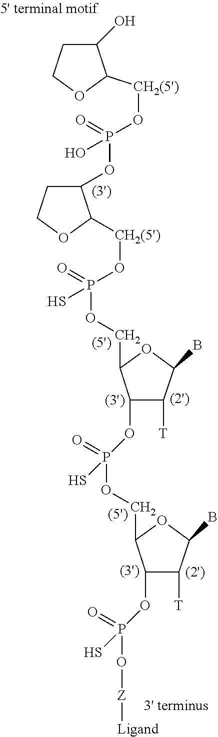

wherein:
T represents a 2′Me ribose modification,
B represents the nucleoside bases of the first two basic nucleosides in the 5′ terminal region of SEQ ID NO: 776, and
Z represents the remaining 19 contiguous basic nucleosides of SEQ ID NO: 776.

### Vector and Cell

In one aspect, the invention provides a cell containing a nucleic acid, such as inhibitory RNA [RNAi] as described herein.

In one aspect, the invention provides a cell comprising a vector as described herein.

### Pharmaceutically Acceptable Compositions

In one aspect, the invention provides a pharmaceutical composition for inhibiting expression of a target gene, the composition comprising a nucleic acid as disclosed herein.

The pharmaceutically acceptable composition may comprise an excipient and or carrier.

Some examples of materials which can serve as pharmaceutically-acceptable carriers include: (1) sugars, such as lactose, glucose and sucrose: (2) starches, such as corn starch and potato starch: (3) cellulose, and its derivatives, such as sodium carboxymethyl cellulose, ethyl cellulose and cellulose acetate: (4) powdered tragacanth: (5) malt: (6) gelatin: (7) lubricating agents, such as magnesium stearate, sodium lauryl sulfate and talc: (8) excipients, such as cocoa butter and suppository waxes: (9) oils, such as peanut oil, cottonseed oil, safflower oil, sesame oil, olive oil, corn oil and soybean oil: (10) glycols, such as propylene glycol: (11) polyols, such as glycerin, sorbitol, mannitol and polyethylene glycol: (12) esters, such as ethyl oleate and ethyl laurate: (13) agar: (14) buffering agents, such as magnesium hydroxide and aluminum hydroxide: (15) alginic acid: (16) pyrogen-free water: (17) isotonic saline: (18) Ringer's solution: (19) ethyl alcohol: (20) pH buffered solutions: (21) polyesters, polycarbonates and/or poly anhydrides: (22) bulking agents, such as polypeptides and amino acids (23) serum component, such as serum albumin, HDL and LDL; and (22) other non-toxic compatible substances employed in pharmaceutical formulations.

Typical pharmaceutical carriers include, but are not limited to, binding agents (e.g., pregelatinized maize starch, polyvinylpyrrolidone or hydroxypropyl methylcellulose, etc.): fillers (e.g., lactose and other sugars, microcrystalline cellulose, pectin, gelatin, calcium sulfate, ethyl cellulose, polyacrylates or calcium hydrogen phosphate, etc.): lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.): disintegrants (e.g., starch, sodium starch glycolate, etc.); and wetting agents (e.g., sodium lauryl sulphate, etc.).

Pharmaceutically acceptable organic or inorganic excipients suitable for non-parenteral administration which do not deleteriously react with nucleic acids can also be used to formulate the compositions of the present invention. Suitable pharmaceutically acceptable excipients include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone, and the like.

Formulations for topical administration of nucleic acids can include sterile and non-sterile aqueous solutions, non-aqueous solutions in common solvents such as alcohols, or solutions of the nucleic acids in liquid or solid oil bases. The solutions can also contain buffers, diluents and other suitable additives. Pharmaceutically acceptable organic or inorganic excipients suitable for non-parenteral administration which do not deleteriously react with nucleic acids can be used.

In one embodiment, the nucleic acid or composition is administered in an unbuffered solution. In certain embodiments, the unbuffered solution is saline or water. In other embodiments, the nucleic acid e.g. siRNA agent is administered in a buffered solution. In such embodiments, the buffer solution can comprise acetate, citrate, prolamine, carbonate, or phosphate, or any combination thereof. For example, the buffer solution can be phosphate buffered saline (PBS).

### Dosages

The pharmaceutical compositions of the invention may be administered in dosages sufficient to inhibit expression of a gene. In general, a suitable dose of a nucleic acid e.g. an siRNA of the invention will be in the range of about 0.001 to about 200.0 milligrams per kilogram body weight of the recipient per day, generally in the range of about 1 to 50 mg per kilogram body weight per day. Typically, a suitable dose of a nucleic acid e.g. an siRNA of the invention will be in the range of about 0.1 mg/kg to about 5.0 mg/kg. e.g., about 0.3 mg/kg and about 3.0 mg/kg.

A repeat-dose regimen may include administration of a therapeutic amount of a nucleic acid e.g. siRNA on a regular basis, such as every other day or once a year. In certain embodiments, the nucleic acid e.g. siRNA is administered about once per month to about once per quarter (i.e., about once every three months).

In various embodiments, the nucleic acid e.g. siRNA agent is administered at a dose of about 0.01 mg/kg to about 10 mg/kg or about 0.5 mg/kg to about 50 mg/kg. In some embodiments, the nucleic acid e.g. siRNA agent is administered at a dose of about 10 mg/kg to about 30 mg/kg. In certain embodiments, the nucleic acid e.g. siRNA agent is administered at a dose selected from about 0.5 mg/kg 1 mg/kg. 1.5 mg/kg. 3 mg/kg. 5 mg/kg. 10 mg/kg, and 30) mg/kg. In certain embodiments, the nucleic acid e.g. agent is administered about once per week. once per month, once every other two months, or once a quarter (i.e., once every three months) at a dose of about 0.1 mg/kg to about 5.0 mg/kg. In certain embodiments, the nucleic acid e.g. siRNA agent is administered to the subject once a week. In certain embodiments, the nucleic acid e.g. siRNA agent is administered to the subject once a month. In certain embodiments, the nucleic acid e.g. siRNA agent is administered once per quarter (i.e., every three months).

After an initial treatment regimen, the treatments can be administered on a less frequent basis. For example, after administration weekly or biweekly for three months, administration can be repeated once per month, for six months, or a year: or longer.

The pharmaceutical composition can be administered once daily, or administered as two, three, or more sub-doses at appropriate intervals throughout the day or even using continuous infusion or delivery through a controlled release formulation. In that case, the nucleic acid e.g. siRNA contained in each sub-dose must be correspondingly smaller in order to achieve the total daily dosage. The dosage unit can also be compounded for delivery over several days, e.g., using a conventional sustained release formulation which provides sustained release of the nucleic acid e.g. siRNA over a several day period. Sustained release formulations are well known in the art and are particularly useful for delivery of agents at a particular site, such as could be used with the agents of the present invention. In this embodiment, the dosage unit contains a corresponding multiple of the daily dose.

In other embodiments, a single dose of the pharmaceutical compositions can be long lasting, such that subsequent doses are administered at not more than 3, 4, or 5 day intervals, or at not more than 1, 2, 3, or 4 week intervals. In some embodiments of the invention, a single dose of the pharmaceutical compositions of the invention is administered once per week. In other embodiments of the invention, a single dose of the pharmaceutical compositions of the invention is administered bimonthly. In certain embodiments, the siRNA is administered about once per month to about once per quarter (i.e., about once every three months), or even every 6 months or 12 months.

Estimates of effective dosages and in vivo half-lives for the individual nucleic acid e.g. siRNAs encompassed by the invention can be made using conventional methodologies or on the basis of in vivo testing using an appropriate animal model, as known in the art.

The pharmaceutical compositions of the present invention can be administered in a number of ways depending upon whether local or systemic treatment is desired and upon the area to be treated. Administration can be topical (e.g., by a transdermal patch), pulmonary, e.g., by inhalation or insufflation of powders or aerosols, including by nebulizer: intratracheal, intranasal, epidermal and transdermal, oral or parenteral. Parenteral administration includes intravenous, intraarterial, subcutaneous, intraperitoneal, or intramuscular injection or infusion: subdermal, e.g., via an implanted device: or intracranial, e.g., by intraparenchymal, intrathecal or intraventricular administration. In certain preferred embodiments, the compositions are administered by intravenous infusion or injection. In certain embodiments, the compositions are administered by subcutaneous injection.

In one embodiment, the nucleic acid e.g. agent is administered to the subject subcutaneously.

The nucleic acid e.g. siRNA can be delivered in a manner to target a particular tissue (e.g. in particular liver cells).

### Methods for Inhibiting ZPI Gene Expression

The present invention also provides methods of inhibiting expression of ZPI gene in a cell. The methods include contacting a cell with a nucleic acid of the invention e.g. siRNA agent, such as double stranded siRNA agent, in an amount effective to inhibit expression of the ZPI gene in the cell, thereby inhibiting expression of the ZPI gene in the cell. It is to be noted that a nucleic acid “for inhibiting the expression of ZPI” is a nucleic acid that is capable of inhibiting ZPI expression, preferably as described herein below.

Contacting of a cell with the nucleic acid e.g. an siRNA, such as a double stranded siRNA agent, may be done in vitro or in vivo. Contacting a cell in vivo with nucleic acid e.g. includes contacting a cell or group of cells within a subject, e.g., a human subject, with the nucleic acid e.g. siRNA. Combinations of in vitro and in vivo methods of contacting a cell are also possible. Contacting a cell may be direct or indirect, as discussed above. Furthermore, contacting a cell may be accomplished via a targeting ligand moiety, including any ligand moiety described herein or known in the art. In preferred embodiments, the targeting ligand moiety is a carbohydrate moiety, e.g. a GalNAc3 ligand, or any other ligand moiety that directs the siRNA agent to a site of interest.

The term “inhibiting,” as used herein, is used interchangeably with “reducing,” “silencing,” “downregulating”, “suppressing”, and other similar terms, and includes any level of inhibition.

In some embodiments of the methods of the invention, expression of ZPI gene is inhibited by at least 30%, 35%, 40%, 45%, 50%, 55%, 60%, 65%, 70%, 75%, 80%, 85%, 90%, or 95%, or to below the level of detection of the assay, preferably when determined by qPCR as described herein and/or when the siRNA is introduced into the target cell by transfection. In certain embodiments, the methods include a clinically relevant inhibition of expression of ZPI target gene e.g. as demonstrated by a clinically relevant outcome after treatment of a subject with an agent to reduce the expression of the gene.

In some embodiments, when transfected into the cells, the nucleic acid of the invention inhibits expression of the ZPI gene with an IC50 value lower than 2500 pM, 2400 pM, 2300 pM, 2200 pM, 2100 pM, 2000 pM, 1900 pM, 1800 pM, 1700 pM, 1600 pM, 1500 pM, 1400 pM, 1300 pM, 1200 pM, 1100 pM, 1000 pM, 900 pM, 800 pM, 700 pM, 600 pM, 500 pM, 400 pM, 300 pM, 200 pM or 100 pM, preferably when determined by qPCR, more preferably by reverse transcriptase (RT)-qPCR, as described herein.

In a preferred embodiment, when transfected into the cells, the nucleic acid of the invention inhibits expression of the ZPI gene with an IC50 value lower than 2500 pM. In a more preferred embodiment, when transfected into the cells, the nucleic acid of the invention inhibits expression of the ZPI gene with an IC50 value lower than 1000 pM. In an even more preferred embodiment, when transfected into the cells, the nucleic acid of the invention inhibits expression of the ZPI gene with an IC50 value lower than 500 pM. In a most preferred embodiment, when transfected into the cells, the nucleic acid of the invention inhibits expression of the ZPI gene with an IC50 value lower than 100 pM.

Inhibition of expression of the ZPI gene may be quantified by the following method:

Huh7 cells (human hepatocyte-derived cell line, obtained from JCRB Cell Bank) may be maintained in Dulbecco's Modified Eagle Medium (DMEM) supplemented with 10% FBS at 37° C. in an atmosphere of 5% CO₂. Cells may then be transfected with siRNA duplexes targeting ZPI mRNA or a negative control siRNA (siRNA-control: sense strand 5′-UUCUCCGAACGUGUCACGUTT-3′ (SEQ ID NO: 794), antisense strand 5′-ACGUGACACGUUCGGAGAATT-3′ (SEQ ID NO: 790)) using 10×3-fold serial dilutions over a final duplex concentration range of 20 nM to 1 pM. Transfection may be carried out by adding 9.7 μL Opti-MEM (ThermoFisher) plus 0.3 μL Lipofectamine RNAiMAX (ThermoFisher) to 10 μL of each siRNA duplex. The mixture may be incubated at room temperature for 15 minutes before being added to 100 μL of complete growth medium containing 20,000 Huh7 cells. Cells may be incubated for 24 hours at 37° C./5% CO₂ prior to total RNA purification using a RNeasy 96 Kit (Qiagen). Each duplex may be tested by transfection in duplicate wells in a single experiment.

cDNA synthesis may be performed using FastQuant RT (with gDNase) Kit (Tiangen). Real-time quantitative PCR (qPCR) may be performed on an ABI Prism 7900HT or ABI QuantStudio 7 with primers specific for human ZPI (Hs01547819_m1) and human GAPDH (Hs02786624_g1) using FastStart Universal Probe Master Kit (Roche).

qPCR may be performed in duplicate on cDNA derived from each well and the mean cycle threshold (Ct) calculated. Relative ZPI expression may be calculated from mean Ct values using the comparative Ct (ΔΔCt) method, normalised to GAPDH and relative to untreated cells. Maximum percent inhibition of ZPI expression and IC50 values may be calculated using a four parameter (variable slope) model using GraphPad Prism 9.

Alternatively or in addition, inhibition of expression of the ZPI gene may be characterized by a reduction of mean relative expression of the ZPI gene.

In some embodiments, when cells are transfected with 0.1 nM of the nucleic acid of the invention, the mean relative expression of ZPI is below 1, 0.9, 0.8, 0.7, 0.6, 0.5, or 0.4, preferably when determined by qPCR, more preferably by reverse transcriptase (RT)-qPCR, as described herein.

In some embodiments, when cells are transfected with 5 nM of the nucleic acid of the invention, the mean relative expression of ZPI is below 1, 0.9, 0.8, 0.7, 0.6, 0.5, 0.4 or 0.3, preferably when determined by qPCR, more preferably by reverse transcriptase (RT)-qPCR, as described herein.

Mean relative expression of the ZPI gene may be quantified by the following method:

Huh7 cells (human hepatocyte-derived cell line, obtained from JCRB Cell Bank) may be maintained in Dulbecco's Modified Eagle Medium (DMEM) supplemented with 10% FBS at 37° C. in at atmosphere of 5% CO₂. Cells may be transfected with siRNA duplexes targeting ZPI mRNA or a negative control siRNA (siRNA-control: sense strand 5′-UUCUCCGAACGUGUCACGUTT-3′ (SEQ ID NO: 794), antisense strand 5′-ACGUGACACGUUCGGAGAATT-3′ (SEQ ID NO: 790)) at a final duplex concentration of 5 nM and 0.1 nM. Transfection may be carried out by adding 9.7 μL Opti-MEM (ThermoFisher) plus 0.3 μL Lipofectamine RNAiMAX (ThermoFisher) to 10 μL of each siRNA duplex. The mixture may be incubated at room temperature for 15 minutes before being added to 100 μL of complete growth medium containing 20,000 Huh7 cells. Cells may be incubated for 24 hours at 37° C./5% CO₂ prior to total RNA purification using a RNeasy 96 Kit (Qiagen). Each duplex may be tested by transfection in duplicate wells in two independent experiments.

cDNA synthesis may be performed using FastQuant RT (with gDNase) Kit (Tiangen). Real-time quantitative PCR (qPCR) may be performed on an ABI Prism 7900HT or ABI QuantStudio 7 with primers specific for human ZPI (Hs01547819_m1) and human GAPDH (Hs02786624_g1) using FastStart Universal Probe Master Kit (Roche).

qPCR may be performed in duplicate on cDNA derived from each well and the mean Ct calculated. Relative ZPI expression may be calculated from mean Ct values using the comparative Ct (ΔΔCt) method, normalised to GAPDH and relative to untreated cells.

Inhibition of the expression of ZPI gene may be manifested by a reduction of the amount of mRNA of the target ZPI gene in comparison to a suitable control.

In other embodiments, inhibition of the expression of ZPI gene may be assessed in terms of a reduction of a parameter that is functionally linked to gene expression, e.g., protein expression or signaling pathways.

Methods of Treating or Preventing Diseases Associated with ZPI Gene Expression

The present invention also provides methods of using nucleic acid e.g. an siRNA of the invention or a composition containing nucleic acid e.g. an siRNA of the invention to reduce or inhibit ZPI gene expression in a cell. The methods include contacting the cell with a nucleic acid e.g., dsiRNA of the invention and maintaining the cell for a time sufficient to obtain degradation of the mRNA transcript of ZPI, thereby inhibiting expression of the ZPI gene in the cell. Reduction in gene expression can be assessed by any methods known in the art.

In the methods of the invention the cell may be contacted in vitro or in vivo, i.e., the cell may be within a subject.

A cell suitable for treatment using the methods of the invention may be any cell that expresses a gene of interest associated with disease related to a disorder of haemostasis, such as a disease related to a disorder of haemostasis, such as haemophilia.

The in vivo methods of the invention may include administering to a subject a composition containing a nucleic acid of the invention, e.g., an siRNA, where the nucleic acid, e.g., siRNA includes a nucleoside sequence that is complementary to at least a part of an RNA transcript of ZPI gene of the mammal to be treated.

The present invention further provides methods of treatment of a subject in need thereof. The treatment methods of the invention include administering a nucleic acid such as an siRNA of the invention to a subject, e.g., a subject that would benefit from a reduction or inhibition of the expression of ZPI gene, in a therapeutically effective amount e.g. a nucleic acid such as an siRNA targeting ZPI or a pharmaceutical composition comprising the nucleic acid targeting ZPI. The disease to be treated is related to a disorder of haemostasis, such as a disease related to a disorder of haemostasis, such as haemophilia.

Haemophilia, or hemophilia is a mostly inherited genetic disorder that impairs the body's ability to make blood clots, a process needed to stop bleeding. This results in subjects bleeding for a longer time after an injury, easy bruising, and an increased risk of bleeding inside joints or the brain. Subjects with a mild case of the disease may have symptoms only after an accident or during surgery. Bleeding into a joint, also referred to as haemarthrosis, can result in permanent damage while bleeding in the brain can result in long term headaches, seizures, or a decreased level of consciousness.

There are two main types of haemophilia: haemophilia A, which occurs due to low amounts of clotting factor VIII, and haemophilia B, which occurs due to low levels of clotting factor IX. They are typically inherited from one's parents through an X chromosome carrying a nonfunctional gene. Rarely a new mutation may occur during early development or haemophilia may develop later in life due to antibodies forming against a clotting factor. Other types include haemophilia C, which occurs due to low levels of factor XI, Von Willebrand disease, which occurs due to low levels of a substance called von Willebrand factor, and parahaemophilia, which occurs due to low levels of factor V. Haemophilia A, B, and C prevent the intrinsic pathway from functioning properly: this clotting pathway is necessary when there is damage to the endothelium of a blood vessel. Acquired haemophilia is associated with cancers, autoimmune disorders, and pregnancy. Diagnosis is by testing the blood for its ability to clot and its levels of clotting factors.

In certain embodiments, the nucleic acid of the present invention is suitable for treatment, or for treatment of haemophilia A, B and/or C. In certain embodiments, the nucleic acid of the present invention is suitable for treatment, or for treatment of haemophilia A and/or B. In certain embodiments, the nucleic acid of the present invention is suitable for treatment, or for treatment of acquired haemophilia. In certain embodiments, the nucleic acid of the present invention is suitable for treatment, or for treatment of Willebrand disease. In certain embodiments, the nucleic acid of the present invention is suitable for treatment, or for treatment of parahaemophilia.

Without wishing to being bound by theory, treatment with the nucleic acid of the invention results in a boost of clotting factor levels such that bleeding can be reduced or prevented, as demonstrated herein in FIG. 12. Thus, in a preferred embodiment, treatment with the nucleic acid of the invention reduces or prevents bleeding episodes in a subject suffering from haemophilia. In another preferred embodiment, treatment with the nucleic acid of the invention reduces or prevents bleeding into a joint of a subject suffering from haemophilia. In certain embodiments, treatment with the nucleic acid of the invention reduces or prevents bleeding into a muscle or into the brain of a subject suffering from haemophilia.

Alternatively or in addition, treatment of a subject, preferably a subject having a disorder of haemostasis, such as haemophilia, with the nucleic acid of the invention may result in one or more of more of the following:

In certain embodiments, treatment of a subject, preferably a subject having a disorder of haemostasis, such as haemophilia, with the nucleic acid of the invention results in reduced bone marrow hyperplasia. As shown in FIG. 14A, treatment of Haem A mice with a nucleic acid of the invention significantly reduced bone marrow hyperplasia in said mice.

In certain embodiments, treatment of a subject, preferably a subject having a disorder of haemostasis, such as haemophilia, with the nucleic acid of the invention results in reduced osteoarthritis. As shown in FIG. 14B, treatment of Haem A mice with a nucleic acid of the invention significantly reduced osteoarthritis in said mice.

In certain embodiments, treatment of a subject, preferably a subject having a disorder of haemostasis, such as haemophilia, with the nucleic acid of the invention results in reduced chondrocyte degeneration/necrosis. As shown in FIG. 14C, treatment of Haem A mice with a nucleic acid of the invention significantly reduced chondrocyte degeneration/necrosis in said mice.

In certain embodiments, treatment of a subject, preferably a subject having a disorder of haemostasis, such as haemophilia, with the nucleic acid of the invention results in reduced haemorrhage. As shown in FIG. 14D, treatment of Haem A mice with a nucleic acid of the invention significantly reduced haemorrhage in said mice.

In certain embodiments, treatment of a subject, preferably a subject having a disorder of haemostasis, such as haemophilia, with the nucleic acid of the invention results in reduced haemosiderin deposition. As shown in FIG. 14E, treatment of Haem A mice with a nucleic acid of the invention significantly reduced haemosiderin deposition in said mice.

In certain embodiments, treatment of a subject, preferably a subject having a disorder of haemostasis, such as haemophilia, with the nucleic acid of the invention results in reduced occurrence of haematoma. As shown in FIG. 14F, treatment of Haem A mice with a nucleic acid of the invention significantly reduced haematoma in said mice.

In certain embodiments, treatment of a subject, preferably a subject having a disorder of haemostasis, such as haemophilia, with the nucleic acid of the invention results in reduced osteoclastogenic bone resorption. As shown in FIG. 14G, treatment of Haem A mice with a nucleic acid of the invention significantly reduced osteoclastogenic bone resorption in said mice.

In certain embodiments, treatment of a subject, preferably a subject having a disorder of haemostasis, such as haemophilia, with the nucleic acid of the invention results in reduced osteolysis. As shown in FIG. 14H, treatment of Haem A mice with a nucleic acid of the invention significantly reduced osteolysis in said mice.

In certain embodiments, treatment of a subject, preferably a subject having a disorder of haemostasis, such as haemophilia, with the nucleic acid of the invention results in reduced periostitis. As shown in FIG. 14I, treatment of Haem A mice with a nucleic acid of the invention significantly reduced periostitis in said mice.

In certain embodiments, treatment of a subject, preferably a subject having a disorder of haemostasis, such as haemophilia, with the nucleic acid of the invention results in reduced sub-chondral bone sclerosis. As shown in FIG. 14J, treatment of Haem A mice with a nucleic acid of the invention significantly reduced sub-chondral bone sclerosis in said mice.

In certain embodiments, treatment of a subject, preferably a subject having a disorder of haemostasis, such as haemophilia, with the nucleic acid of the invention results in reduced tendon degeneration. As shown in FIG. 14K, treatment of Haem A mice with a nucleic acid of the invention significantly reduced tendon degeneration in said mice.

In certain embodiments, treatment of a subject, preferably a subject having a disorder of haemostasis, such as haemophilia, with the nucleic acid of the invention results in reduced tendonitis. As shown in FIG. 14L, treatment of Haem A mice with a nucleic acid of the invention significantly reduced tendonitis in said mice.

In certain embodiments, treatment of a subject, preferably a subject having a disorder of haemostasis, such as haemophilia, with the nucleic acid of the invention results in reduced tenosynovitis. As shown in FIG. 14M, treatment of Haem A mice with a nucleic acid of the invention significantly reduced tenosynovitis in said mice.

Thus, in a particular embodiment, the invention relates to a nucleic acid suitable for use, or for use, in treatment of haemophilia, wherein the treatment of haemophilia is characterized by reduced bleeding and one or more of: reduced bone marrow hyperplasia, reduced osteoarthritis, reduced chondrocyte degeneration/necrosis, reduced haemorrhage, reduced haemosiderin deposition, reduced haematoma, reduced osteoclastogenic bone resorption, reduced osteolysis, reduced periostitis, reduced sub-chondral bone sclerosis, reduced tendon degeneration, reduced tendonitis, and/or reduced tenosynovitis. An nucleic acid e.g. siRNA of the invention may be administered as a “free” nucleic acid or “free siRNA, administered in the absence of a pharmaceutical composition. The naked nucleic acid may be in a suitable buffer solution. The buffer solution may comprise acetate, citrate, prolamine, carbonate, or phosphate, or any combination thereof. In one embodiment, the buffer solution is phosphate buffered saline (PBS). The pH and osmolarity of the buffer solution can be adjusted such that it is suitable for administering to a subject.

Alternatively, a nucleic acid e.g. siRNA of the invention may be administered as a pharmaceutical composition, such as a dsiRNA liposomal formulation.

In one embodiment, the method includes administering a composition featured herein such that expression of ZPI gene is decreased, such as for about 1, 2, 3, 4, 5, 6, 7, 8, 12, 16, 18, 24 hours, 28, 32, or about 36 hours. In one embodiment, expression of ZPI target gene is decreased for an extended duration, e.g., at least about two, three, four days or more, e.g., about one week, two weeks, three weeks, or four weeks or longer, e.g., about 1 month, 2 months, or 3 months.

Subjects can be administered a therapeutic amount of nucleic acid, e.g., siRNA, such as about 0.01 mg/kg to about 200 mg/kg, so as to treat disease related to a disorder of haemostasis, such as a disease related to a disorder of haemostasis, such as haemophilia.

The nucleic acid e.g. siRNA can be administered by intravenous infusion over a period of time, on a regular basis. In certain embodiments, after an initial treatment regimen, the treatments can be administered on a less frequent basis. Administration of the siRNA can reduce gene product levels of ZPI target gene, e.g., in a cell or tissue of the patient by at least about 20%, 25%, 30%, 35%, 40%, 45%, 50%, 55%, 60%, 65%, 70%, 75%, 80%, 85%, 90%, or 95%, or below the level of detection of the assay method used. In certain embodiments, administration results in clinical stabilization or preferably clinically relevant reduction of at least one sign or symptom of a ZPI gene-associated disorder.

Alternatively, the nucleic acid e.g. siRNA can be administered subcutaneously, i.e., by subcutaneous injection. One or more injections may be used to deliver the desired daily dose of nucleic acid e.g. siRNA to a subject. The injections may be repeated over a period of time. The administration may be repeated on a regular basis. In certain embodiments, after an initial treatment regimen, the treatments can be administered on a less frequent basis. A repeat-dose regimen may include administration of a therapeutic amount of nucleic acid on a regular basis, such as every other day or to once a year. In certain embodiments, the nucleic acid is administered about once per month to about once per quarter (i.e., about once every three months).

In one aspect the present invention may be applied in the compounds, processes, compositions or uses of the following Sentences numbered 1-101 wherein reference to any Formula in the Sentences 1-101 refers only to those Formulas that are defined within Sentences 1-101. These formulae are reproduced in FIG. 5. Specifically, an oligonucleoside moiety as represented by Z in any of the following sentences can comprise a nucleic acid for inhibiting expression of ZPI as defined in any of the claims hereinafter.

1. A compound comprising the following structure:

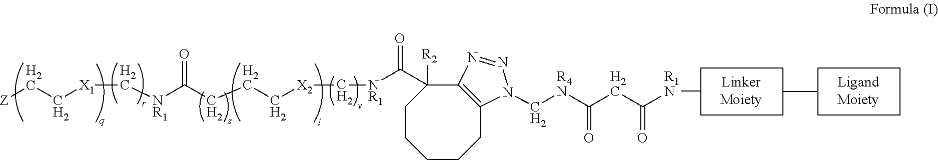

wherein:
R₁ at each occurrence is independently selected from the group consisting of hydrogen, methyl and ethyl:
R₂ is selected from the group consisting of hydrogen, hydroxy, —OC₁₋₃alkyl, —C(═O)OC₁₋₃alkyl, halo and nitro;
X₁ and X₂ at each occurrence are independently selected from the group consisting of methylene, oxygen and sulfur;
m is an integer of from 1 to 6;
n is an integer of from 1 to 10;
q, r, s, t, v are independently integers from 0 to 4, with the proviso that:
(i) q and r cannot both be 0 at the same time; and
(ii) s, t and v cannot all be 0 at the same time;
Z is an oligonucleoside moiety.
   2. A compound according to Sentence 1, wherein R₁ is hydrogen at each occurrence.
   3. A compound according to Sentence 1, wherein R₁ is methyl.
   4. A compound according to Sentence 1, wherein R₁ is ethyl.
   5. A compound according to any of Sentences 1 to 4, wherein R₂ is hydroxy.
   6. A compound according to any of Sentences 1 to 4, wherein R₂ is halo.
   7. A compound according to Sentence 6, wherein R₂ is fluoro.
   8. A compound according to Sentence 6, wherein R₂ is chloro.
   9. A compound according to Sentence 6, wherein R₂ is bromo.
   10. A compound according to Sentence 6, wherein R₂ is iodo.
   11. A compound according to Sentence 6, wherein R₂ is nitro.
   12. A compound according to any of Sentences 1 to 11, wherein X₁ is methylene.
   13. A compound according to any of Sentences 1 to 11, wherein X₁ is oxygen.
   14. A compound according to any of Sentences 1 to 11, wherein X₁ is sulfur.
   15. A compound according to any of Sentences 1 to 14, wherein X₂ is methylene.
   16. A compound according to any of Sentences 1 to 15, wherein X₂ is oxygen.
   17. A compound according to any of Sentences 1 to 16, wherein X₂ is sulfur.
   18. A compound according to any of Sentences 1 to 17, wherein m=3.
   19. A compound according to any of Sentences 1 to 18, wherein n=6.
   20. A compound according to Sentences 13 and 15, wherein X₁ is oxygen and X₂ is methylene, and preferably wherein:
q=1,
r=2,
s=1,
t=1,
v=1.
   21. A compound according to Sentences 12 and 15, wherein both X₁ and X₂ are methylene, and preferably wherein:
q=1,
r=3,
s=1,
t=1,
v=1.
   22. A compound according to any of Sentences 1 to 21, wherein Z is:

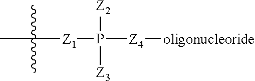

wherein:
Z₁, Z₂, Z₃, Z₄ are independently at each occurrence oxygen or sulfur; and
one the bonds between P and Z₂, and P and Z₃ is a single bond and the other bond is a double bond.
   23. A compound according to Sentence 22, wherein said oligonucleoside is an RNA compound capable of modulating, preferably inhibiting, expression of a target gene.
   24. A compound according to Sentence 23, wherein said RNA compound comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends.
   25. A compound according to Sentence 24, wherein the RNA compound is attached at the 5′ end of its second strand to the adjacent phosphate.
   26. A compound according to Sentence 24, wherein the RNA compound is attached at the 3′ end of its second strand to the adjacent phosphate.
   27. A compound of Formula (II):

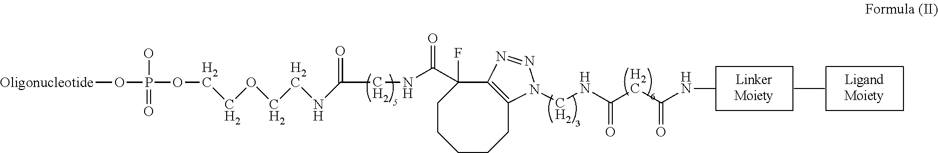

28. A compound of Formula (III):

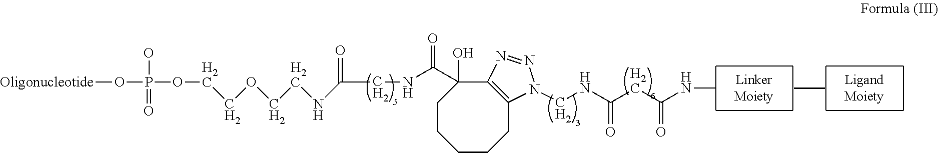

29. A compound according to Sentence 27 or 28, wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 5′ end of its second strand to the adjacent phosphate.
30. A composition comprising a compound of Formula (II) as defined in Sentence 27, and a compound of Formula (III) as defined in Sentence 28, optionally dependent on Sentence 29.
31. A composition according to Sentence 30, wherein said compound of Formula (III) as defined in Sentence 28 is present in an amount in the range of 10 to 15% by weight of said composition.
32. A compound of Formula (IV):

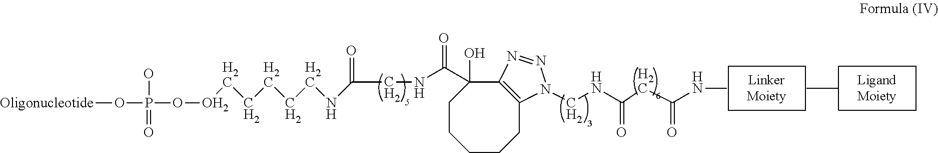

33. A compound of Formula (V):

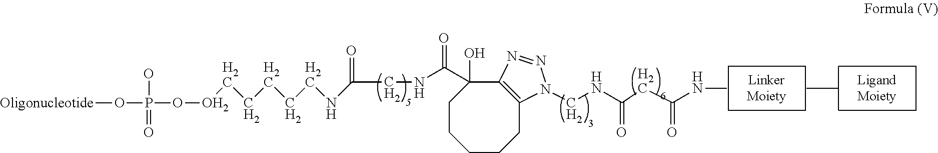

34. A compound according to Sentence 32 or 33, wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 3′ end of its second strand to the adjacent phosphate.
35. A composition comprising a compound of Formula (IV) as defined in Sentence 32, and a compound of Formula (V) as defined in Sentence 33, optionally dependent on Sentence 34.
36. A composition according to Sentence 35, wherein said compound of Formula (V) as defined in Sentence 33 is present in an amount in the range of 10 to 15% by weight of said composition.
37. A compound as defined in any of Sentences 1 to 29, or 32 to 34, wherein the oligonucleoside comprises an RNA duplex which further comprises one or more riboses modified at the 2′ position, preferably a plurality of riboses modified at the 2′ position.
38. A compound according to Sentence 37, wherein the modifications are chosen from 2′-O-methyl, 2′-deoxy-fluoro, and 2′-deoxy.
39. A compound according to any of Sentences 1 to 29, or 32 to 34, or 37 to 38, wherein the oligonucleoside further comprises one or more degradation protective moieties at one or more ends.
40. A compound according to Sentence 39, wherein said one or more degradation protective moieties are not present at the end of the oligonucleoside strand that carries the ligand moieties, and/or wherein said one or more degradation protective moieties is selected from phosphorothioate internucleoside linkages, phosphorodithioate internucleoside linkages and inverted abasic nucleosides, wherein said inverted abasic nucleosides are present at the distal end of the strand that carries the ligand moieties.
41. A compound according to any of Sentences 1 to 29, or 32 to 34, or 37 to 40, wherein said ligand moiety as depicted in Formula (I) in Sentence 1 comprises one or more ligands.
42. A compound according to Sentence 41, wherein said ligand moiety as depicted in Formula (I) in Sentence 1 comprises one or more carbohydrate ligands.
43. A compound according to Sentence 42, wherein said one or more carbohydrates can be a monosaccharide, disaccharide, trisaccharide, tetrasaccharide, oligosaccharide or polysaccharide.
44. A compound according to Sentence 43, wherein said one or more carbohydrates comprise one or more galactose moieties, one or more lactose moieties, one or more N-AcetylGalactosamine moieties, and/or one or more mannose moieties.
45. A compound according to Sentence 44, wherein said one or more carbohydrates comprise one or more N-Acetyl-Galactosamine moieties.
46. A compound according to Sentence 45, which comprises two or three N-AcetylGalactosamine moieties.
47. A compound according to any of Sentences 41 to 46, wherein said one or more ligands are attached in a linear configuration, or in a branched configuration.
48. A compound according to Sentence 47, wherein said one or more ligands are attached as a biantennary or triantennary branched configuration.
49. A compound according to Sentences 46 to 48, wherein said moiety:

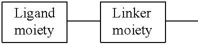

as depicted in Formula (I) in Sentence 1 is any of Formulae (VIa), (VIb) or (VIc), preferably Formula (VIa):

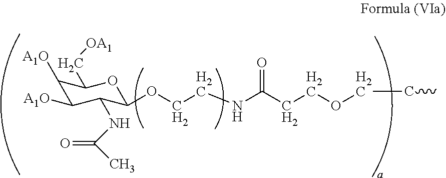

wherein:
A_{I} is hydrogen, or a suitable hydroxy protecting group;
a is an integer of 2 or 3; and
b is an integer of 2 to 5; or

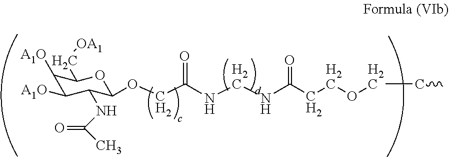

wherein:
A_{I} is hydrogen, or a suitable hydroxy protecting group;
a is an integer of 2 or 3; and
c and d are independently integers of 1 to 6; or

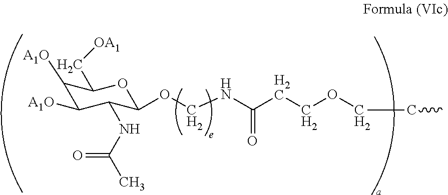

wherein:
A_{I} is hydrogen, or a suitable hydroxy protecting group;
a is an integer of 2 or 3; and
e is an integer of 2 to 10.
   50. A compound according to Sentences 46 to 48, wherein said moiety:

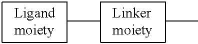

as depicted in Formula (I) in Sentence 1 is Formula (VII):

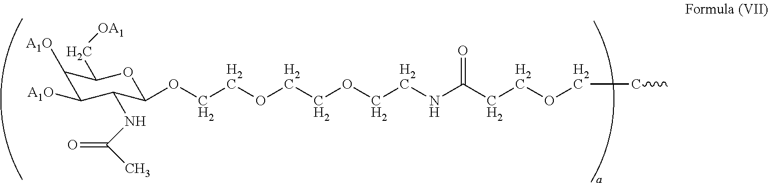

wherein:
A_{I} is hydrogen;
a is an integer of 2 or 3.
   51. A compound according to Sentence 49 or 50, wherein a=2.
   52. A compound according to Sentence 49 or 50, wherein a=3.
   53. A compound according to Sentence 49, wherein b=3.
   54. A compound of Formula (VIII):

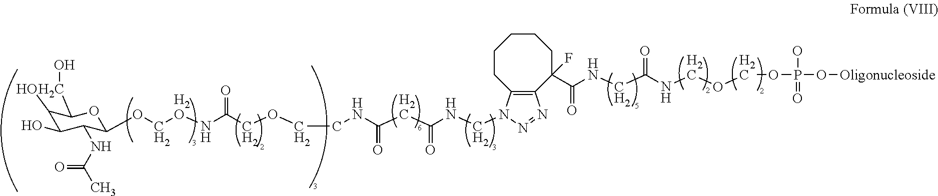

55. A compound of Formula (IX):

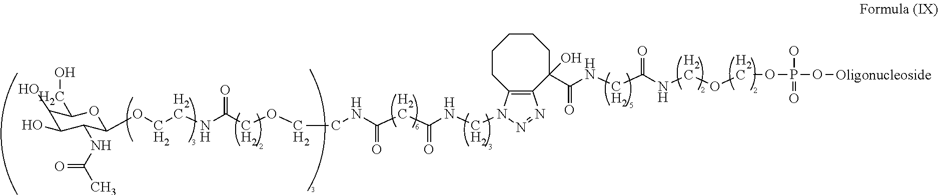

56. A compound according to Sentence 54 or 55, wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 5′ end of its second strand to the adjacent phosphate.
57. A composition comprising a compound of Formula (VIII) as defined in Sentence 54, and a compound of Formula (IX) as defined in Sentence 55, optionally dependent on Sentence 56.
58. A composition according to Sentence 57, wherein said compound of Formula (IX) as defined in Sentence 55 is present in an amount in the range of 10 to 15% by weight of said composition.
59. A compound of Formula (X):

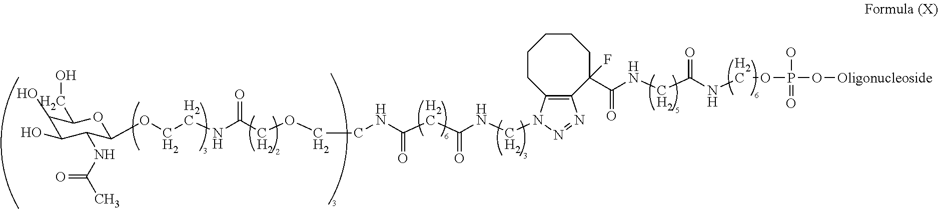

60. A compound of Formula (XI):

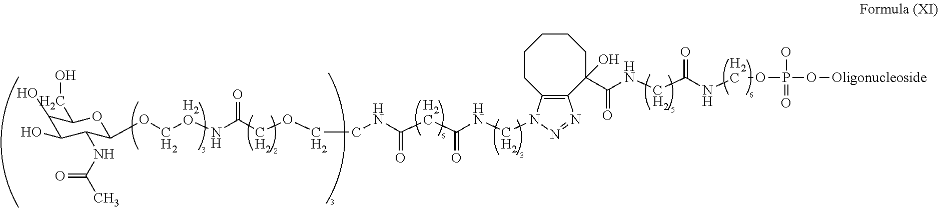

61. A compound according to Sentence 59 or 60, wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 3′ end of its second strand to the adjacent phosphate.
62. A composition comprising a compound of Formula (X) as defined in Sentence 59, and a compound of Formula (XI) as defined in Sentence 60, optionally dependent on Sentence 61.
63. A composition according to Sentence 62, wherein said compound of Formula (XI) as defined in Sentence 60 is present in an amount in the range of 10 to 15% by weight of said composition.
64. A compound as defined in any of Sentences 54 to 63, wherein the oligonucleoside comprises an RNA duplex which further comprises one or more riboses modified at the 2′ position, preferably a plurality of riboses modified at the 2′ position.
65. A compound according to Sentence 64, wherein the modifications are chosen from 2′-O-methyl, 2′-deoxy-fluoro, and 2′-deoxy.
66. A compound according to any of Sentences 54 to 65, wherein the oligonucleoside further comprises one or more degradation protective moieties at one or more ends.
67. A compound according to Sentence 66, wherein said one or more degradation protective moieties are not present at the end of the oligonucleoside strand that carries the ligand moieties, and/or wherein said one or more degradation protective moieties is selected from phosphorothioate internucleoside linkages, phosphorodithioate internucleoside linkages and inverted abasic nucleosides, wherein said inverted abasic nucleosides are present at the distal end of the strand that carries the ligand moieties, as shown in any of Formulae (VIII), (IX), (X) or (XI) in any of Sentences 54, 55, 59 or 60.
68. A process of preparing a compound according to any of Sentences 1 to 29, 32 to 34, 37 to 56, 59 to 61, and 64 to 67, and/or a composition according to any of Sentences 30, 31, 35, 36, 57, 58, 62, 63, which comprises reacting compounds of Formulae (XII) and (XIII):

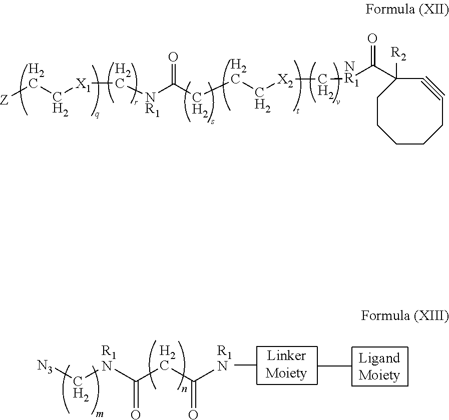

herein:
R₁ at each occurrence is independently selected from the group consisting of hydrogen, methyl and ethyl;
R₂ is selected from the group consisting of hydrogen, hydroxy, —OC₁₋₃alkyl, —C(═O)OC₁₋₃alkyl, halo and nitro;
X₁ and X₂ at each occurrence are independently selected from the group consisting of methylene, oxygen and sulfur;
m is an integer of from 1 to 6;
n is an integer of from 1 to 10;
q, r, s, t, v are independently integers from 0 to 4, with the proviso that:
(i) q and r cannot both be 0 at the same time; and
(ii) s, t and v cannot all be 0 at the same time;
Z is an oligonucleoside moiety;
and where appropriate carrying out deprotection of the ligand and/or annealing of a second strand for the oligonucleoside moiety.
   69. A process according to Sentence 68, wherein a compound of Formula (XII) is prepared by reacting compounds of Formulae (XIV) and (XV):

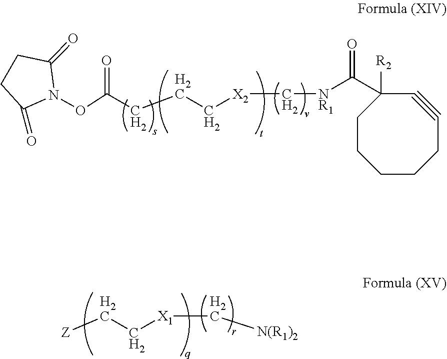

R₁ at each occurrence is independently selected from the group consisting of hydrogen, methyl and ethyl;
R₂ is selected from the group consisting of hydrogen, hydroxy, —OC₁₋₃alkyl, —C(═O)OC₁₋₃alkyl, halo and nitro;
X₁ and X₂ at each occurrence are independently selected from the group consisting of methylene, oxygen and sulfur;
q, r, s, t, v are independently integers from 0 to 4, with the proviso that:
(i) q and r cannot both be 0 at the same time; and
(ii) s, t and v cannot all be 0 at the same time;
Z is an oligonucleoside moiety.
   70. A process according to Sentence 68, to prepare a compound according to any of Sentences 20, 25, 27, 29, 54, 56, and/or a composition according to any of Sentences 30, 31, 57, 58, wherein:
compound of Formula (XII) is Formula (XIIa):

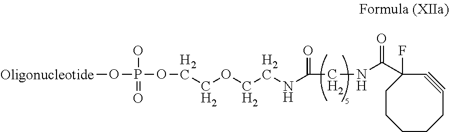

and compound of Formula (XIII) is Formula (XIIIa):

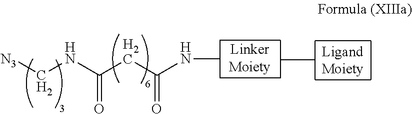

wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 5′ end of its second strand to the adjacent phosphate.
   71. A process according to Sentence 68, to prepare a compound according to any of Sentences 20, 25, 28, 29, 55, 56, and/or a composition according to any of Sentences 30, 31, 57, 58, wherein:
compound of Formula (XII) is Formula (XIIb):

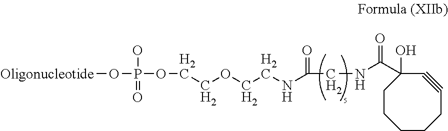

and compound of Formula (XIII) is Formula (XIIIa):

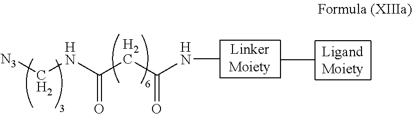

wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 5′ end of its second strand to the adjacent phosphate.
   72. A process according to Sentence 68, to prepare a compound according to any of Sentences 21, 26, 32, 34, 59, 61, and/or a composition according to any of Sentences 35, 36, 62, 63, wherein: compound of Formula (XII) is Formula (XIIc):

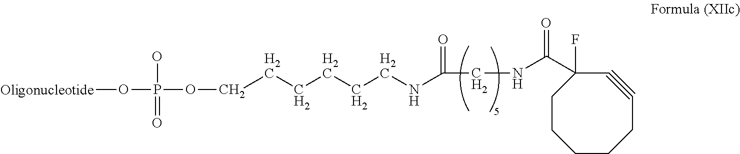

and compound of Formula (XIII) is Formula (XIIIa):

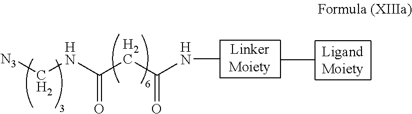

wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 3′ end of its second strand to the adjacent phosphate.
   73. A process according to Sentence 68, to prepare a compound according to any of Sentences 21, 26, 33, 34, 60, 61, and/or a composition according to any of Sentences 35, 36, 62, 63, wherein:
compound of Formula (XII) is Formula (XIId):

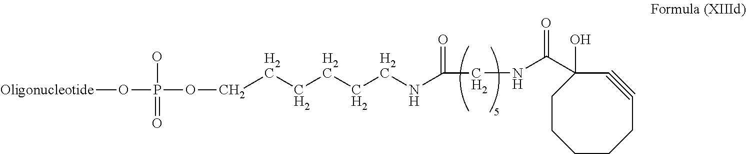

and compound of Formula (XIII) is Formula (XIIIa):

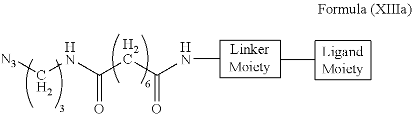

wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 3′ end of its second strand to the adjacent phosphate.
   74. A process according to any of Sentences 70 to 73, wherein:
compound of Formula (XIIIa) is Formula (XIIIb):

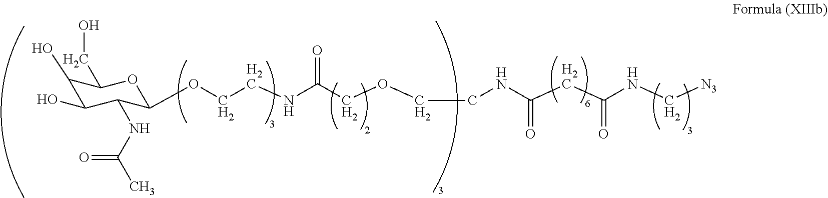

75. A process according to Sentences 69, as dependent on Sentences 70 to 73, wherein:
compound of Formula (XIV) is either Formula (XIVa) or Formula (XIVb):

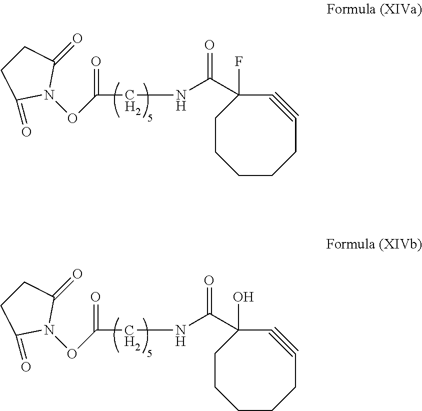

and compound of Formula (XV) is either Formula (XVa) or Formula (XIVb):

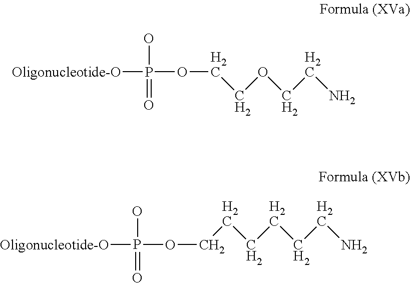

wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein (i) said RNA duplex is attached at the 5′ end of its second strand to the adjacent phosphate in Formula (XVa), or (ii) said RNA duplex is attached at the 3′ end of its second strand to the adjacent phosphate in Formula (XVb).
   76. A compound of Formula (XII):

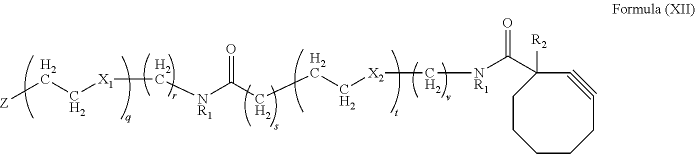

wherein:
R₁ at each occurrence is independently selected from the group consisting of hydrogen, methyl and ethyl;
R₂ is selected from the group consisting of hydrogen, hydroxy, —OC₁₋₃alkyl, —C(═O)OC₁₋₃alkyl, halo and nitro;
X₁ and X₂ at each occurrence are independently selected from the group consisting of methylene, oxygen and sulfur;
q, r, s, t, v are independently integers from 0 to 4, with the proviso that:
(i) q and r cannot both be 0 at the same time; and
(ii) s, t and v cannot all be 0 at the same time;
Z is an oligonucleoside moiety.
   77. A compound of Formula (XIIa):

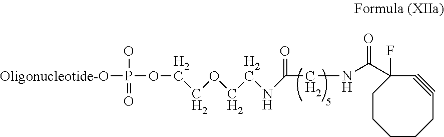

78. A compound of Formula (XIIb):

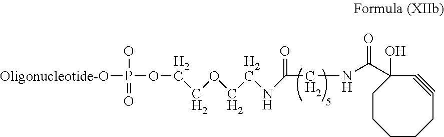

79. A compound of Formula (XIIc):

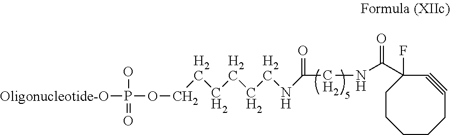

80. A compound of Formula (XIId):

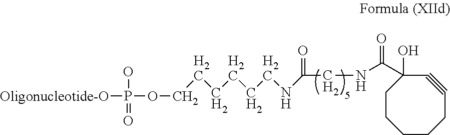

81. A compound of Formula (XIII):

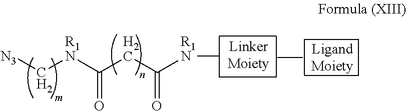

wherein:
R₁ at each occurrence is independently selected from the group consisting of hydrogen, methyl and ethyl;
m is an integer of from 1 to 6;
n is an integer of from 1 to 10.
   82. A compound of Formula (XIIIa):

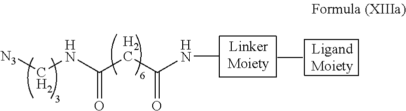

83. A compound of Formula (XIIIb):

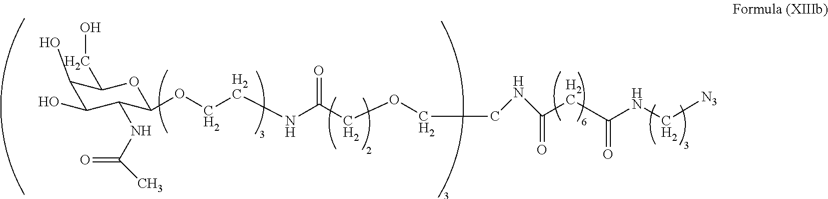

84. A compound of Formula (XIV):

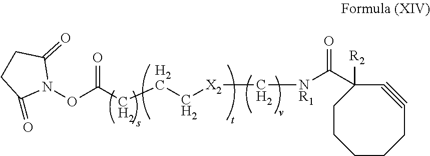

wherein:
R₁ is selected from the group consisting of hydrogen, methyl and ethyl;
R₂ is selected from the group consisting of hydrogen, hydroxy, —OC₁₋₃alkyl, —C(═O)OC₁₋₃alkyl, halo and nitro;
X₂ is selected from the group consisting of methylene, oxygen and sulfur,
s, t, v are independently integers from 0 to 4, with the proviso that s, t and v cannot all be 0 at the same time.
   85. A compound of Formula (XIVa):

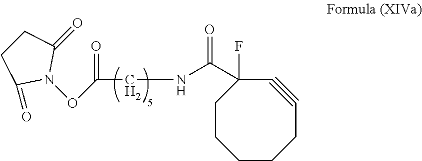

86. A compound of Formula (XIVb):

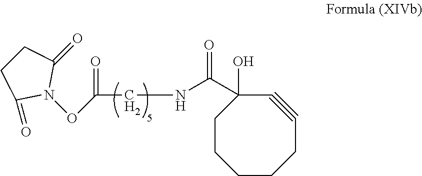

87. A compound of Formula (XV):

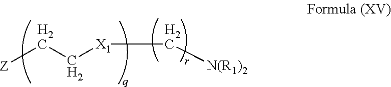

wherein:
R₁ at each occurrence is independently selected from the group consisting of hydrogen, methyl and ethyl;
X₁ is selected from the group consisting of methylene, oxygen and sulfur;
q and r are independently integers from 0 to 4, with the proviso that q and r cannot both be 0 at the same time;
Z is an oligonucleoside moiety.
   88. A compound of Formula (XVa):

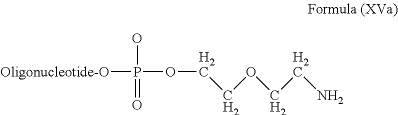

89. A compound of Formula (XVb):

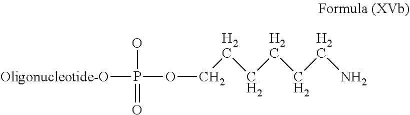

90. Use of a compound according to any of Sentences 76, 81 to 84, 87, for the preparation of a compound according to any of Sentences 1 to 29, 32 to 34, 37 to 56, 59 to 61, and 64 to 67, and/or a composition according to any of Sentences 30, 31, 35, 36, 57, 58, 62 and 63.
91. Use of a compound according to Sentence 85, for the preparation of a compound according to any of Sentences 1 to 29, 32 to 34, 37 to 56, 59 to 61, and 64 to 67, and/or a composition according to any of Sentences 30, 31, 35, 36, 57, 58, 62 and 63, wherein R₂═F.
92. Use of a compound according to Sentence 86, for the preparation of a compound according to any of Sentences 1 to 29, 32 to 34, 37 to 56, 59 to 61, and 64 to 67, and/or a composition according to any of Sentences 30, 31, 35, 36, 57, 58, 62 and 63, wherein R₂═OH.
93. Use of a compound according to Sentence 77, for the preparation of a compound according to any of Sentences 20, 25, 27, 29, 54, 56, and/or a composition according to any of Sentences 30, 31, 57, 58.
94. Use of a compound according to Sentence 78, for the preparation of a compound according to any of Sentences 20, 25, 28, 29, 55, 56, and/or a composition according to any of Sentences 30, 31, 57, 58.
95. Use of a compound according to Sentence 79, for the preparation of a compound according to any of Sentences 21, 26, 32, 34, 59, 61, and/or a composition according to any of Sentences 35, 36, 62, 63.
96. Use of a compound according to Sentence 80, for the preparation of a compound according to any of Sentences 21, 26, 33, 34, 60, 61, and/or a composition according to any of Sentences 35, 36, 62, 63.
97. Use of a compound according to Sentence 88, for the preparation of a compound according to any of Sentences 20, 25, 27 to 29, 54 to 56, and/or a composition according to any of Sentences 30, 31, 57, 58.
98. Use of a compound according to Sentence 89, for the preparation of a compound according to any of Sentences 21, 26, 32 to 34, 59 to 61, and/or a composition according to any of Sentences 35, 36, 62, 63.
99. A compound or composition obtained, or obtainable by a process according to any of Sentences 68 to 75.
100. A pharmaceutical composition comprising of a compound according to any of Sentences 1 to 29, 32 to 34, 37 to 56, 59 to 61, and 64 to 67, and/or a composition according to any of Sentences 30, 31, 35, 36, 57, 58, 62 and 63, together with a pharmaceutically acceptable carrier, diluent or excipient.
101. A compound according to any of Sentences 1 to 29, 32 to 34, 37 to 56, 59 to 61, and 64 to 67, and/or a composition according to any of Sentences 30, 31, 35, 36, 57, 58, 62 and 63, for use in therapy.

In another aspect the present invention may be applied in the compounds, processes, compositions or uses of the following Clauses numbered 1-56 wherein reference to any Formula in the Clauses refers only to those Formulas that are defined within Clause 1-56. These formulae are reproduced in FIG. 6. Specifically, an oligonucleoside moiety as represented by Z in any of the following clauses can comprise a nucleic acid for inhibiting expression of ZPI as defined in any of the claims hereinafter.

1. A compound comprising the following structure:

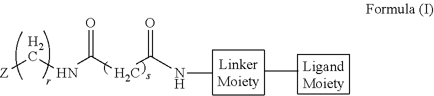

wherein:
r and s are independently an integer selected from 1 to 16; and
Z is an oligonucleoside moiety.
   2. A compound according to Clause 1, wherein s is an integer selected from 4 to 12.
   3. A compound according to Clause 2, wherein s is 6.
   4. A compound according to any of Clauses 1 to 3, wherein r is an integer selected from 4 to 14.
   5. A compound according to Clause 4, wherein r is 6.
   6. A compound according to Clause 4, wherein r is 12.
   7. A compound according to Clause 5, which is dependent on Clause 3.
   8. A compound according to Clause 6, which is dependent on Clause 3.
   9. A compound according to any of Clauses 1 to 8, wherein Z is:

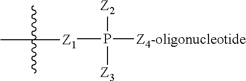

wherein:
Z₁, Z₂, Z₃, Z₄ are independently at each occurrence oxygen or sulfur; and
one the bonds between P and Z₂, and P and Z₃ is a single bond and the other bond is a double bond.
   10. A compound according to any of Clauses 1 to 9, wherein said oligonucleoside is an RNA compound capable of modulating, preferably inhibiting, expression of a target gene.
   11. A compound according to any of Clause 10, wherein said RNA compound comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends.
   12. A compound according to Clause 11, preferably also dependent on Clauses 3 and 6, wherein the RNA compound is attached at the 5′ end of its second strand to the adjacent phosphate.
   13. A compound according to Clause 11, preferably also dependent on Clauses 3 and 5, wherein the RNA compound is attached at the 3′ end of its second strand to the adjacent phosphate.
   14. A compound of Formula (II), preferably dependent on Clause 12:

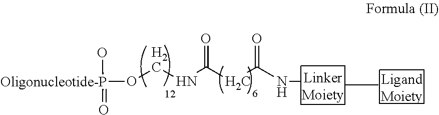

15. A compound of Formula (III), preferably dependent on Clause 13:

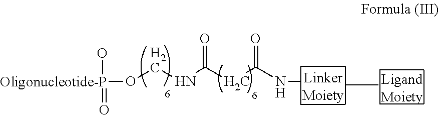

16. A compound as defined in any of Clauses 1 to 15, wherein the oligonucleoside comprises an RNA duplex which further comprises one or more riboses modified at the 2′ position, preferably a plurality of riboses modified at the 2′ position.
17. A compound according to Clause 16, wherein the modifications are chosen from 2′-O-methyl, 2′-deoxy-fluoro, and 2′-deoxy.
18. A compound according to any of Clauses 1 to 17, wherein the oligonucleoside further comprises one or more degradation protective moieties at one or more ends.
19. A compound according to Clause 18, wherein said one or more degradation protective moieties are not present at the end of the oligonucleoside strand that carries the linker/ligand moieties, and/or wherein said one or more degradation protective moieties is selected from phosphorothioate internucleoside linkages, phosphorodithioate internucleoside linkages and inverted abasic nucleosides, wherein said inverted abasic nucleosides are present at the distal end of the same strand to the end that carries the linker/ligand moieties.
20. A compound according to any of Clauses 1 to 19, wherein said ligand moiety as depicted in Formula (I) in Clause 1 comprises one or more ligands.
21. A compound according to Clause 20, wherein said ligand moiety as depicted in Formula (I) in Clause 1 comprises one or more carbohydrate ligands.
22. A compound according to Clause 21, wherein said one or more carbohydrates can be a monosaccharide, disaccharide, trisaccharide, tetrasaccharide, oligosaccharide or polysaccharide.
23. A compound according to Clause 22, wherein said one or more carbohydrates comprise one or more galactose moieties, one or more lactose moieties, one or more N-AcetylGalactosamine moieties, and/or one or more mannose moieties.
24. A compound according to Clause 23, wherein said one or more carbohydrates comprise one or more N-Acetyl-Galactosamine moieties.
25. A compound according to Clause 24, which comprises two or three N-AcetylGalactosamine moieties.
26. A compound according to any of the preceding Clauses, wherein said one or more ligands are attached in a linear configuration, or in a branched configuration.
27. A compound according to Clause 26, wherein said one or more ligands are attached as a biantennary or triantennary branched configuration.
28. A compound according to Clauses 20 to 27, wherein said moiety:

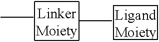

as depicted in Formula (I) in Clause 1 is any of Formulae (IV), (V) or (VI), preferably Formula (IV):

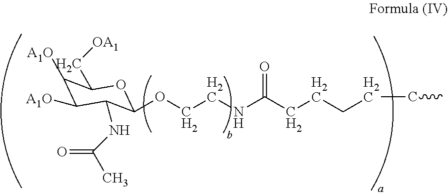

wherein:
A_{I} is hydrogen, or a suitable hydroxy protecting group;
a is an integer of 2 or 3; and
b is an integer of 2 to 5; or

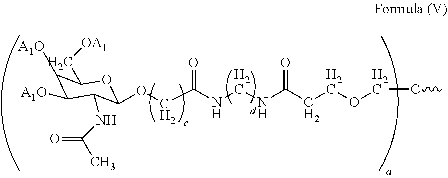

wherein:
A_{I} is hydrogen, or a suitable hydroxy protecting group;
a is an integer of 2 or 3; and
c and d are independently integers of 1 to 6; or

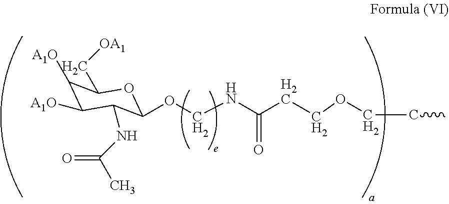

wherein:
A_{I} is hydrogen, or a suitable hydroxy protecting group;
a is an integer of 2 or 3; and
e is an integer of 2 to 10.
   29. A compound according to any of Clauses 1 to 28, wherein said moiety:

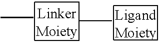

as depicted in Formula (I) in Clause 1 is Formula (VII):

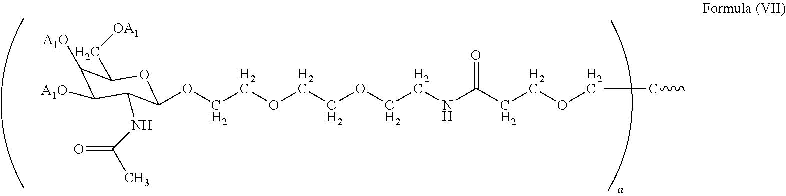

wherein:
A_{I} is hydrogen;
a is an integer of 2 or 3.
   30. A compound according to Clause 28 or 29, wherein a=2.
   31. A compound according to Clause 28 or 29, wherein a=3.
   32. A compound according to Clause 28, wherein b=3.
   33. A compound of Formula (VIII):

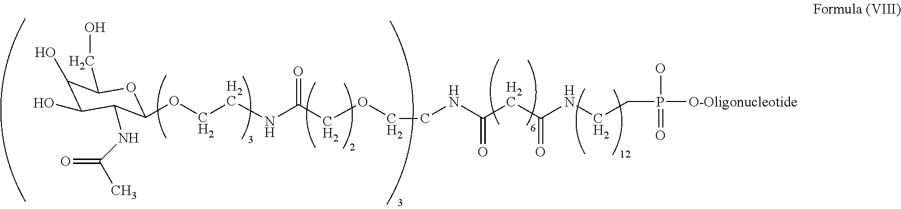

34. A compound of Formula (IX):

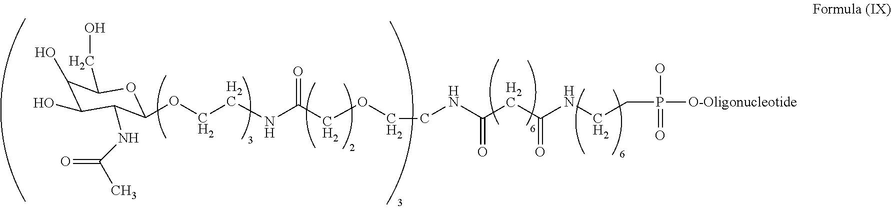

35. A compound according to Clause 33 or 34, wherein the oligonucleoside comprises an RNA duplex which further comprises one or more riboses modified at the 2′ position, preferably a plurality of riboses modified at the 2′ position.
36. A compound according to Clause 35, wherein the modifications are chosen from 2′-O-methyl, 2′-deoxy-fluoro, and 2′-deoxy.
37. A compound according to any of Clauses 33 to 36, wherein the oligonucleoside further comprises one or more degradation protective moieties at one or more ends.
38. A compound according to Clause 37, wherein said one or more degradation protective moieties are not present at the end of the oligonucleoside strand that carries the linker/ligand moieties, and/or wherein said one or more degradation protective moieties is selected from phosphorothioate internucleoside linkages, phosphorodithioate internucleoside linkages and inverted abasic nucleosides, wherein said inverted abasic nucleosides are present at the distal end of the same strand to the end that carries the linker/ligand moieties.
39. A compound according to Clause 33, wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 5′ end of its second strand to the adjacent phosphate.
40. A compound according to Clause 34, wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 3′ end of its second strand to the adjacent phosphate.
41. A process of preparing a compound according to any of Clauses 1 to 40, which comprises reacting compounds of Formulae (X) and (XI):

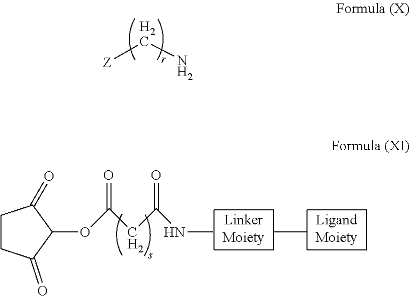

wherein:
r and s are independently an integer selected from 1 to 16; and
Z is an oligonucleoside moiety;
and where appropriate carrying out deprotection of the ligand and/or annealing of a second strand for the oligonucleoside.
   42. A process according to Clause 41, to prepare a compound according to any of Clauses 6, 8 to 14, 16 to 33, and 35 to 40, wherein:
compound of Formula (X) is Formula (Xa):

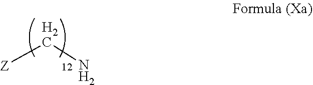

and compound of Formula (XI) is Formula (Xia):

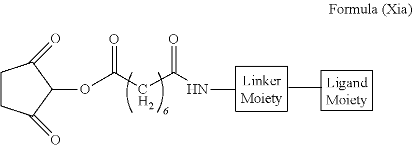

wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 5′ end of its second strand to the adjacent phosphate.
   43. A process according to Clause 41, to prepare a compound according to any of Clauses 5, 7, 9 to 13, 15 to 32, and 34 to 40, wherein:
compound of Formula (X) is Formula (Xb):

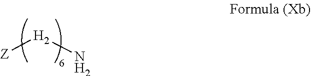

and compound of Formula (XI) is Formula (Xia):

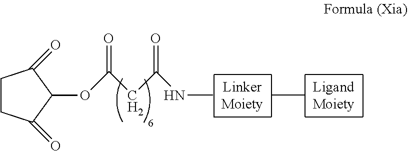

wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 3′ end of its second strand to the adjacent phosphate.
   44. A process according to Clauses 42 or 43, wherein:
compound of Formula (Xia) is Formula (Xib):

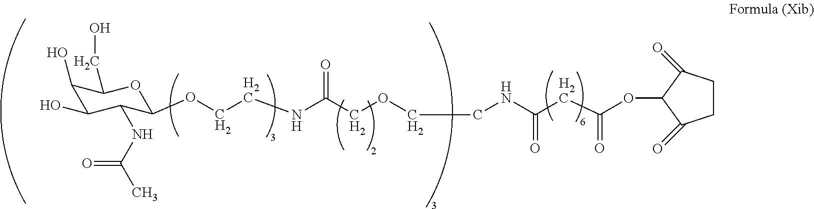

45. A compound of Formula (X):

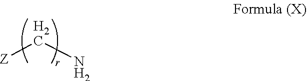

wherein:
r is independently an integer selected from 1 to 16; and
Z is an oligonucleoside moiety.
   46. A compound of Formula (Xa):

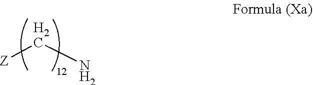

47. A compound of Formula (Xb):

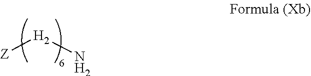

48. A compound of Formula (XI):

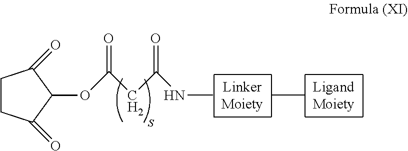

wherein:
s is independently an integer selected from 1 to 16; and
Z is an oligonucleoside moiety.
   49. A compound of Formula (Xia):

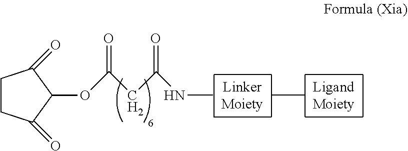

50. A compound of Formula (Xib):

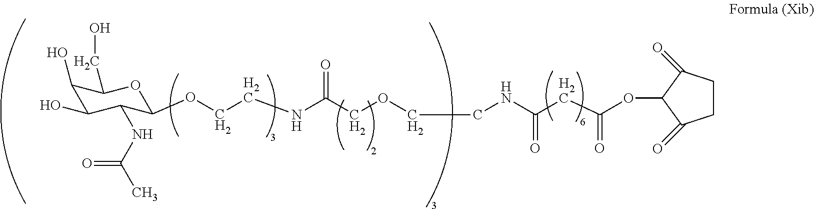

51. Use of a compound according to any of Clauses 45 and 48 to 50, for the preparation of a compound according to any of Clauses 1 to 40.
52. Use of a compound according to Clause 46, for the preparation of a compound according to any of Clauses 6, 8 to 14, 16 to 33, and 35 to 40.
53. Use of a compound according to Clause 47, for the preparation of a compound according to any of Clauses 5, 7, 9 to 13, 15 to 32, and 34 to 40.
54. A compound or composition obtained, or obtainable by a process according to any of Clauses 41 to 44.
55. A pharmaceutical composition comprising of a compound according to any of Clauses 1 to 40, together with a pharmaceutically acceptable carrier, diluent or excipient.
56. A compound according to any of Clauses 1 to 40, for use in therapy.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended Clauses.

### Example 1: Synthesis of Tether 1

### General Experimental Conditions

Thin layer chromatography (TLC) was performed on silica-coated aluminium plates with fluorescence indicator 254 nm from Macherey-Nagel. Compounds were visualized under UV light (254 nm), or after spraying with the 5% H₂SO₄ in methanol (MeOH) or ninhydrin reagent according to Stahl (from Sigma-Aldrich), followed by heating. Flash chromatography was performed with a Biotage Isolera One flash chromatography instrument equipped with a dual variable UV wavelength detector (200-400 nm) using Biotage Sfär Silica 10, 25, 50 or 100 g columns (Uppsala, Sweden).

All moisture-sensitive reactions were carried out under anhydrous conditions using dry glassware, anhydrous solvents, and argon atmosphere. All commercially available reagents were purchased from Sigma-Aldrich and solvents from Carl Roth GmbH+Co. KG. D-Galactosamine pentaacetate was purchased from AK scientific.

HPLC/ESI-MS was performed on a Dionex UltiMate 3000 RS UHPLC system and Thermo Scientific MSQ Plus Mass spectrometer using an Acquity UPLC Protein BEH C4 column from Waters (300 Å, 1.7 μm, 2.1×100 mm) at 60° C. The solvent system consisted of solvent A with H₂O containing 0.1% formic acid and solvent B with acetonitrile (ACN) containing 0.1% formic acid. A gradient from 5-100% of B over 15 min with a flow rate of 0.4 mL/min was employed. Detector and conditions: Corona ultra-charged aerosol detection (from esa). Nebulizer Temp.: 25° C. N₂ pressure: 35.1 psi. Filter: Corona.

¹H and ¹³C NMR spectra were recorded at room temperature on a Varian spectrometer at 500 MHz (¹H NMR) and 125 MHz (¹³C NMR). Chemical shifts are given in ppm referenced to the solvent residual peak (CDCl₃—¹H NMR: δ at 7.26 ppm and ¹³C NMR δ at 77.2 ppm: DMSO-d₆—1H NMR: δ at 2.50 ppm and ¹³C NMR δ at 39.5 ppm). Coupling constants are given in Hertz. Signal splitting patterns are described as singlet(s), doublet (d), triplet (t) or multiplet (m).

### Synthesis Route for the Conjugate Building Block TriGalNAc_Tether1:

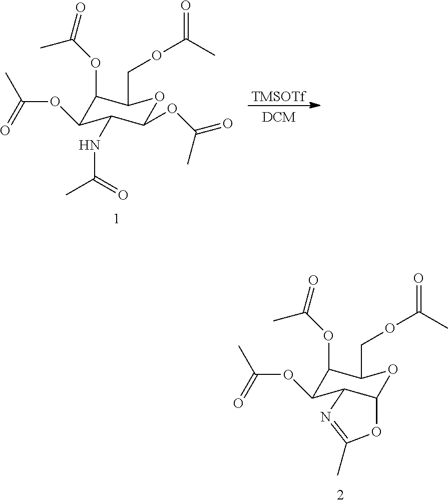 [SMILES: "TMSOTf" ; CC(=O)NC1C(OC(C)=O)OC(COC(C)=O)C(OC(C)=O)C1OC(C)=O ; CC(=O)OCC1OC2OC(C)=NC2C(OC(C)=O)C1OC(C)=O]

Preparation of compound 2: D-Galactosamine pentaacetate (3.00 g, 7.71 mmol, 1.0 eq.) was dissolved in anhydrous dichloromethane (DCM) (30 mL) under argon and trimethylsilyl trifluoromethanesulfonate (TMSOTf, 4.28 g, 19.27 mmol, 2.5 eq.) was added. The reaction was stirred at room temperature for 3 h. The reaction mixture was diluted with DCM (50 mL) and washed with cold saturated aq. NaHCO₃ (100 mL) and water (100 mL). The organic layer was separated, dried over Na₂SO₄ and concentrated to afford the title compound as yellow oil, which was purified by flash chromatography (gradient elution: 0-10% MeOH in DCM in 10 CV). The product was obtained as colourless oil (2.5 g, 98%, rf=0.45 (2% MeOH in DCM)).

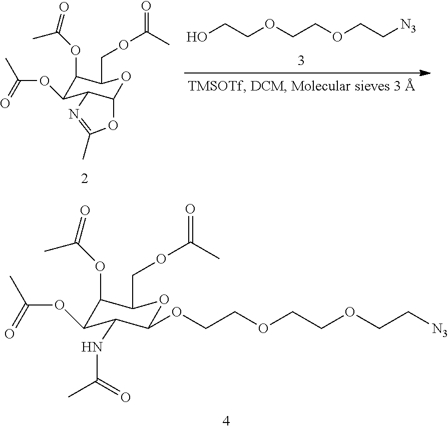

Preparation of compound 4: Compound 2 (2.30 g, 6.98 mmol, 1.0 eq.) and azido-PEG3-OH (1.83 g, 10.5 mmol, 1.5 eq.) were dissolved in anhydrous DCM (40 mL) under argon and molecular sieves 3 Å (5 g) were added to the solution. The mixture was stirred at room temperature for 1 h. TMSOTf (0.77 g, 3.49 mmol, 0.5 eq.) was then added to the mixture and the reaction was stirred overnight. The molecular sieves were filtered, the filtrate was diluted with DCM (100 mL) and washed with cold saturated aq. NaHCO₃ (100 mL) and water (100 mL). The organic layer was separated, dried over Na₂SO₄ and the solvent was removed under reduced pressure. The crude material was purified by flash chromatography (gradient elution: 0-3% MeOH in DCM in 10 CV) to afford the title product as light yellow oil (3.10 g, 88%, rf=0.25 (2% MeOH in DCM)). MS: calculated for C₂₀H₃₂N₄O₁₁, 504.21. Found 505.4. ¹H NMR (500 MHz, CDCl₃) δ 6.21-6.14 (m, 1H), 5.30 (dd, J=3.4, 1.1 Hz, 1H), 5.04 (dd, J=11.2, 3.4 Hz, 1H), 4.76 (d, J=8.6 Hz, 1H), 4.23-4.08 (m, 3H), 3.91-3.80 (m, 3H), 3.74-3.59 (m, 9H), 3.49-3.41 (m, 2H), 2.14 (s, 3H), 2.02 (s, 3H), 1.97 (d, J=4.2 Hz, 6H). ¹³C NMR (125 MHz, CDCl₃) δ 170.6©, 170.5 (C), 170.4 (C), 170.3 (C), 102.1 (CH), 71.6 (CH), 70.8 (CH), 70.6 (CH), 70.5 (CH), 70.3 (CH₂), 69.7 (CH₂), 68.5 (CH₂), 66.6 (CH₂), 61.5 (CH₂), 23.1 (CH₃), 20.7 (3×CH3).

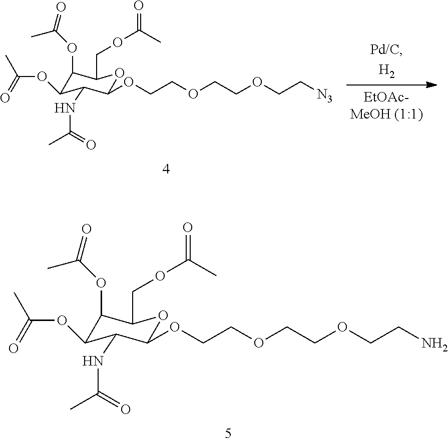 [SMILES: CC(=O)NC1C(OCCOCCOCCN=[N+]=[N-])OC(COC(C)=O)C(OC(C)=O)C1OC(C)=O ; CC(=O)NC1C(OCCOCCOCCN)OC(COC(C)=O)C(OC(C)=O)C1OC(C)=O]

Preparation of compound 5: Compound 4 (1.00 g, 1.98 mmol, 1.0 eq.) was dissolved in a mixture of ethyl acetate (EtOAc) and MeOH (30 mL 1:1 v/v) and Pd/C (100 mg) was added. The reaction mixture was degassed using vacuum/argon cycles (3×) and hydrogenated under balloon pressure overnight. The reaction mixture was filtered through celite and washed with EtOAc (30 mL). The solvent was removed under reduced pressure to afford the title compound as colourless oil (0.95 g, quantitative yield, rf=0.25 (10% MeOH in DCM)). The compound was used without further purification. MS: calculated for C₂₀H₃₄N₂O₁₁, 478.2. Found 479.4.

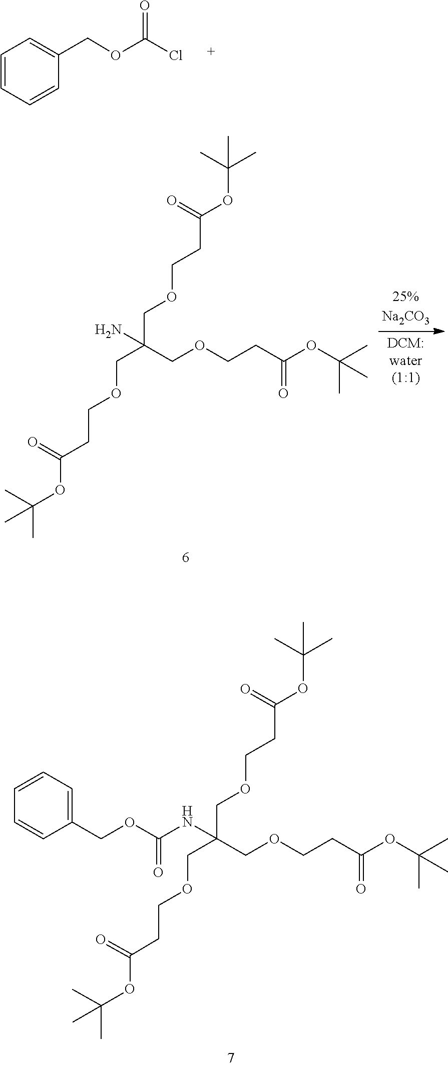 [SMILES: O=C(Cl)OCc1ccccc1 ; CC(C)(C)OC(=O)CCOCC(N)(COCCC(=O)OC(C)(C)C)COCCC(=O)OC(C)(C)C ; CC(C)(C)OC(=O)CCOCC(COCCC(=O)OC(C)(C)C)(COCCC(=O)OC(C)(C)C)NC(=O)OCc1ccccc1]

Preparation of compound 7: Tris {[2-(tert-butoxycarbonyl) ethoxy]methyl}-methylamine 6 (3.37 g, 6.67 mmol, 1.0 eq.) was dissolved in a mixture of DCM/water (40 mL 1:1 v/v) and Na₂CO₃ (0.18 g, 1.7 mmol, 0.25 eq.) was added while stirring vigorously. Benzyl chloroformate (2.94 mL, 20.7 mmol, 3.10 eq.) was added dropwise to the previous mixture and the reaction was stirred at room temperature for 24 h. The reaction mixture was diluted with CH₂Cl₂ (100 mL) and washed with water (100 mL). The organic layer was separated and dried over Na₂SO₄. The solvent was removed under reduced pressure and the resulting crude material was purified by flash chromatography (gradient elution: 0-10% EtOAc in cyclohexane in 12 CV) to afford the title compound as pale yellowish oil (3.9 g, 91%, rf=0.56 (10% EtOAc in cyclohexane)). MS: calculated for C₃₃H₅₃NO₁₁, 639.3. Found 640.9. ¹H NMR (500 MHz, DMSO-d₆) δ 7.38-7.26 (m, 5H), 4.97 (s, 2H), 3.54 (t, 6H), 3.50 (s, 6H), 2.38 (t, 6H), 1.39 (s, 27H). ¹³C NMR (125 MHz, DMSO-d₆) δ 170.3 (3×C), 154.5 (C), 137.1 (C), 128.2 (2×CH), 127.7 (CH), 127.6 (2×CH), 79.7 (3×C), 68.4 (3×CH2), 66.8 (3×CH2), 64.9 (C), 58.7 (CH₂), 35.8 (3×CH2), 27.7 (9×CH3).

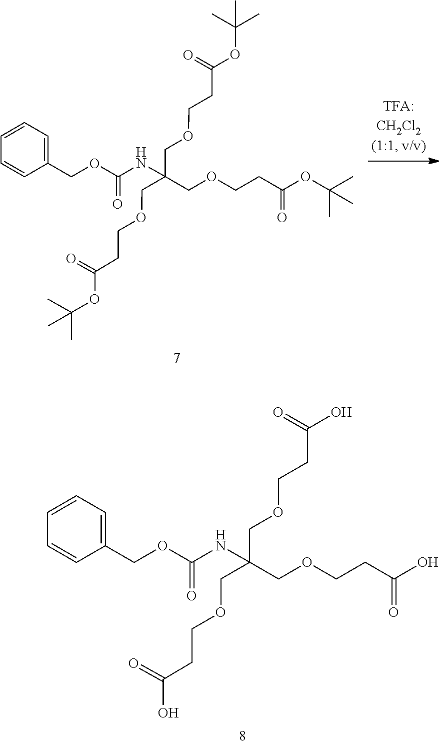 [SMILES: CC(C)(C)OC(=O)CCOCC(COCCC(=O)OC(C)(C)C)(COCCC(=O)OC(C)(C)C)NC(=O)OCc1ccccc1 ; O=C(O)CCOCC(COCCC(=O)O)(COCCC(=O)O)NC(=O)OCc1ccccc1]

Preparation of compound 8: Cbz-NH-tris-Boc-ester 7 (0.20 g, 0.39 mmol, 1.0 eq.) was dissolved in CH₂Cl₂ (1 mL) under argon, trifluoroacetic acid (TFA, 1 mL) was added and the reaction was stirred at room temperature for 1 h. The solvent was removed under reduced pressure, the residue was co-evaporated 3 times with toluene (5 mL) and dried under high vacuum to get the compound as its TFA salt (0.183 g, 98%). The compound was used without further purification. MS: calculated for C₂₁H₂₉NO₁₁, 471.6. Found 472.4.

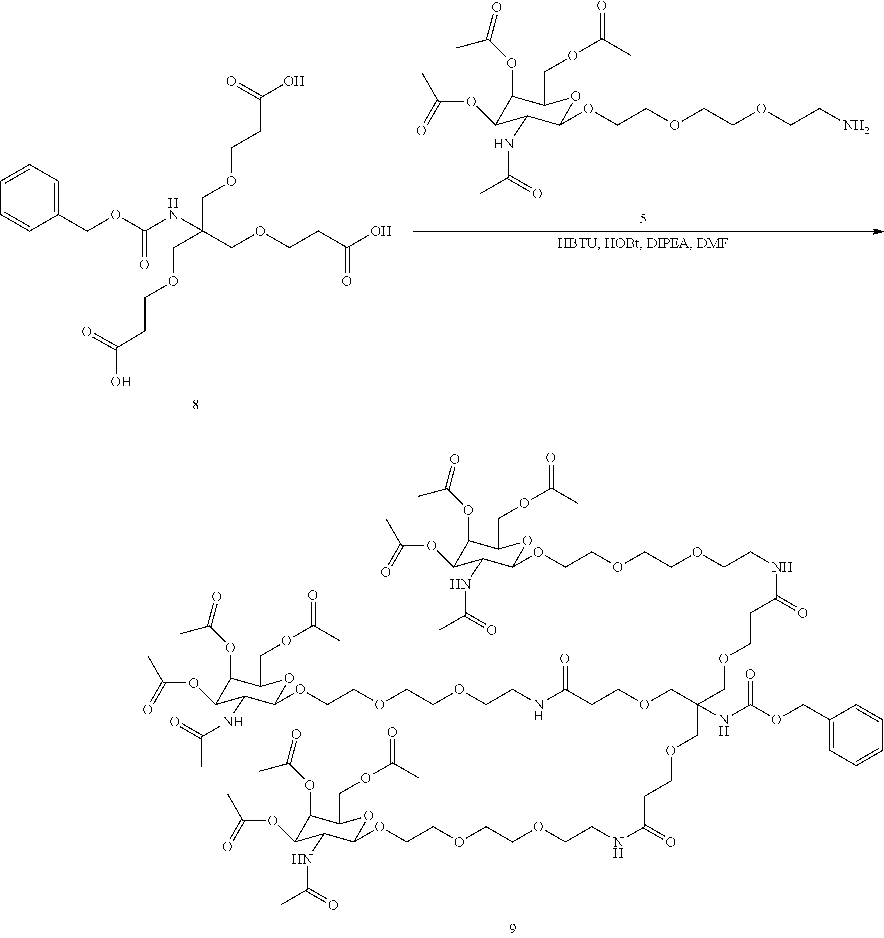 [SMILES: O=C(O)CCOCC(COCCC(=O)O)(COCCC(=O)O)NC(=O)OCc1ccccc1 ; CC(=O)NC1C(OCCOCCOCCN)OC(COC(C)=O)C(OC(C)=O)C1OC(C)=O ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)OCc2ccccc2)OC(COC(C)=O)C(OC(C)=O)C1OC(C)=O]

Preparation of compound 9: CbzNH-tris-COOH 8 (0.72 g, 1.49 mmol, 1.0 eq.) and GalNAc-PEG3-NH₂ 5 (3.56 g, 7.44 mmol, 5.0 eq.) were dissolved in N,N-dimethylformamide (DMF) (25 mL). Then N,N,N′,N′-tetramethyl-O-(1H-benzotriazol-1-131ynthesium hexafluorophosphate (HBTU) (2.78 g, 7.44 mmol, 5.0 eq.), 1-hydroxy benzotriazole hydrate (HOBt) (1.05 g, 7.44 mmol, 5.0 eq.) and N,N-diisopropylethylamine (DIPEA) (2.07 mL, 11.9 mmol, 8.0 eq.) were added to the solution and the reaction was stirred for 72 h. The solvent was removed under reduced pressure, the residue was dissolved in DCM (100 mL) and washed with saturated aq. NaHCO₃ (100 mL). The organic layer was dried over Na₂SO₄, the solvent evaporated and the crude material was purified by flash chromatography (gradient elution: 0-5% MeOH in DCM in 14 CV). The product was obtained as pale yellowish oil (1.2 g, 43%, rf=0.20 (5% MeOH in DCM)). MS: calculated for C₈₁H₁₂₅N₇O₄₁, 1852.9. Found 1854.7. ¹H NMR (500 MHz, DMSO-d₆) δ 7.90-7.80 (m, 10H), 7.65-7.62 (m, 4H), 7.47-7.43 (m, 3H), 7.38-7.32 (m, 8H), 5.24-5.22 (m, 3H), 5.02-4.97 (m, 4H), 4.60-4.57 (m, 3H), 4.07-3.90 (m 10H), 3.67-3.36 (m, 70H), 3.23-3.07 (m, 25H), 2.18 (s, 10H), 2.00 (s, 13H), 1.89 (s, 11H), 1.80-1.78 (m, 17H). ¹³C NMR (125 MHz, DMSO-d₆) δ 170.1 (C), 169.8 (C), 169.7 (C), 169.4 (C), 169.2 (C), 169.1 (C), 142.7 (C), 126.3 (CH), 123.9 (CH), 118.7 (CH), 109.7 (CH), 100.8 (CH), 70.5 (CH), 69.8 (CH), 69.6 (CH), 69.5 (CH), 69.3 (CH₂), 69.0 (CH₂), 68.2 (CH₂), 67.2 (CH₂), 66.7 (CH₂), 61.4 (CH₂), 22.6 (CH₂), 22.4 (3×CH3), 20.7 (9×CH3).

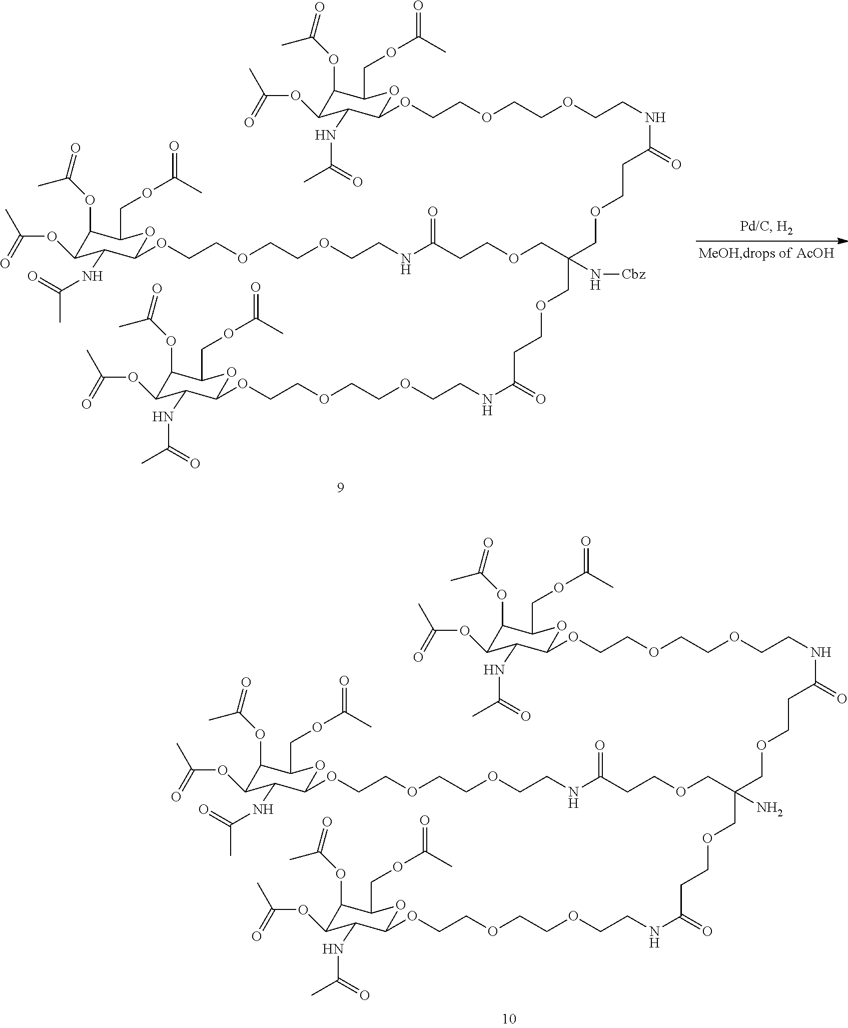 [SMILES: CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)OCc2ccccc2)OC(COC(C)=O)C(OC(C)=O)C1OC(C)=O ; CC(=O)NC1C(OCCOCCOCCNC(=O)CCOCC(N)(COCCC(=O)NCCOCCOCCOC2OC(COC(C)=O)C(OC(C)=O)C(OC(C)=O)C2NC(C)=O)COCCC(=O)NCCOCCOCCOC2OC(COC(C)=O)C(OC(C)=O)C(OC(C)=O)C2NC(C)=O)OC(COC(C)=O)C(OC(C)=O)C1OC(C)=O]

Preparation of compound 10: Triantennary GalNAc compound 9 (0.27 g, 0.14 mmol, 1.0 eq.) was dissolved in MeOH (15 mL), 3 drops of acetic acid (AcOH) and Pd/C (30 mg) was added. The reaction mixture was degassed using vacuum/argon cycles (3×) and hydrogenated under balloon pressure overnight. The completion of the reaction was followed by mass spectrometry and the resulting mixture was filtered through a thin pad of celite. The solvent was evaporated and the residue obtained was dried under high vacuum and used for the next step without further purification. The product was obtained as pale yellowish oil (0.24 g, quantitative yield). MS: calculated for C₇₃H₁₁₉N₇O₃₉, 1718.8. Found 1719.3.

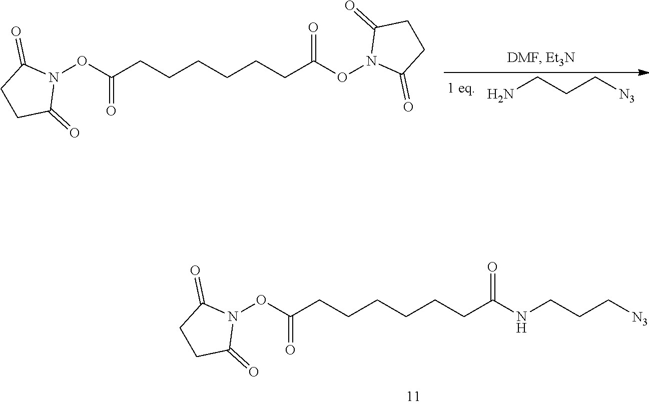 [SMILES: O=C(CCCCCCC(=O)ON1C(=O)CCC1=O)ON1C(=O)CCC1=O ; [N-]=[N+]=NCCCN ; [N-]=[N+]=NCCCNC(=O)CCCCCCC(=O)ON1C(=O)CCC1=O]

Preparation of compound 11: Commercially available suberic acid bis(N-hydroxysuccinimide ester) (3.67 g, 9.9 mmol, 1.0 eq.) was dissolved in DMF (5 mL) and triethylamine (1.2 mL) was added. To this solution was added dropwise a solution of 3-azido-1-propylamine (1.0 g, 9.9 mmol, 1.0 eq.) in DMF (5 mL). The reaction was stirred at room temperature for 3 h. The reaction mixture was diluted with EtOAc (100 mL) and washed with water (50 mL). The organic layer was separated, dried over Na₂SO₄ and the solvent was removed under reduced pressure. The crude material was purified by flash chromatography (gradient elution: 0-5% MeOH in DCM in 16 CV). The product was obtained as white solid (1.54 g, 43%, rf=0.71 (5% MeOH in DCM)). MS: calculated for C₁₅H₂₃N₅O₅, 353.4. Found 354.3.

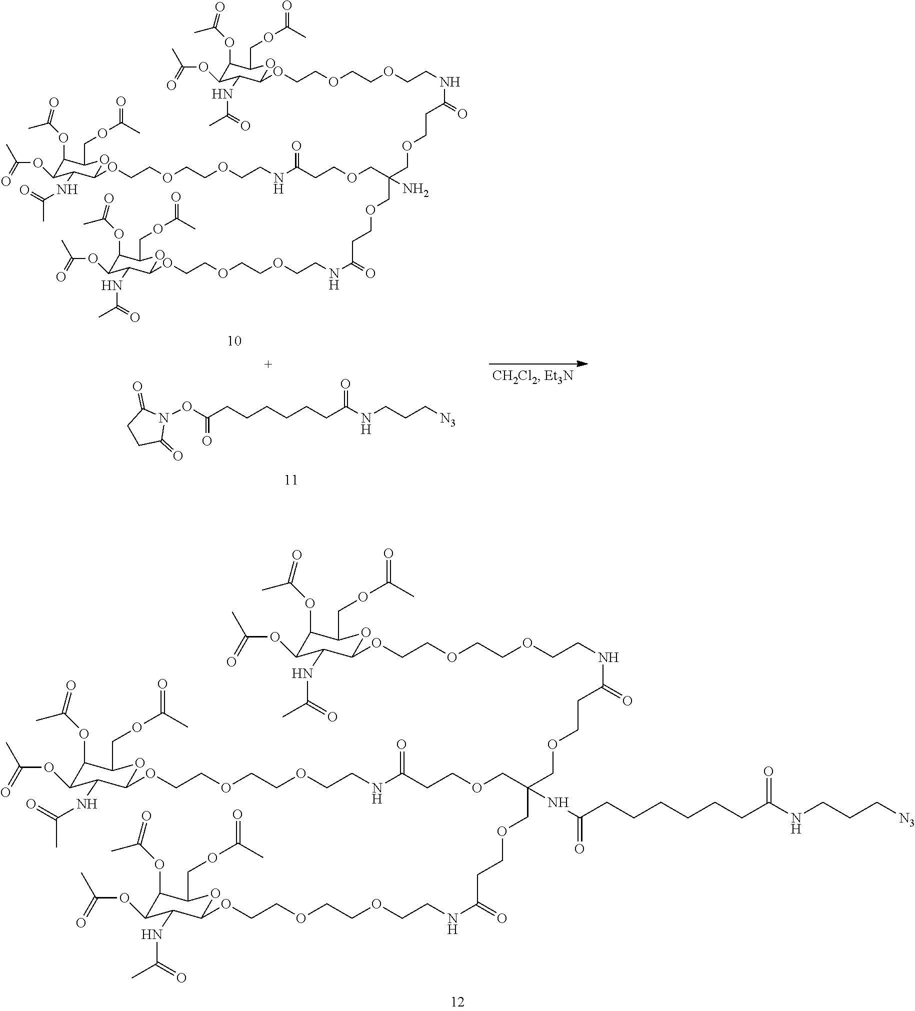 [SMILES: CC(=O)NC1C(OCCOCCOCCNC(=O)CCOCC(N)(COCCC(=O)NCCOCCOCCOC2OC(COC(C)=O)C(OC(C)=O)C(OC(C)=O)C2NC(C)=O)COCCC(=O)NCCOCCOCCOC2OC(COC(C)=O)C(OC(C)=O)C(OC(C)=O)C2NC(C)=O)OC(COC(C)=O)C(OC(C)=O)C1OC(C)=O ; [N-]=[N+]=NCCCNC(=O)CCCCCCC(=O)ON1C(=O)CCC1=O ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)CCCCCCC(=O)NCCCN=[N+]=[N-])OC(COC(C)=O)C(OC(C)=O)C1OC(C)=O]

Preparation of TriGalNAc (12): Triantennary GalNAc compound 10 (0.35 g, 0.24 mmol, 1.0 eq.) and compound 11 (0.11 g, 0.31 mmol, 1.5 eq.) were dissolved in DCM (5 mL) under argon and triethylamine (0.1 mL, 0.61 mmol, 3.0 eq.) was added. The reaction was stirred at room temperature overnight. The solvent was removed under reduced pressure, the residue was dissolved in EtOAc (100 mL) and washed with water (100 mL). The organic layer was separated and dried over Na₂SO₄. The solvent was evaporated and the resulting crude material was purified by flash chromatography (elution gradient: 0-10% MeOH in DCM in 20 CV) to afford the title compound as white fluffy solid (0.27 g, 67%, rf=0.5 (10% MeOH in DCM)). MS: calculated for C₈₄H₁₃₇N₁₁O₄₁, 1957.1. Found 1959.6.

Conjugation of Tether 1 to a siRNA Strand: Monofluoro Cyclooctyne (MFCO) Conjugation at 5′- or 3′-End

### 5′-End MFCO Conjugation

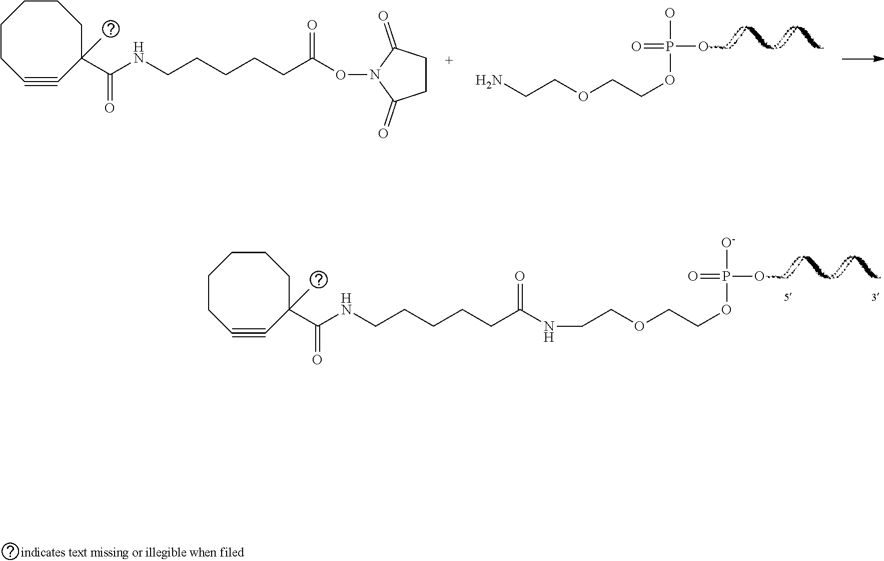 [SMILES: CC1(C(=O)NCCCCCC(=O)ON2C(=O)CCC2=O)C#CCCCCC1 ; NCCOCCOP(=O)(O)O ; CC1(C(=O)NCCCCCC(=O)NCCOCCOP(=O)([O-])O)C#CCCCCC1]

[symbol]

### 3′-End MFCO Conjugation

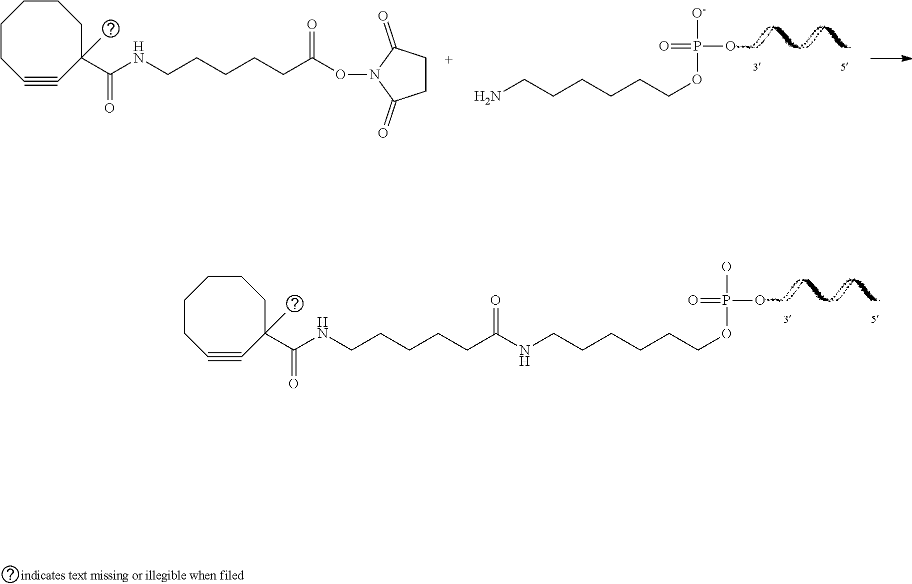 [SMILES: CC1(C(=O)NCCCCCC(=O)ON2C(=O)CCC2=O)C#CCCCCC1 ; NCCCCCCOP(=O)([O-])O ; CC1(C(=O)NCCCCCC(=O)NCCCCCCOP(=O)(O)O)C#CCCCCC1]

[symbol]

General conditions for MFCO conjugation: Amine-modified single strand was dissolved at 700 OD/mL in 50 mM carbonate/bicarbonate buffer pH 9.6/dimethyl sulfoxide (DMSO) 4:6 (v/v) and to this solution was added one molar equivalent of a 35 mM solution of MFCO-C6-NHS ester (Berry & Associates, Cat. #LK 4300) in DMF. The reaction was carried out at room temperature and after 1 h another molar equivalent of the MFCO solution was added. The reaction was allowed to proceed for an additional hour and was monitored by LC/MS. At least two molar equivalent excess of the MFCO NHS ester reagent relative to the amino modified oligonucleotide were needed to achieve quantitative consumption of the starting material. The reaction mixture was diluted 15-fold with water, filtered through a 1.2 μm filter from Sartorius and then purified by reserve phase (RP HPLC) on an Äkta Pure instrument (GE Healthcare).

Purification was performed using a XBridge C18 Prep 19×50 mm column from Waters. Buffer A was 100 mM TEAAc pH 7 and buffer B contained 95% acetonitrile in buffer A. A flow rate of 10 mL/min and a temperature of 60° C. were employed. UV traces at 280 nm were recorded. A gradient of 0-100% B within 60 column volumes was employed.

Fractions containing full length conjugated oligonucleotide were pooled, precipitated in the freezer with 3 M NaOAc, pH 5.2 and 85% ethanol and the collected pellet was dissolved in water. Samples were desalted by size exclusion chromatography and concentrated using a speed-vac concentrator to yield the conjugated oligonucleotide in an isolated yield of 40-80%.

### 5′-GalNAc-T1 Conjugates

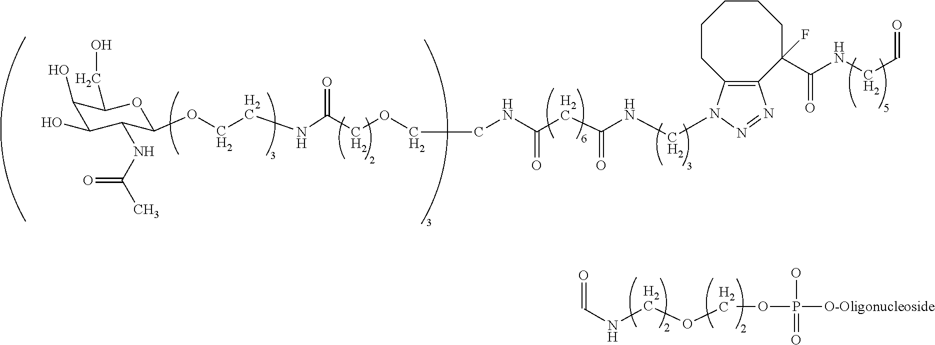

### 3′-GalNAc-T1 Conjugates

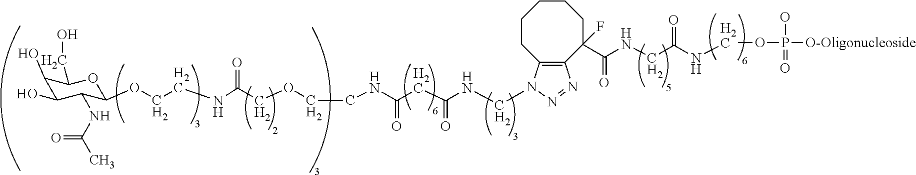

General procedure for TriGalNAc conjugation: MFCO-modified single strand was dissolved at 2000 OD/mL in water and to this solution was added one equivalent solution of compound 12 (10 mM) in DMF. The reaction was carried out at room temperature and after 3 h 0.7 molar equivalent of the compound 12 solution was added. The reaction was allowed to proceed overnight and completion was monitored by LCMS. The conjugate was diluted 15-fold in water, filtered through a 1.2 μm filter from Sartorius and then purified by RP HPLC on an Äkta Pure instrument (GE Healthcare).

RP HPLC purification was performed using a XBridge C18 Prep 19×50 mm column from Waters. Buffer A was 100 mM triethylammonium acetate pH 7 and buffer B contained 95% acetonitrile in buffer A. A flow rate of 10 mL/min and a temperature of 60° C. were employed. UV traces at 280 nm were recorded. A gradient of 0-100% B within 60 column volumes was employed.

Fractions containing full-length conjugated oligonucleotide were pooled, precipitated in the freezer with 3 M NaOAc, pH 5.2 and 85% ethanol and the collected pellet was dissolved in water to give an oligonucleotide solution of about 1000 OD/mL. The O-acetates were removed by adding 20% aqueous ammonia. Quantitative removal of these protecting groups was verified by LC-MS.

The conjugates were desalted by size exclusion chromatography using Sephadex G25 Fine resin (GE Healthcare) on an Äkta Pure (GE Healthcare) instrument to yield the conjugated oligonucleotides in an isolated yield of 50-70%.

The following schemes further set out the routes of synthesis:

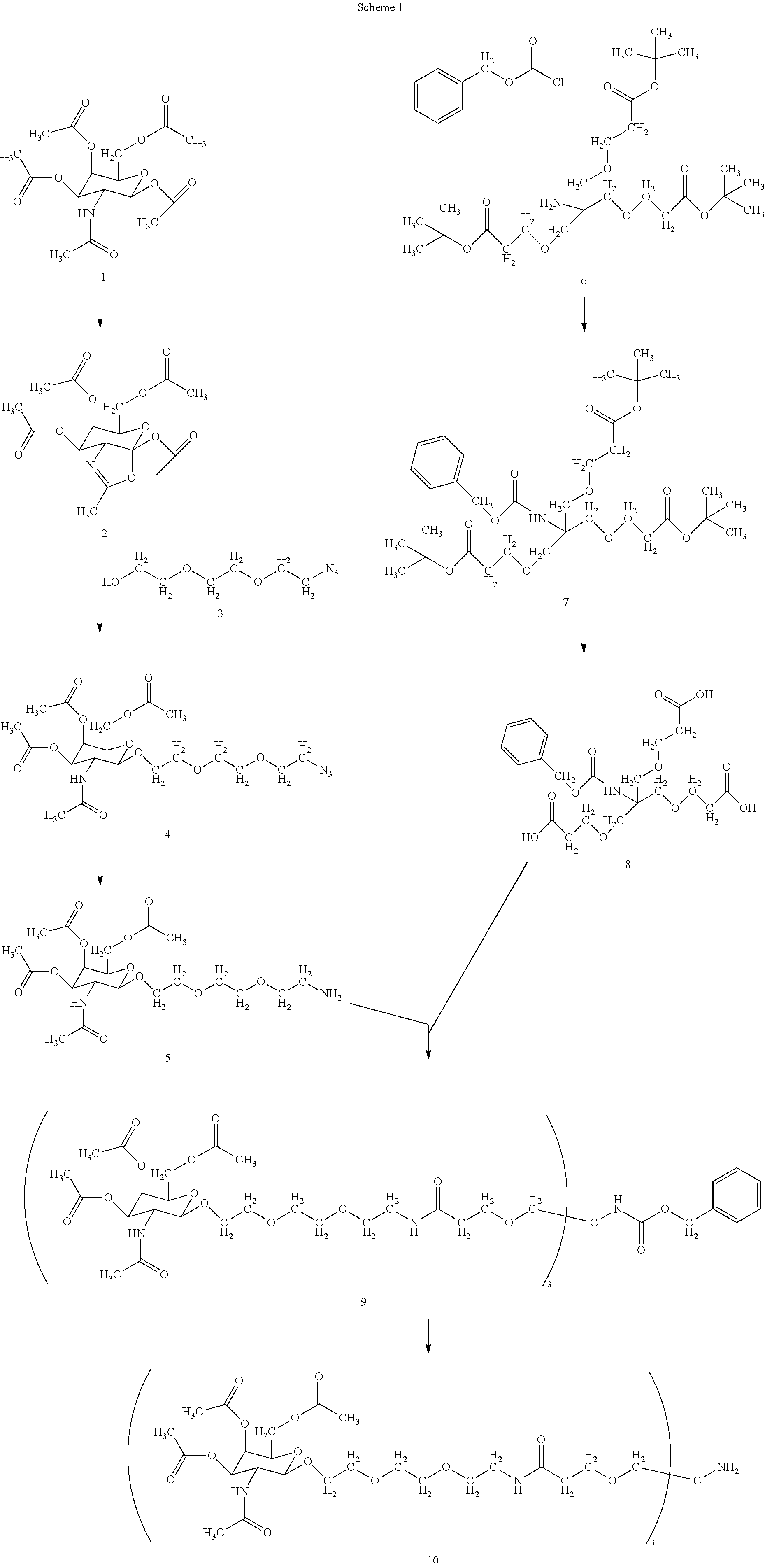

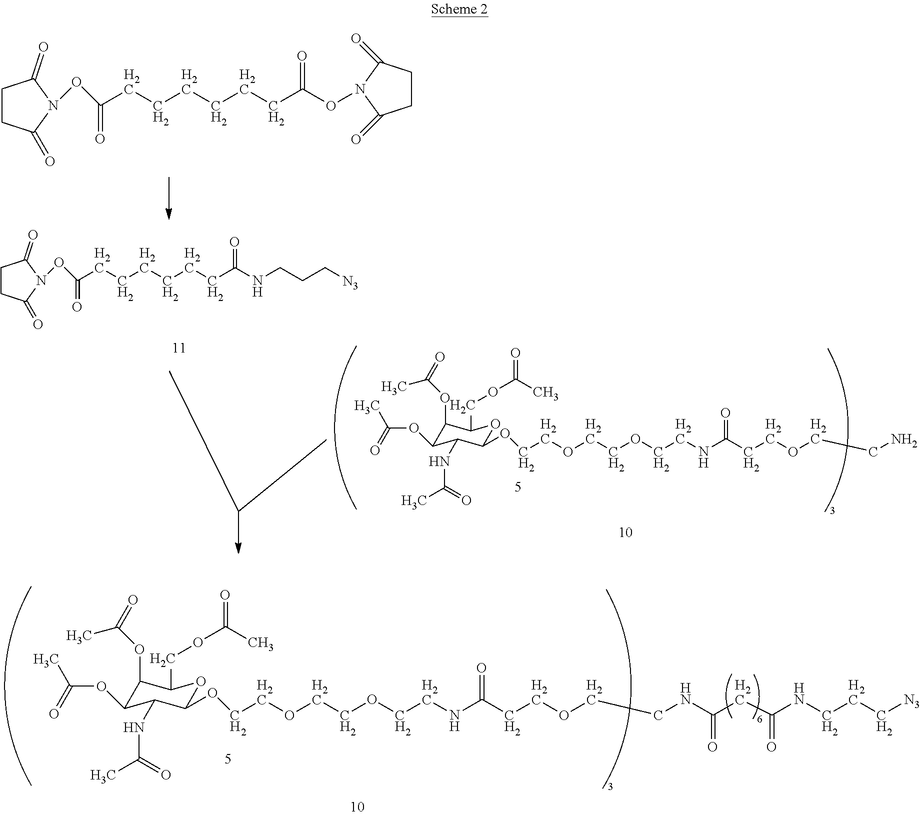

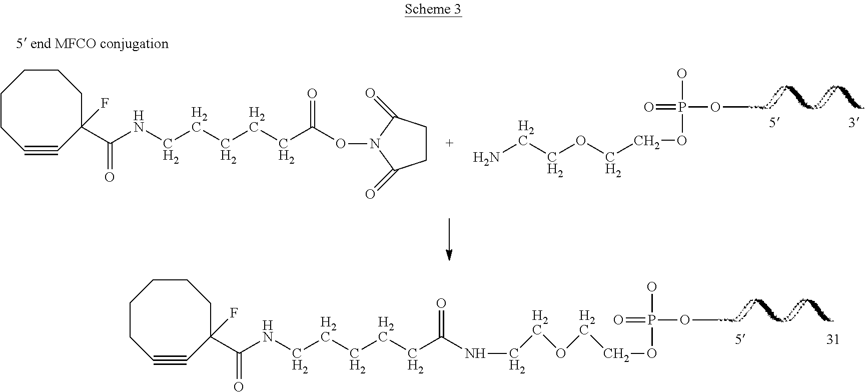 [SMILES: O=C(CCCCCNC(=O)C1(F)C#CCCCCC1)ON1C(=O)CCC1=O ; COP(=O)(O)OCCOCCN ; COP(=O)(O)OCCOCCNC(=O)CCCCCNC(=O)C1(F)C#CCCCCC1]

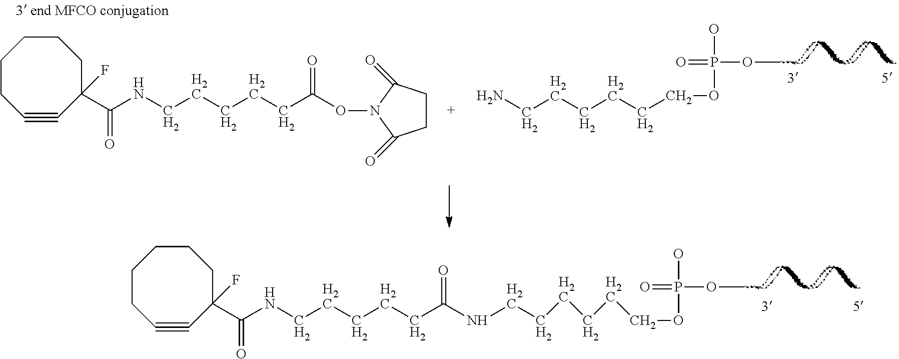 [SMILES: O=C(CCCCCNC(=O)C1(F)C#CCCCCC1)ON1C(=O)CCC1=O ; COP(=O)(O)OCCCCCCN ; COP(=O)(O)OCCCCCCNC(=O)CCCCCNC(=O)C1(F)C#CCCCCC1]

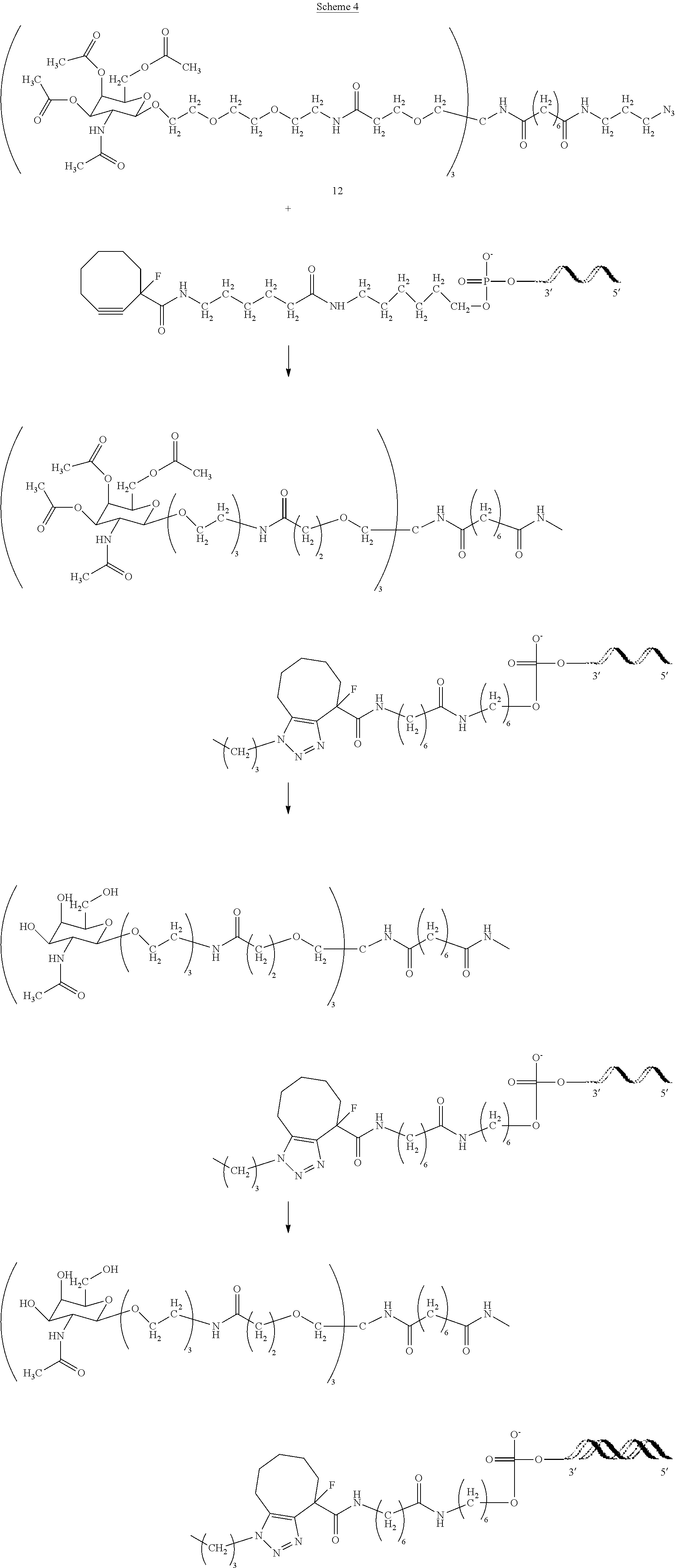

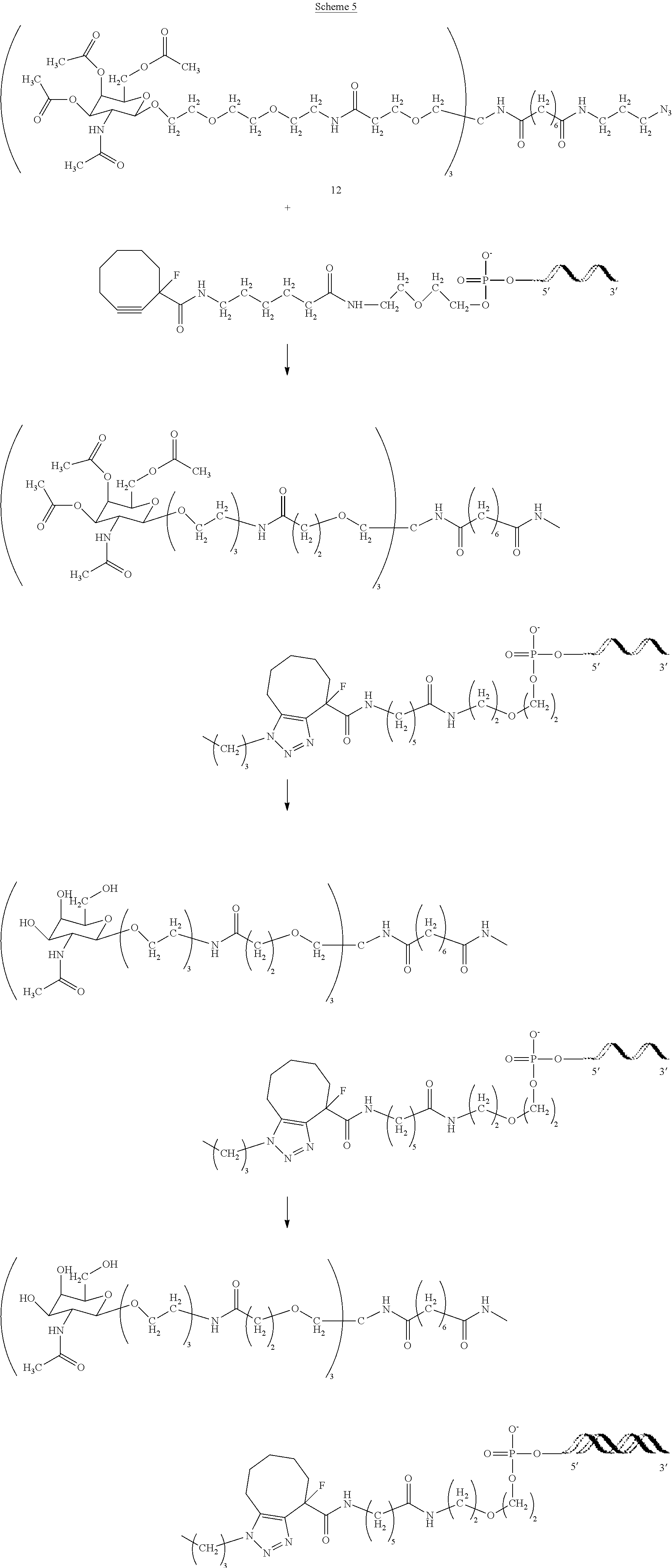

### Example 2: Duplex Annealing

To generate the desired siRNA duplex, the two complementary strands were annealed by combining equimolar aqueous solutions of both strands. The mixtures were placed into a water bath at 70° C. for 5 minutes and subsequently allowed to cool to ambient temperature within 2 h. The duplexes were lyophilized for 2 days and stored at −20° C.

The duplexes were analyzed by analytical SEC HPLC on Superdex™ 75 Increase 5/150 GL column 5×153-158 mm (Cytiva) on a Dionex Ultimate 3000 (Thermo Fisher Scientific) HPLC system. Mobile phase consisted of 1×PBS containing 10% acetonitrile. An isocratic gradient was run in 10 min at a flow rate of 1.5 mL/min at room temperature. UV traces at 260 and 280 nm were recorded. Water (LC-MS grade) was purchased from Sigma-Aldrich and Phosphate-buffered saline (PBS: 10×, pH 7.4) was purchased from GIBCO (Thermo Fisher Scientific).

### Example 3: Synthesis of Tether 2

### General Experimental Conditions

Thin layer chromatography (TLC) was performed on silica-coated aluminium plates with fluorescence indicator 254 nm from Macherey-Nagel. Compounds were visualized under UV light (254 nm), or after spraying with the 5% H₂SO₄ in methanol (MeOH) or ninhydrin reagent according to Stahl (from Sigma-Aldrich), followed by heating. Flash chromatography was performed with a Biotage Isolera One flash chromatography instrument equipped with a dual variable UV wavelength detector (200-400 nm) using Biotage Sfär Silica 10, 25, 50 or 100 g columns (Uppsala, Sweden).

All moisture-sensitive reactions were carried out under anhydrous conditions using dry glassware, anhydrous solvents, and argon atmosphere. All commercially available reagents were purchased from Sigma-Aldrich and solvents from Carl Roth GmbH+Co. KG. D-Galactosamine pentaacetate was purchased from AK scientific.

HPLC/ESI-MS was performed on a Dionex UltiMate 3000 RS UHPLC system and Thermo Scientific MSQ Plus Mass spectrometer using an Acquity UPLC Protein BEH C4 column from Waters (300 Å, 1.7 μm, 2.1×100 mm) at 60° C. The solvent system consisted of solvent A with H₂O containing 0.1% formic acid and solvent B with acetonitrile (ACN) containing 0.1% formic acid. A gradient from 5-100% of B over 15 min with a flow rate of 0.4 mL/min was employed. Detector and conditions: Corona ultra-charged aerosol detection (from esa). Nebulizer Temp.: 25° C. N₂ pressure: 35.1 psi. Filter: Corona.

¹H and ¹³C NMR spectra were recorded at room temperature on a Varian spectrometer at 500 MHz (¹H NMR) and 125 MHz (¹³C NMR). Chemical shifts are given in ppm referenced to the solvent residual peak (CDCl₃—¹H NMR: δ at 7.26 ppm and ¹³C NMR δ at 77.2 ppm: DMSO-d₆—¹H NMR: δ at 2.50 ppm and ¹³C NMR δ at 39.5 ppm). Coupling constants are given in Hertz. Signal splitting patterns are described as singlet(s), doublet (d), triplet (t) or multiplet (m).

### Synthesis Route for the Conjugate Building Block TriGalNAc_Tether2:

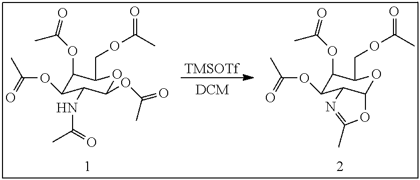 [SMILES: "TMSOTf" ; CC(=O)NC1C(OC(C)=O)OC(COC(C)=O)C(OC(C)=O)C1OC(C)=O ; CC(=O)OCC1OC2OC(C)=NC2C(OC(C)=O)C1OC(C)=O]

Preparation of compound 2: D-Galactosamine pentaacetate (3.00 g, 7.71 mmol, 1.0 eq.) was dissolved in anhydrous dichloromethane (DCM) (30 mL) under argon and trimethylsilyl trifluoromethanesulfonate (TMSOTf, 4.28 g, 19.27 mmol, 2.5 eq.) was added. The reaction was stirred at room temperature for 3 h. The reaction mixture was diluted with DCM (50 mL) and washed with cold saturated aq. NaHCO₃ (100 mL) and water (100 mL). The organic layer was separated, dried over Na₂SO₄, and concentrated to afford the title compound as yellow oil, which was purified by flash chromatography (gradient elution: 0-10% MeOH in DCM in 10 CV). The product was obtained as colourless oil (2.5 g, 98%, rf=0.45 (2% MeOH in DCM)).

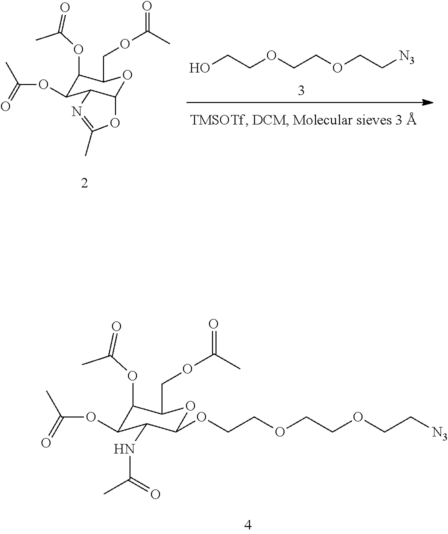 [SMILES: CC(=O)OCC1OC2OC(C)=NC2C(OC(C)=O)C1OC(C)=O ; [N-]=[N+]=NCCOCCOCCO ; CC(=O)NC1C(OCCOCCOCCN=[N+]=[N-])OC(COC(C)=O)C(OC(C)=O)C1OC(C)=O]

Preparation of compound 4: Compound 2 (2.30 g, 6.98 mmol, 1.0 eq.) and azido-PEG3-OH (1.83 g, 10.5 mmol, 1.5 eq.) were dissolved in anhydrous DCM (40 mL) under argon and molecular sieves 3 Å (5 g) were added to the solution. The mixture was stirred at room temperature for 1 h. TMSOTf (0.77 g, 3.49 mmol, 0.5 eq.) was then added to the mixture and the reaction was stirred overnight. The molecular sieves were filtered, the filtrate was diluted with DCM (100 mL) and washed with cold saturated aq. NaHCO₃ (100 mL) and water (100 mL). The organic layer was separated, dried over Na₂SO₄ and the solvent was removed under reduced pressure. The crude material was purified by flash chromatography (gradient elution: 0-3% MeOH in DCM in 10 CV) to afford the title product as light-yellow oil (3.10 g, 88%, rf=0.25 (2% MeOH in DCM)). MS: calculated for C₂₀H₃₂N₄O₁₁, 504.21. Found 505.4. ¹H NMR (500 MHz, CDCl₃) δ 6.21-6.14 (m, 1H), 5.30 (dd, J=3.4, 1.1 Hz, 1H), 5.04 (dd, J=11.2, 3.4 Hz, 1H), 4.76 (d, J=8.6 Hz, 1H), 4.23-4.08 (m, 3H), 3.91-3.80 (m, 3H), 3.74-3.59 (m, 9H), 3.49-3.41 (m, 2H), 2.14 (s, 3H), 2.02 (s, 3H), 1.97 (d, J=4.2 Hz, 6H). ¹³C NMR (125 MHz, CDCl₃) δ 170.6 (C), 170.5 (C), 170.4 (C), 170.3 (C), 102.1 (CH), 71.6 (CH), 70.8 (CH), 70.6 (CH), 70.5 (CH), 70.3 (CH₂), 69.7 (CH₂), 68.5 (CH₂), 66.6 (CH₂), 61.5 (CH₂), 23.1 (CH₃), 20.7 (3×CH3).

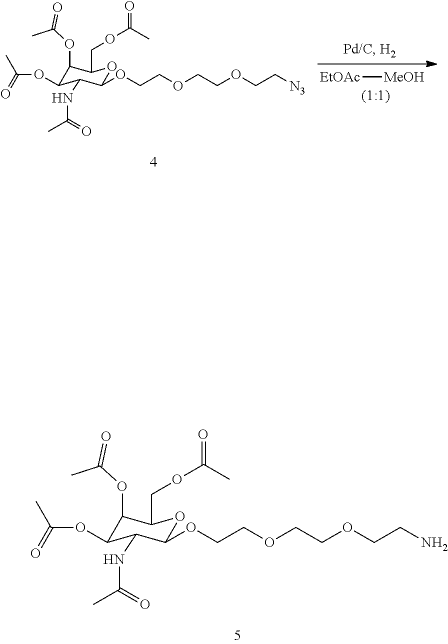

Preparation of compound 5: Compound 4 (1.00 g, 1.98 mmol, 1.0 eq.) was dissolved in a mixture of ethyl acetate (EtOAc) and MeOH (30 mL 1:1 v/v) and Pd/C (100 mg) was added. The reaction mixture was degassed using vacuum/argon cycles (3×) and hydrogenated under balloon pressure overnight. The reaction mixture was filtered through celite and washed with EtOAc (30 mL). The solvent was removed under reduced pressure to afford the title compound as colourless oil (0.95 g, quantitative yield, rf=0.25 (10% MeOH in DCM)). The compound was used without further purification. MS: calculated for C₂₀H₃₄N₂O₁₁, 478.2. Found 479.4.

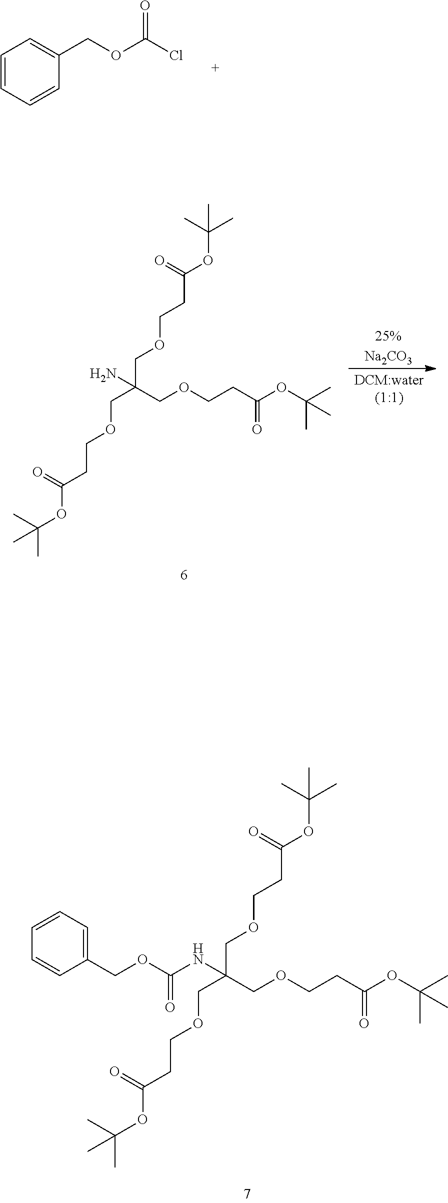 [SMILES: O=C(Cl)OCc1ccccc1 ; CC(C)(C)OC(=O)CCOCC(N)(COCCC(=O)OC(C)(C)C)COCCC(=O)OC(C)(C)C ; CC(C)(C)OC(=O)CCOCC(COCCC(=O)OC(C)(C)C)(COCCC(=O)OC(C)(C)C)NC(=O)OCc1ccccc1]

Preparation of compound 7: Tris {[2-(tert-butoxycarbonyl) ethoxy]methyl}-methylamine 6 (3.37 g, 6.67 mmol, 1.0 eq.) was dissolved in a mixture of DCM/water (40 mL 1:1 v/v) and Na₂CO₃ (0.18 g, 1.7 mmol, 0.25 eq.) was added while stirring vigorously. Benzyl chloroformate (2.94 mL, 20.7 mmol, 3.10 eq.) was added dropwise to the previous mixture and the reaction was stirred at room temperature for 24 h. The reaction mixture was diluted with CH₂Cl₂ (100 mL) and washed with water (100 mL). The organic layer was separated and dried over Na₂SO₄. The solvent was removed under reduced pressure and the resulting crude material was purified by flash chromatography (gradient elution: 0-10% EtOAc in cyclohexane in 12 CV) to afford the title compound as pale yellowish oil (3.9 g, 91%, rf=0.56 (10% EtOAc in cyclohexane)). MS: calculated for C₃₃H₅₃NO₁₁, 639.3. Found 640.9. ¹H NMR (500 MHz, DMSO-d₆) δ 7.38-7.26 (m, 5H), 4.97 (s, 2H), 3.54 (t, 6H), 3.50 (s, 6H), 2.38 (t, 6H), 1.39 (s, 27H). ¹³C NMR (125 MHz, DMSO-d₆) δ 170.3 (3×C), 154.5 (C), 137.1 (C), 128.2 (2×CH), 127.7 (CH), 127.6 (2×CH), 79.7 (3×C), 68.4 (3×CH2), 66.8 (3×CH2), 64.9 (C), 58.7 (CH₂), 35.8 (3×CH2), 27.7 (9×CH3).

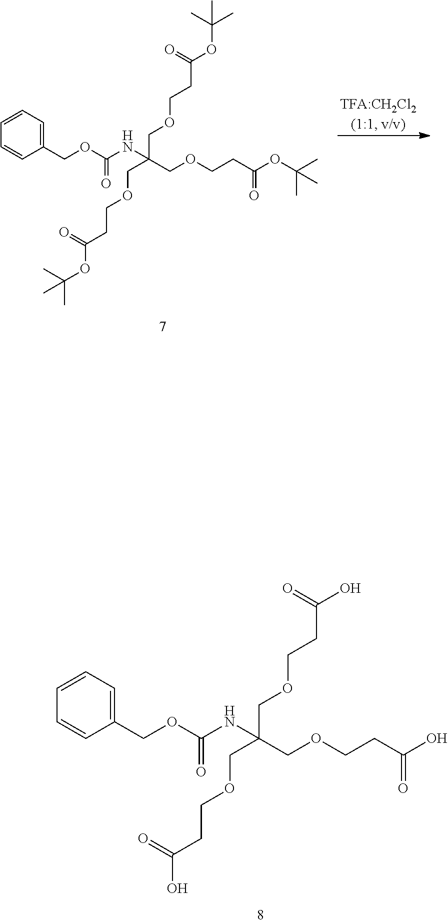 [SMILES: CC(C)(C)OC(=O)CCOCC(COCCC(=O)OC(C)(C)C)(COCCC(=O)OC(C)(C)C)NC(=O)OCc1ccccc1 ; O=C(O)CCOCC(COCCC(=O)O)(COCCC(=O)O)NC(=O)OCc1ccccc1]

Preparation of compound 8: Cbz-NH-tris-Boc-ester 7 (0.20 g, 0.39 mmol, 1.0 eq.) was dissolved in CH₂Cl₂ (1 mL) under argon, trifluoroacetic acid (TFA, 1 mL) was added and the reaction was stirred at room temperature for 1 h. The solvent was removed under reduced pressure, the residue was co-evaporated 3 times with toluene (5 mL) and dried under high vacuum to get the compound as its TFA salt (0.183 g, 98%). The compound was used without further purification. MS: calculated for C₂₁H₂₉NO₁₁, 471.6. Found 472.4.

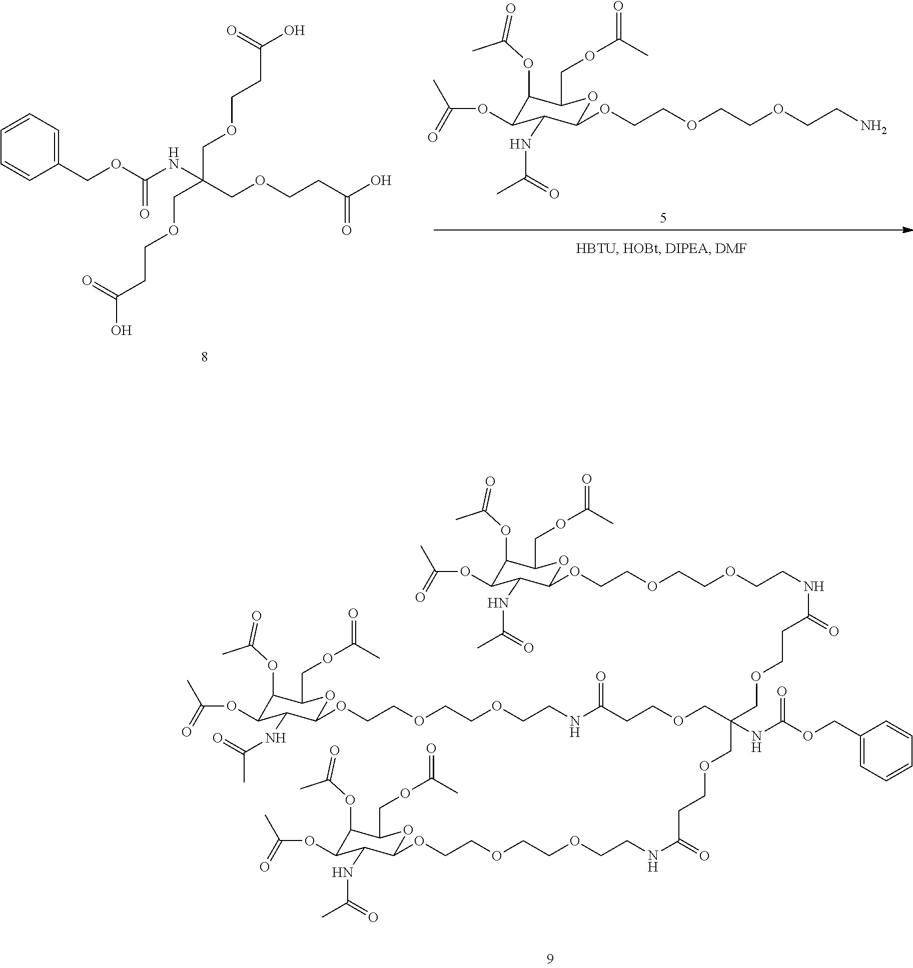 [SMILES: O=C(O)CCOCC(COCCC(=O)O)(COCCC(=O)O)NC(=O)OCc1ccccc1 ; CC(=O)NC1C(OCCOCCOCCN)OC(COC(C)=O)C(OC(C)=O)C1OC(C)=O ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)OCc2ccccc2)OC(COC(C)=O)C(OC(C)=O)C1OC(C)=O]

Preparation of compound 9: CbzNH-tris-COOH 8 (0.72 g, 1.49 mmol, 1.0 eq.) and GalNAc-PEG3-NH₂ 5 (3.56 g, 7.44 mmol, 5.0 eq.) were dissolved in N,N-dimethylformamide (DMF) (25 mL). Then N,N,N′,N′-tetramethyl-O-(1H-benzotriazol-1-146ynthesium hexafluorophosphate (HBTU) (2.78 g, 7.44 mmol, 5.0 eq.), 1-hydroxybenzotriazole hydrate (HOBt) (1.05 g, 7.44 mmol, 5.0 eq.) and N,N-diisopropylethylamine (DIPEA) (2.07 mL, 11.9 mmol, 8.0 eq.) were added to the solution and the reaction was stirred for 72 h. The solvent was removed under reduced pressure, the residue was dissolved in DCM (100 mL) and washed with saturated aq. NaHCO₃ (100 mL). The organic layer was dried over Na₂SO₄. the solvent evaporated and the crude material was purified by flash chromatography (gradient elution: 0-5% MeOH in DCM in 14 CV). The product was obtained as pale yellowish oil (1.2 g, 43%, rf=0.20 (5% MeOH in DCM)). MS: calculated for C₈₁H₁₂₅N₇O₄₁, 1852.9. Found 1854.7. ¹H NMR (500 MHz, DMSO-d₆) δ 7.90-7.80 (m, 10H), 7.65-7.62 (m, 4H), 7.47-7.43 (m, 3H), 7.38-7.32 (m, 8H), 5.24-5.22 (m, 3H), 5.02-4.97 (m, 4H), 4.60-4.57 (m, 3H), 4.07-3.90 (m 10H), 3.67-3.36 (m, 70H), 3.23-3.07 (m, 25H), 2.18 (s, 10H), 2.00 (s, 13H), 1.89 (s, 11H), 1.80-1.78 (m, 17H). ¹³C NMR (125 MHz, DMSO-d₆) δ 170.1 (C), 169.8 (C), 169.7 (C), 169.4 (C), 169.2 (C), 169.1 (C), 142.7 (C), 126.3 (CH), 123.9 (CH), 118.7 (CH), 109.7 (CH), 100.8 (CH), 70.5 (CH), 69.8 (CH), 69.6 (CH), 69.5 (CH), 69.3 (CH₂), 69.0 (CH₂), 68.2 (CH₂), 67.2 (CH₂), 66.7 (CH₂), 61.4 (CH₂), 22.6 (CH₂), 22.4 (3×CH3), 20.7 (9×CH3).

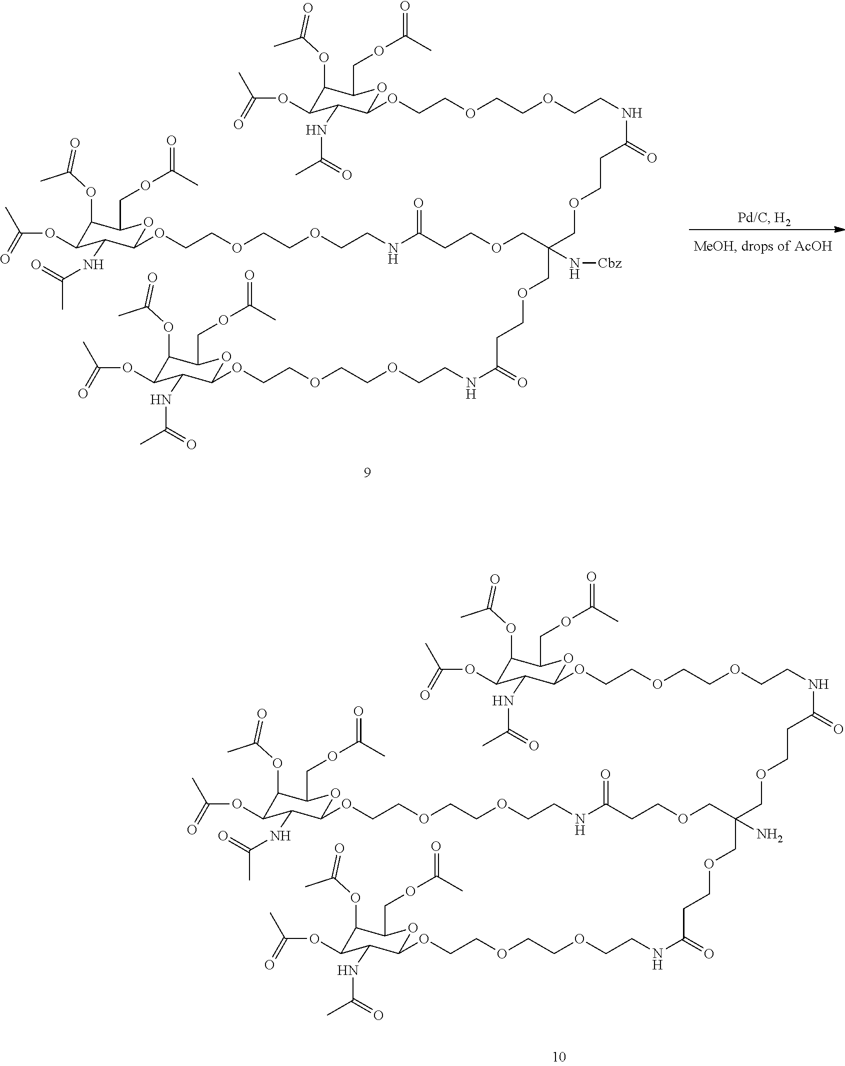 [SMILES: CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)OCc2ccccc2)OC(COC(C)=O)C(OC(C)=O)C1OC(C)=O ; CC(=O)NC1C(OCCOCCOCCNC(=O)CCOCC(N)(COCCC(=O)NCCOCCOCCOC2OC(COC(C)=O)C(OC(C)=O)C(OC(C)=O)C2NC(C)=O)COCCC(=O)NCCOCCOCCOC2OC(COC(C)=O)C(OC(C)=O)C(OC(C)=O)C2NC(C)=O)OC(COC(C)=O)C(OC(C)=O)C1OC(C)=O]

[symbol]

Preparation of compound 10: Triantennary GalNAc compound 9 (0.27 g, 0.14 mmol, 1.0 eq.) was dissolved in MeOH (15 mL), 3 drops of acetic acid (AcOH) and Pd/C (30 mg) was added. The reaction mixture was degassed using vacuum/argon cycles (3×) and hydrogenated under balloon pressure overnight. The completion of the reaction was followed by mass spectrometry and the resulting mixture was filtered through a thin pad of celite. The solvent was evaporated, and the residue obtained was dried under high vacuum and used for the next step without further purification. The product was obtained as pale yellowish oil (0.24 g, quantitative yield). MS: calculated for C₇₃H₁₁₉N₇O₃₉, 1718.8. Found 1719.3.

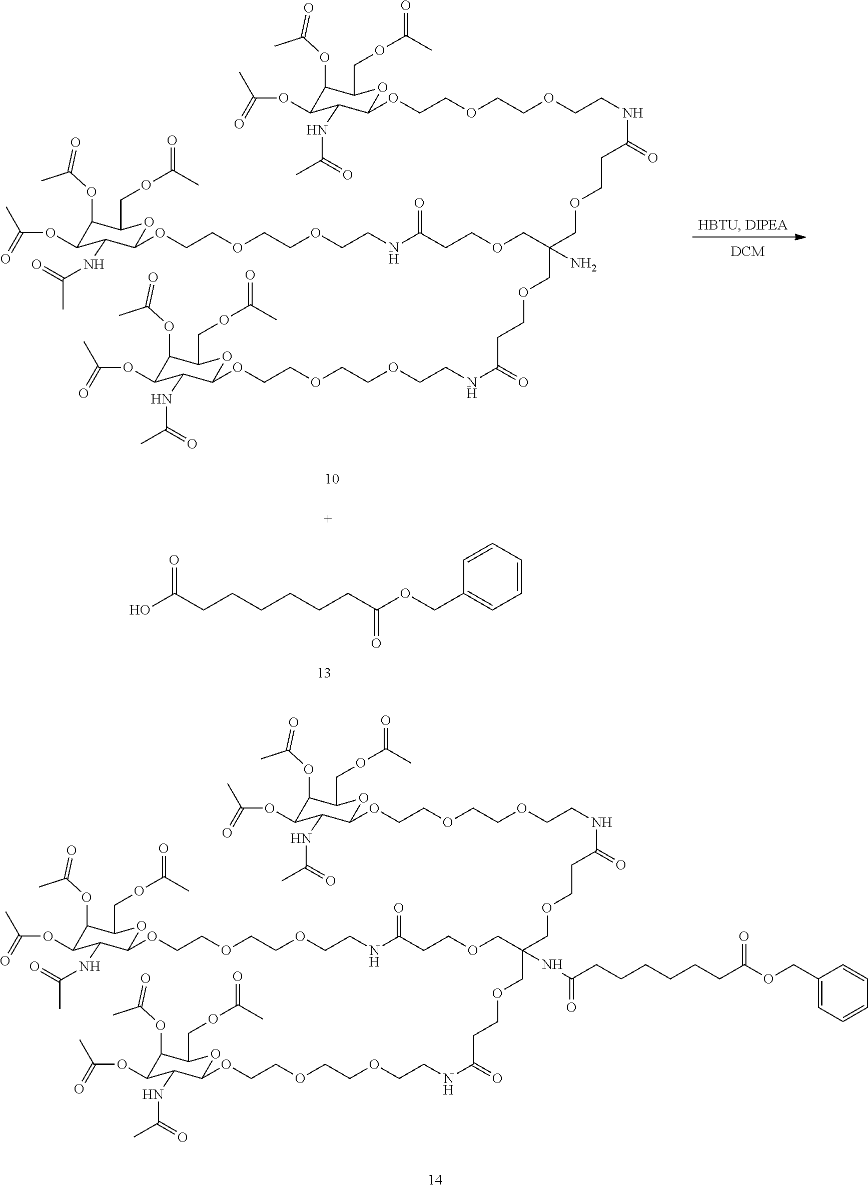 [SMILES: CC(=O)NC1C(OCCOCCOCCNC(=O)CCOCC(N)(COCCC(=O)NCCOCCOCCOC2OC(COC(C)=O)C(OC(C)=O)C(OC(C)=O)C2NC(C)=O)COCCC(=O)NCCOCCOCCOC2OC(COC(C)=O)C(OC(C)=O)C(OC(C)=O)C2NC(C)=O)OC(COC(C)=O)C(OC(C)=O)C1OC(C)=O ; O=C(O)CCCCCCC(=O)OCc1ccccc1 ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)CCCCCCC(=O)OCc2ccccc2)OC(COC(C)=O)C(OC(C)=O)C1OC(C)=O]

[symbol]

Preparation of compound 14: Triantennary GalNAc compound 10 (0.45 g, 0.26 mmol, 1.0 eq.), HBTU (0.19 g, 0.53 mmol, 2.0 eq.) and DIPEA (0.23 mL, 1.3 mmol, 5.0 eq.) were dissolved in DCM (10 mL) under argon. To this mixture, it was added dropwise a solution of compound 13 (0.14 g, 0.53 mmol, 2.0 eq.) in DCM (5 mL). The reaction was stirred at room temperature overnight. The solvent was removed, and the residue was dissolved in EtOAc (50 mL), washed with water (50 mL) and dried over Na₂SO₄. The solvent was evaporated, and the crude material was purified by flash chromatography (gradient elution: 0-5% MeOH in DCM in 20 CV). The product was obtained as white fluffy solid (0.25 g, 48%, rf=0.4 (10% MeOH in DCM)). MS: calculated for C₈₈H₁₃₇N₇O₄₂, 1965.1. Found 1965.6.

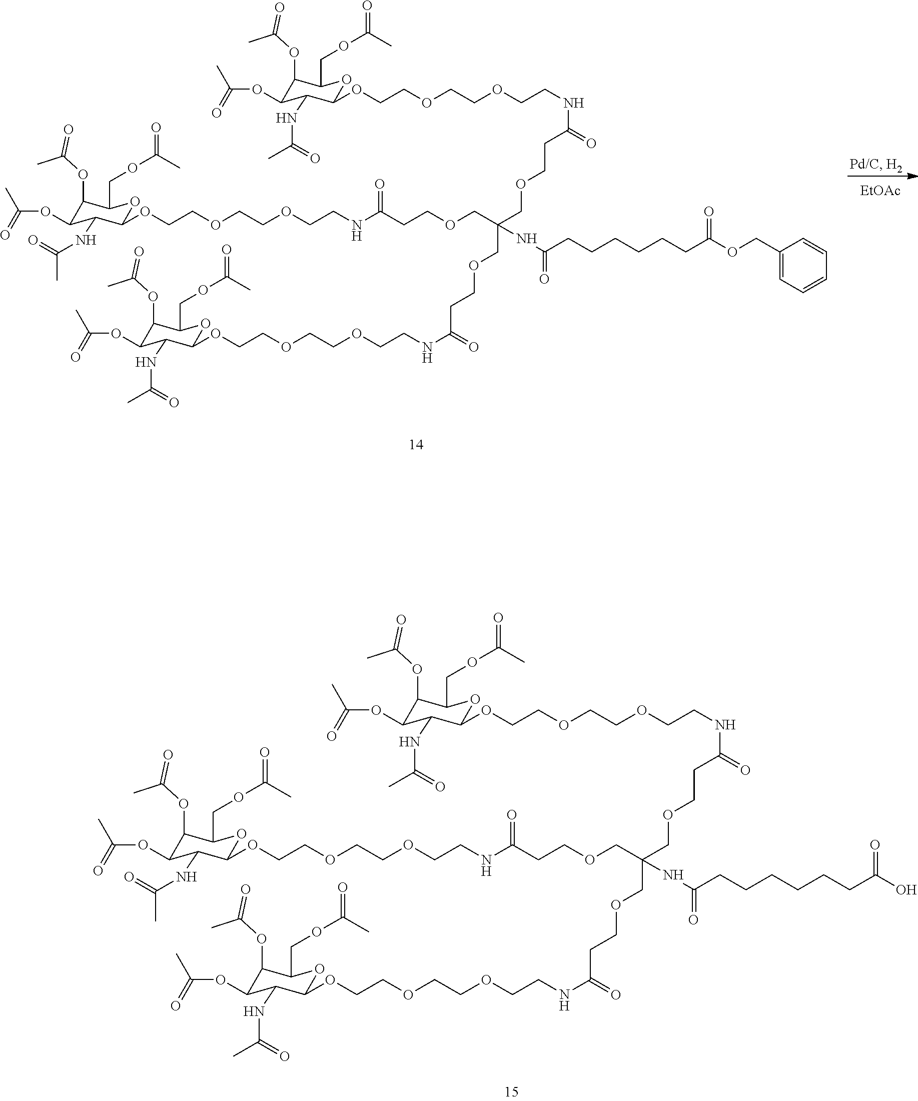 [SMILES: "EtOAc" ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)CCCCCCC(=O)OCc2ccccc2)OC(COC(C)=O)C(OC(C)=O)C1OC(C)=O ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)CCCCCCC(=O)O)OC(COC(C)=O)C(OC(C)=O)C1OC(C)=O]

[symbol]

Preparation of TriGalNAc (15): Triantennary GalNAc compound 14 (0.31 g, 0.15 mmol, 1.0 eq.) was dissolved in EtOAc (15 mL) and Pd/C (40 mg) was added. The reaction mixture was degassed by using vacuum/argon cycles (3×) and hydrogenated under balloon pressure overnight. The completion of the reaction was monitored by mass spectrometry and the resulting mixture was filtered through a thin pad of celite. The solvent was removed under reduced pressure and the resulting residue was dried under high vacuum overnight. The residue was used for conjugations to oligonucleosides without further purification (0.28 g, quantitative yield). MS: calculated for C₈₁H₁₃₁N₇O₄₂, 1874.9. Found 1875.3.

Conjugation of Tether 2 to a siRNA Strand: TriGalNAc Tether 2 (GalNAc-T2) Conjugation at 5′-End or 3′-End

### 5′-GalNAc-T2 Conjugates

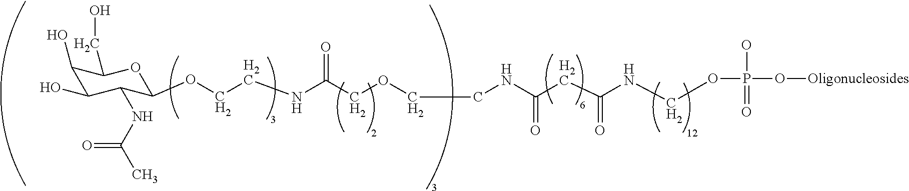

### 3′-GalNAc-T2 Conjugates

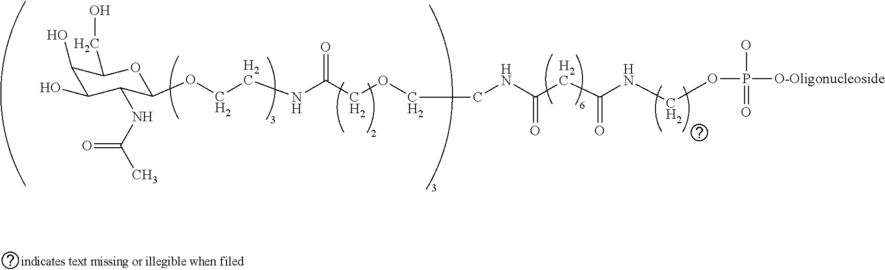

Preparation of TriGalNAc tether 2 NHS ester: To a solution of carboxylic acid tether 2 (compound 15, 227 mg, 121 μmol) in DMF (2.1 mL), N-hydroxysuccinimide (NHS) (15.3 mg, 133 μmol) and N,N′-diisopropylcarbodiimide (DIC) (19.7 μL, 127 μmol) were added. The solution was stirred at room temperature for 18 h and used without purification for the subsequent conjugation reactions.

General procedure for triGalNAc tether 2 conjugation: Amine-modified single strand was dissolved at 700 OD/mL in 50 mM carbonate/bicarbonate buffer pH 9.6/DMSO 4:6 (v/v) and to this solution was added one molar equivalent of Tether 2 NHS ester (57 mM) solution in DMF. The reaction was carried out at room temperature and after 1 h another molar equivalent of the NHS ester solution was added. The reaction was allowed to proceed for one more hour and reaction progress was monitored by LCMS. At least two molar equivalent excess of the NHS ester reagent relative to the amino modified oligonucleoside were needed to achieve quantitative consumption of the starting material. The reaction mixture was diluted 15-fold with water, filtered once through 1.2 μm filter from Sartorius and then purified by reserve phase (RP HPLC) on an Äkta Pure (GE Healthcare) instrument.

The purification was performed using a XBridge C18 Prep 19×50 mm column from Waters. Buffer A was 100 mM TEAA pH 7 and buffer B contained 95% acetonitrile in buffer A. A flow rate of 10 mL/min and a temperature of 60° C. were employed. UV traces at 280 nm were recorded. A gradient of 0-100% B within 60 column volumes was employed.

Fractions containing full-length conjugated oligonucleosides were pooled together, precipitated in the freezer with 3 M NaOAc, pH 5.2 and 85% ethanol and then dissolved at 1000 OD/mL in water. The O-acetates were removed with 20% ammonium hydroxide in water until completion (monitored by LC-MS).

The conjugates were desalted by size exclusion chromatography using Sephadex G25 Fine resin (GE Healthcare) on an Äkta Pure (GE Healthcare) instrument to yield the conjugated oligonucleotides in an isolated yield of 60-80%.

The conjugates were characterized by HPLC-MS analysis with a 2.1×50 mm XBridge C18 column (Waters) on a Dionex Ultimate 3000 (Thermo Fisher Scientific) HPLC system equipped with a Compact ESI-Qq-TOF mass spectrometer (Bruker Daltonics). Buffer A was 16.3 mM triethylamine, 100 mM HFIP in 1% MeOH in H2O and buffer B contained 95% MeOH in buffer A. A flow rate of 250 μL/min and a temperature of 60° C. were employed. UV traces at 260 and 280 nm were recorded. A gradient of 1-100% B within 31 min was employed.

The following schemes further set out the routes of synthesis:

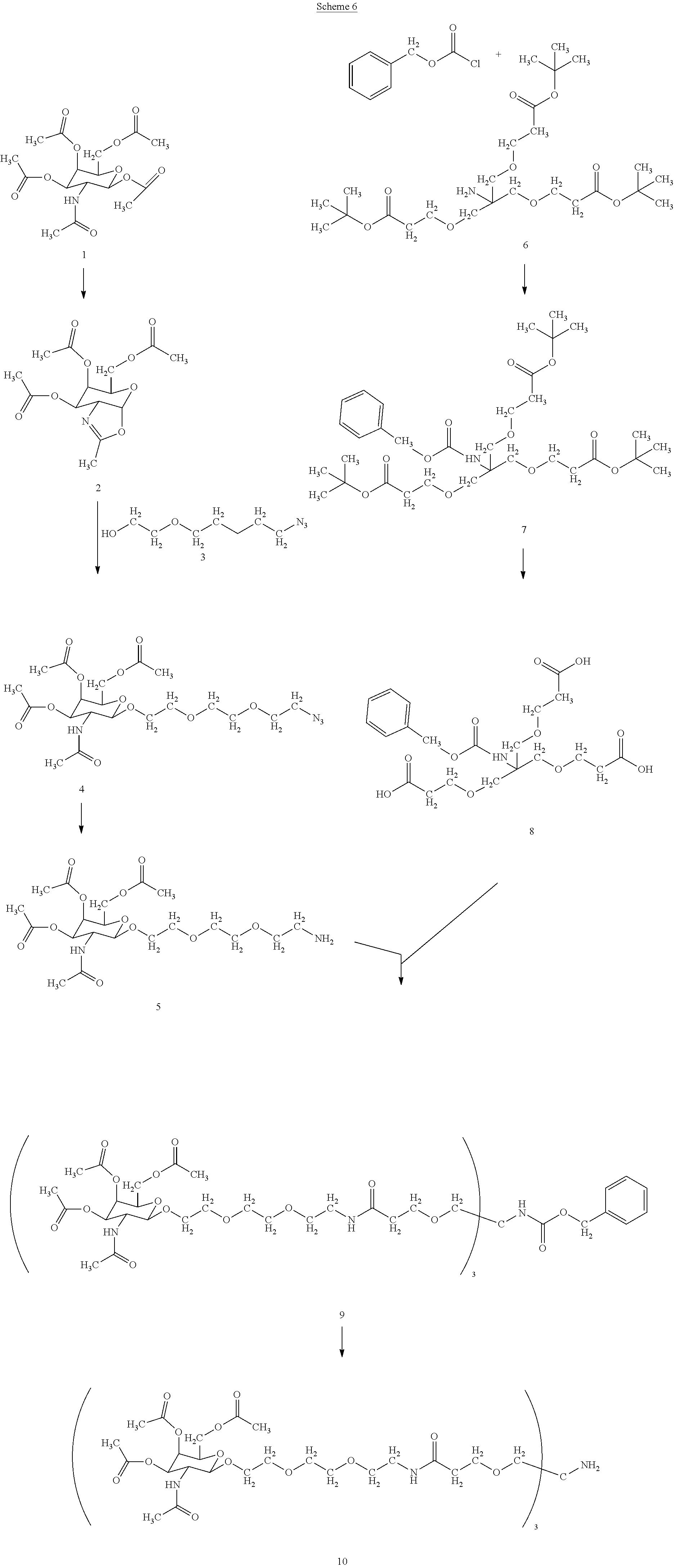

[symbol]

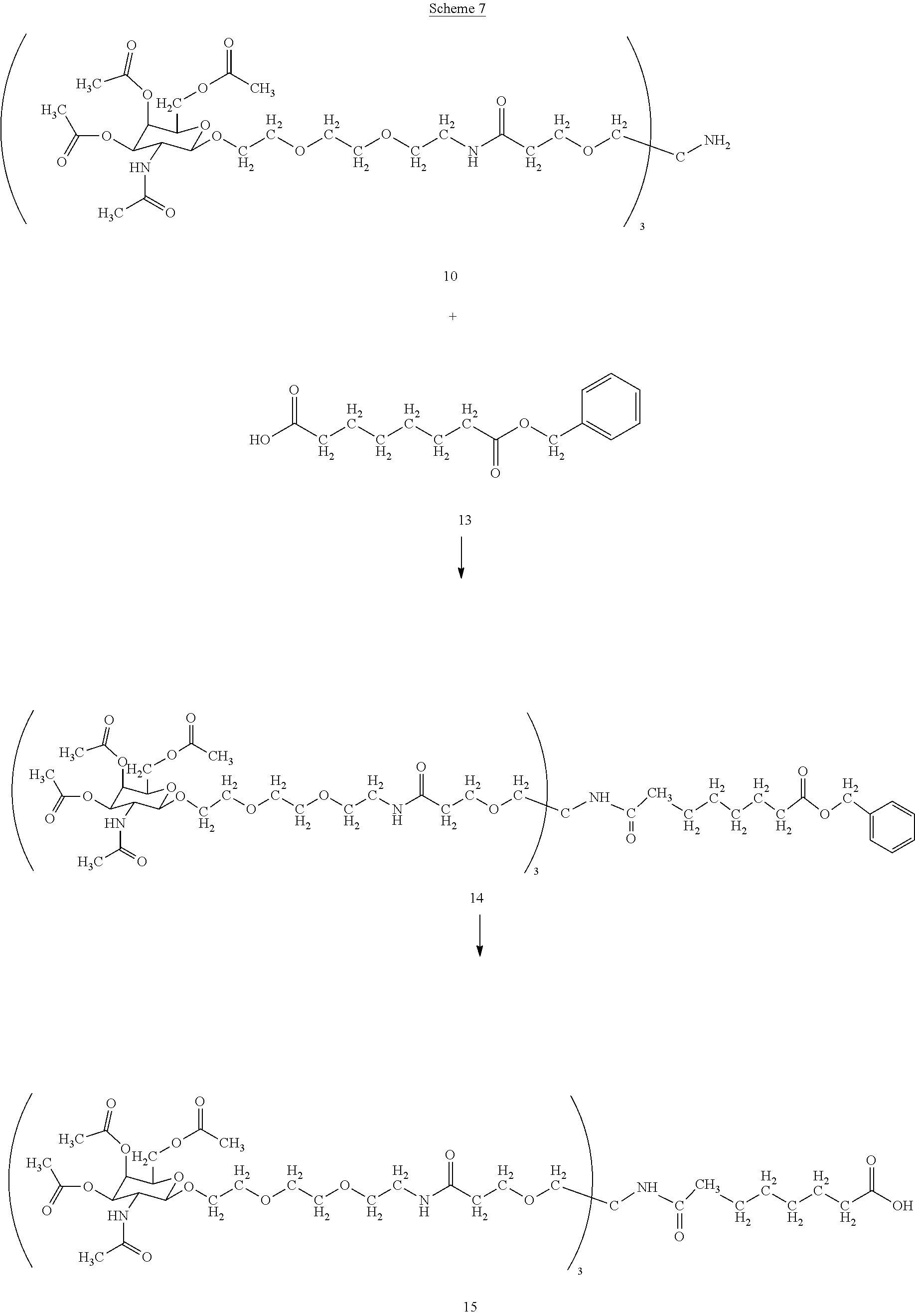

[symbol]

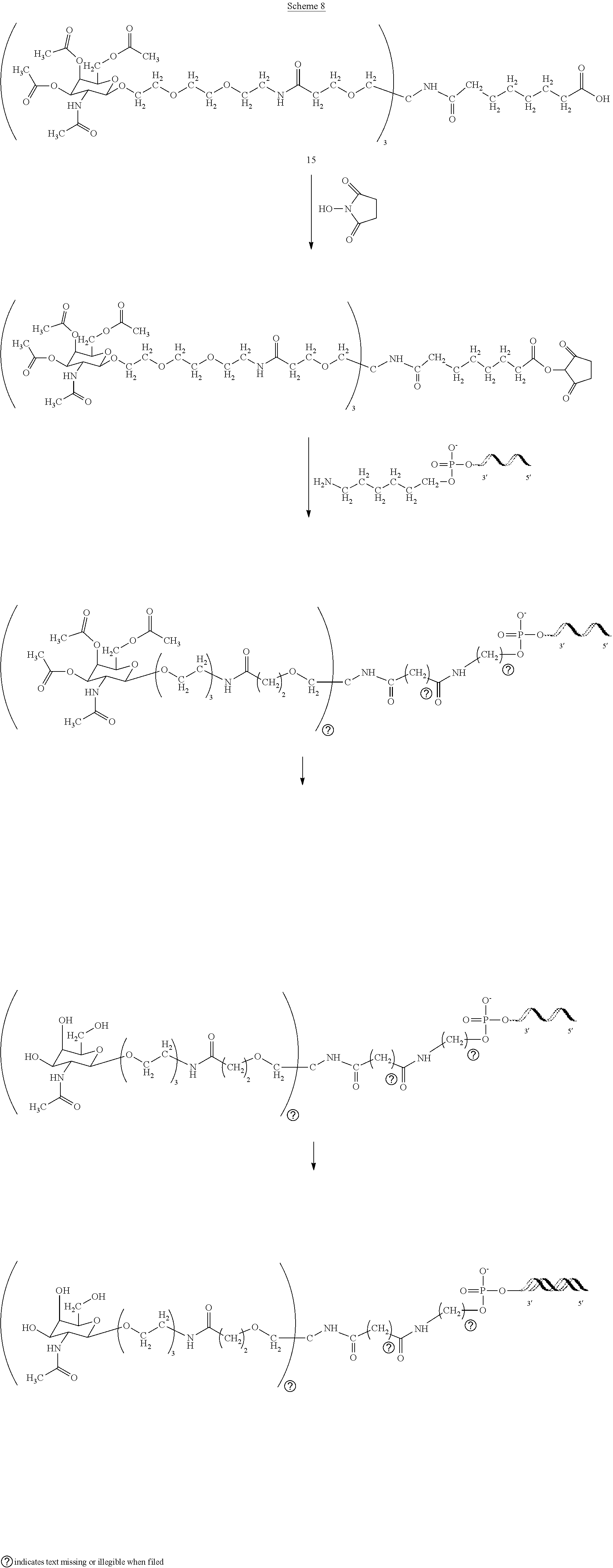

[symbol]

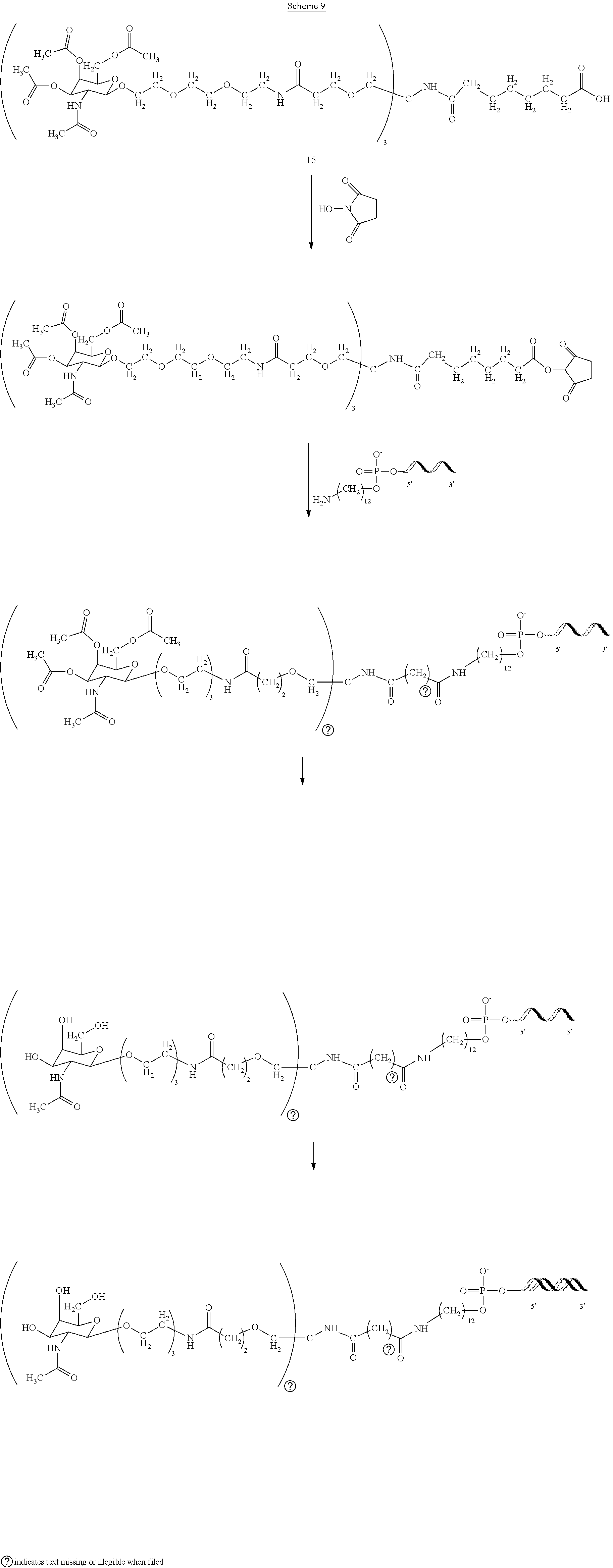

[symbol]

### Example 4: Duplex Annealing

To generate the desired siRNA duplex, the two complementary strands were annealed by combining equimolar aqueous solutions of both strands. The mixtures were placed into a water bath at 70° C. for 5 minutes and subsequently allowed to cool to ambient temperature within 2 h. The duplexes were lyophilized for 2 days and stored at −20° C.

The duplexes were analyzed by analytical SEC HPLC on Superdex™ 75 Increase 5/150 GL column 5×153-158 mm (Cytiva) on a Dionex Ultimate 3000 (Thermo Fisher Scientific) HPLC system. Mobile phase consisted of 1×PBS containing 10% acetonitrile. An isocratic gradient was run in 10 min at a flow rate of 1.5 mL/min at room temperature. UV traces at 260 and 280 nm were recorded. Water (LC-MS grade) was purchased from Sigma-Aldrich and Phosphate-buffered saline (PBS; 10×, pH 7.4) was purchased from GIBCO (Thermo Fisher Scientific).

### Example 5: Alternative Synthesis Route for the Conjugate Building Block TriGalNAc_Tether2

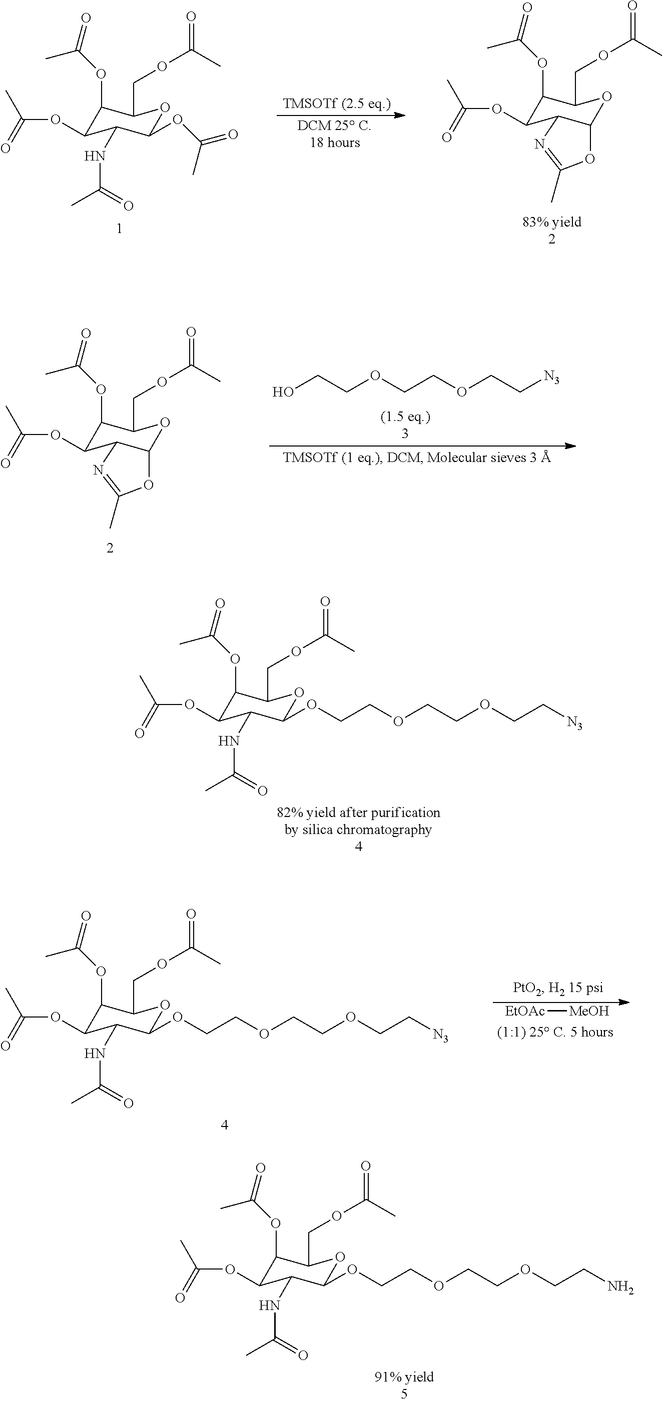

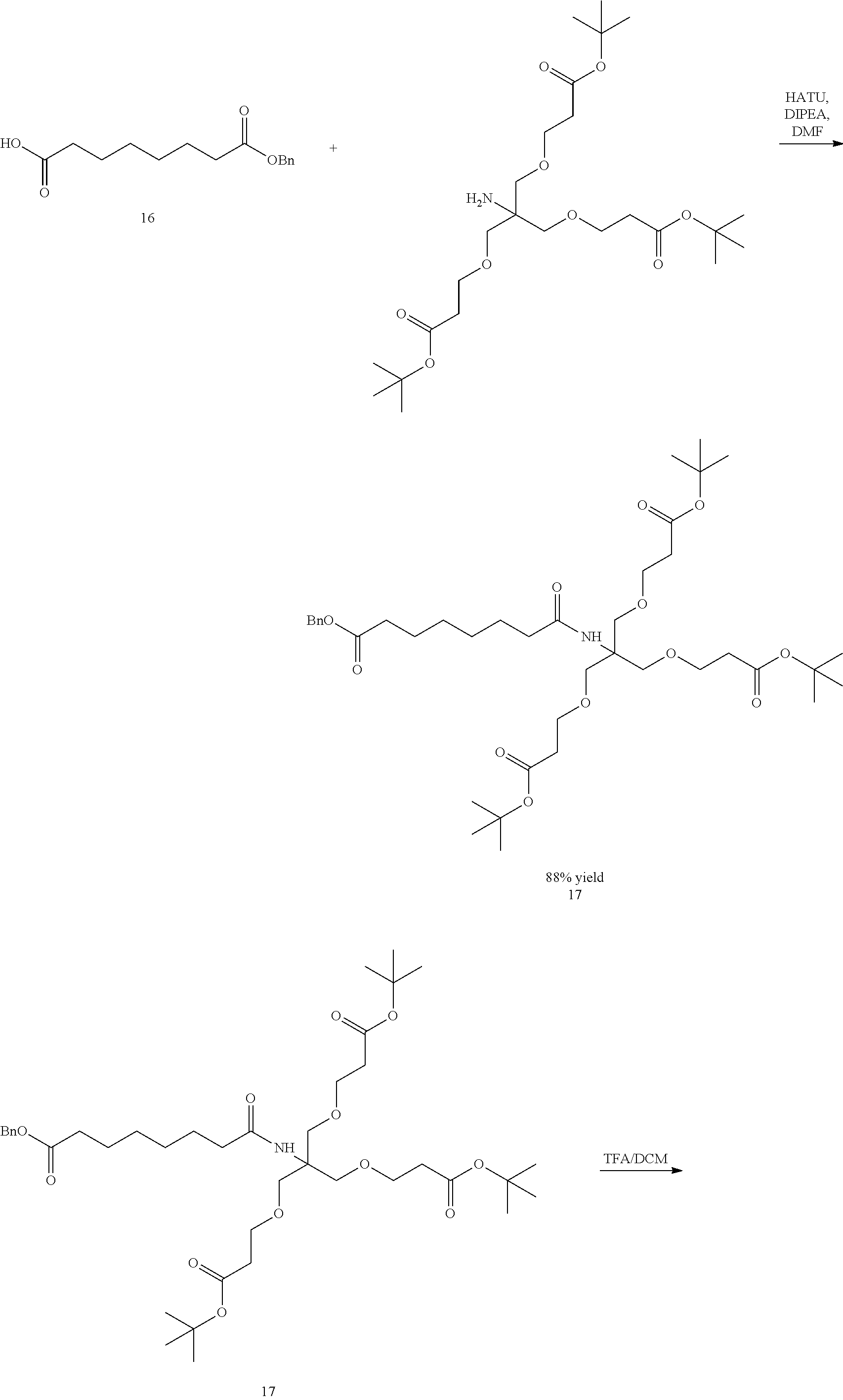 [SMILES: O=C(O)CCCCCCC(=O)OCc1ccccc1 ; CC(C)(C)OC(=O)CCOCC(N)(COCCC(=O)OC(C)(C)C)COCCC(=O)OC(C)(C)C ; CC(C)(C)OC(=O)CCOCC(COCCC(=O)OC(C)(C)C)(COCCC(=O)OC(C)(C)C)NC(=O)CCCCCCC(=O)OCc1ccccc1 ; CC(C)(C)OC(=O)CCOCC(COCCC(=O)OC(C)(C)C)(COCCC(=O)OC(C)(C)C)NC(=O)CCCCCCC(=O)OCc1ccccc1]

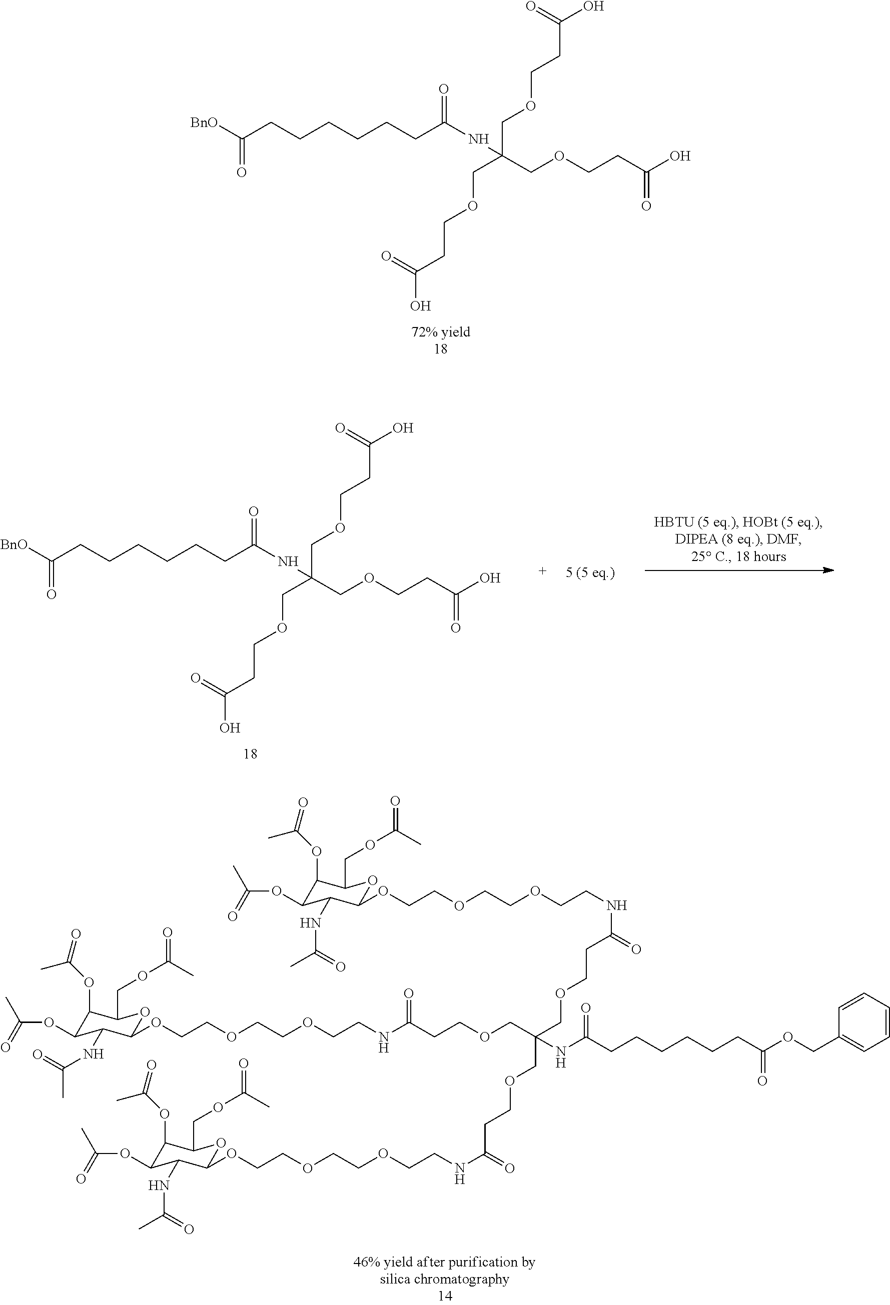 [SMILES: O=C(O)CCOCC(COCCC(=O)O)(COCCC(=O)O)NC(=O)CCCCCCC(=O)OCc1ccccc1 ; O=C(O)CCOCC(COCCC(=O)O)(COCCC(=O)O)NC(=O)CCCCCCC(=O)OCc1ccccc1 ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)CCCCCCC(=O)OCc2ccccc2)OC(COC(C)=O)C(OC(C)=O)C1OC(C)=O]

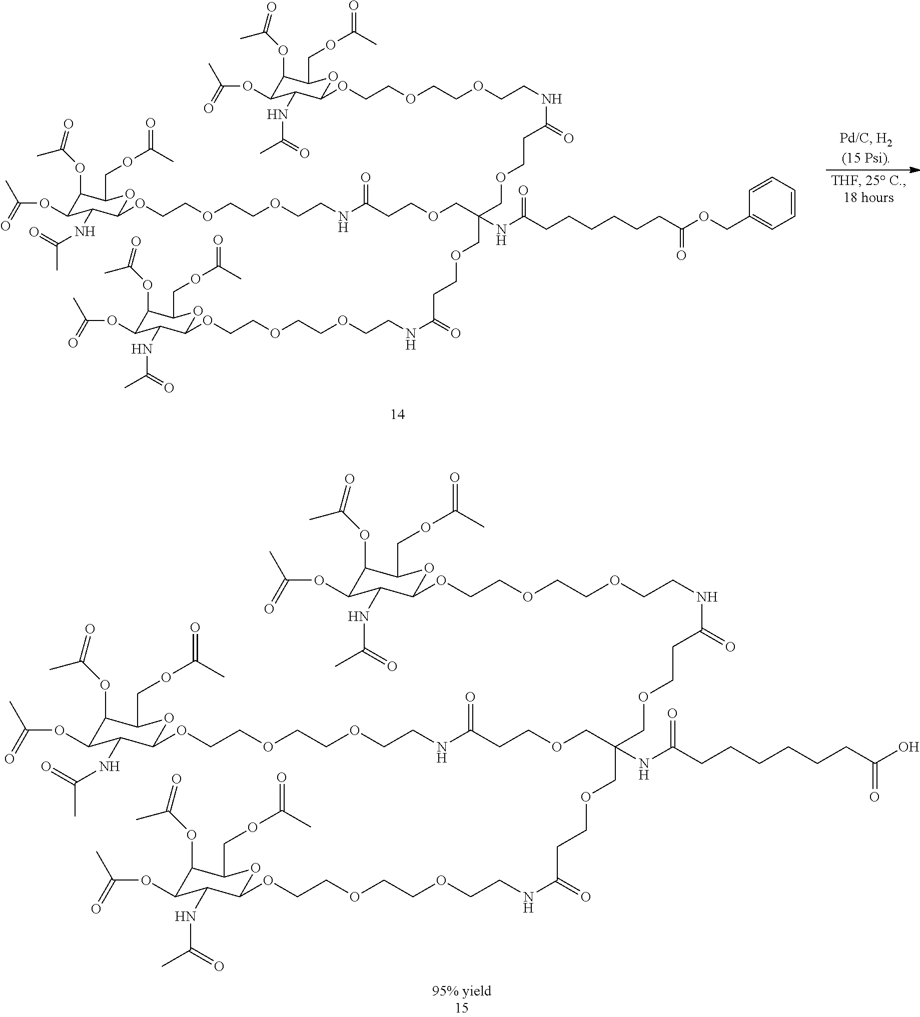 [SMILES: CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)CCCCCCC(=O)OCc2ccccc2)OC(COC(C)=O)C(OC(C)=O)C1OC(C)=O ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)CCCCCCC(=O)O)OC(COC(C)=O)C(OC(C)=O)C1OC(C)=O]

Conjugation of Tether 2 to a siRNA Strand: TriGalNAc Tether 2 (GalNAc-T2) Conjugation at 5′-End or 3′-End

### Conjugation Conditions

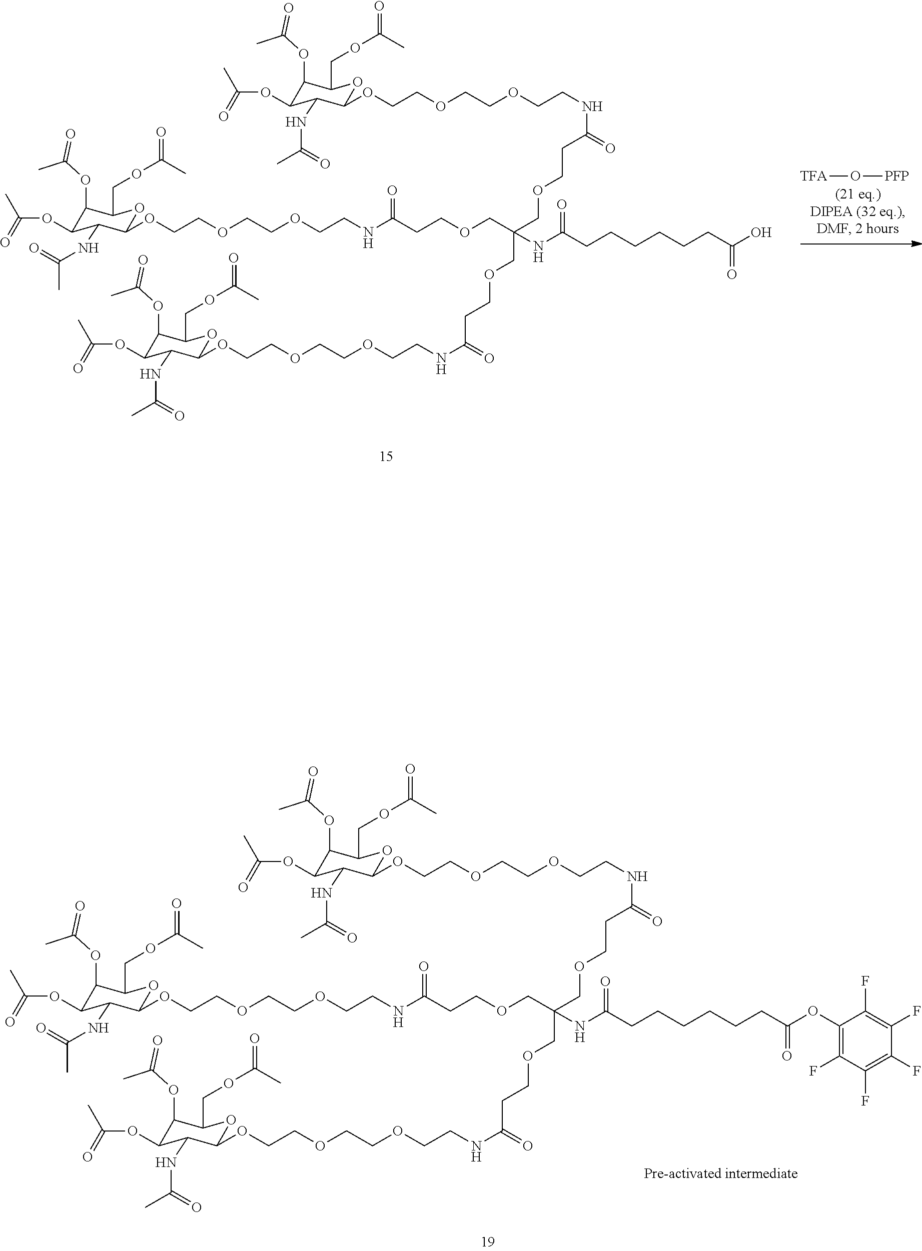

Pre-activation: To a solution of compound 15 (16 umol, 4 eq.) in DMF (160 μL) was added TFA-O-PFP (15 μl, 21 eq.) followed by DIPEA (23 μl, 32 eq.) at 25° C. The tube was shaken for 2 h at 25° C. The reaction was quenched with H₂O (10 μL).

Coupling: The resulting mixture was diluted with DMF (400 μl), followed by addition of oligo-amine solution (4.0 μmol in 10×PBS, pH 7.4, 500 μL; final oligo concentration in organic and aqueous solution: 4 μmol/ml=4 mM). The tube was shaken at 25° C. for 16 h and the reaction was analysed by LCMS. The resulting mixture was treated with 28% NH₄OH (4.5 ml) and shaken for 2 h at 25° C. The mixture was analysed by LCMS, concentrated, and purified by IP-RP HPLC to produce the oligonucleotides conjugated to tether 2 GalNAc.

### 5′-GalNAc-T2 Conjugates

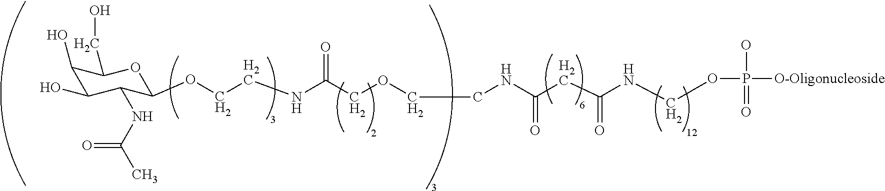

### 3′-GalNAc-T2 Conjugates

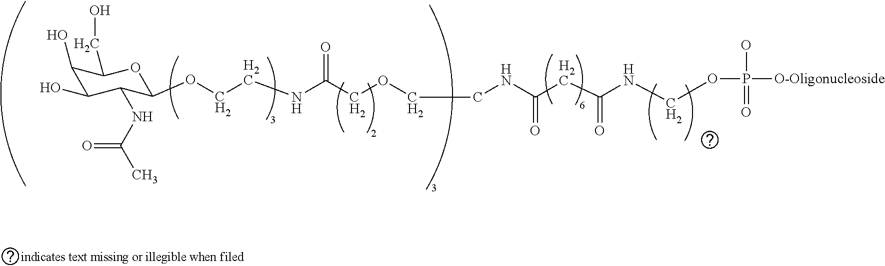

### Example 6: Solid Phase Synthesis Method: Scale ≤1 μmol

Syntheses of siRNA sense and antisense strands were performed on a MerMade192X synthesiser with commercially available solid supports made of controlled pore glass with universal linker (Universal CPG, with a loading of 40 μmol/g: LGC Biosearch or Glen Research).

RNA phosphoramidites were purchased from ChemGenes or Hongene.

The 2′-O-Methyl phosphoramidites used were the following: 5′-(4,4′-dimethoxytrityl)-N-benzoyl-adenosine 2′-O-methyl-3′-[(2-cyanoethyl)-(N,N-diisopropyl)]-phosphoramidite, 5′-(4,4′-dimethoxytrityl)-N-acetyl-cytidine 2′-O-methyl-3′-[(2-cyanoethyl)-(N,N-diisopropyl)]-phosphoramidite, 5′-(4,4′-dimethoxytrityl)-N-isobutyryl-guanosine 2′-O-methyl-3′-[(2-cyanoethyl)-(N,N-diisopropyl)]-phosphoramidite, 5′-(4,4′-dimethoxytrityl)-uridine 2′-O-methyl-3′-[(2-cyanoethyl)-(N,N-diisopropyl)]-phosphoramidite.

The 2′-F phosphoramidites used were the following: 5′-dimethoxytrityl-N-benzoyl-deoxyadenosine 2′-fluoro-3′-[(2-cyanoethyl)-(N,N-diisopropyl)]-phosphoramidite, 5′-dimethoxytrityl-N-acetyl-deoxycytidine 2′-fluoro-3′-[(2-cyanoethyl)-(N,N-diisopropyl)]-phosphoramidite, 5′-dimethoxytrityl-N-isobutyryl-deoxyguanosine 2′-fluoro-3′-[(2-cyanoethyl)-(N,N-diisopropyl)]-phosphoramidite and 5′-dimethoxytrityl-deoxyuridine 2′-fluoro-3′-[(2-cyanoethyl)-(N,N-diisopropyl)]-phosphoramidite.

All phosphoramidites were dissolved in anhydrous acetonitrile (Honeywell Research Chemicals) at a concentration of 0.05M, except 2′-O-methyl-uridine phosphoramidite which was dissolved in DMF/MeCN (1:4, v/v). Iodine at 0.02M in acetonitrile/Pyridine/H2O (DNAchem) was used as oxidizing reagent. Thiolation for phosphorothioate linkages was performed with 0.2 M PADS (TCI) in acetonitrile/pyridine 1:1 v/v. 5-Ethyl thiotetrazole (ETT), 0.25M mM in acetonitrile was used as activator solution.

Inverted abasic phosphoramidite, 3-O-Dimethoxytrityl-2-deoxyribose-5-[(2-cyanoethyl)-(N, N-diisopropyl)]-phosphoramidite were purchased from Chemgenes (ANP-1422) or Hongene (OP-040).

At each cycle, the DMT was removed by deblock solution, 3% TCA in DCM (DNAchem).

The coupling time was 180 seconds. The oxidizer contact time was set to 80 seconds and thiolation time was 2*100 seconds.

At the end of the synthesis, the oligonucleotides were cleaved from the solid support using a NH₄OH: EtOH solution 4:1 (v/v) for 20 hours at 45° C. (TCI). The solid support was then filtered off, the filter was thoroughly washed with H₂O and the volume of the combined solution was reduced by evaporation under reduced pressure.

Oligonucleotide were treated to form the sodium salt by ultracentrifugation using Amicon Ultra-2 Centrifugal Filter Unit: PBS buffer (10×, Teknova, pH 7.4, Sterile) or by EtOH precipitation from 1M sodium acetate.

The single strands identity were assessed by MS ESI- and then, were annealed in water to form the final duplex siRNA and duplex purity were assessed by size exclusion chromatography.

### Example 7: Solid Phase Synthesis Method: Scale ≥5 μmol

Syntheses of siRNA sense and antisense strands were performed on a MerMade12 synthesiser with commercially available solid supports made of controlled pore glass with universal linker (Universal CPG, with a loading of 40 μmol/g: LGC Biosearch or Glen Research) at 5 μmol scale. Sense strand destined t′ 3′ conjugation were synthesised at 12 μmol o′ 3′-PT-Amino-Modifier C6 CPG 500 Å solid support with a loading of 86 μmol/g (LGC).

RNA phosphoramidites were purchased from ChemGenes or Hongene.

The 2′-O-Methyl phosphoramidites used were the following: 5′-(4,4′-dimethoxytrityl)-N-benzoyl-adenosine 2′-O-methyl-3′-[(2-cyanoethyl)-(N,N-diisopropyl)]-phosphoramidite, 5′-(4,4′-dimethoxytrityl)-N-acetyl-cytidine 2′-O-methyl-3′-[(2-cyanoethyl)-(N,N-diisopropyl)]-phosphoramidite, 5′-(4,4′-dimethoxy trityl)-N-isobutyryl-guanosine 2′-O-methyl-3′-[(2-cyanoethyl)-(N,N-diisopropyl)]-phosphoramidite, 5′-(4,4′-dimethoxytrityl)-uridine 2′-O-methyl-3′-[(2-cyanoethyl)-(N,N-diisopropyl)]-phosphoramidite.

The 2′-F phosphoramidites used were the following: 5′-dimethoxytrityl-N-benzoyl-deoxyadenosine 2′-fluoro-3′-[(2-cyanoethyl)-(N,N-diisopropyl)]-phosphoramidite, 5′-dimethoxytrityl-N-acetyl-deoxycytidine 2′-fluoro-3′-[(2-cyanoethyl)-(N,N-diisopropyl)]-phosphoramidite, 5′-dimethoxytrityl-N-isobutyryl-deoxyguanosine 2′-fluoro-3′-[(2-cyanoethyl)-(N,N-diisopropyl)]-phosphoramidite and 5′-dimethoxytrityl-deoxyuridine 2′-fluoro-3′-[(2-cyanoethyl)-(N,N-diisopropyl)]-phosphoramidite.

Inverted abasic phosphoramidite, 3-O-Dimethoxytrityl-2-deoxyribose-5-[(2-cyanoethyl)-(N, N-diisopropyl)]-phosphoramidite were purchased from Chemgenes (ANP-1422) or Hongene (OP-040).

All phosphoramidites were dissolved in anhydrous acetonitrile (Honeywell Research Chemicals) at a concentration of 0.05M, except 2′-O-methyl-uridine phosphoramidite which was dissolved in DMF/MeCN (1:4, v/v). Iodine at 0.02M in acetonitrile/Pyridine/H₂O (DNAchem) was used as oxidizing reagent. Thiolation for phosphorothioate linkages was performed with 0.2 M PADS (TCI) in acetonitrile/pyridine 1:1 v/v. 5-Ethyl thiotetrazole (ETT), 0.25M mM in acetonitrile was used as activator solution.

At each cycle, the DMT was removed by deblock solution, 3% TCA in DCM (DNAchem).

For strands synthesised on universal CPG the coupling was performed with 8 eq. of amidite for 130 seconds. The oxidation time was 47 seconds, the thiolation time was 210 seconds.

For strands synthesised on 3′-PT-Amino-Modifier C6 CPG the coupling was performed with 8 eq. of amidite for 2*150 seconds. The oxidation time was 47 seconds, the thiolation time was 250 seconds

At the end of the synthesis, the oligonucleotides were cleaved from the solid support using a NH₄OH: EtOH solution 4:1 (v/v) for 20 hours at 45° C. (TCI). The solid support was then filtered off, the filter was thoroughly washed with H₂O and the volume of the combined solution was reduced by evaporation under reduced pressure.

Oligonucleotide were treated to form the sodium salt by EtOH precipitation from 1M sodium acetate.

The single strand oligonucleotides were purified by IP-RP HPLC on Xbridge BEH C18 5 μm, 130 Å, 19×150 mm (Waters) column with an increasing gradient of B in A. Mobile phase A: 240 mM HFIP, 7 mM TEA and 5% methanol in water; mobile phase B: 240 mM HFIP, 7 mM TEA in methanol.

The single strands purity and identity were assessed by UPLC/MS ESI-on Xbridge BEH C18 2.5 μm, 3×50 mm (Waters) column with an increasing gradient of B in A. Mobile phase A: 100 mM HFIP, 5 mM TEA in water; mobile phase B: 20% mobile phase A: 80% Acetonitrile (v/v).

Sense strand were conjugated as per protocols provided in any of examples 1, 3 or 5.

Sense and Antisense strands were then annealed in water to form the final duplex siRNA and duplex purity were assessed by size exclusion chromatography.

### Example 8: Nucleic Acid Sequences

siRNA oligonucleosides according to the present invention target ZPI. The full DNA sequence of the ZPI target is as follows (SEQ ID NO: 1):

AATGTGGGTTGGAGCCCCCATACAGAATCTCTATGGGGGCACTGCCTAGTGGAGCTGTGAGAAGACG
GCCACCGTCCTCCAGACCCCTGAATGGTAGATCCACCGACAGCTTGCGCCATTTATCCGGAAAAGCC
ACAGACACTCAACGCCAGCCCGTGAAAGCAGCCAGGAGGGAGGCTGTACCCTGCAAAGCCACAGGGG
CAGAGCTGCCCAAGACCAAGGGAAGCTACCTTTTGCATCAACGTGACCTGGACTCAAAGGAGATCAT
TTTGGAGCTTTAAAATTTGACTGACCTGCTGGATTTCAGACTTGCATGGGCCCTGTAACCACTTCGT
TTAGGCCAATTTCTCCCATTTGGAACAGCCGTATTTACCCAATACCTGTAACCCCATTGTATCTAGG
CAGTAACTAGCTTGCTTTTGATTTTACAGGCTCATAGGCAGAAGGGACTTGCCTTATCTCAGGTGAG
ACTTTGGATTGTGGACTTTTGGGTTAATGATGAAATGAGTTAAGACTTTGGGGGACTGTTGAGAAGG
CATGATTGGTTTTGAAATGTGAGGACATGAGATTTGGCAGGGCCAGAGGCGGAATGATATGGTTTGG
CTCTGTATCCCCACCCAAATCTCATCTTGAATTGTACTCCCATAATTCCCACATGTTGTGGGAAGGG
ACCCAGTGGGAGATAATTTGAATCATGGGGGTGGTTCCGCCATACTGTTCTTGTGATAGTGAATAAG
TCTCACAAGATCTGATGCCTTTATTGGGGGTTTCTGCTTTTGCGTCTTCCTCATTTTCTCTTGCCGC
CACCAGGTAAGCAGTGCCTTTTGCCTCCCACCATGATTCTGAGGCCTCCCCAGCCACGTGGAGCTGT
AAGTGCATTTAAACCTCTTTCTCTTCCCAGTCTCGGGTATGTCTTTATCAGCGGCGTGAAAATGGAC
TAATACACTGTGGTTATGTATTATAGTCATATGATATTTTCATATTTTTGGAAGCTGGGTGAAGGGT
AGATGTGGAGACCATGATTTTTGCAAATTTTTTTAAGTTTAAAGTTATTTCTAAATTAGAAGTTTAA
AAAGAAGAAATCACATAAGCCATAACACAATAGAAAGATGTCTTTAAAGTTCAAGGCAGGAGGGATG
TCTGGAAATCAGCGAGAAATTTGCACCTGTGTGTGCATGTGCATATGTGTGTGTGTATGTTGCAAGG
ACTTGGAAAGCCCTTTTTTTCCTACCTCTGTACTACTGTGGGGGGAGGCTAAACTTGACTTCTTCCC
ATCTTAGTTCTTTTTTGGGATAGACTCCTGTAACAAAAGACAGACAAGAGAAAAATCAGCTTACAAC
ATGGGCCATGCACTTCACACAGGAGAAACCTGCATGAAAAGTAACTCAAAATGGTGCCTTAGAACTC
CACTTACCTTTAGTAAAGAGCAATAAATTAGCAGGAAAATCATGGATCGGGACAAGGGAAGTGGTTT
TATGCTTCCAAGGGCAGGAAATCATGGAAGGTAAATATATGGGAGGAAACTAAAGGAATAAGGCTTG
TTTGCATATTCCTCTGATGCCATCTCTGGGTTGATAAGAGTCTAGAGTCATTTCCAGTAAAGATGAA
TTTTTATCTGTCTTTAGGAAGAAAGGGGGAAAGATAGAGAAAACTATTTCTCCATTTGCTGTTTCTT
AATTACCTTCAGTTCAAAAATAATTTTTATATCAGAAAGGCATATTTAGAGGTATGTTAGTTTATTT
TCACACTGCTAATAAAGACATACCCAAGACTGGGTAATTTATAAAGAAAAAGAGGTTTAATGGACTC
ACCGTTCCACATGGTTGGAGAGGCCTCACAATCAAGGCAGGTCTTACATGGCAGCAGGCAAGAGGGA
GAATGAGAGCCAAGCGAAAGGAATTTCCCCTTAAAAATCCCCTTATAAAACCATCAGATCTCGTGAG
ACTTACTCACTACCACAAGAACAGTATGGGGGAAACCACCTCTATGATTCAATGATCTCCCACTGGG
TACCCCCCAACAACACGTGGGAATTATGGGAGCTACAATTCAAGATAAGATTTGGGTGGGGACACAG
ACAGACCATATCAAGGGGTAACATAGTCTGGTTTCCTTTACTACCCACCTACCCAAACACCCCCTTC
ATCTGATCCACACAAAGTAAACTCTTGCAGTTCTCTCACTGTTTCCTGGAGTCTGCTTTTGGTCTCA
TAGGACTGCCCTAACGCTTGTTTTTCAGACGTTTAACCCTGTAGGTCTCTGGACAAATTTGCTTTAG
AAGCCCCTCGATGTCGCCCTGAAGAGTGGCTTTCAGAAGTTGTGCCTCCTGCCTGAGGGGAGTTCCA
GGAAGGGTTCTGCATCGCCTATGAGTTTATCTGGATCACCAGAGGCCTTCCCGTCAGAGCTTTCCCA
ATCGTTTTTGGCCAAGGAGTGTGAGAAGCTAAAGTTCATAACAACTGGAAGTCAGACAGCCTGGTCT
ATTCTGCTTTAACTCTAGCAGGAAAGGCCTTCATGGTGGGGCCTGAATATCTTCCTTTATAAAATCA
AAGCCTGGGGACAGGGTTACTTACTTCTGAGGTTCAATCTGGCTCTAAAATTATGCAACAAATGCCA
TTCCTTTAGCACTTCCTTCCTACCGGGCGAGATACTCAACTCCACAGGCACCACCTCAGTTCATCCT
CTCAGAAGTCCTAACAGCTCAGCCTGGGGCACCCCATTTTACAGATTAGTAAACTGAGGCTAAGAGA
GGTTAGGTAGCTTGTTCAGGGTCATGCTGCTGGTAAAAGAGCTCAGGCTACAGTGCTATGCATTGAG
TTTTCTCACTTTCCCATCTAACTGGAGGGCTAAAGGTCAAAGAGTGGGCAGCTCCCTTGTTGGGAGC
TGTACAGGAATAATGTCCTCCCTGAAGGAGGGGGACTTCTGAGCCACACCCTGGGGTCCAGGGCTCA
CAGCCTTAGGAGCAAAATCGTCCACCCCCTTCCTGGTTCCTCGGTGCTGCAGAGATATTCATAGGAC
AGAGTCTGAGTTCTGGCCACTTAACAGAGGAAGAAAGGCTGGCTCGGTGAGGTTAACTTACATCCCA
GCAGCTAGGAACCGGGAGCAGAGGACCTCAGATTCACACCAGGGCAGGAGGCAATGGCCTGGCTGAA
GCCTTCACAATCTTCCCAATATACTCCGCTGCCTTCCTTTATAAGGATCCATTTCTGAAACCCTGTG
CCCTGGCCAGGCACGGTGGCTCACACCTGTAATTCCAGTACTTTGGGAGGCCAAGGCAGGAGGACCA
CGAGGTCAGGAGTTTGAGACCAGCCTGGCCAATATGGTGAAACCCCGTCTCTACTAAAAATAGAAAA
ATTAGCGTGGTGGCAGGCGCCTGTAATCCCAGCTACTCGGGAGGCTGAGGCAGGAGAACTGTTTGAA
CCTGGGAGGTGGAGGTCTCAGTGAGCTGAGACAGACAGTGCCTGGGTGACAGACAGAGACTCCGTCT
CAAAAAAAAAAAAAAAAAGAAAGAAACCCTGTGCCCTAAGACCTGCACACTCGCTGGCTCCGCTCAG
ACATTTAGCAAAGCAGACACCTTCCCAGGCCTGGAGGAAACAGCCCCTGCTTTTTGGGAATCCACAA
GCCCGCAGCTGCAGAGCTCGACCTGGATGGGCAGGCAAAGGCTGACTCCTGTGCGTGGTGTGAGTCC
AGCCTGGCCCCTCTACACCCTCACTTTCACCTCTTAAAGAACTGCCTATTAACAGAGCAGGTACTGC
CCAAAAGGAACACTCTGGAAACTTGTTGGGACACTTCTGCCTTTCACAAACGTTTGGGGGGAGTACT
ACTAGCATTTAAGGATTGAGGGTTAGCAATGCCAGACATACCAGAACACGCAGGGCAGTCTCCCATG
ATGAAGAGGCCGCCGGGTTCCCCAGGACTCACATGTCCACCTCAAGTTCACGTGGGATTATCTGAGC
CTAGACTGTCAGTCCTGGGGCTGCTTTATTTCATATAAAAATATAATATTTATCCAAGGTTTTACTA
CACACTGCATTTTCTGTGAAGACAATGACCGTGTAAATCAGGGAAAGATCTATATTTTATTTTGTTT
GAAACTTTACCAAGCATTATTTACCATTTCAAAAGCTCTATCCCTGGTAGTACCATTGGTTTTCTTG
TTCACCGGCCAGCAGTGAGCAGCACACAAGCGACCTCCCGTGGGCTCCACATTGGACAGCCTCACTG
CACCTGCCCAGGCCCTTAGGCCACAGCACTGCCATATTCAGGGACACATTATTCTCTTTTATTATGC
CTCCATATTATCATTACAGCATTATCTTTTTTTTAATTTTGTGGGTAGATTATATTAGCTATACGTT
TCACTTCAATGGTAGTAGTAAGGGGCACATAACAAAATATTTACTTATATATATTAAAAAAGAGAGT
CTGAGAAGTCTGAAAAGTTTTGCCATAAACGGTCTCCACCAGCCTCAACTCTGAGTGCCCGAGGATT
CAGTCTCAAGTCCAGCAACATTGTGAAGCAGGAAATTTACCTTGAAAGGAGCTATGTACTCTAAGTA
GTGATTTACCTGTCTGCCTCCCCCACTGGATTGACCAGTTCCTTGGGGGCTGAGAGAACAGGTCCTG
AACATTTCTGCTGTGCCCCCCAACCCACATCCTCATAGTGTCCAGTACCAGGCTGGGGACTCAGGAA
GCATCCATGGGATCCCCCAGTGCCTTCTTTCTCGAGGTGTTCAGCACCTAGAACAGCTCAAGACAAA
TTCCCCACACCCCACCCAGACAGAGCTGAATCTTACTGGGGCGAAGCCTTGAGTTGCAAGGCAGAAG
CTCTCGTGATGGGATTTGGGTCATATTCCGGGTTATAGGAGGAGCTGGGGAGTATGGGAAGCCTCCC
ACTTGGTCTTTGGTTTTCCAGAAACTCCACCATCACAAGCAGGATGTTAATCAGTAACCGTCCCACA
GGGGATCATACTTTGGAATAGCAAATATTTGCTGAAGGTTCTGGGCTGCAAAGCTGAAGCTTTGGTT
TCTGCTCTAAATGAAGGACTTTTCCAGGACCCAAGGCCACACACTGGTAAGAGGCAGTGGGTTACAG
GAGACCTTCAATGAGTCTAATCAGGGAGGGACCGGGAAGGATGGTATCATCCCTGGGCGGGCTCCAA
CGTGAGGGCTGTGTGGCTGAGCAGTGCAAAGACCTCCATCCTACACTCCACAGGGACTGTACATACA
GATTGGGAGCTGGAGTGGGGTAAGAGGCGAATTATAGACACAAGGGGCTCCTCTGCAGGAAGGAGGC
CAAGGGAAAGAGGCTTGAAAGGCTTGATATTTCACCCACCACCACTCACTGCCGGAGTAAGCAGGTC
TCCCCTTCCCAGGGCTGAGGGGAGGCAGGGATGTGTGCTGTCCCAGGGCTGAGAAGTGGCAGGTGAG
CTGGTGATTCCTTACTGCCCAGGTTCTGTCTAGGAAGGTGCGTCCTCACCATGCTGGATGGTGTCCT
AGTCCAGGAGCACCCCCTGAGCTCCTGGCCTAGACTCCAAAGGGTTGGGTAGATGAGCAAAGACTTT
ACAAAGACCTTAGGCGATATATGTCCAGGAGCACCCAGGAATTACTGGGCTACCACTGCAGACTGCA
GGACAAGCTCCAAGAACAGGAAGGTAAGACTCAGCATTTGGAGGTGGTGACATCTAGTTGGCGTGCT
GGGCTAATTTCCTGACCATTGTACAGGGAGAAGTAACCTTGAATTCAGGAGTATTCTGTGTGGTCTT
AATGTAGAAAGTAGCACTAAATGATGCCACGTAATCGTTTTAGCTCAGGCTCCTCTAACAAAACACC
ACAGGCTGGGTGGCTCCAACAGCCATTGATTTTTCACAGTTTTGGAGGCTGAAAGTCCGAGTCAGGG
TGCCAGCGTGGCCGGATTCTGGTAGGGCTGTCTTCTTGGCTTGCAGATGGCCACCTTCGCACCGTGT
CCTCCCATGGAGAGGAGGTGCGGAGGGGGACTCTGCTCTCTTCTTATGACAGCACTAGTGCTATCAC
AGGGGCCTTGCCCTCACGACCTCATCTAAACCTAATCACCTCCCAAGCGCCCCAACTCTATTGCCAT
CACAATGGTGGTTCGGGCTTCAACTTATTAATTCTCAGGGGACACATTCAGTCCATAACAATAAAAG
CGTGAAACTGGGCTGCGTTTACACTGAAAGAGCTATTTACCCAACGTTTACAATACTTGGGTGACCT
GTTGAATGCAGGCTTGCCATTTAGAGTCAAAAAGAGCTTCCTCAACAGTGTCCTTTGGGAAACACAG
TGGAAGTATTTCACTGCTTCTACAGGGGAGAGGGTAGTGCCGTTCAGACTGCAGAGTGAGGCCCTGA
ATTCCGGGGTGCCATTCAGCCCGAGCAAGGGGCAACATGCTGGGCCCTGGCGCTGGAGGCGGTTTTG
TCCCAGGCATAGATAAGGACTCAGCCCCTGCATCAGGAAGAGGCCTGGCAGCACCGCCTGTCAATAC
ATTTTGCCGCAGGTGACCTTGGTCAAGAATAAGGGTCTCTGCTGATGGGAACTACTGTGAGGCCGGC
AGCATCCACCCTGCGCTCACTGGGCTGGGTGGCCTACCCCACCCAGACCCTCCCAGGGCAGTGGGCC
CAGAGAGAGGATGAGGGAGGGCAGGTGTCCCAGGGGTTCTGCCCAGCCAGCCTCTGGGATCAGGCCT
GCAGTGTGGCTGAACACCAGAACTGAGTTTGGACACAGCCAGGTGGCCCAGGCCAGTCCCAAGCCAT
GTATTTGGATGGAAAACATGGAAGTATTCAGGAGCCAGGCTCTGTGTCCAAGGATGTGGAGGGAGCC
TAAAAGGCGACAGAGAAGGGGACAGCTAACGGTGAAGAAGTGTAGCTCCCACACTGCAGCCTAGGAC
AGTGAGAACCGGCATGCAGCCCAGGTGGCTGAGGGCTCTATGAAGCCACAGTGGAGGGAGCCCAGAA
GTGGGTTGTATGAATTGCGGGGCCTCCTGCTACCCGGGAGCTGCAGCTATAGGAAGGAAGGAAGGAA
GGAAGACCTCCAAGGAACTGTGTAGCAGAGGTGCAGTGCAAAGAGAATTTTGATAAAAAATCCAGGA
AAGCTCCAATACTTTCCCCCTTCCTTGCCTAACGGGCATGCAGGCACTCCAATCCCCAGCCAAACAG
GGCACTGGGCAAGGCCGGCCACCCATCTGGATGGGCAGCCTGACGACCAGATGGTCAGGGCAGTGAA
TGAAGCAGATCAAGGAAAGGTGTGTGAGGACCCCTGATTCCACCTGCTTGGACCCCCACCTTCTGTG
CTGCCTCCTGCTCCCAGAGTGGACTCTCTTGCCCTGGCCCTCAGGGAGGAGACGGGATGAATGAAAA
CGGGGTCAGGACTGAGAGCTGCCTGCCGGCCTGGCAGGGAATGGGAACTGGAGGAGGTTTTGCTCTG
TGAAATAATGTCCCCTCTTTGGGTGAGCAAATGTCACCCACACTTGCTCTAGGTCTCCCTGGGGCAG
GGCTAACCTACTTGAGCCACAGGAAGGAGGCAGGGTCCCTGAAGAAGCTTTTACTATCCACAAAGAC
ATTTTAGGAGGCATTAAAACCATCTCTATCCTCTCCTCTCCACAGGAAGTCTTGCAGCTGAAGGGAG
GCACTCCTTGGCCTCCGCAGCCGATCACATGAAGGTGGTGCCAAGTCTCCTGCTCTCCGTCCTCCTG
GCACAGGTGTGGCTGGTACCCGGCTTGGCCCCCAGTCCTCAGTCGCCAGAGACCCCAGCCCCTCAGA
ACCAGACCAGCAGGGTAGTGCAGGCTCCCAAGGAGGAAGAGGAAGATGAGCAGGAGGCCAGCGAGGA
GAAGGCCAGTGAGGAAGAGAAAGCCTGGCTGATGGCCAGCAGGCAGCAGCTTGCCAAGGAGACTTCA
AACTTCGGATTCAGCCTGCTGCGAAAGATCTCCATGAGGCACGATGGCAACATGGTCTTCTCTCCAT
TTGGCATGTCCTTGGCCATGACAGGCTTGATGCTGGGGGCCACAGGGCCGACTGAAACCCAGATCAA
GAGAGGGCTCCACTTGCAGGCCCTGAAGCCCACCAAGCCCGGGCTCCTGCCTTCCCTCTTTAAGGGA
CTCAGAGAGACCCTCTCCCGCAACCTGGAACTGGGCCTCACACAGGGGAGTTTTGCCTTCATCCACA
AGGATTTTGATGTCAAAGAGACTTTCTTCAATTTATCCAAGAGGTATTTTGATACAGAGTGCGTGCC
TATGAATTTTCGCAATGCCTCACAGGCCAAAAGGCTCATGAATCATTACATTAACAAAGAGACTCGG
GGGAAAATTCCCAAACTGTTTGATGAGATTAATCCTGAAACCAAATTAATTCTTGTGGATTACATCT
TGTTCAAAGGTACTTTGATAATGTTCTGCTCTCCCAAGGCCACAGGGCCCTACGATTGTCTCTCCCT
TTCCTTTCGTTAGGCCAGCATATGATTAACGCTACGTGATTTTCTATGAATGTGTTTTCACGTTTCA
AAAACAGATTGATACACATATTGAACAGTGCCAGACGCTGTCATTTGAGGCCCTTCCCTGGTATCCT
ATGTGCTTGTAGTCCTTATTATTTTCAGAGCACTCTACATAGCTCCCCTCTGACACTTAGAAGCATA
GGGTCTTTCCAAAAAACAGGGGGCTGGGGGATTATCTGGGGGATTTAGGATTGCATCATTGCTCCTT
CATTTTTACTTTTTGACCAACTCTCTGCCCTTAGATTCCTATTATAGAAAATAGGGACACTCCACCT
ACTACAGTGTTAGAGGCTAAATGAGACAATGAATGTAAAGTGCCCAGATGGGCTTGGCACATAGCAG
ACACTGAGTATCTATTGTTTACTTGTTCTTCCAAACTGCCAATCAGCAGGTAGAGCAGGAGTTGTCT
CCTTTCTAAAGATGAAACCAGCTCAGAGACGTTAGCTTGATCAAGGTCACACAGTAAGTGGCAGAGG
CAAAACCCAAACAAGGGCCTCCTGACCCCCTGATCCTAGGTTCTGTCCAGCCCTGCCTCCCTAATGG
GGCACTGGACGTGGGTTGGATGCCACTTTCGCAGAGCTGGCACCAGACTTACAAAGCCCCGGCAGGG
GAAGCCACTTTACAACCAGCCAGGCCACACCCCCAGGGCAGACGTTTATGTAGAGAGTAATGTACCT
GCCTGCTAGTAGCCTCTGCATTGTGGGGCCTTCTCTCAGAACCACACTAAACAGTGGGTGGGTGAGA
AGTGTCACTCCTGCCACCTTGGACTCTGCATGTGCTTGTGCCTGGTGTGAATGAGACAAAGTGGCAG
TCAGAGGTGCCAGGCAAAGGCTTTTCTCTAAGCTGGAGCCAACTATGAGGGAACGACTGTGAATTCC
GTTCAGGTCCAGGACAATGAGAGGAGCCAGGGATTGTTAGGAAACATTTCCCTGCTTTCGTGTGCGA
TTCCCAATAGGGCCTGCGAGTGGAGCTGCATTTTGCTAGCTGGGCTAGAGGACGGGGAAAATTTTGG
GGAAATTTATTTTGCCTGCCTGAGCTGTGGAAAAGCCAACCCAATTAGGGAACGCCTTTCCTAGTTG
GAACGAGAAGACGAGAAGTGAGAGAAGTGAGATAGAAGGCTCCCTCTCTATTATTTGAGCAAGAACA
ATGCTTTTCAAAGAGGGAATTTCTGCAATGAGTTCTTCTCTTACTTGTTCAGGGAAATGGTTGACCC
CATTTGACCCTGTCTTCACCGAAGTCGACACTTTCCACCTGGACAAGTACAAGACCATTAAGGTGCC
CATGATGTACGGTGCAGGCAAGTTTGCCTCCACCTTTGACAAGAATTTTCGTTGTCATGTCCTCAAA
CTGCCCTACCAAGGAAATGCCACCATGCTGGTGGTCCTCATGGAGAAAATGGGTGACCACCTCGCCC
TTGAAGACTACCTGACCACAGACTTGGTGGAGACATGGCTCAGAAACATGAAAACCAGGTACAACTC
TTGCCCACACCCTATACAAACTCTACCTTTCTGTACTGGCAAACGCTCAGCACAATTTCATTGAATG
CACCGTGATTTAATGTCTCCTCCAGTGAGCTATAAGTTTCCTGAAGGCAGGGCAGCATTTGTCTTTT
TTTCCACTCTATCCCCAGCATCTGTCACAGGGTGCCTGGCTGATTCATTCATTGAGTCCATCAGTAT
TTTACGTTCTGCGACTGTGATAAATATATGATGCCAGGGATCCATCAGCAAACAAAACAGGCAAAAT
TAGTCTGCCCTCATGCAGCTTACATTCTATTGAAGGAAGACAAAGAGTAAATTAAAAATAGGTAATA
ATGCAGGGAAGGGGACAAGAAGCATCATCAGGATGCAGATGGAGGTTAGACAAGGCCTCTCCAAGAA
GGTAACAGTAAGCAAACATCTGAAGATGAAGGATAAACCATGTGGATATATTCGGGGAGAGAAGTGT
TATGTTACAGGCAGAAGTGTACAAGTTCTGGGATGGGAGTGTACCTGGTGGGTTTGAAGAACATCAA
GGAGACAAGTGTGGCTTCAGCAGTTGGAGATAAAATCAGAGAGGAAACAGGGGCCCAGTCCCCAGAA
AAGACTTGGGCTTTCCTGAGAGAGGCAGGAAGCCACTGGATGGTTCTGAGTAGAGGAGCAACCTGAT
TTTGACTTCTGTTTTTAAAGGATCACATAAGCTCCTGTGTTGAGAAAAGACACTAGGGGGTAAGGAT
GGAAGCAAGGGAGAGTGGTTAGAAAGTTACTAGCAATCCAGGTAGAGATGCTGCTACCTGGACTGCG
GTGGTGGTAGTGGAAGTGGTGAGAAGTGGCTGGATTCTGGATCTATTAGGAAGTGCAGGATCTGCTA
ATCGATTGGATGTGGGTGAGAGAGGTGTCAAAGGTGATCACAAAGTTTTTGGCCTTAGCAACTGGAA
AGACGGATTTGCCATTTACTGAAAGGGGGAGGAACAGGTCTGGGGTAAGTGCAGAAGTTCAGTCTTA
AACACTTGGATCAGAAATATCTATTAGACATCCAAGTTGAGATGTCAAGACGACAGGTGGATCTGGA
GTCTAGGGTGAGGTCCAGGCCGGAGATATAAATTCGGTCATCAACACAGAACTAGAATCTAGACACA
TGACAGGGTTGGGGTCTGTAAATATAGAGGAGAGGAAAAGAAAGCACAGAGTGGGCACTGAAATGTC
TGCCCAATAAATTAATCCACCTATTGGAGTACAAGGAAAATGGCTGCAATACGAATTCCATGGCTAT
GGCTTCTGAATCCTGTGACTCAGATTTTGGCAGACAAGTGCAGCTAAAGGTCCCCAGGGTTAGTTTT
ATCTTCATTATTCTTCTTTCATTTTTCTTCATATCTTTAGCACCTAACAATGAACCCCAAACATCAT
AAGCCCTCAAGTAATGTTTGCTGAATGAATAACTTTTTAAATTAATCTTCAAGACACGTCATGTCCT
CAATTATTTTTAAATAAATAAAAAAATTTTATTTTGAGCCACAGAACTCATCTTTTCAAGCAACATA
TTTTCAAAGGAGGACTCCAGTATACAAAATAGATGGTATCAGAGCTTCTCTGGCTAAAGACGGGTAG
GGGTTGAAAGTTTTCTTTGCTCCCCTCCCCATCCATCCCCAGACTCCTCGGGTCTGCAGAATCCAGG
AGCTGAAAACAGCCATCATCCAGGAGGCTGCAGGACTGCTGAAAGCAGCTGTTAACTCAGGTTTTTT
TTAAAATATAGGGAAATGAACACATAAGTACTTTGCTAAAGAAAACGTGAGTCACTGGCTGAGGAAT
AAAACTCATTCACTGAAGCTGAAGTACTATTTGATAAGCTAGAAATATTTTCCCTGAGTAGACCACT
GTAAAAGAATGGCATGAACTACATAGTCAACTGAAAGACTCATTAATGGAAATAATCTTAAAGAACA
AAAATTGTGACCTTTTTGGTGTCCACAGACTAGGGCTTTGTCTACATTTCACCATCATCTGTTCTTG
TACCACAGAAACATGGAAGTTTTCTTTCCGAAGTTCAAGCTAGATCAGAAGTATGAGATGCATGAGC
TGCTTAGGCAGATGGGAATCAGAAGAATCTTCTCACCCTTTGCTGACCTTAGTGAACTCTCAGCTAC
TGGAAGAAATCTCCAAGTATCCAGGGTAAGTCAGGATCTTTCATCAGAGCCCAACCTCAGCATGAAA
TGTCACCAAAACAAATGCTTTTACAAACCATTTAACTTTGATAAAATACCTAATTGTAGTGGAAAAT
TAGATTTAAGTCCCAAATACTTGAAATAGCACCCAGGTTGGATGTTTTAAGAATTTCAAGCAACTTC
ATTAAAATAACTTTTCAACTAATTTATTTTAAGCAGACCTCTCCCCCTCTGCTTAAAGTGCTCAGGG
AGAAATTTGACCCTGAAATAGAACTGGTTTACAGAGGCATCATCATTTATGTTGAATACAACTTGAA
TAGTTCATGAAATTACACCACCTTTACAATGAAACAAACCCCTAGACATCATCTAGCCCAACTTCTC
CCTCCTTGTGGAAATCCCCTCCATAGCCCTACGAAATAGCCCTCCAACTTCTCTTCCTCTTCATGCT
TCCAGTGACATCAAACTCACCATTTCTTTGAAGAGCTGCCCAATCCACAAATAGCTAAAATTGTTAT
ATGTATATATATATATGTGTGTATATATATGTATATATGTATGTGTGTATAAATGTATATGTGTGTA
TATGTGTGTGTGTATATATATATACACACACATATATATATATATGGAGAGAGACATACATATATAT
ATGGAGAGAGAGAGAGAGAGAGTCCTGTAACTTCTGATTCATACTTTTTGGTCCTAGTTCTATCTCT
AAAACTTCTAAGAACAAGTTTAGTCACCATCCACATAGAATCCCTTCAGTTACTCAGTGTTTCTCAG
TGGAAGGGTTCTTGGTTTTGAGGGGAACTGCTTGTTGTCCAGAGCAGTTGTGCATGTTGCAGGGAAC
TGGTTAGCATTGCTGGCCCATGTTCACTAATGCCAGTAGGAAACTCCAGTCATCACTATAAAAATGC
TCCCACACATTTCCAAATGGCAGCTACATCTCTCTACATTCTTCCTTAGCTGTGTGGTTTAATATTT
TCTTATACAATTGCAATTTTCAATTCCAAGAGAGACTAAAAATGGCATCCACTTAAGTAGGACACAG
TAGGGTAACTGTGGCCTGGAATCAGGTCTTACAACCTCAAGAGAGGTAAGACAATTAAATAAAACAA
TCCGTCAGACCAGCACCTGAAAGTGTTTCTGCTATGAACACATGAAAAACTGAAATGCGCTGCTGCT
TTATGAAGGGTCATCATGAAATTTAAACTGTAAATGATTAAATATTCTCCCTCTGTTTGCTCTGGGG
AATTAATTTTCCTCTAGGAAATCAGGGAATTTCCTGGAGTGAAAATCAGTGTAATTACATGTTATGT
TTTCATTATCTCTTATAACACAGTAATTATATAGGTACATCACTCATATCACATCTTGTTTCTGTAA
AAAAGGGCCTCCCAAACATAGCAAGCAGCCACAGTATAGGCAGCCAGAATTCAGGAAGGCTCCAGGG
ACCCCTGGGCTTGGCCCAGAAAAATGCCTCAGAGTAGTACCAGGTGCTGGGAAGCTGCTACAGAAGA
CTAGCCATTCCCTGCCTCCACCTTGCCTGCCAAAAGGAAAGTCAGAGGACTCAAGGGATCCAGGGAT
CAAGGGATCCAGGCAGCTTGAAAACCTTTTAGGAGCACCAGCTCAGCTCAAGAATTAGTAGCATAAA
TTACATGCTCAATAAAGATTTGATGCATGAGTGCATCCTGAGTCCATGCCCGGAATGTGTTTCACAT
ATTCCACAATACTTCACATTGGGTTCCTGAGGTCTCCTGGTATTGTTTAAGACTCCTGTGGCAGTCC
CTGGTGCAACCCCAGACCACTCCTCTTAACGTAGATGGGCCTGCTCCACTAAATCCCAGGAGCATGA
CCCCATGGGTAGGACCACTGTGAAGAATTTCAAGGGGCTCATTTAATTCCTCCTTTGCACTGCCACA
CAAATGGTTTTTCACATTATTTCCTTTTTCCAGGTTTTACAAAGAACAGTGATTGAAGTTGATGAAA
GGGGCACTGAGGCAGTGGCAGGAATCTTGTCAGAAATTACTGCTTATTCCATGCCTCCTGTCATCAA
AGTGGACCGGCCATTTCATTTCATGATCTATGAAGAAACCTCTGGAATGCTTCTGTTTCTGGGCAGG
GTGGTGAATCCGACTCTCCTATAATTCAGGACACGCATAAGCACTTCGTGCTGTAGTAGATGCTGAA
TCTGAGGTATCAAACACACACAGGATACCAGCAATGGATGGCAGGGGAGAGTGTTCCTTTTGTTCTT
AACTAGTTTAGGGTGTTCTCAAATAAATACAGTAGTCCCCACTTATCTGAGGGGGATACATTCAAAG
ACCCCCAGCAGATGCCTGAAACGGTGGACAGTGCTGAACCTTATATATATTTTTTCCTACACATACA
TACCTATGATAAAGTTTAATTTATAAATTAGGCACAGTAAGAGATTAACAATAATAACAACATTAAG
TAAAATGAGTTACTTGAATGCAAGCACTGCAATACCATAACAGTCAAACTGATTATAGAGAAGGCTA
CTAAGTGACTCATGGGCGAGGAGCATAGACAGTGTGGAGACATTGGGCAAGGGGAGAATTCACATCC
TGGGTGGGACAGAGCAGGACAATGCAAGATTCCATCCCACTACTCAGAATGGCATGCTGCTTAAGAC
TTTTAGATTGTTTATTTCTGGAATTTTTCATTTAATGTTTTTGGACCATGGTTGACCATGGTTAACT
GAGACTGCAGAAAGCAAAACCATGGATAAGGGAGGACTACTACAAAAGCATTAAATTGATACATATT
TTTTAAGATGTTTGTGCAATCTGTCTGGTATTTTAAGCTTGTTTCTAAGAACCTTAGTTACTTGGCT
AAAGACTAGCTGGGTAGAATATCTTTTCTCTGTTGCTCACATATTTTCATTTTTAAAAAGTTGCAGA
TGAGAACACTATGTCAAGATAAAGCCTTTGGGAGGAACACATGTAAACATTCTCCTTGAGTCATGTG
CTTCTCTCTCTTTCCTTCTCTCTGGTGCAAAATAAGTGTTTTATTTTAATCTATTACGGAGTCATTT
CTTGCTGACTGACATCAGAAGAAAATAGCTCTAACCAGTCCTGATCACAGCATCTGCTTCCATGGTG
CATCAAATCGCTTGGCAGAGGCATTGGCTGAATCACAGATCATCTAGTTCAATACCTTCATTTTACA
AAGGAAAGAAAGAGGGACCCAGAAACAGGTCCATATTCTTACTTTCATGGGCCCTAGGCACGTTTAA
CCTTGTAGACTCCTCCTTCCTTCATGAAGATATATATGTTCTATGGCTGCATTGGTAGAAAGATGAA
TATATTCGTCTTTCAAAGTTGCATATCTAGCTTCAAAGTTATATGTCTAGCATATGGCAATAAGCAA
AACACCTTCATGGGCCCTTACAGTACTGTCAGCCTTGGGCACTGTGTCTTCTGCATCTAGTGGATAA
GTCATACCTTATATACCAGTGGGAACAAAATACTTGTCCAAGGTCTTCCAGTGTGGCAATGGCAGAG
TCAGAAGCCTACCTTTCCTGAGTCTAGTCTCCAAGCCCTTTTTACTCTTCCTTCCATCTAAAACATC
TGATGGGGACCAGGTAAACAGCATGCACTACAGCTACCCATGGGGGTTAAACAGAATATAAGCATGA
ACTTTGTCCCAGGGTGAAAAGGAAAATCGTAAATATCCCTGATCTTCCTTAGGCAGTTATTTTCTGT
CACAGAAACAGAAAAGACTATATTCAGAGAATCCTGAATAGAGCTGATTTACAGTGTGAACTATGTT
AACTAAATGCCTAATTGGATTTCTGTCTGTCTGCTATCTAATGTTTAAAAAAACCTAAAATTCATTT
ATTGATTAGTTGTTTAATATAATTCAGAGTAATGTGAATAGGTAATAATATTAATATGCAGTCTAAA
TACTGACTTTTCATCATTCCATAACCTGGACTGATGAAAAGTCAGTATTTAGACTGCATATTAATAA
AATAAAATTCATTCCTGTATTCATTCCAAGAGTACTAATTGACACTTATGAAGGGACAGGCAATTCT
AGGCCCTAGAGGGCCAAAGACAGAGGACTAACTCTATCTGACATTCTTAAGTCACCTTGTTTGTGTT
CAATTAGTCAGATTTGTTTGTGGAAAAATAGTAGAAAGAGGAATAAAGTAGCATCCAGTCCAATTTC
CCACTTTTAAGAGATGAAATCTGGAAAAATAAGTCTGTGAGAGCACAATACTCACTGAAATCAATAT
GGCCAAACCCAGTAATAAAAAGGTACATTATTATTGAAGGATTCATATAGCATGCAGATAAAAAACT
CCTGCCTTCTTCCCACCACATACACTGCAAAGCAACAACAGCATAATAATTGTATTTAATATACTAC
TCTTTAAGGTAGAAAATGGACCTATTCTATATTTTAAATATACTTTTTAATGTTCCCTCACATTTGC
TTTAAGAAGTTCCTAAGACACTCAGTTTCAGATTTCCCAAGTACACAGGCATGACAGAAAAACGCAG
ACCAATAAAAAATGTAACTTACCTTACACAAATACATACACACAAATTCAGGGTTTCCAACCGAGCG
GGGGAAATCTTAACATTGTAGAAGTCTTCACTATATATGTGTCGAGTTTTTGTTTTTGTTTTTGTTT
TTGTTTTGAGACAGAGTCTTGCTCTGTCACCCAGGCTGGAGTGCAGTGGTGCGATCTCAGCTCACTG
CAACCTCCACCTCCCGGGTTCGTGCCATTCTCCTGCCTCAGCCTCCCGAGTAGCTGGGACTACAGGC
ACCTGCCACCACGACCGGCTAATTTTTTGTATTTTAAGTAGAGATGGGGTTTCACTGTGTTAGTCAG
GATGGTCTTGATCTCCTGACCTTGTGATCTGCCCGCCTCGGCCTCCCAAAGTGCTGGGATTACAGGC
ATGAGCCACCACGCCCGGCCAAGTGTCGAGTCTTAAAAATTGTTCCTACACAGACACACTCAACCAC
ACGTTCTCACATATATATGCTGTAACAACTGAGAACAGGTTACTGACTTAATCTAATTCATTCTATC
TTCATTGTAAAACTTCCACTCCAGCTGAAGAGCCTGTTTCATTTCAATTCAAAGATTTCTCATATAT
CCACTAATTGTATGGCAAAACTGACTCATCTCCAGACTAAGATATTCAAGCTCAGGAAGTCAAATAA
TAGAAATGATTTTTTAAATGTGTAAGAGGTTATAAAGAAAAACTTTATGTGCTCCTTATTTAACCTC
TATTAAGTAAAATCCTTTATAGACCTATCTCCATTTCTGCAGTAAAAGTGAGCTCTACAGTTAGCTT
GTAAGGCTAACTAGTGAAATTCCTGGACTTGTTCTTAAAAATGCAAGTTTTAGTAATTAACAAAATG
ATGATGAAGATGTCCCTTTTCCCTACAACTACAGATGGAGGGAGATTTTTCTTTGCCATACAACTAG
CTTAAAGGATTAATTTGATAAGTTGTTAAACTGAGAACTTTCACAAAAGTATCCATCTTGTTTTTGA
TATAAATGGAGATACATGTAGTTATTCATAACTGTCAGTAATTTGCTGTTTATCCTGTTTCTATATA
TCTGTCCTTGAGAGTATAATTTTAATAAATATTTCAAAGATTTTAGGAAATGTCATGTTCTGTTAAA
AAACTTCCAAAAGTAATTTTGATGAACAGTTTTGATAACTTAGTACTAACTAGGACTAAGACTGCAA
TTGACTGCTCTACATTCCTGAACTTTATAAGCAGTAGTTGTTTCTCTCTGTCAAATCAGTGTCCCCT
TTTCCCATTTGCATCATGGGAAAGTGAAACCTTATAATTCTGCTAAATTTATTATAACAAATACATT
GAAATTCTCCATTTTATTAAATTAATAGAATGTTATGAATCAAAGCACCAAAAAAACTGATGCAATT
TTGATGTCTCGTTCTGTACCACATTCTCCAGATCTTAATATATTCAGTTCCACATTATTGGTGCTAG
TAGGAGACATAATGAAAACAGTTAAATGAAATCCACAGCGAGTATACTGATTAACCAGTACTGTCAA
ATTTCTCATACCTATTGAATTTTAACTACTGACAAAATGAGCAGTAACAATTCCATTTACCTGATTG
TCCTTTGGCAAAGGATATTATTAAGAATCACTAAAAATAGCCATAAAGAAGCCATATGGAAGGAAGA
AGGAAAACAAATGGCATGAAAAGGTCTCTCACTGAGTAACTATGCTCTTATAGTTGACGCTGGTATA
TTTCTTTTATTCACTACCTAAAAATGAACTATCTTACTCTTTAATTATAGAATAAAAACTGCAGGAA
AGTATTTAAGACTTTTTTTCACAAACACAGGTATCTCATTAACCTATGTTTTATTTTGAGTAAATTC
ATTATTCATTATTTCACATTATAAAAAGTAACCACACATACATATGCATTCACAAATTAGATCATCT
TTATCATACATCAATATATTTTAAAAAACAAATATCTTCTAATATCAATATAGTTATATGCTGATTG
CATTTTGAAATAGAGAAGCTGACAATAGCTTCACACGGTATATCTCAAGAACTGACAGTTTAAAATT
AAGAACTGTATATATTCCACAGGCAAATTTTGATGGAAATATTAGCATTAGTACAAATAAATGCTGT
TGACATAGCTTAAGCATGATAGCTTGGAATAACAGCTGATTCAGACTAGATTCATCATTTTAAATAA
AGACAAGTACAATCTAAAATGTAAACAAAGTATTTATAAAATAAATTCTCTAGGAAATAAAGAAAAT
CATCAATCTATTATTTTTAAGGTATTTATAGCTCAAAGTTACCAGAAATCTTTGTGGAATTTTCACT
GCCAAATTTAAATTTGGGAATGTCCGGGTACAACATATTGTCACCACAATCCGGAGGGCCGCCAAAA
TCGCAGACGGCTATTTGCATCCTTTCAGTGTGACTTTTCAAGTGGGCTTGGAGACTCATGAGAAAAT
GCAGTATCTTTCTCACCTTCCAAGTCCCCCTCCAAGTGCTTATCAAGCTAGGACAATTCAGCTGATG
TAGACTTTCATACGATTTTTAAATGCTAAAACTCTAGAACAATTAAATGGCTGGTTTCCTGCACAAA
TAAATGCAGACTTGTCTCTTTTGCAGCAGTGGTTAAAGCACATTCCTAGAGATGTTTTTCATTACAC
TTCACTATAACATTGGAATTCCGTAACCACATTATTACTCAAGAAATATATATTATACCTCCTAGGG
AATCTAATTTGAAATATGAAAAGTTTAACATCAGCTGTCATTATGTCTCTCTTTCTGCTCATTAACA
ACAACAAAAAAAAAAACCCAAAATTTAAAAACAAAGCCCCAGCCACTGCTTTAGCTTTTGTGTACCA
ATCACATTATCTCCTGCTGCCTTTGTTTTGCCTCCTTCATCAAGCAGTTGATTTAAGGATTGGATTT
TCTGGATTTTCTTTGGGAAGAAAGAAATGAAGGAAGAGAGGGAGGGTGGGGAAGGAGGGAGTGAGAA
AGGGAGAAAAAGAAAAAAATATGAAAAATGTTATTCATATAATGTGTACAAAGTAAATTAAAAATAT
ATAGATACTCTACTTTGAATAATTCTAATATATGAGAAGT

Following Table 1 provides oligonucleoside mRNA target sequences of ZPI. together with the corresponding positions in transcript NM_016186.3. It is to be understood that SEQ ID NOs: 2 to 121 refer to human (Homo sapiens) mRNA sequences.

TABLE 1

| | Oligonucleoside mRNA | Starting |
|---|---|---|
| | target sequence | position on |
| SEQ ID NO | 5′ → 3′ | NM_016186.3 |
| SEQ ID NO: 2 | UUUGCCUUCAUCCACAAGGAUUU | 991 |
| SEQ ID NO: 3 | GCUGCGAAAGAUCUCCAUGAGGC | 756 |
| SEQ ID NO: 4 | AUGCUGGUGGUCCUCAUGGAGAA | 1390 |
| SEQ ID NO: 5 | CUGCGAAAGAUCUCCAUGAGGCA | 757 |
| SEQ ID NO: 6 | AAGUAUGAGAUGCAUGAGCUGCU | 1531 |
| SEQ ID NO: 7 | CUGUUUGAUGAGAUUAAUCCUGA | 1156 |
| SEQ ID NO: 8 | GAUGAGAUUAAUCCUGAAACCAA | 1162 |
| SEQ ID NO: 9 | UUUGAUGAGAUUAAUCCUGAAAC | 1159 |
| SEQ ID NO: 10 | UGAUGAGAUUAAUCCUGAAACCA | 1161 |
| SEQ ID NO: 11 | UUGAUGAGAUUAAUCCUGAAACC | 1160 |
| SEQ ID NO: 12 | AACUGUUUGAUGAGAUUAAUCCU | 1154 |
| SEQ ID NO: 13 | AGUUUUGCCUUCAUCCACAAGGA | 988 |
| SEQ ID NO: 14 | UGCGAAAGAUCUCCAUGAGGCAC | 758 |
| SEQ ID NO: 15 | CAUGCUGGUGGUCCUCAUGGAGA | 1389 |
| SEQ ID NO: 16 | UGCCUUCAUCCACAAGGAUUUUG | 993 |
| SEQ ID NO: 17 | GCGAAAGAUCUCCAUGAGGCACG | 759 |
| SEQ ID NO: 18 | CCUUCAUCCACAAGGAUUUUGAU | 995 |
| SEQ ID NO: 19 | UGUUUGAUGAGAUUAAUCCUGAA | 1157 |
| SEQ ID NO: 20 | UUUUGCCUUCAUCCACAAGGAUU | 990 |
| SEQ ID NO: 21 | AAGAUCUCCAUGAGGCACGAUGG | 763 |
| SEQ ID NO: 22 | ACCAUGCUGGUGGUCCUCAUGGA | 1387 |
| SEQ ID NO: 23 | GUUUUGCCUUCAUCCACAAGGAU | 989 |
| SEQ ID NO: 24 | UUGCCUUCAUCCACAAGGAUUUU | 992 |
| SEQ ID NO: 25 | CCUACCAAGGAAAUGCCACCAUG | 1370 |
| SEQ ID NO: 26 | GUUUGAUGAGAUUAAUCCUGAAA | 1158 |
| SEQ ID NO: 27 | GCCUUCAUCCACAAGGAUUUUGA | 994 |
| SEQ ID NO: 28 | CGAAAGAUCUCCAUGAGGCACGA | 760 |
| SEQ ID NO: 29 | ACUGUUUGAUGAGAUUAAUCCUG | 1155 |
| SEQ ID NO: 30 | CCAUGCUGGUGGUCCUCAUGGAG | 1388 |
| SEQ ID NO: 31 | GAGUUUUGCCUUCAUCCACAAGG | 987 |
| SEQ ID NO: 32 | UGCCACCAUGCUGGUGGUCCUCA | 1383 |
| SEQ ID NO: 33 | GCCACCAUGCUGGUGGUCCUCAU | 1384 |
| SEQ ID NO: 34 | GAAAGAUCUCCAUGAGGCACGAU | 761 |
| SEQ ID NO: 35 | GGAGUUUUGCCUUCAUCCACAAG | 986 |
| SEQ ID NO: 36 | CCACCAUGCUGGUGGUCCUCAUG | 1385 |
| SEQ ID NO: 37 | AAAGAUCUCCAUGAGGCACGAUG | 762 |
| SEQ ID NO: 38 | CACCAUGCUGGUGGUCCUCAUGG | 1386 |
| SEQ ID NO: 39 | UUUGCCUCCACCUUUGACAAGAA | 1321 |
| SEQ ID NO: 40 | UGCCUCCACCUUUGACAAGAAUU | 1323 |
| SEQ ID NO: 41 | ACCAUUAAGGUGCCCAUGAUGUA | 1285 |
| SEQ ID NO: 42 | AACUGCCCUACCAAGGAAAUGCC | 1364 |
| SEQ ID NO: 43 | CUCAAACUGCCCUACCAAGGAAA | 1360 |
| SEQ ID NO: 44 | CCUCAAACUGCCCUACCAAGGAA | 1359 |
| SEQ ID NO: 45 | GCCUCCACCUUUGACAAGAAUUU | 1324 |
| SEQ ID NO: 46 | GUCGACACUUUCCACCUGGACAA | 1255 |
| SEQ ID NO: 47 | ACACUUUCCACCUGGACAAGUAC | 1259 |
| SEQ ID NO: 48 | UCAAACUGCCCUACCAAGGAAAU | 1361 |
| SEQ ID NO: 49 | AAACUGCCCUACCAAGGAAAUGC | 1363 |
| SEQ ID NO: 50 | GACACUUUCCACCUGGACAAGUA | 1258 |
| SEQ ID NO: 51 | GACCAUUAAGGUGCCCAUGAUGU | 1284 |
| SEQ ID NO: 52 | GUUUGCCUCCACCUUUGACAAGA | 1320 |
| SEQ ID NO: 53 | ACUUUCCACCUGGACAAGUACAA | 1261 |
| SEQ ID NO: 54 | UGUCCUCAAACUGCCCUACCAAG | 1356 |
| SEQ ID NO: 55 | CAAACUGCCCUACCAAGGAAAUG | 1362 |
| SEQ ID NO: 56 | GUCCUCAAACUGCCCUACCAAGG | 1357 |
| SEQ ID NO: 57 | AUGUCCUCAAACUGCCCUACCAA | 1355 |
| SEQ ID NO: 58 | UCGACACUUUCCACCUGGACAAG | 1256 |
| SEQ ID NO: 59 | AGUUUGCCUCCACCUUUGACAAG | 1319 |
| SEQ ID NO: 60 | CGACACUUUCCACCUGGACAAGU | 1257 |
| SEQ ID NO: 61 | CUUUCCACCUGGACAAGUACAAG | 1262 |
| SEQ ID NO: 62 | CACUUUCCACCUGGACAAGUACA | 1260 |
| SEQ ID NO: 63 | UCCUCAAACUGCCCUACCAAGGA | 1358 |
| SEQ ID NO: 64 | GAUUACAUCUUGUUCAAAGGGAA | 1198 |
| SEQ ID NO: 65 | AAUGCCACCAUGCUGGUGGUCCU | 1381 |
| SEQ ID NO: 66 | UUUAUCCAAGAGGUAUUUUGAUA | 1038 |
| SEQ ID NO: 67 | GGAAAUGCCACCAUGCUGGUGGU | 1378 |
| SEQ ID NO: 68 | GGAUUACAUCUUGUUCAAAGGGA | 1197 |
| SEQ ID NO: 69 | AUCUCCAUGAGGCACGAUGGCAA | 766 |
| SEQ ID NO: 70 | AUUCCAUGCCUCCUGUCAUCAAA | 1721 |
| SEQ ID NO: 71 | UUAUUCCAUGCCUCCUGUCAUCA | 1719 |
| SEQ ID NO: 72 | ACCAAGGAAAUGCCACCAUGCUG | 1373 |
| SEQ ID NO: 73 | GCUGGUGGUCCUCAUGGAGAAAA | 1392 |
| SEQ ID NO: 74 | ACAUCUUGUUCAAAGGGAAAUGG | 1202 |
| SEQ ID NO: 75 | CCAAGGAAAUGCCACCAUGCUGG | 1374 |
| SEQ ID NO: 76 | UUGCCUCCACCUUUGACAAGAAU | 1322 |
| SEQ ID NO: 77 | GGGAGUUUUGCCUUCAUCCACAA | 985 |
| SEQ ID NO: 78 | CUGCUGCGAAAGAUCUCCAUGAG | 754 |
| SEQ ID NO: 79 | CAAGUUUGCCUCCACCUUUGACA | 1317 |
| SEQ ID NO: 80 | AAGUUUGCCUCCACCUUUGACAA | 1318 |
| SEQ ID NO: 81 | UACAUCUUGUUCAAAGGGAAAUG | 1201 |
| SEQ ID NO: 82 | GGGGAGUUUUGCCUUCAUCCACA | 984 |
| SEQ ID NO: 83 | UGCUGCGAAAGAUCUCCAUGAGG | 755 |
| SEQ ID NO: 84 | UUCCAUGCCUCCUGUCAUCAAAG | 1722 |
| SEQ ID NO: 85 | UCUGUUUCUGGGCAGGGUGGUGA | 1794 |
| SEQ ID NO: 86 | CAGCCUGCUGCGAAAGAUCUCCA | 750 |
| SEQ ID NO: 87 | CAAACUGUUUGAUGAGAUUAAUC | 1152 |
| SEQ ID NO: 88 | CCAUGCCUCCUGUCAUCAAAGUG | 1724 |
| SEQ ID NO: 89 | GAAGUAUGAGAUGCAUGAGCUGC | 1530 |
| SEQ ID NO: 90 | AGAAGUAUGAGAUGCAUGAGCUG | 1529 |
| SEQ ID NO: 91 | CAUGUCCUCAAACUGCCCUACCA | 1354 |
| SEQ ID NO: 92 | AAACUGUUUGAUGAGAUUAAUCC | 1153 |
| SEQ ID NO: 93 | AAGACCAUUAAGGUGCCCAUGAU | 1282 |
| SEQ ID NO: 94 | AGACCAUUAAGGUGCCCAUGAUG | 1283 |
| SEQ ID NO: 95 | CUGUUUCUGGGCAGGGUGGUGAA | 1795 |
| SEQ ID NO: 96 | GCUUCUGUUUCUGGGCAGGGUGG | 1791 |
| SEQ ID NO: 97 | AGUUUUCUUUCCGAAGUUCAAGC | 1500 |
| SEQ ID NO: 98 | GUUUUCUUUCCGAAGUUCAAGCU | 1501 |
| SEQ ID NO: 99 | CUUCUGUUUCUGGGCAGGGUGGU | 1792 |
| SEQ ID NO: 100 | UCAUGUCCUCAAACUGCCCUACC | 1353 |
| SEQ ID NO: 101 | AGUCGACACUUUCCACCUGGACA | 1254 |
| SEQ ID NO: 102 | CCUUCAUCCACAAGGAUUU | 995 |
| SEQ ID NO: 103 | CGAAAGAUCUCCAUGAGGC | 760 |
| SEQ ID NO: 104 | UGGUGGUCCUCAUGGAGAA | 1394 |
| SEQ ID NO: 105 | GAAAGAUCUCCAUGAGGCA | 761 |
| SEQ ID NO: 106 | AUGAGAUGCAUGAGCUGCU | 1535 |
| SEQ ID NO: 107 | UUGAUGAGAUUAAUCCUGA | 1160 |
| SEQ ID NO: 108 | AGAUUAAUCCUGAAACCAA | 1166 |
| SEQ ID NO: 109 | AUGAGAUUAAUCCUGAAAC | 1163 |
| SEQ ID NO: 110 | GAGAUUAAUCCUGAAACCA | 1165 |
| SEQ ID NO: 111 | UGAGAUUAAUCCUGAAACC | 1164 |
| SEQ ID NO: 112 | GUUUGAUGAGAUUAAUCCU | 1158 |
| SEQ ID NO: 113 | UUGCCUUCAUCCACAAGGA | 992 |
| SEQ ID NO: 114 | AAAGAUCUCCAUGAGGCAC | 762 |
| SEQ ID NO: 115 | CUGGUGGUCCUCAUGGAGA | 1393 |
| SEQ ID NO: 116 | UUCAUCCACAAGGAUUUUG | 997 |
| SEQ ID NO: 117 | AAGAUCUCCAUGAGGCACG | 763 |
| SEQ ID NO: 118 | CAUCCACAAGGAUUUUGAU | 999 |
| SEQ ID NO: 119 | UGAUGAGAUUAAUCCUGAA | 1161 |
| SEQ ID NO: 120 | GCCUUCAUCCACAAGGAUU | 994 |
| SEQ ID NO: 121 | UCUCCAUGAGGCACGAUGG | 767 |

Table 2 provides the unmodified first (antisense) and corresponding unmodified second (sense) strand sequences for siRNA oligonucleosides according to the present invention, together with the corresponding positions in the overall gene sequence of SEQ ID NO: 1 as follows.

TABLE 2

| | First (Antisense) | | Second (Sense) | |
|---|---|---|---|---|
| | Strand | | Strand Base | |
| | Base Sequence | | Sequence | |
| | 5′ → 3′ | | 5′ → 3′ | |
| | (Shown as | | (Shown as | |
| | an Unmodified | | an Unmodified | Corresponding |
| SEQ ID | Nucleoside | SEQ ID | Nucleoside | positions on |
| NO (AS) | Sequence) | NO (SS) | Sequence) | NM_016186.3 |
| SEQ ID | CAUGGUGGCAUUUCCUUGG | SEQ ID | UACCAAGGAAAUGCCAC | 1370-1391 |
| NO: 145 | UAGG | NO: 265 | CAUG | |
| SEQ ID | UCGUGCCUCAUGGAGAUCU | SEQ ID | AAAGAUCUCCAUGAGGC | 760-781 |
| NO: 148 | UUCG | NO: 268 | ACGA | |
| SEQ ID | AAAUCCUUGUGGAUGAAGG | SEQ ID | UGCCUUCAUCCACAAGG | 991-1012 |
| NO: 122 | CAAA | NO: 242 | AUUU | |
| SEQ ID | GCCUCAUGGAGAUCUUUCG | SEQ ID | UGCGAAAGAUCUCCAUG | 756-777 |
| NO: 123 | CAGC | NO: 243 | AGGC | |
| SEQ ID | UUCUCCAUGAGGACCACCA | SEQ ID | GCUGGUGGUCCUCAUGG | 1390-1411 |
| NO: 124 | GCAU | NO: 244 | AGAA | |
| SEQ ID | UGCCUCAUGGAGAUCUUUC | SEQ ID | GCGAAAGAUCUCCAUGA | 757-778 |
| NO: 125 | GCAG | NO: 245 | GGCA | |
| SEQ ID | AGCAGCUCAUGCAUCUCAU | SEQ ID | GUAUGAGAUGCAUGAGC | 1531-1552 |
| NO: 126 | ACUU | NO: 246 | UGCU | |
| SEQ ID | UCAGGAUUAAUCUCAUCAA | SEQ ID | GUUUGAUGAGAUUAAUC | 1156-1177 |
| NO: 127 | ACAG | NO: 247 | CUGA | |
| SEQ ID | UUGGUUUCAGGAUUAAUCU | SEQ ID | UGAGAUUAAUCCUGAAA | 1162-1183 |
| NO: 128 | CAUC | NO: 248 | CCAA | |
| SEQ ID | GUUUCAGGAUUAAUCUCAU | SEQ ID | UGAUGAGAUUAAUCCUG | 1159-1180 |
| NO: 129 | CAAA | NO: 249 | AAAC | |
| SEQ ID | UGGUUUCAGGAUUAAUCUC | SEQ ID | AUGAGAUUAAUCCUGAA | 1161-1182 |
| NO: 130 | AUCA | NO: 250 | ACCA | |
| SEQ ID | GGUUUCAGGAUUAAUCUCA | SEQ ID | GAUGAGAUUAAUCCUGA | 1160-1181 |
| NO: 131 | UCAA | NO: 251 | AACC | |
| SEQ ID | AGGAUUAAUCUCAUCAAAC | SEQ ID | CUGUUUGAUGAGAUUAA | 1154-1175 |
| NO: 132 | AGUU | NO: 252 | UCCU | |
| SEQ ID | UCCUUGUGGAUGAAGGCAA | SEQ ID | UUUUGCCUUCAUCCACA | 988-1009 |
| NO: 133 | AACU | NO: 253 | AGGA | |
| SEQ ID | GUGCCUCAUGGAGAUCUUU | SEQ ID | CGAAAGAUCUCCAUGAG | 758-779 |
| NO: 134 | CGCA | NO: 254 | GCAC | |
| SEQ ID | UCUCCAUGAGGACCACCAG | SEQ ID | UGCUGGUGGUCCUCAUG | 1389-1410 |
| NO: 135 | CAUG | NO: 255 | GAGA | |
| SEQ ID | CAAAAUCCUUGUGGAUGAA | SEQ ID | CCUUCAUCCACAAGGAU | 993-1014 |
| NO: 136 | GGCA | NO: 256 | UUUG | |
| SEQ ID | CGUGCCUCAUGGAGAUCUU | SEQ ID | GAAAGAUCUCCAUGAGG | 759-780 |
| NO: 137 | UCGC | NO: 257 | CACG | |
| SEQ ID | AUCAAAAUCCUUGUGGAUG | SEQ ID | UUCAUCCACAAGGAUUU | 995-1016 |
| NO: 138 | AAGG | NO: 258 | UGAU | |
| SEQ ID | UUCAGGAUUAAUCUCAUCA | SEQ ID | UUUGAUGAGAUUAAUCC | 1157-1178 |
| NO: 139 | AACA | NO: 259 | UGAA | |
| SEQ ID | AAUCCUUGUGGAUGAAGGC | SEQ ID | UUGCCUUCAUCCACAAG | 990-1011 |
| NO: 140 | AAAA | NO: 260 | GAUU | |
| SEQ ID | CCAUCGUGCCUCAUGGAGA | SEQ ID | GAUCUCCAUGAGGCACG | 763-784 |
| NO: 141 | UCUU | NO: 261 | AUGG | |
| SEQ ID | UCCAUGAGGACCACCAGCA | SEQ ID | CAUGCUGGUGGUCCUCA | 1387-1408 |
| NO: 142 | UGGU | NO: 262 | UGGA | |
| SEQ ID | AUCCUUGUGGAUGAAGGCA | SEQ ID | UUUGCCUUCAUCCACAA | 989-1010 |
| NO: 143 | AAAC | NO: 263 | GGAU | |
| SEQ ID | AAAAUCCUUGUGGAUGAAG | SEQ ID | GCCUUCAUCCACAAGGA | 992-1013 |
| NO: 144 | GCAA | NO: 264 | UUUU | |
| SEQ ID | UUUCAGGAUUAAUCUCAUC | SEQ ID | UUGAUGAGAUUAAUCCU | 1158-1179 |
| NO: 146 | AAAC | NO: 266 | GAAA | |
| SEQ ID | UCAAAAUCCUUGUGGAUGA | SEQ ID | CUUCAUCCACAAGGAUU | 994-1015 |
| NO: 147 | AGGC | NO: 267 | UUGA | |
| SEQ ID | CAGGAUUAAUCUCAUCAAA | SEQ ID | UGUUUGAUGAGAUUAAU | 1155-1176 |
| NO: 149 | CAGU | NO: 269 | CCUG | |
| SEQ ID | CUCCAUGAGGACCACCAGC | SEQ ID | AUGCUGGUGGUCCUCAU | 1388-1409 |
| NO: 150 | AUGG | NO: 270 | GGAG | |
| SEQ ID | CCUUGUGGAUGAAGGCAAA | SEQ ID | GUUUUGCCUUCAUCCAC | 987-1008 |
| NO: 151 | ACUC | NO: 271 | AAGG | |
| SEQ ID | UGAGGACCACCAGCAUGGU | SEQ ID | CCACCAUGCUGGUGGUC | 1383-1404 |
| NO: 152 | GGCA | NO: 272 | CUCA | |
| SEQ ID | AUGAGGACCACCAGCAUGG | SEQ ID | CACCAUGCUGGUGGUCC | 1384-1405 |
| NO: 153 | UGGC | NO: 273 | UCAU | |
| SEQ ID | AUCGUGCCUCAUGGAGAUC | SEQ ID | AAGAUCUCCAUGAGGCA | 761-782 |
| NO: 154 | UUUC | NO: 274 | CGAU | |
| SEQ ID | CUUGUGGAUGAAGGCAAAA | SEQ ID | AGUUUUGCCUUCAUCCA | 986-1007 |
| NO: 155 | CUCC | NO: 275 | CAAG | |
| SEQ ID | CAUGAGGACCACCAGCAUG | SEQ ID | ACCAUGCUGGUGGUCCU | 1385-1406 |
| NO: 156 | GUGG | NO: 276 | CAUG | |
| SEQ ID | CAUCGUGCCUCAUGGAGAU | SEQ ID | AGAUCUCCAUGAGGCAC | 762-783 |
| NO: 157 | CUUU | NO: 277 | GAUG | |
| SEQ ID | CCAUGAGGACCACCAGCAU | SEQ ID | CCAUGCUGGUGGUCCUC | 1386-1407 |
| NO: 158 | GGUG | NO: 278 | AUGG | |
| SEQ ID | UUCUUGUCAAAGGUGGAGG | SEQ ID | UGCCUCCACCUUUGACA | 1321-1342 |
| NO: 159 | CAAA | NO: 279 | AGAA | |
| SEQ ID | AAUUCUUGUCAAAGGUGGA | SEQ ID | CCUCCACCUUUGACAAG | 1323-1344 |
| NO: 160 | GGCA | NO: 280 | AAUU | |
| SEQ ID | UACAUCAUGGGCACCUUAA | SEQ ID | CAUUAAGGUGCCCAUGA | 1285-1306 |
| NO: 161 | UGGU | NO: 281 | UGUA | |
| SEQ ID | GGCAUUUCCUUGGUAGGGC | SEQ ID | CUGCCCUACCAAGGAAA | 1364-1385 |
| NO: 162 | AGUU | NO: 282 | UGCC | |
| SEQ ID | UUUCCUUGGUAGGGCAGUU | SEQ ID | CAAACUGCCCUACCAAG | 1360-1381 |
| NO: 163 | UGAG | NO: 283 | GAAA | |
| SEQ ID | UUCCUUGGUAGGGCAGUUU | SEQ ID | UCAAACUGCCCUACCAA | 1359-1380 |
| NO: 164 | GAGG | NO: 284 | GGAA | |
| SEQ ID | AAAUUCUUGUCAAAGGUGG | SEQ ID | CUCCACCUUUGACAAGA | 1324-1345 |
| NO: 165 | AGGC | NO: 285 | AUUU | |
| SEQ ID | UUGUCCAGGUGGAAAGUGU | SEQ ID | CGACACUUUCCACCUGG | 1255-1276 |
| NO: 166 | CGAC | NO: 286 | ACAA | |
| SEQ ID | GUACUUGUCCAGGUGGAAA | SEQ ID | ACUUUCCACCUGGACAA | 1259-1280 |
| NO: 167 | GUGU | NO: 287 | GUAC | |
| SEQ ID | AUUUCCUUGGUAGGGCAGU | SEQ ID | AAACUGCCCUACCAAGG | 1361-1382 |
| NO: 168 | UUGA | NO: 288 | AAAU | |
| SEQ ID | GCAUUUCCUUGGUAGGGCA | SEQ ID | ACUGCCCUACCAAGGAA | 1363-1384 |
| NO: 169 | GUUU | NO: 289 | AUGC | |
| SEQ ID | UACUUGUCCAGGUGGAAAG | SEQ ID | CACUUUCCACCUGGACA | 1258-1279 |
| NO: 170 | UGUC | NO: 290 | AGUA | |
| SEQ ID | ACAUCAUGGGCACCUUAAU | SEQ ID | CCAUUAAGGUGCCCAUG | 1284-1305 |
| NO: 171 | GGUC | NO: 291 | AUGU | |
| SEQ ID | UCUUGUCAAAGGUGGAGGC | SEQ ID | UUGCCUCCACCUUUGAC | 1320-1341 |
| NO: 172 | AAAC | NO: 292 | AAGA | |
| SEQ ID | UUGUACUUGUCCAGGUGGA | SEQ ID | UUUCCACCUGGACAAGU | 1261-1282 |
| NO: 173 | AAGU | NO: 293 | ACAA | |
| SEQ ID | CUUGGUAGGGCAGUUUGAG | SEQ ID | UCCUCAAACUGCCCUAC | 1356-1377 |
| NO: 174 | GACA | NO: 294 | CAAG | |
| SEQ ID | CAUUUCCUUGGUAGGGCAG | SEQ ID | AACUGCCCUACCAAGGA | 1362-1383 |
| NO: 175 | UUUG | NO: 295 | AAUG | |
| SEQ ID | CCUUGGUAGGGCAGUUUGA | SEQ ID | CCUCAAACUGCCCUACC | 1357-1378 |
| NO: 176 | GGAC | NO: 296 | AAGG | |
| SEQ ID | UUGGUAGGGCAGUUUGAGG | SEQ ID | GUCCUCAAACUGCCCUA | 1355-1376 |
| NO: 177 | ACAU | NO: 297 | CCAA | |
| SEQ ID | CUUGUCCAGGUGGAAAGUG | SEQ ID | GACACUUUCCACCUGGA | 1256-1277 |
| NO: 178 | UCGA | NO: 298 | CAAG | |
| SEQ ID | CUUGUCAAAGGUGGAGGCA | SEQ ID | UUUGCCUCCACCUUUGA | 1319-1340 |
| NO: 179 | AACU | NO: 299 | CAAG | |
| SEQ ID | ACUUGUCCAGGUGGAAAGU | SEQ ID | ACACUUUCCACCUGGAC | 1257-1278 |
| NO: 180 | GUCG | NO: 300 | AAGU | |
| SEQ ID | CUUGUACUUGUCCAGGUGG | SEQ ID | UUCCACCUGGACAAGUA | 1262-1283 |
| NO: 181 | AAAG | NO: 301 | CAAG | |
| SEQ ID | UGUACUUGUCCAGGUGGAA | SEQ ID | CUUUCCACCUGGACAAG | 1260-1281 |
| NO: 182 | AGUG | NO: 302 | UACA | |
| SEQ ID | UCCUUGGUAGGGCAGUUUG | SEQ ID | CUCAAACUGCCCUACCA | 1358-1379 |
| NO: 183 | AGGA | NO: 303 | AGGA | |
| SEQ ID | UUCCCUUUGAACAAGAUGU | SEQ ID | UUACAUCUUGUUCAAAG | 1198-1219 |
| NO: 184 | AAUC | NO: 304 | GGAA | |
| SEQ ID | AGGACCACCAGCAUGGUGG | SEQ ID | UGCCACCAUGCUGGUGG | 1381-1402 |
| NO: 185 | CAUU | NO: 305 | UCCU | |
| SEQ ID | UAUCAAAAUACCUCUUGGA | SEQ ID | UAUCCAAGAGGUAUUUU | 1038-1059 |
| NO: 186 | UAAA | NO: 306 | GAUA | |
| SEQ ID | ACCACCAGCAUGGUGGCAU | SEQ ID | AAAUGCCACCAUGCUGG | 1378-1399 |
| NO: 187 | UUCC | NO: 307 | UGGU | |
| SEQ ID | UCCCUUUGAACAAGAUGUA | SEQ ID | AUUACAUCUUGUUCAAA | 1197-1218 |
| NO: 188 | AUCC | NO: 308 | GGGA | |
| SEQ ID | UUGCCAUCGUGCCUCAUGG | SEQ ID | CUCCAUGAGGCACGAUG | 766-787 |
| NO: 189 | AGAU | NO: 309 | GCAA | |
| SEQ ID | UUUGAUGACAGGAGGCAUG | SEQ ID | UCCAUGCCUCCUGUCAU | 1721-1742 |
| NO: 190 | GAAU | NO: 310 | CAAA | |
| SEQ ID | UGAUGACAGGAGGCAUGGA | SEQ ID | AUUCCAUGCCUCCUGUC | 1719-1740 |
| NO: 191 | AUAA | NO: 311 | AUCA | |
| SEQ ID | CAGCAUGGUGGCAUUUCCU | SEQ ID | CAAGGAAAUGCCACCAU | 1373-1394 |
| NO: 192 | UGGU | NO: 312 | GCUG | |
| SEQ ID | UUUUCUCCAUGAGGACCAC | SEQ ID | UGGUGGUCCUCAUGGAG | 1392-1413 |
| NO: 193 | CAGC | NO: 313 | AAAA | |
| SEQ ID | CCAUUUCCCUUUGAACAAG | SEQ ID | AUCUUGUUCAAAGGGAA | 1202-1223 |
| NO: 194 | AUGU | NO: 314 | AUGG | |
| SEQ ID | CCAGCAUGGUGGCAUUUCC | SEQ ID | AAGGAAAUGCCACCAUG | 1374-1395 |
| NO: 195 | UUGG | NO: 315 | CUGG | |
| SEQ ID | AUUCUUGUCAAAGGUGGAG | SEQ ID | GCCUCCACCUUUGACAA | 1322-1343 |
| NO: 196 | GCAA | NO: 316 | GAAU | |
| SEQ ID | UUGUGGAUGAAGGCAAAAC | SEQ ID | GAGUUUUGCCUUCAUCC | 985-1006 |
| NO: 197 | UCCC | NO: 317 | ACAA | |
| SEQ ID | CUCAUGGAGAUCUUUCGCA | SEQ ID | GCUGCGAAAGAUCUCCA | 754-775 |
| NO: 198 | GCAG | NO: 318 | UGAG | |
| SEQ ID | UGUCAAAGGUGGAGGCAAA | SEQ ID | AGUUUGCCUCCACCUUU | 1317-1338 |
| NO: 199 | CUUG | NO: 319 | GACA | |
| SEQ ID | UUGUCAAAGGUGGAGGCAA | SEQ ID | GUUUGCCUCCACCUUUG | 1318-1339 |
| NO: 200 | ACUU | NO: 320 | ACAA | |
| SEQ ID | CAUUUCCCUUUGAACAAGA | SEQ ID | CAUCUUGUUCAAAGGGA | 1201-1222 |
| NO: 201 | UGUA | NO: 321 | AAUG | |
| SEQ ID | UGUGGAUGAAGGCAAAACU | SEQ ID | GGAGUUUUGCCUUCAUC | 984-1005 |
| NO: 202 | CCCC | NO: 322 | CACA | |
| SEQ ID | CCUCAUGGAGAUCUUUCGC | SEQ ID | CUGCGAAAGAUCUCCAU | 755-776 |
| NO: 203 | AGCA | NO: 323 | GAGG | |
| SEQ ID | CUUUGAUGACAGGAGGCAU | SEQ ID | CCAUGCCUCCUGUCAUC | 1722-1743 |
| NO: 204 | GGAA | NO: 324 | AAAG | |
| SEQ ID | UCACCACCCUGCCCAGAAA | SEQ ID | UGUUUCUGGGCAGGGUG | 1794-1815 |
| NO: 205 | CAGA | NO: 325 | GUGA | |
| SEQ ID | UGGAGAUCUUUCGCAGCAG | SEQ ID | GCCUGCUGCGAAAGAUC | 750-771 |
| NO: 206 | GCUG | NO: 326 | UCCA | |
| SEQ ID | GAUUAAUCUCAUCAAACAG | SEQ ID | AACUGUUUGAUGAGAUU | 1152-1173 |
| NO: 207 | UUUG | NO: 327 | AAUC | |
| SEQ ID | CACUUUGAUGACAGGAGGC | SEQ ID | AUGCCUCCUGUCAUCAA | 1724-1745 |
| NO: 208 | AUGG | NO: 328 | AGUG | |
| SEQ ID | GCAGCUCAUGCAUCUCAUA | SEQ ID | AGUAUGAGAUGCAUGAG | 1530-1551 |
| NO: 209 | CUUC | NO: 329 | CUGC | |
| SEQ ID | CAGCUCAUGCAUCUCAUAC | SEQ ID | AAGUAUGAGAUGCAUGA | 1529-1550 |
| NO: 210 | UUCU | NO: 330 | GCUG | |
| SEQ ID | UGGUAGGGCAGUUUGAGGA | SEQ ID | UGUCCUCAAACUGCCCU | 1354-1375 |
| NO: 211 | CAUG | NO: 331 | ACCA | |
| SEQ ID | GGAUUAAUCUCAUCAAACA | SEQ ID | ACUGUUUGAUGAGAUUA | 1153-1174 |
| NO: 212 | GUUU | NO: 332 | AUCC | |
| SEQ ID | AUCAUGGGCACCUUAAUGG | SEQ ID | GACCAUUAAGGUGCCCA | 1282-1303 |
| NO: 213 | UCUU | NO: 333 | UGAU | |
| SEQ ID | CAUCAUGGGCACCUUAAUG | SEQ ID | ACCAUUAAGGUGCCCAU | 1283-1304 |
| NO: 214 | GUCU | NO: 334 | GAUG | |
| SEQ ID | UUCACCACCCUGCCCAGAA | SEQ ID | GUUUCUGGGCAGGGUGG | 1795-1816 |
| NO: 215 | ACAG | NO: 335 | UGAA | |
| SEQ ID | CCACCCUGCCCAGAAACAG | SEQ ID | UUCUGUUUCUGGGCAGG | 1791-1812 |
| NO: 216 | AAGC | NO: 336 | GUGG | |
| SEQ ID | GCUUGAACUUCGGAAAGAA | SEQ ID | UUUUCUUUCCGAAGUUC | 1500-1521 |
| NO: 217 | AACU | NO: 337 | AAGC | |
| SEQ ID | AGCUUGAACUUCGGAAAGA | SEQ ID | UUUCUUUCCGAAGUUCA | 1501-1522 |
| NO: 218 | AAAC | NO: 338 | AGCU | |
| SEQ ID | ACCACCCUGCCCAGAAACA | SEQ ID | UCUGUUUCUGGGCAGGG | 1792-1813 |
| NO: 219 | GAAG | NO: 339 | UGGU | |
| SEQ ID | GGUAGGGCAGUUUGAGGAC | SEQ ID | AUGUCCUCAAACUGCCC | 1353-1374 |
| NO: 220 | AUGA | NO: 340 | UACC | |
| SEQ ID | UGUCCAGGUGGAAAGUGUC | SEQ ID | UCGACACUUUCCACCUG | 1254-1275 |
| NO: 221 | GACU | NO: 341 | GACA | |
| SEQ ID | AAAUCCUUGUGGAUGAAGG | SEQ ID | CCUUCAUCCACAAGGAU | 995-1014 |
| NO: 222 | | NO: 342 | UU | |
| SEQ ID | GCCUCAUGGAGAUCUUUCG | SEQ ID | CGAAAGAUCUCCAUGAG | 760-779 |
| NO: 223 | | NO: 343 | GC | |
| SEQ ID | UUCUCCAUGAGGACCACCA | SEQ ID | UGGUGGUCCUCAUGGAG | 1394-1413 |
| NO: 224 | | NO: 344 | AA | |
| SEQ ID | UGCCUCAUGGAGAUCUUUC | SEQ ID | GAAAGAUCUCCAUGAGG | 761-780 |
| NO: 225 | | NO: 345 | CA | |
| SEQ ID | AGCAGCUCAUGCAUCUCAU | SEQ ID | AUGAGAUGCAUGAGCUG | 1535-1554 |
| NO: 226 | | NO: 346 | CU | |
| SEQ ID | UCAGGAUUAAUCUCAUCAA | SEQ ID | UUGAUGAGAUUAAUCCU | 1160-1179 |
| NO: 227 | | NO: 347 | GA | |
| SEQ ID | UUGGUUUCAGGAUUAAUCU | SEQ ID | AGAUUAAUCCUGAAACC | 1166-1185 |
| NO: 228 | | NO: 348 | AA | |
| SEQ ID | GUUUCAGGAUUAAUCUCAU | SEQ ID | AUGAGAUUAAUCCUGAA | 1163-1182 |
| NO: 229 | | NO: 349 | AC | |
| SEQ ID | UGGUUUCAGGAUUAAUCUC | SEQ ID | GAGAUUAAUCCUGAAAC | 1165-1184 |
| NO: 230 | | NO: 350 | CA | |
| SEQ ID | GGUUUCAGGAUUAAUCUCA | SEQ ID | UGAGAUUAAUCCUGAAA | 1164-1183 |
| NO: 231 | | NO: 351 | CC | |
| SEQ ID | AGGAUUAAUCUCAUCAAAC | SEQ ID | GUUUGAUGAGAUUAAUC | 1158-1177 |
| NO: 232 | | NO: 352 | CU | |
| SEQ ID | UCCUUGUGGAUGAAGGCAA | SEQ ID | UUGCCUUCAUCCACAAG | 992-1011 |
| NO: 233 | | NO: 353 | GA | |
| SEQ ID | GUGCCUCAUGGAGAUCUUU | SEQ ID | AAAGAUCUCCAUGAGGC | 762-781 |
| NO: 234 | | NO: 354 | AC | |
| SEQ ID | UCUCCAUGAGGACCACCAG | SEQ ID | CUGGUGGUCCUCAUGGA | 1393-1412 |
| NO: 235 | | NO: 355 | GA | |
| SEQ ID | CAAAAUCCUUGUGGAUGAA | SEQ ID | UUCAUCCACAAGGAUUU | 997-1016 |
| NO: 236 | | NO: 356 | UG | |
| SEQ ID | CGUGCCUCAUGGAGAUCUU | SEQ ID | AAGAUCUCCAUGAGGCA | 763-782 |
| NO: 237 | | NO: 357 | CG | |
| SEQ ID | AUCAAAAUCCUUGUGGAUG | SEQ ID | CAUCCACAAGGAUUUUG | 999-1018 |
| NO: 238 | | NO: 358 | AU | |
| SEQ ID | UUCAGGAUUAAUCUCAUCA | SEQ ID | UGAUGAGAUUAAUCCUG | 1161-1180 |
| NO: 239 | | NO: 359 | AA | |
| SEQ ID | AAUCCUUGUGGAUGAAGGC | SEQ ID | GCCUUCAUCCACAAGGA | 994-1013 |
| NO: 240 | | NO: 360 | UU | |
| SEQ ID | CCAUCGUGCCUCAUGGAGA | SEQ ID | UCUCCAUGAGGCACGAU | 767-786 |
| NO: 241 | | NO: 361 | GG | |
| SEQ ID | AAAGUCCUUGUGGAUGAAG | SEQ ID | GCCUUCAUCCACAAGGA | NA |
| NO: 787 | GCAA | NO: 791 | CUUU | |
| SEQ ID | AACUUCUUGUCAAAGGUGG | SEQ ID | CUCCACCUUUGACAAGA | NA |
| NO: 788 | AGGC | NO: 792 | AGUU | |
| SEQ ID | UGUGGAUGAAGGCAAAGCU | SEQ ID | GUAGCUUUGCCUUCAUC | NA |
| NO: 789 | ACCC | NO: 793 | CACA | |

Table 3 provides the modified first (antisense) sequences. together with the corresponding unmodified first (antisense) sequences for siRNA oligonucleosides according to the present invention as follows.

TABLE 3

| | | | Underlying | |
|---|---|---|---|---|
| | | | Base Sequence | |
| | | | 5′ → 3′ | |
| | | | (Shown as | |
| | Modified First | SEQ ID | an Unmodified | SEQ ID |
| Antisense | (Antisense) Strand | NO (AS- | Nucleoside | NO (AS- |
| strand ID | 5′ → 3′ | mod) | Sequence) | unmod) |
| ETXS1036 | CmsAfsUmGfGmUfGmGmCmAmUmUm | SEQ ID | CAUGGUGGCAUUUCC | SEQ ID |
| | UmCfCmUfUmGmGmUmAmsGmsGm | NO: 764 | UUGGUAGG | NO: 145 |
| ETXS1040 | UmsCfsGmUfGmCfCmUmCmAmUmGm | SEQ ID | UCGUGCCUCAUGGAG | SEQ ID |
| | GmAfGmAfUmCmUmUmUmsCmsGm | NO: 766 | AUCUUUCG | NO: 148 |
| ETXS632 | AmsAfsAmUmCmCfUmUfGfUmGmGm | SEQ ID | AAAUCCUUGUGGAUG | SEQ ID |
| | AmUfGmAfAmGmGmCmAmsAmsAm | NO: 362 | AAGGCAAA | NO: 122 |
| ETXS634 | GmsCfsCmUmCmAfUmGfGfAmGmAm | SEQ ID | GCCUCAUGGAGAUCU | SEQ ID |
| | UmCfUmUfUmCmGmCmAmsGmsCm | NO: 363 | UUCGCAGC | NO: 123 |
| ETXS636 | UmsUfsCmUmCmCfAmUfGfAmGmGm | SEQ ID | UUCUCCAUGAGGACC | SEQ ID |
| | AmCfCmAfCmCmAmGmCmsAmsUm | NO: 364 | ACCAGCAU | NO: 124 |
| ETXS638 | UmsGfsCmCmUmCfAmUfGfGmAmGm | SEQ ID | UGCCUCAUGGAGAUC | SEQ ID |
| | AmUfCmUfUmUmCmGmCmsAmsGm | NO: 365 | UUUCGCAG | NO: 125 |
| ETXS640 | AmsGfsCmAmGmCfUmCfAfUmGmCm | SEQ ID | AGCAGCUCAUGCAUC | SEQ ID |
| | AmUfCmUfCmAmUmAmCmsUmsUm | NO: 366 | UCAUACUU | NO: 126 |
| ETXS642 | UmsCfsAmGmGmAfUmUfAfAmUmCm | SEQ ID | UCAGGAUUAAUCUCA | SEQ ID |
| | UmCfAmUfCmAmAmAmCmsAmsGm | NO: 367 | UCAAACAG | NO: 127 |
| ETXS644 | UmsUfsGmGmUmUfUmCfAfGmGmAm | SEQ ID | UUGGUUUCAGGAUUA | SEQ ID |
| | UmUfAmAfUmCmUmCmAmsUmsCm | NO: 368 | AUCUCAUC | NO: 128 |
| ETXS646 | GmsUfsUmUmCmAfGmGfAfUmUmAm | SEQ ID | GUUUCAGGAUUAAUC | SEQ ID |
| | AmUfCmUfCmAmUmCmAmsAmsAm | NO: 369 | UCAUCAAA | NO: 129 |
| ETXS648 | UmsGfsGmUmUmUfCmAfGfGmAmUm | SEQ ID | UGGUUUCAGGAUUAA | SEQ ID |
| | UmAfAmUfCmUmCmAmUmsCmsAm | NO: 370 | UCUCAUCA | NO: 130 |
| ETXS650 | GmsGfsUmUmUmCfAmGfGfAmUmUm | SEQ ID | GGUUUCAGGAUUAAU | SEQ ID |
| | AmAfUmCfUmCmAmUmCmsAmsAm | NO: 371 | CUCAUCAA | NO: 131 |
| ETXS652 | AmsGfsGmAmUmUfAmAfUfCmUmCm | SEQ ID | AGGAUUAAUCUCAUC | SEQ ID |
| | AmUfCmAfAmAmCmAmGmsUmsUm | NO: 372 | AAACAGUU | NO: 132 |
| ETXS654 | UmsCfsCmUmUmGfUmGfGfAmUmGm | SEQ ID | UCCUUGUGGAUGAAG | SEQ ID |
| | AmAfGmGfCmAmAmAmAmsCmsUm | NO: 373 | GCAAAACU | NO: 133 |
| ETXS656 | GmsUfsGmCmCmUfCmAfUfGmGmAm | SEQ ID | GUGCCUCAUGGAGAU | SEQ ID |
| | GmAfUmCfUmUmUmCmGmsCmsAm | NO: 374 | CUUUCGCA | NO: 134 |
| ETXS658 | UmsCfsUmCmCmAfUmGfAfGmGmAm | SEQ ID | UCUCCAUGAGGACCA | SEQ ID |
| | CmCfAmCfCmAmGmCmAmsUmsGm | NO: 375 | CCAGCAUG | NO: 135 |
| ETXS660 | CmsAfsAmAmAmUfCmCfUfUmGmUm | SEQ ID | CAAAAUCCUUGUGGA | SEQ ID |
| | GmGfAmUfGmAmAmGmGmsCmsAm | NO: 376 | UGAAGGCA | NO: 136 |
| ETXS662 | CmsGfsUmGmCmCfUmCfAfUmGmGm | SEQ ID | CGUGCCUCAUGGAGA | SEQ ID |
| | AmGfAmUfCmUmUmUmCmsGmsCm | NO: 377 | UCUUUCGC | NO: 137 |
| ETXS664 | AmsUfsCmAmAmAfAmUfCfCmUmUm | SEQ ID | AUCAAAAUCCUUGUG | SEQ ID |
| | GmUfGmGfAmUmGmAmAmsGmsGm | NO: 378 | GAUGAAGG | NO: 138 |
| ETXS666 | UmsUfsCmAmGmGfAmUfUfAmAmUm | SEQ ID | UUCAGGAUUAAUCUC | SEQ ID |
| | CmUfCmAfUmCmAmAmAmsCmsAm | NO: 379 | AUCAAACA | NO: 139 |
| ETXS668 | AmsAfsUmCmCmUfUmGfUfGmGmAm | SEQ ID | AAUCCUUGUGGAUGA | SEQ ID |
| | UmGfAmAfGmGmCmAmAmsAmsAm | NO: 380 | AGGCAAAA | NO: 140 |
| ETXS670 | CmsCfsAmUmCmGfUmGfCfCmUmCm | SEQ ID | CCAUCGUGCCUCAUG | SEQ ID |
| | AmUfGmGfAmGmAmUmCmsUmsUm | NO: 381 | GAGAUCUU | NO: 141 |
| ETXS672 | UmsCfsCmAmUmGfAmGfGfAmCmCm | SEQ ID | UCCAUGAGGACCACC | SEQ ID |
| | AmCfCmAfGmCmAmUmGmsGmsUm | NO: 382 | AGCAUGGU | NO: 142 |
| ETXS674 | AmsUfsCmCmUmUfGmUfGfGmAmUm | SEQ ID | AUCCUUGUGGAUGAA | SEQ ID |
| | GmAfAmGfGmCmAmAmAmsAmsCm | NO: 383 | GGCAAAAC | NO: 143 |
| ETXS676 | AmsAfsAmAmUmCfCmUfUfGmUmGm | SEQ ID | AAAAUCCUUGUGGAU | SEQ ID |
| | GmAfUmGfAmAmGmGmCmsAmsAm | NO: 384 | GAAGGCAA | NO: 144 |
| ETXS678 | CmsAfsUmGmGmUfGmGfCfAmUmUm | SEQ ID | CAUGGUGGCAUUUCC | SEQ ID |
| | UmCfCmUfUmGmGmUmAmsGmsGm | NO: 385 | UUGGUAGG | NO: 145 |
| ETXS680 | UmsUfsUmCmAmGfGmAfUfUmAmAm | SEQ ID | UUUCAGGAUUAAUCU | SEQ ID |
| | UmCfUmCfAmUmCmAmAmsAmsCm | NO: 386 | CAUCAAAC | NO: 146 |
| ETXS682 | UmsCfsAmAmAmAfUmCfCfUmUmGm | SEQ ID | UCAAAAUCCUUGUGG | SEQ ID |
| | UmGfGmAfUmGmAmAmGmsGmsCm | NO: 387 | AUGAAGGC | NO: 147 |
| ETXS684 | UmsCfsGmUmGmCfCmUfCfAmUmGm | SEQ ID | UCGUGCCUCAUGGAG | SEQ ID |
| | GmAfGmAfUmCmUmUmUmsCmsGm | NO: 388 | AUCUUUCG | NO: 148 |
| ETXS686 | CmsAfsGmGmAmUfUmAfAfUmCmUm | SEQ ID | CAGGAUUAAUCUCAU | SEQ ID |
| | CmAfUmCfAmAmAmCmAmsGmsUm | NO: 389 | CAAACAGU | NO: 149 |
| ETXS688 | CmsUfsCmCmAmUfGmAfGfGmAmCm | SEQ ID | CUCCAUGAGGACCAC | SEQ ID |
| | CmAfCmCfAmGmCmAmUmsGmsGm | NO: 390 | CAGCAUGG | NO: 150 |
| ETXS690 | CmsCfsUmUmGmUfGmGfAfUmGmAm | SEQ ID | CCUUGUGGAUGAAGG | SEQ ID |
| | AmGfGmCfAmAmAmAmCmsUmsCm | NO: 391 | CAAAACUC | NO: 151 |
| ETXS692 | UmsGfsAmGmGmAfCmCfAfCmCmAm | SEQ ID | UGAGGACCACCAGCA | SEQ ID |
| | GmCfAmUfGmGmUmGmGmsCmsAm | NO: 392 | UGGUGGCA | NO: 152 |
| ETXS694 | AmsUfsGmAmGmGfAmCfCfAmCmCm | SEQ ID | AUGAGGACCACCAGC | SEQ ID |
| | AmGfCmAfUmGmGmUmGmsGmsCm | NO: 393 | AUGGUGGC | NO: 153 |
| ETXS696 | AmsUfsCmGmUmGfCmCfUfCmAmUm | SEQ ID | AUCGUGCCUCAUGGA | SEQ ID |
| | GmGfAmGfAmUmCmUmUmsUmsCm | NO: 394 | GAUCUUUC | NO: 154 |
| ETXS698 | CmsUfsUmGmUmGfGmAfUfGmAmAm | SEQ ID | CUUGUGGAUGAAGGC | SEQ ID |
| | GmGfCmAfAmAmAmCmUmsCmsCm | NO: 395 | AAAACUCC | NO: 155 |
| ETXS700 | CmsAfsUmGmAmGfGmAfCfCmAmCm | SEQ ID | CAUGAGGACCACCAG | SEQ ID |
| | CmAfGmCfAmUmGmGmUmsGmsGm | NO: 396 | CAUGGUGG | NO: 156 |
| ETXS702 | CmsAfsUmCmGmUfGmCfCfUmCmAm | SEQ ID | CAUCGUGCCUCAUGG | SEQ ID |
| | UmGfGmAfGmAmUmCmUmsUmsUm | NO: 397 | AGAUCUUU | NO: 157 |
| ETXS704 | CmsCfsAmUmGmAfGmGfAfCmCmAm | SEQ ID | CCAUGAGGACCACCA | SEQ ID |
| | CmCfAmGfCmAmUmGmGmsUmsGm | NO: 398 | GCAUGGUG | NO: 158 |
| ETXS706 | UmsUfsCmUmUmGfUmCfAfAmAmGm | SEQ ID | UUCUUGUCAAAGGUG | SEQ ID |
| | GmUfGmGfAmGmGmCmAmsAmsAm | NO: 399 | GAGGCAAA | NO: 159 |
| ETXS708 | AmsAfsUmUmCmUfUmGfUfCmAmAm | SEQ ID | AAUUCUUGUCAAAGG | SEQ ID |
| | AmGfGmUfGmGmAmGmGmsCmsAm | NO: 400 | UGGAGGCA | NO: 160 |
| ETXS710 | UmsAfsCmAmUmCfAmUfGfGmGmCm | SEQ ID | UACAUCAUGGGCACC | SEQ ID |
| | AmCfCmUfUmAmAmUmGmsGmsUm | NO: 401 | UUAAUGGU | NO: 161 |
| ETXS712 | GmsGfsCmAmUmUfUmCfCfUmUmGm | SEQ ID | GGCAUUUCCUUGGUA | SEQ ID |
| | GmUfAmGfGmGmCmAmGmsUmsUm | NO: 402 | GGGCAGUU | NO: 162 |
| ETXS714 | UmsUfsUmCmCmUfUmGfGfUmAmGm | SEQ ID | UUUCCUUGGUAGGGC | SEQ ID |
| | GmGfCmAfGmUmUmUmGmsAmsGm | NO: 403 | AGUUUGAG | NO: 163 |
| ETXS716 | UmsUfsCmCmUmUfGmGfUfAmGmGm | SEQ ID | UUCCUUGGUAGGGCA | SEQ ID |
| | GmCfAmGfUmUmUmGmAmsGmsGm | NO: 404 | GUUUGAGG | NO: 164 |
| ETXS718 | AmsAfsAmUmUmCfUmUfGfUmCmAm | SEQ ID | AAAUUCUUGUCAAAG | SEQ ID |
| | AmAfGmGfUmGmGmAmGmsGmsCm | NO: 405 | GUGGAGGC | NO: 165 |
| ETXS720 | UmsUfsGmUmCmCfAmGfGfUmGmGm | SEQ ID | UUGUCCAGGUGGAAA | SEQ ID |
| | AmAfAmGfUmGmUmCmGmsAmsCm | NO: 406 | GUGUCGAC | NO: 166 |
| ETXS722 | GmsUfsAmCmUmUfGmUfCfCmAmGm | SEQ ID | GUACUUGUCCAGGUG | SEQ ID |
| | GmUfGmGfAmAmAmGmUmsGmsUm | NO: 407 | GAAAGUGU | NO: 167 |
| ETXS724 | AmsUfsUmUmCmCfUmUfGfGmUmAm | SEQ ID | AUUUCCUUGGUAGGG | SEQ ID |
| | GmGfGmCfAmGmUmUmUmsGmsAm | NO: 408 | CAGUUUGA | NO: 168 |
| ETXS726 | GmsCfsAmUmUmUfCmCfUfUmGmGm | SEQ ID | GCAUUUCCUUGGUAG | SEQ ID |
| | UmAfGmGfGmCmAmGmUmsUmsUm | NO: 409 | GGCAGUUU | NO: 169 |
| ETXS728 | UmsAfsCmUmUmGfUmCfCfAmGmGm | SEQ ID | UACUUGUCCAGGUGG | SEQ ID |
| | UmGfGmAfAmAmGmUmGmsUmsCm | NO: 410 | AAAGUGUC | NO: 170 |
| ETXS730 | AmsCfsAmUmCmAfUmGfGfGmCmAm | SEQ ID | ACAUCAUGGGCACCU | SEQ ID |
| | CmCfUmUfAmAmUmGmGmsUmsCm | NO: 411 | UAAUGGUC | NO: 171 |
| ETXS732 | UmsCfsUmUmGmUfCmAfAfAmGmGm | SEQ ID | UCUUGUCAAAGGUGG | SEQ ID |
| | UmGfGmAfGmGmCmAmAmsAmsCm | NO: 412 | AGGCAAAC | NO: 172 |
| ETXS734 | UmsUfsGmUmAmCfUmUfGfUmCmCm | SEQ ID | UUGUACUUGUCCAGG | SEQ ID |
| | AmGfGmUfGmGmAmAmAmsGmsUm | NO: 413 | UGGAAAGU | NO: 173 |
| ETXS736 | CmsUfsUmGmGmUfAmGfGfGmCmAm | SEQ ID | CUUGGUAGGGCAGUU | SEQ ID |
| | GmUfUmUfGmAmGmGmAmsCmsAm | NO: 414 | UGAGGACA | NO: 174 |
| ETXS738 | CmsAfsUmUmUmCfCmUfUfGmGmUm | SEQ ID | CAUUUCCUUGGUAGG | SEQ ID |
| | AmGfGmGfCmAmGmUmUmsUmsGm | NO: 415 | GCAGUUUG | NO: 175 |
| ETXS740 | CmsCfsUmUmGmGfUmAfGfGmGmCm | SEQ ID | CCUUGGUAGGGCAGU | SEQ ID |
| | AmGfUmUfUmGmAmGmGmsAmsCm | NO: 416 | UUGAGGAC | NO: 176 |
| ETXS742 | UmsUfsGmGmUmAfGmGfGfCmAmGm | SEQ ID | UUGGUAGGGCAGUUU | SEQ ID |
| | UmUfUmGfAmGmGmAmCmsAmsUm | NO: 417 | GAGGACAU | NO: 177 |
| ETXS744 | CmsUfsUmGmUmCfCmAfGfGmUmGm | SEQ ID | CUUGUCCAGGUGGAA | SEQ ID |
| | GmAfAmAfGmUmGmUmCmsGmsAm | NO: 418 | AGUGUCGA | NO: 178 |
| ETXS746 | CmsUfsUmGmUmCfAmAfAfGmGmUm | SEQ ID | CUUGUCAAAGGUGGA | SEQ ID |
| | GmGfAmGfGmCmAmAmAmsCmsUm | NO: 419 | GGCAAACU | NO: 179 |
| ETXS748 | AmsCfsUmUmGmUfCmCfAfGmGmUm | SEQ ID | ACUUGUCCAGGUGGA | SEQ ID |
| | GmGfAmAfAmGmUmGmUmsCmsGm | NO: 420 | AAGUGUCG | NO: 180 |
| ETXS750 | CmsUfsUmGmUmAfCmUfUfGmUmCm | SEQ ID | CUUGUACUUGUCCAG | SEQ ID |
| | CmAfGmGfUmGmGmAmAmsAmsGm | NO: 421 | GUGGAAAG | NO: 181 |
| ETXS752 | UmsGfsUmAmCmUfUmGfUfCmCmAm | SEQ ID | UGUACUUGUCCAGGU | SEQ ID |
| | GmGfUmGfGmAmAmAmGmsUmsGm | NO: 422 | GGAAAGUG | NO: 182 |
| ETXS754 | UmsCfsCmUmUmGfGmUfAfGmGmGm | SEQ ID | UCCUUGGUAGGGCAG | SEQ ID |
| | CmAfGmUfUmUmGmAmGmsGmsAm | NO: 423 | UUUGAGGA | NO: 183 |
| ETXS756 | UmsUfsCmCmCmUfUmUfGfAmAmCm | SEQ ID | UUCCCUUUGAACAAG | SEQ ID |
| | AmAfGmAfUmGmUmAmAmsUmsCm | NO: 424 | AUGUAAUC | NO: 184 |
| ETXS758 | AmsGfsGmAmCmCfAmCfCfAmGmCm | SEQ ID | AGGACCACCAGCAUG | SEQ ID |
| | AmUfGmGfUmGmGmCmAmsUmsUm | NO: 425 | GUGGCAUU | NO: 185 |
| ETXS760 | UmsAfsUmCmAmAfAmAfUfAmCmCm | SEQ ID | UAUCAAAAUACCUCU | SEQ ID |
| | UmCfUmUfGmGmAmUmAmsAmsAm | NO: 426 | UGGAUAAA | NO: 186 |
| ETXS762 | AmsCfsCmAmCmCfAmGfCfAmUmGm | SEQ ID | ACCACCAGCAUGGUG | SEQ ID |
| | GmUfGmGfCmAmUmUmUmsCmsCm | NO: 427 | GCAUUUCC | NO: 187 |
| ETXS764 | UmsCfsCmCmUmUfUmGfAfAmCmAm | SEQ ID | UCCCUUUGAACAAGA | SEQ ID |
| | AmGfAmUfGmUmAmAmUmsCmsCm | NO: 428 | UGUAAUCC | NO: 188 |
| ETXS766 | UmsUfsGmCmCmAfUmCfGfUmGmCm | SEQ ID | UUGCCAUCGUGCCUC | SEQ ID |
| | CmUfCmAfUmGmGmAmGmsAmsUm | NO: 429 | AUGGAGAU | NO: 189 |
| ETXS768 | UmsUfsUmGmAmUfGmAfCfAmGmGm | SEQ ID | UUUGAUGACAGGAGG | SEQ ID |
| | AmGfGmCfAmUmGmGmAmsAmsUm | NO: 430 | CAUGGAAU | NO: 190 |
| ETXS770 | UmsGfsAmUmGmAfCmAfGfGmAmGm | SEQ ID | UGAUGACAGGAGGCA | SEQ ID |
| | GmCfAmUfGmGmAmAmUmsAmsAm | NO: 431 | UGGAAUAA | NO: 191 |
| ETXS772 | CmsAfsGmCmAmUfGmGfUfGmGmCm | SEQ ID | CAGCAUGGUGGCAUU | SEQ ID |
| | AmUfUmUfCmCmUmUmGmsGmsUm | NO: 432 | UCCUUGGU | NO: 192 |
| ETXS774 | UmsUfsUmUmCmUfCmCfAfUmGmAm | SEQ ID | UUUUCUCCAUGAGGA | SEQ ID |
| | GmGfAmCfCmAmCmCmAmsGmsCm | NO: 433 | CCACCAGC | NO: 193 |
| ETXS776 | CmsCfsAmUmUmUfCmCfCfUmUmUm | SEQ ID | CCAUUUCCCUUUGAA | SEQ ID |
| | GmAfAmCfAmAmGmAmUmsGmsUm | NO: 434 | CAAGAUGU | NO: 194 |
| ETXS778 | CmsCfsAmGmCmAfUmGfGfUmGmGm | SEQ ID | CCAGCAUGGUGGCAU | SEQ ID |
| | CmAfUmUfUmCmCmUmUmsGmsGm | NO: 435 | UUCCUUGG | NO: 195 |
| ETXS780 | AmsUfsUmCmUmUfGmUfCfAmAmAm | SEQ ID | AUUCUUGUCAAAGGU | SEQ ID |
| | GmGfUmGfGmAmGmGmCmsAmsAm | NO: 436 | GGAGGCAA | NO: 196 |
| ETXS782 | UmsUfsGmUmGmGfAmUfGfAmAmGm | SEQ ID | UUGUGGAUGAAGGCA | SEQ ID |
| | GmCfAmAfAmAmCmUmCmsCmsCm | NO: 437 | AAACUCCC | NO: 197 |
| ETXS784 | CmsUfsCmAmUmGfGmAfGfAmUmCm | SEQ ID | CUCAUGGAGAUCUUU | SEQ ID |
| | UmUfUmCfGmCmAmGmCmsAmsGm | NO: 438 | CGCAGCAG | NO: 198 |
| ETXS786 | UmsGfsUmCmAmAfAmGfGfUmGmGm | SEQ ID | UGUCAAAGGUGGAGG | SEQ ID |
| | AmGfGmCfAmAmAmCmUmsUmsGm | NO: 439 | CAAACUUG | NO: 199 |
| ETXS788 | UmsUfsGmUmCmAfAmAfGfGmUmGm | SEQ ID | UUGUCAAAGGUGGAG | SEQ ID |
| | GmAfGmGfCmAmAmAmCmsUmsUm | NO: 440 | GCAAACUU | NO: 200 |
| ETXS790 | CmsAfsUmUmUmCfCmCfUfUmUmGm | SEQ ID | CAUUUCCCUUUGAAC | SEQ ID |
| | AmAfCmAfAmGmAmUmGmsUmsAm | NO: 441 | AAGAUGUA | NO: 201 |
| ETXS792 | UmsGfsUmGmGmAfUmGfAfAmGmGm | SEQ ID | UGUGGAUGAAGGCAA | SEQ ID |
| | CmAfAmAfAmCmUmCmCmsCmsCm | NO: 442 | AACUCCCC | NO: 202 |
| ETXS794 | CmsCfsUmCmAmUfGmGfAfGmAmUm | SEQ ID | CCUCAUGGAGAUCUU | SEQ ID |
| | CmUfUmUfCmGmCmAmGmsCmsAm | NO: 443 | UCGCAGCA | NO: 203 |
| ETXS796 | CmsUfsUmUmGmAfUmGfAfCmAmGm | SEQ ID | CUUUGAUGACAGGAG | SEQ ID |
| | GmAfGmGfCmAmUmGmGmsAmsAm | NO: 444 | GCAUGGAA | NO: 204 |
| ETXS798 | UmsCfsAmCmCmAfCmCfCfUmGmCm | SEQ ID | UCACCACCCUGCCCA | SEQ ID |
| | CmCfAmGfAmAmAmCmAmsGmsAm | NO: 445 | GAAACAGA | NO: 205 |
| ETXS800 | UmsGfsGmAmGmAfUmCfUfUmUmCm | SEQ ID | UGGAGAUCUUUCGCA | SEQ ID |
| | GmCfAmGfCmAmGmGmCmsUmsGm | NO: 446 | GCAGGCUG | NO: 206 |
| ETXS802 | GmsAfsUmUmAmAfUmCfUfCmAmUm | SEQ ID | GAUUAAUCUCAUCAA | SEQ ID |
| | CmAfAmAfCmAmGmUmUmsUmsGm | NO: 447 | ACAGUUUG | NO: 207 |
| ETXS804 | CmsAfsCmUmUmUfGmAfUfGmAmCm | SEQ ID | CACUUUGAUGACAGG | SEQ ID |
| | AmGfGmAfGmGmCmAmUmsGmsGm | NO: 448 | AGGCAUGG | NO: 208 |
| ETXS806 | GmsCfsAmGmCmUfCmAfUfGmCmAm | SEQ ID | GCAGCUCAUGCAUCU | SEQ ID |
| | UmCfUmCfAmUmAmCmUmsUmsCm | NO: 449 | CAUACUUC | NO: 209 |
| ETXS808 | CmsAfsGmCmUmCfAmUfGfCmAmUm | SEQ ID | CAGCUCAUGCAUCUC | SEQ ID |
| | CmUfCmAfUmAmCmUmUmsCmsUm | NO: 450 | AUACUUCU | NO: 210 |
| ETXS810 | UmsGfsGmUmAmGfGmGfCfAmGmUm | SEQ ID | UGGUAGGGCAGUUUG | SEQ ID |
| | UmUfGmAfGmGmAmCmAmsUmsGm | NO: 451 | AGGACAUG | NO: 211 |
| ETXS812 | GmsGfsAmUmUmAfAmUfCfUmCmAm | SEQ ID | GGAUUAAUCUCAUCA | SEQ ID |
| | UmCfAmAfAmCmAmGmUmsUmsUm | NO: 452 | AACAGUUU | NO: 212 |
| ETXS814 | AmsUfsCmAmUmGfGmGfCfAmCmCm | SEQ ID | AUCAUGGGCACCUUA | SEQ ID |
| | UmUfAmAfUmGmGmUmCmsUmsUm | NO: 453 | AUGGUCUU | NO: 213 |
| ETXS816 | CmsAfsUmCmAmUfGmGfGfCmAmCm | SEQ ID | CAUCAUGGGCACCUU | SEQ ID |
| | CmUfUmAfAmUmGmGmUmsCmsUm | NO: 454 | AAUGGUCU | NO: 214 |
| ETXS818 | UmsUfsCmAmCmCfAmCfCfCmUmGm | SEQ ID | UUCACCACCCUGCCC | SEQ ID |
| | CmCfCmAfGmAmAmAmCmsAmsGm | NO: 455 | AGAAACAG | NO: 215 |
| ETXS820 | CmsCfsAmCmCmCfUmGfCfCmCmAm | SEQ ID | CCACCCUGCCCAGAA | SEQ ID |
| | GmAfAmAfCmAmGmAmAmsGmsCm | NO: 456 | ACAGAAGC | NO: 216 |
| ETXS822 | GmsCfsUmUmGmAfAmCfUfUmCmGm | SEQ ID | GCUUGAACUUCGGAA | SEQ ID |
| | GmAfAmAfGmAmAmAmAmsCmsUm | NO: 457 | AGAAAACU | NO: 217 |
| ETXS824 | AmsGfsCmUmUmGfAmAfCfUmUmCm | SEQ ID | AGCUUGAACUUCGGA | SEQ ID |
| | GmGfAmAfAmGmAmAmAmsAmsCm | NO: 458 | AAGAAAAC | NO: 218 |
| ETXS826 | AmsCfsCmAmCmCfCmUfGfCmCmCm | SEQ ID | ACCACCCUGCCCAGA | SEQ ID |
| | AmGfAmAfAmCmAmGmAmsAmsGm | NO: 459 | AACAGAAG | NO: 219 |
| ETXS828 | GmsGfsUmAmGmGfGmCfAfGmUmUm | SEQ ID | GGUAGGGCAGUUUGA | SEQ ID |
| | UmGfAmGfGmAmCmAmUmsGmsAm | NO: 460 | GGACAUGA | NO: 220 |
| ETXS830 | UmsGfsUmCmCmAfGmGfUfGmGmAm | SEQ ID | UGUCCAGGUGGAAAG | SEQ ID |
| | AmAfGmUfGmUmCmGmAmsCmsUm | NO: 461 | UGUCGACU | NO: 221 |
| ETXS832 | AmsAfsAmUfCmCfUmUfGmUfGmGf | SEQ ID | AAAUCCUUGUGGAUG | SEQ ID |
| | AmUfGmAfAmsGfsGm | NO: 462 | AAGG | NO: 222 |
| ETXS834 | GmsCfsCmUfCmAfUmGfGmAfGmAf | SEQ ID | GCCUCAUGGAGAUCU | SEQ ID |
| | UmCfUmUfUmsCfsGm | NO: 463 | UUCG | NO: 223 |
| ETXS836 | UmsUfsCmUfCmCfAmUfGmAfGmGf | SEQ ID | UUCUCCAUGAGGACC | SEQ ID |
| | AmCfCmAfCmsCfsAm | NO: 464 | ACCA | NO: 224 |
| ETXS838 | UmsGfsCmCfUmCfAmUfGmGfAmGf | SEQ ID | UGCCUCAUGGAGAUC | SEQ ID |
| | AmUfCmUfUmsUfsCm | NO: 465 | UUUC | NO: 225 |
| ETXS840 | AmsGfsCmAfGmCfUmCfAmUfGmCf | SEQ ID | AGCAGCUCAUGCAUC | SEQ ID |
| | AmUfCmUfCmsAfsUm | NO: 466 | UCAU | NO: 226 |
| ETXS842 | UmsCfsAmGfGmAfUmUfAmAfUmCf | SEQ ID | UCAGGAUUAAUCUCA | SEQ ID |
| | UmCfAmUfCmsAfsAm | NO: 467 | UCAA | NO: 227 |
| ETXS844 | UmsUfsGmGfUmUfUmCfAmGfGmAf | SEQ ID | UUGGUUUCAGGAUUA | SEQ ID |
| | UmUfAmAfUmsCfsUm | NO: 468 | AUCU | NO: 228 |
| ETXS846 | GmsUfsUmUfCmAfGmGfAmUfUmAf | SEQ ID | GUUUCAGGAUUAAUC | SEQ ID |
| | AmUfCmUfCmsAfsUm | NO: 469 | UCAU | NO: 229 |
| ETXS848 | UmsGfsGmUfUmUfCmAfGmGfAmUf | SEQ ID | UGGUUUCAGGAUUAA | SEQ ID |
| | UmAfAmUfCmsUfsCm | NO: 470 | UCUC | NO: 230 |
| ETXS850 | GmsGfsUmUfUmCfAmGfGmAfUmUf | SEQ ID | GGUUUCAGGAUUAAU | SEQ ID |
| | AmAfUmCfUmsCfsAm | NO: 471 | CUCA | NO: 231 |
| ETXS852 | AmsGfsGmAfUmUfAmAfUmCfUmCf | SEQ ID | AGGAUUAAUCUCAUC | SEQ ID |
| | AmUfCmAfAmsAfsCm | NO: 472 | AAAC | NO: 232 |
| ETXS854 | UmsCfsCmUfUmGfUmGfGmAfUmGf | SEQ ID | UCCUUGUGGAUGAAG | SEQ ID |
| | AmAfGmGfCmsAfsAm | NO: 473 | GCAA | NO: 233 |
| ETXS856 | GmsUfsGmCfCmUfCmAfUmGfGmAf | SEQ ID | GUGCCUCAUGGAGAU | SEQ ID |
| | GmAfUmCfUmsUfsUm | NO: 474 | CUUU | NO: 234 |
| ETXS858 | UmsCfsUmCfCmAfUmGfAmGfGmAf | SEQ ID | UCUCCAUGAGGACCA | SEQ ID |
| | CmCfAmCfCmsAfsGm | NO: 475 | CCAG | NO: 235 |
| ETXS860 | CmsAfsAmAfAmUfCmCfUmUfGmUf | SEQ ID | CAAAAUCCUUGUGGA | SEQ ID |
| | GmGfAmUfGmsAfsAm | NO: 476 | UGAA | NO: 236 |
| ETXS862 | CmsGfsUmGfCmCfUmCfAmUfGmGf | SEQ ID | CGUGCCUCAUGGAGA | SEQ ID |
| | AmGfAmUfCmsUfsUm | NO: 477 | UCUU | NO: 237 |
| ETXS864 | AmsUfsCmAfAmAfAmUfCmCfUmUf | SEQ ID | AUCAAAAUCCUUGUG | SEQ ID |
| | GmUfGmGfAmsUfsGm | NO: 478 | GAUG | NO: 238 |
| ETXS866 | UmsUfsCmAfGmGfAmUfUmAfAmUf | SEQ ID | UUCAGGAUUAAUCUC | SEQ ID |
| | CmUfCmAfUmsCfsAm | NO: 479 | AUCA | NO: 239 |
| ETXS868 | AmsAfsUmCfCmUfUmGfUmGfGmAf | SEQ ID | AAUCCUUGUGGAUGA | SEQ ID |
| | UmGfAmAfGmsGfsCm | NO: 480 | AGGC | NO: 240 |
| ETXS870 | CmsCfsAmUfCmGfUmGfCmCfUmCf | SEQ ID | CCAUCGUGCCUCAUG | SEQ ID |
| | AmUfGmGfAmsGfsAm | NO: 481 | GAGA | NO: 241 |
| ETXS872 | AmsAfsAmUfCmCfUmUfGfUmGmGm | SEQ ID | AAAUCCUUGUGGAUG | SEQ ID |
| | AmUfGmAfAmGmGmCmAmsAmsAm | NO: 482 | AAGGCAAA | NO: 122 |
| ETXS874 | GmsCfsCmUfCmAfUmGfGfAmGmAm | SEQ ID | GCCUCAUGGAGAUCU | SEQ ID |
| | UmCfUmUfUmCmGmCmAmsGmsCm | NO: 483 | UUCGCAGC | NO: 123 |
| ETXS876 | UmsUfsCmUfCmCfAmUfGfAmGmGm | SEQ ID | UUCUCCAUGAGGACC | SEQ ID |
| | AmCfCmAfCmCmAmGmCmsAmsUm | NO: 484 | ACCAGCAU | NO: 124 |
| ETXS878 | UmsGfsCmCfUmCfAmUfGfGmAmGm | SEQ ID | UGCCUCAUGGAGAUC | SEQ ID |
| | AmUfCmUfUmUmCmGmCmsAmsGm | NO: 485 | UUUCGCAG | NO: 125 |
| ETXS880 | AmsGfsCmAfGmCfUmCfAfUmGmCm | SEQ ID | AGCAGCUCAUGCAUC | SEQ ID |
| | AmUfCmUfCmAmUmAmCmsUmsUm | NO: 486 | UCAUACUU | NO: 126 |
| ETXS882 | UmsCfsAmGfGmAfUmUfAfAmUmCm | SEQ ID | UCAGGAUUAAUCUCA | SEQ ID |
| | UmCfAmUfCmAmAmAmCmsAmsGm | NO: 487 | UCAAACAG | NO: 127 |
| ETXS884 | UmsUfsGmGfUmUfUmCfAfGmGmAm | SEQ ID | UUGGUUUCAGGAUUA | SEQ ID |
| | UmUfAmAfUmCmUmCmAmsUmsCm | NO: 488 | AUCUCAUC | NO: 128 |
| ETXS886 | GmsUfsUmUfCmAfGmGfAfUmUmAm | SEQ ID | GUUUCAGGAUUAAUC | SEQ ID |
| | AmUfCmUfCmAmUmCmAmsAmsAm | NO: 489 | UCAUCAAA | NO: 129 |
| ETXS888 | UmsGfsGmUfUmUfCmAfGfGmAmUm | SEQ ID | UGGUUUCAGGAUUAA | SEQ ID |
| | UmAfAmUfCmUmCmAmUmsCmsAm | NO: 490 | UCUCAUCA | NO: 130 |
| ETXS890 | GmsGfsUmUfUmCfAmGfGfAmUmUm | SEQ ID | GGUUUCAGGAUUAAU | SEQ ID |
| | AmAfUmCfUmCmAmUmCmsAmsAm | NO: 491 | CUCAUCAA | NO: 131 |
| ETXS892 | AmsGfsGmAfUmUfAmAfUfCmUmCm | SEQ ID | AGGAUUAAUCUCAUC | SEQ ID |
| | AmUfCmAfAmAmCmAmGmsUmsUm | NO: 492 | AAACAGUU | NO: 132 |
| ETXS894 | UmsCfsCmUfUmGfUmGfGfAmUmGm | SEQ ID | UCCUUGUGGAUGAAG | SEQ ID |
| | AmAfGmGfCmAmAmAmAmsCmsUm | NO: 493 | GCAAAACU | NO: 133 |
| ETXS896 | GmsUfsGmCfCmUfCmAfUfGmGmAm | SEQ ID | GUGCCUCAUGGAGAU | SEQ ID |
| | GmAfUmCfUmUmUmCmGmsCmsAm | NO: 494 | CUUUCGCA | NO: 134 |
| ETXS898 | UmsCfsUmCfCmAfUmGfAfGmGmAm | SEQ ID | UCUCCAUGAGGACCA | SEQ ID |
| | CmCfAmCfCmAmGmCmAmsUmsGm | NO: 495 | CCAGCAUG | NO: 135 |
| ETXS900 | CmsAfsAmAfAmUfCmCfUfUmGmUm | SEQ ID | CAAAAUCCUUGUGGA | SEQ ID |
| | GmGfAmUfGmAmAmGmGmsCmsAm | NO: 496 | UGAAGGCA | NO: 136 |
| ETXS902 | CmsGfsUmGfCmCfUmCfAfUmGmGm | SEQ ID | CGUGCCUCAUGGAGA | SEQ ID |
| | AmGfAmUfCmUmUmUmCmsGmsCm | NO: 497 | UCUUUCGC | NO: 137 |
| ETXS904 | AmsUfsCmAfAmAfAmUfCfCmUmUm | SEQ ID | AUCAAAAUCCUUGUG | SEQ ID |
| | GmUfGmGfAmUmGmAmAmsGmsGm | NO: 498 | GAUGAAGG | NO: 138 |
| ETXS906 | UmsUfsCmAfGmGfAmUfUfAmAmUm | SEQ ID | UUCAGGAUUAAUCUC | SEQ ID |
| | CmUfCmAfUmCmAmAmAmsCmsAm | NO: 499 | AUCAAACA | NO: 139 |
| ETXS908 | AmsAfsUmCfCmUfUmGfUfGmGmAm | SEQ ID | AAUCCUUGUGGAUGA | SEQ ID |
| | UmGfAmAfGmGmCmAmAmsAmsAm | NO: 500 | AGGCAAAA | NO: 140 |
| ETXS910 | CmsCfsAmUfCmGfUmGfCfCmUmCm | SEQ ID | CCAUCGUGCCUCAUG | SEQ ID |
| | AmUfGmGfAmGmAmUmCmsUmsUm | NO: 501 | GAGAUCUU | NO: 141 |
| ETXS912 | AmsAfsAmUfCmCfUmUfGfUmGmGm | SEQ ID | AAAUCCUUGUGGAUG | SEQ ID |
| | AmUfGmAfAmGmGmCmAmsAmsAm | NO: 502 | AAGGCAAA | NO: 122 |
| ETXS914 | GmsCfsCmUfCmAfUmGfGfAmGmAm | SEQ ID | GCCUCAUGGAGAUCU | SEQ ID |
| | UmCfUmUfUmCmGmCmAmsGmsCm | NO: 503 | UUCGCAGC | NO: 123 |
| ETXS916 | UmsUfsCmUfCmCfAmUfGfAmGmGm | SEQ ID | UUCUCCAUGAGGACC | SEQ ID |
| | AmCfCmAfCmCmAmGmCmsAmsUm | NO: 504 | ACCAGCAU | NO: 124 |
| ETXS918 | UmsGfsCmCfUmCfAmUfGfGmAmGm | SEQ ID | UGCCUCAUGGAGAUC | SEQ ID |
| | AmUfCmUfUmUmCmGmCmsAmsGm | NO: 505 | UUUCGCAG | NO: 125 |
| ETXS920 | AmsGfsCmAfGmCfUmCfAfUmGmCm | SEQ ID | AGCAGCUCAUGCAUC | SEQ ID |
| | AmUfCmUfCmAmUmAmCmsUmsUm | NO: 506 | UCAUACUU | NO: 126 |
| ETXS922 | UmsCfsAmGfGmAfUmUfAfAmUmCm | SEQ ID | UCAGGAUUAAUCUCA | SEQ ID |
| | UmCfAmUfCmAmAmAmCmsAmsGm | NO: 507 | UCAAACAG | NO: 127 |
| ETXS924 | UmsUfsGmGfUmUfUmCfAfGmGmAm | SEQ ID | UUGGUUUCAGGAUUA | SEQ ID |
| | UmUfAmAfUmCmUmCmAmsUmsCm | NO: 508 | AUCUCAUC | NO: 128 |
| ETXS926 | GmsUfsUmUfCmAfGmGfAfUmUmAm | SEQ ID | GUUUCAGGAUUAAUC | SEQ ID |
| | AmUfCmUfCmAmUmCmAmsAmsAm | NO: 509 | UCAUCAAA | NO: 129 |
| ETXS928 | UmsGfsGmUfUmUfCmAfGfGmAmUm | SEQ ID | UGGUUUCAGGAUUAA | SEQ ID |
| | UmAfAmUfCmUmCmAmUmsCmsAm | NO: 510 | UCUCAUCA | NO: 130 |
| ETXS930 | GmsGfsUmUfUmCfAmGfGfAmUmUm | SEQ ID | GGUUUCAGGAUUAAU | SEQ ID |
| | AmAfUmCfUmCmAmUmCmsAmsAm | NO: 511 | CUCAUCAA | NO: 131 |
| ETXS932 | AmsGfsGmAfUmUfAmAfUfCmUmCm | SEQ ID | AGGAUUAAUCUCAUC | SEQ ID |
| | AmUfCmAfAmAmCmAmGmsUmsUm | NO: 512 | AAACAGUU | NO: 132 |
| ETXS934 | UmsCfsCmUfUmGfUmGfGfAmUmGm | SEQ ID | UCCUUGUGGAUGAAG | SEQ ID |
| | AmAfGmGfCmAmAmAmAmsCmsUm | NO: 513 | GCAAAACU | NO: 133 |
| ETXS936 | GmsUfsGmCfCmUfCmAfUfGmGmAm | SEQ ID | GUGCCUCAUGGAGAU | SEQ ID |
| | GmAfUmCfUmUmUmCmGmsCmsAm | NO: 514 | CUUUCGCA | NO: 134 |
| ETXS938 | UmsCfsUmCfCmAfUmGfAfGmGmAm | SEQ ID | UCUCCAUGAGGACCA | SEQ ID |
| | CmCfAmCfCmAmGmCmAmsUmsGm | NO: 515 | CCAGCAUG | NO: 135 |
| ETXS940 | CmsAfsAmAfAmUfCmCfUfUmGmUm | SEQ ID | CAAAAUCCUUGUGGA | SEQ ID |
| | GmGfAmUfGmAmAmGmGmsCmsAm | NO: 516 | UGAAGGCA | NO: 136 |
| ETXS942 | CmsGfsUmGfCmCfUmCfAfUmGmGm | SEQ ID | CGUGCCUCAUGGAGA | SEQ ID |
| | AmGfAmUfCmUmUmUmCmsGmsCm | NO: 517 | UCUUUCGC | NO: 137 |
| ETXS944 | AmsUfsCmAfAmAfAmUfCfCmUmUm | SEQ ID | AUCAAAAUCCUUGUG | SEQ ID |
| | GmUfGmGfAmUmGmAmAmsGmsGm | NO: 518 | GAUGAAGG | NO: 138 |
| ETXS946 | UmsUfsCmAfGmGfAmUfUfAmAmUm | SEQ ID | UUCAGGAUUAAUCUC | SEQ ID |
| | CmUfCmAfUmCmAmAmAmsCmsAm | NO: 519 | AUCAAACA | NO: 139 |
| ETXS948 | AmsAfsUmCfCmUfUmGfUfGmGmAm | SEQ ID | AAUCCUUGUGGAUGA | SEQ ID |
| | UmGfAmAfGmGmCmAmAmsAmsAm | NO: 520 | AGGCAAAA | NO: 140 |
| ETXS950 | CmsCfsAmUfCmGfUmGfCfCmUmCm | SEQ ID | CCAUCGUGCCUCAUG | SEQ ID |
| | AmUfGmGfAmGmAmUmCmsUmsUm | NO: 521 | GAGAUCUU | NO: 141 |
| ETXS952 | AmsAfsAmUfCmCfUmUfGfUmGmGm | SEQ ID | AAAUCCUUGUGGAUG | SEQ ID |
| | AmUfGmAfAmGmGmCmAmsAmsAm | NO: 522 | AAGGCAAA | NO: 122 |
| ETXS954 | GmsCfsCmUfCmAfUmGfGfAmGmAm | SEQ ID | GCCUCAUGGAGAUCU | SEQ ID |
| | UmCfUmUfUmCmGmCmAmsGmsCm | NO: 523 | UUCGCAGC | NO: 123 |
| ETXS956 | UmsUfsCmUfCmCfAmUfGfAmGmGm | SEQ ID | UUCUCCAUGAGGACC | SEQ ID |
| | AmCfCmAfCmCmAmGmCmsAmsUm | NO: 524 | ACCAGCAU | NO: 124 |
| ETXS958 | UmsGfsCmCfUmCfAmUfGfGmAmGm | SEQ ID | UGCCUCAUGGAGAUC | SEQ ID |
| | AmUfCmUfUmUmCmGmCmsAmsGm | NO: 525 | UUUCGCAG | NO: 125 |
| ETXS960 | AmsGfsCmAfGmCfUmCfAfUmGmCm | SEQ ID | AGCAGCUCAUGCAUC | SEQ ID |
| | AmUfCmUfCmAmUmAmCmsUmsUm | NO: 526 | UCAUACUU | NO: 126 |
| ETXS962 | UmsCfsAmGfGmAfUmUfAfAmUmCm | SEQ ID | UCAGGAUUAAUCUCA | SEQ ID |
| | UmCfAmUfCmAmAmAmCmsAmsGm | NO: 527 | UCAAACAG | NO: 127 |
| ETXS964 | UmsUfsGmGfUmUfUmCfAfGmGmAm | SEQ ID | UUGGUUUCAGGAUUA | SEQ ID |
| | UmUfAmAfUmCmUmCmAmsUmsCm | NO: 528 | AUCUCAUC | NO: 128 |
| ETXS966 | GmsUfsUmUfCmAfGmGfAfUmUmAm | SEQ ID | GUUUCAGGAUUAAUC | SEQ ID |
| | AmUfCmUfCmAmUmCmAmsAmsAm | NO: 529 | UCAUCAAA | NO: 129 |
| ETXS968 | UmsGfsGmUfUmUfCmAfGfGmAmUm | SEQ ID | UGGUUUCAGGAUUAA | SEQ ID |
| | UmAfAmUfCmUmCmAmUmsCmsAm | NO: 530 | UCUCAUCA | NO: 130 |
| ETXS970 | GmsGfsUmUfUmCfAmGfGfAmUmUm | SEQ ID | GGUUUCAGGAUUAAU | SEQ ID |
| | AmAfUmCfUmCmAmUmCmsAmsAm | NO: 531 | CUCAUCAA | NO: 131 |
| ETXS972 | AmsGfsGmAfUmUfAmAfUfCmUmCm | SEQ ID | AGGAUUAAUCUCAUC | SEQ ID |
| | AmUfCmAfAmAmCmAmGmsUmsUm | NO: 532 | AAACAGUU | NO: 132 |
| ETXS974 | UmsCfsCmUfUmGfUmGfGfAmUmGm | SEQ ID | UCCUUGUGGAUGAAG | SEQ ID |
| | AmAfGmGfCmAmAmAmAmsCmsUm | NO: 533 | GCAAAACU | NO: 133 |
| ETXS976 | GmsUfsGmCfCmUfCmAfUfGmGmAm | SEQ ID | GUGCCUCAUGGAGAU | SEQ ID |
| | GmAfUmCfUmUmUmCmGmsCmsAm | NO: 534 | CUUUCGCA | NO: 134 |
| ETXS978 | UmsCfsUmCfCmAfUmGfAfGmGmAm | SEQ ID | UCUCCAUGAGGACCA | SEQ ID |
| | CmCfAmCfCmAmGmCmAmsUmsGm | NO: 535 | CCAGCAUG | NO: 135 |
| ETXS980 | CmsAfsAmAfAmUfCmCfUfUmGmUm | SEQ ID | CAAAAUCCUUGUGGA | SEQ ID |
| | GmGfAmUfGmAmAmGmGmsCmsAm | NO: 536 | UGAAGGCA | NO: 136 |
| ETXS982 | CmsGfsUmGfCmCfUmCfAfUmGmGm | SEQ ID | CGUGCCUCAUGGAGA | SEQ ID |
| | AmGfAmUfCmUmUmUmCmsGmsCm | NO: 537 | UCUUUCGC | NO: 137 |
| ETXS984 | AmsUfsCmAfAmAfAmUfCfCmUmUm | SEQ ID | AUCAAAAUCCUUGUG | SEQ ID |
| | GmUfGmGfAmUmGmAmAmsGmsGm | NO: 538 | GAUGAAGG | NO: 138 |
| ETXS986 | UmsUfsCmAfGmGfAmUfUfAmAmUm | SEQ ID | UUCAGGAUUAAUCUC | SEQ ID |
| | CmUfCmAfUmCmAmAmAmsCmsAm | NO: 539 | AUCAAACA | NO: 139 |
| ETXS988 | AmsAfsUmCfCmUfUmGfUfGmGmAm | SEQ ID | AAUCCUUGUGGAUGA | SEQ ID |
| | UmGfAmAfGmGmCmAmAmsAmsAm | NO: 540 | AGGCAAAA | NO: 140 |
| ETXS990 | CmsCfsAmUfCmGfUmGfCfCmUmCm | SEQ ID | CCAUCGUGCCUCAUG | SEQ ID |
| | AmUfGmGfAmGmAmUmCmsUmsUm | NO: 541 | GAGAUCUU | NO: 141 |
| ETXS992 | AmsAfsAmUfCmCfUmUmGmUmGmGm | SEQ ID | AAAUCCUUGUGGAUG | SEQ ID |
| | AmUfGmAfAmGmGmCmAmsAmsAm | NO: 542 | AAGGCAAA | NO: 122 |
| ETXS994 | GmsCfsCmUfCmAfUmGmGmAmGmAm | SEQ ID | GCCUCAUGGAGAUCU | SEQ ID |
| | UmCfUmUfUmCmGmCmAmsGmsCm | NO: 543 | UUCGCAGC | NO: 123 |
| ETXS996 | UmsUfsCmUfCmCfAmUmGmAmGmGm | SEQ ID | UUCUCCAUGAGGACC | SEQ ID |
| | AmCfCmAfCmCmAmGmCmsAmsUm | NO: 544 | ACCAGCAU | NO: 124 |
| ETXS998 | UmsGfsCmCfUmCfAmUmGmGmAmGm | SEQ ID | UGCCUCAUGGAGAUC | SEQ ID |
| | AmUfCmUfUmUmCmGmCmsAmsGm | NO: 545 | UUUCGCAG | NO: 125 |
| ETXS1000 | AmsGfsCmAfGmCfUmCmAmUmGmCm | SEQ ID | AGCAGCUCAUGCAUC | SEQ ID |
| | AmUfCmUfCmAmUmAmCmsUmsUm | NO: 546 | UCAUACUU | NO: 126 |
| ETXS1002 | UmsCfsAmGfGmAfUmUmAmAmUmCm | SEQ ID | UCAGGAUUAAUCUCA | SEQ ID |
| | UmCfAmUfCmAmAmAmCmsAmsGm | NO: 547 | UCAAACAG | NO: 127 |
| ETXS1004 | UmsUfsGmGfUmUfUmCmAmGmGmAm | SEQ ID | UUGGUUUCAGGAUUA | SEQ ID |
| | UmUfAmAfUmCmUmCmAmsUmsCm | NO: 548 | AUCUCAUC | NO: 128 |
| ETXS1006 | GmsUfsUmUfCmAfGmGmAmUmUmAm | SEQ ID | GUUUCAGGAUUAAUC | SEQ ID |
| | AmUfCmUfCmAmUmCmAmsAmsAm | NO: 549 | UCAUCAAA | NO: 129 |
| ETXS1008 | UmsGfsGmUfUmUfCmAmGmGmAmUm | SEQ ID | UGGUUUCAGGAUUAA | SEQ ID |
| | UmAfAmUfCmUmCmAmUmsCmsAm | NO: 550 | UCUCAUCA | NO: 130 |
| ETXS1010 | GmsGfsUmUfUmCfAmGmGmAmUmUm | SEQ ID | GGUUUCAGGAUUAAU | SEQ ID |
| | AmAfUmCfUmCmAmUmCmsAmsAm | NO: 551 | CUCAUCAA | NO: 131 |
| ETXS1012 | AmsGfsGmAfUmUfAmAmUmCmUmCm | SEQ ID | AGGAUUAAUCUCAUC | SEQ ID |
| | AmUfCmAfAmAmCmAmGmsUmsUm | NO: 552 | AAACAGUU | NO: 132 |
| ETXS1014 | UmsCfsCmUfUmGfUmGmGmAmUmGm | SEQ ID | UCCUUGUGGAUGAAG | SEQ ID |
| | AmAfGmGfCmAmAmAmAmsCmsUm | NO: 553 | GCAAAACU | NO: 133 |
| ETXS1016 | GmsUfsGmCfCmUfCmAmUmGmGmAm | SEQ ID | GUGCCUCAUGGAGAU | SEQ ID |
| | GmAfUmCfUmUmUmCmGmsCmsAm | NO: 554 | CUUUCGCA | NO: 134 |
| ETXS1018 | UmsCfsUmCfCmAfUmGmAmGmGmAm | SEQ ID | UCUCCAUGAGGACCA | SEQ ID |
| | CmCfAmCfCmAmGmCmAmsUmsGm | NO: 555 | CCAGCAUG | NO: 135 |
| ETXS1020 | CmsAfsAmAfAmUfCmCmUmUmGmUm | SEQ ID | CAAAAUCCUUGUGGA | SEQ ID |
| | GmGfAmUfGmAmAmGmGmsCmsAm | NO: 556 | UGAAGGCA | NO: 136 |
| ETXS1022 | CmsGfsUmGfCmCfUmCmAmUmGmGm | SEQ ID | CGUGCCUCAUGGAGA | SEQ ID |
| | AmGfAmUfCmUmUmUmCmsGmsCm | NO: 557 | UCUUUCGC | NO: 137 |
| ETXS1024 | AmsUfsCmAfAmAfAmUmCmCmUmUm | SEQ ID | AUCAAAAUCCUUGUG | SEQ ID |
| | GmUfGmGfAmUmGmAmAmsGmsGm | NO: 558 | GAUGAAGG | NO: 138 |
| ETXS1026 | UmsUfsCmAfGmGfAmUmUmAmAmUm | SEQ ID | UUCAGGAUUAAUCUC | SEQ ID |
| | CmUfCmAfUmCmAmAmAmsCmsAm | NO: 559 | AUCAAACA | NO: 139 |
| ETXS1028 | AmsAfsUmCfCmUfUmGmUmGmGmAm | SEQ ID | AAUCCUUGUGGAUGA | SEQ ID |
| | UmGfAmAfGmGmCmAmAmsAmsAm | NO: 560 | AGGCAAAA | NO: 140 |
| ETXS1030 | CmsCfsAmUfCmGfUmGmCmCmUmCm | SEQ ID | CCAUCGUGCCUCAUG | SEQ ID |
| | AmUfGmGfAmGmAmUmCmsUmsUm | NO: 561 | GAGAUCUU | NO: 141 |
| ETXS1032 | AmsAfsAmAfUmCfCmUmUmGmUmGm | SEQ ID | AAAAUCCUUGUGGAU | SEQ ID |
| | GmAfUmGfAmAmGmGmCmsAmsAm | NO: 762 | GAAGGCAA | NO: 144 |
| ETXS1034 | AmsAfsAmAmUmCfCmUmUfGmUmGm | SEQ ID | AAAAUCCUUGUGGAU | SEQ ID |
| | GmAfUmGfAmAmGmGmCmsAmsAm | NO: 763 | GAAGGCAA | NO: 144 |
| ETXS1038 | CmsAfsUmGmGmUfGmGmCfAmUmUm | SEQ ID | CAUGGUGGCAUUUCC | SEQ ID |
| | UmCfCmUfUmGmGmUmAmsGmsGm | NO: 765 | UUGGUAGG | NO: 145 |
| ETXS1042 | UmsCfsGmUmGmCfCmUmCfAmUmGm | SEQ ID | UCGUGCCUCAUGGAG | SEQ ID |
| | GmAfGmAfUmCmUmUmUmsCmsGm | NO: 767 | AUCUUUCG | NO: 148 |
| ETXS1044 | AmsAfsAmUfUmCfUmUmGmUmCmAm | SEQ ID | AAAUUCUUGUCAAAG | SEQ ID |
| | AmAfGmGfUmGmGmAmGmsGmsCm | NO: 768 | GUGGAGGC | NO: 165 |
| ETXS1046 | AmsAfsAmUmUmCfUmUmGfUmCmAm | SEQ ID | AAAUUCUUGUCAAAG | SEQ ID |
| | AmAfGmGfUmGmGmAmGmsGmsCm | NO: 769 | GUGGAGGC | NO: 165 |
| ETXS1048 | UmsGfsUmGfGmAfUmGmAmAmGmGm | SEQ ID | UGUGGAUGAAGGCAA | SEQ ID |
| | CmAfAmAfAmCmUmCmCmsCmsCm | NO: 770 | AACUCCCC | NO: 202 |
| ETXS1050 | UmsGfsUmGmGmAfUmGmAfAmGmGm | SEQ ID | UGUGGAUGAAGGCAA | SEQ ID |
| | CmAfAmAfAmCmUmCmCmsCmsCm | NO: 771 | AACUCCCC | NO: 202 |
| ETXS1051 | AmsAfsAmAmUmCfCmUmUmGmUmGm | SEQ ID | AAAAUCCUUGUGGAU | SEQ ID |
| | GmAfUmGfAmAfGmGmCmsAmsAm | NO: 782 | GAAGGCAA | NO: 144 |
| ETXS1052 | CmsAfsUmGmGmUfGmGmCmAmUmUm | SEQ ID | CAUGGUGGCAUUUCC | SEQ ID |
| | UmCfCmUfUmGfGmUmAmsGmsGm | NO: 783 | UUGGUAGG | NO: 145 |
| ETXS1053 | UmsCfsGmUmGmCfCmUmCmAmUmGm | SEQ ID | UCGUGCCUCAUGGAG | SEQ ID |
| | GmAfGmAfUmCfUmUmUmsCmsGm | NO: 784 | AUCUUUCG | NO: 148 |
| ETXS1054 | AmsAfsAmUmUmCfUmUmGmUmCmAm | SEQ ID | AAAUUCUUGUCAAAG | SEQ ID |
| | AmAfGmGfUmGfGmAmGmsGmsCm | NO: 785 | GUGGAGGC | NO: 165 |
| ETXS1055 | UmsGfsUmGmGmAfUmGmAmAmGmGm | SEQ ID | UGUGGAUGAAGGCAA | SEQ ID |
| | CmAfAmAfAmCfUmCmCmsCmsCm | NO: 786 | AACUCCCC | NO: 202 |
| ETXS2128 | AmsAfsAmGmUmCfCmUfUfGmUmGm | SEQ ID | AAAGUCCUUGUGGAU | SEQ ID |
| | GmAfUmGfAmAmGmGmCmsAmsAm | NO: 795 | GAAGGCAA | NO: 787 |
| ETXS2144 | AmsAfsCmUmUmCfUmUfGfUmCmAm | SEQ ID | AACUUCUUGUCAAAG | SEQ ID |
| | AmAfGmGfUmGmGmAmGmsGmsCm | NO: 796 | GUGGAGGC | NO: 788 |
| ETXS2152 | UmsGfsUmGmGmAfUmGfAfAmGmGm | SEQ ID | UGUGGAUGAAGGCAA | SEQ ID |
| | CmAfAmAfGmCmUmAmCmsCmsCm | NO: 797 | AGCUACCC | NO: 789 |
| ETXS2398 | CmsAfsUmGmGmUfGmGmCfAmUmUm | SEQ ID | CAUGGUGGCAUUUCC | SEQ ID |
| | UmCfCmUfUmGmGmUmAmsGmsGm | NO: 801 | UUGGUAGG | NO: 145 |
| ETSX2400 | CmsAfsUmGmGmUfGmGmCmAmUmUm | SEQ ID | CAUGGUGGCAUUUCC | SEQ ID |
| | UmCfCmUfUmGfGmUmAmsGmsGm | NO: 802 | UUGGUAGG | NO: 145 |
| ETXS2402 | CmsAfsUmGmGmUoGmGmCmAmUmUm | SEQ ID | CAUGGUGGCAUUUCC | SEQ ID |
| | UmCfCmUfUmGmGmUmAmsGmsGm | NO: 803 | UUGGUAGG | NO: 145 |
| ETXS2404 | CmsAfsUmGmGmUoGmGfCfAmUmUm | SEQ ID | CAUGGUGGCAUUUCC | SEQ ID |
| | UmCfCmUfUmGmGmUmAmsGmsGm | NO: 804 | UUGGUAGG | NO: 145 |
| ETXS2406 | CmsAfsUmGmGmUfGmGmCmAmUmUm | SEQ ID | CAUGGUGGCAUUUCC | SEQ ID |
| | UmCfCmUfUmGmGmUfAmsGmsGm | NO: 805 | UUGGUAGG | NO: 145 |
| ETXS2408 | CmsAfsUmGfGmUfGmGmCmAmUmUm | SEQ ID | CAUGGUGGCAUUUCC | SEQ ID |
| | UmCfCmUfUmGmGmUmAmsGmsGm | NO: 806 | UUGGUAGG | NO: 145 |
| ETXS2410 | CmsAfsUmGfGmUfGmGfCfAmUmUm | SEQ ID | CAUGGUGGCAUUUCC | SEQ ID |
| | UmCfCmUfUmGmGmUmAmsGmsGm | NO: 807 | UUGGUAGG | NO: 145 |
| ETXS2412 | CmsAfsUmGmGmUfGmGfCfAmUmUm | SEQ ID | CAUGGUGGCAUUUCC | SEQ ID |
| | UmCfCmUfUmGfGmUmAmsGmsGm | NO: 808 | UUGGUAGG | NO: 145 |
| ETXS2414 | CmsAfsUmGmGmUfGmGfCfAmUmUm | SEQ ID | CAUGGUGGCAUUUCC | SEQ ID |
| | UmCfCmUfUmGmGmUfAmsGmsGm | NO: 809 | UUGGUAGG | NO: 145 |
| ETXS2416 | UmsCfsGmUmGmCfCmUmCfAmUmGm | SEQ ID | UCGUGCCUCAUGGAG | SEQ ID |
| | GmAfGmAfUmCmUmUmUmsCmsGm | NO: 810 | AUCUUUCG | NO: 148 |
| ETXS2418 | UmsCfsGmUmGmCfCmUmCmAmUmGm | SEQ ID | UCGUGCCUCAUGGAG | SEQ ID |
| | GmAfGmAfUmCfUmUmUmsCmsGm | NO: 811 | AUCUUUCG | NO: 148 |
| ETXS2420 | UmsCfsGmUmGmCoCmUmCmAmUmGm | SEQ ID | UCGUGCCUCAUGGAG | SEQ ID |
| | GmAfGmAfUmCmUmUmUmsCmsGm | NO: 812 | AUCUUUCG | NO: 148 |
| ETXS2422 | UmsCfsGmUmGmCoCmUfCfAmUmGm | SEQ ID | UCGUGCCUCAUGGAG | SEQ ID |
| | GmAfGmAfUmCmUmUmUmsCmsGm | NO: 813 | AUCUUUCG | NO: 148 |
| ETXS2424 | UmsCfsGmUmGmCfCmUmCmAmUmGm | SEQ ID | UCGUGCCUCAUGGAG | SEQ ID |
| | GmAfGmAfUmCmUmUfUmsCmsGm | NO: 814 | AUCUUUCG | NO: 148 |
| ETXS2426 | UmsCfsGmUfGmCfCmUmCmAmUmGm | SEQ ID | UCGUGCCUCAUGGAG | SEQ ID |
| | GmAfGmAfUmCmUmUmUmsCmsGm | NO: 815 | AUCUUUCG | NO: 148 |
| ETXS2428 | UmsCfsGmUfGmCfCmUfCfAmUmGm | SEQ ID | UCGUGCCUCAUGGAG | SEQ ID |
| | GmAfGmAfUmCmUmUmUmsCmsGm | NO: 816 | AUCUUUCG | NO: 148 |
| ETXS2430 | UmsCfsGmUmGmCfCmUfCfAmUmGm | SEQ ID | UCGUGCCUCAUGGAG | SEQ ID |
| | GmAfGmAfUmCfUm UmUmsCmsGm | NO: 817 | AUCUUUCG | NO: 148 |
| ETXS2432 | UmsCfsGmUmGmCfCmUfCfAmUmGm | SEQ ID | UCGUGCCUCAUGGAG | SEQ ID |
| | GmAfGmAfUmCmUmUfUmsCmsGm | NO: 818 | AUCUUUCG | NO: 148 |

Table 4 provides the modified second (sense) sequences. together with the corresponding unmodified second (sense) sequences for siRNA oligonucleosides according to the present invention as follows.

TABLE 4

| | | | Underlying Base | |
|---|---|---|---|---|
| | | SEQ ID | Sequence 5′ → 3′ | SEQ ID |
| Sense | Modified Second | NO | (Shown as an | NO |
| strand | (Sense) Strand | (SS- | Unmodified | (SS- |
| ID | 5′ → 3′ | mod) | Nucleoside Sequence) | unmod) |
| ETXS1035 | iaiaUmsAmsCmCmAmAmGfGmAfAfA | SEQ ID | UACCAAGGAAAUGCCAC | SEQ ID |
| | fUfGmCmCmAmCmCmAmUmGm | NO: 774 | CAUG | NO: 265 |
| ETXS1039 | iaiaAmsAmsAmGmAmUmCfUmCfCfA | SEQ ID | AAAGAUCUCCAUGAGGC | SEQ ID |
| | fUfGmAmGmGmCmAmCmGmAm | NO: 776 | ACGA | NO: 268 |
| ETXS631 | UmsGmsCmCmUmUmCfAmUfCfCfA | SEQ ID | UGCCUUCAUCCACAAGG | SEQ ID |
| | mCmAmAmGmGmAmUmUmUm | NO: 562 | AUUU | NO: 242 |
| ETXS633 | UmsGmsCmGmAmAmAfGmAfUfCfU | SEQ ID | UGCGAAAGAUCUCCAUG | SEQ ID |
| | mCmCmAmUmGmAmGmGmCm | NO: 563 | AGGC | NO: 243 |
| ETXS635 | GmsCmsUmGmGmUmGfGmUfCfCfU | SEQ ID | GCUGGUGGUCCUCAUGG | SEQ ID |
| | mCmAmUmGmGmAmGmAmAm | NO: 564 | AGAA | NO: 244 |
| ETXS637 | GmsCmsGmAmAmAmGfAmUfCfUfC | SEQ ID | GCGAAAGAUCUCCAUGA | SEQ ID |
| | mCmAmUmGmAmGmGmCmAm | NO: 565 | GGCA | NO: 245 |
| ETXS639 | GmsUmsAmUmGmAmGfAmUfGfCfA | SEQ ID | GUAUGAGAUGCAUGAGC | SEQ ID |
| | mUmGmAmGmCmUmGmCmUm | NO: 566 | UGCU | NO: 246 |
| ETXS641 | GmsUmsUmUmGmAmUfGmAfGfAfU | SEQ ID | GUUUGAUGAGAUUAAUC | SEQ ID |
| | mUmAmAmUmCmCmUmGmAm | NO: 567 | CUGA | NO: 247 |
| ETXS643 | UmsGmsAmGmAmUmUfAmAfUfCfC | SEQ ID | UGAGAUUAAUCCUGAAA | SEQ ID |
| | mUmGmAmAmAmCmCmAmAm | NO: 568 | CCAA | NO: 248 |
| ETXS645 | UmsGmsAmUmGmAmGfAmUfUfAfA | SEQ ID | UGAUGAGAUUAAUCCUG | SEQ ID |
| | mUmCmCmUmGmAmAmAmCm | NO: 569 | AAAC | NO: 249 |
| ETXS647 | AmsUmsGmAmGmAmUfUmAfAfUfC | SEQ ID | AUGAGAUUAAUCCUGAA | SEQ ID |
| | mCmUmGmAmAmAmCmCmAm | NO: 570 | ACCA | NO: 250 |
| ETXS649 | GmsAmsUmGmAmGmAfUmUfAfAfU | SEQ ID | GAUGAGAUUAAUCCUGA | SEQ ID |
| | mCmCmUmGmAmAmAmCmCm | NO: 571 | AACC | NO: 251 |
| ETXS651 | CmsUmsGmUmUmUmGfAmUfGfAfG | SEQ ID | CUGUUUGAUGAGAUUAA | SEQ ID |
| | mAmUmUmAmAmUmCmCmUm | NO: 572 | UCCU | NO: 252 |
| ETXS653 | UmsUmsUmUmGmCmCfUmUfCfAfU | SEQ ID | UUUUGCCUUCAUCCACA | SEQ ID |
| | mCmCmAmCmAmAmGmGmAm | NO: 573 | AGGA | NO: 253 |
| ETXS655 | CmsGmsAmAmAmGmAfUmCfUfCfC | SEQ ID | CGAAAGAUCUCCAUGAG | SEQ ID |
| | mAmUmGmAmGmGmCmAmCm | NO: 574 | GCAC | NO: 254 |
| ETXS657 | UmsGmsCmUmGmGmUfGmGfUfCfC | SEQ ID | UGCUGGUGGUCCUCAUG | SEQ ID |
| | mUmCmAmUmGmGmAmGmAm | NO: 575 | GAGA | NO: 255 |
| ETXS659 | CmsCmsUmUmCmAmUfCmCfAfCfA | SEQ ID | CCUUCAUCCACAAGGAU | SEQ ID |
| | mAmGmGmAmUmUmUmUmGm | NO: 576 | UUUG | NO: 256 |
| ETXS661 | GmsAmsAmAmGmAmUfCmUfCfCfA | SEQ ID | GAAAGAUCUCCAUGAGG | SEQ ID |
| | mUmGmAmGmGmCmAmCmGm | NO: 577 | CACG | NO: 257 |
| ETXS663 | UmsUmsCmAmUmCmCfAmCfAfAfG | SEQ ID | UUCAUCCACAAGGAUUU | SEQ ID |
| | mGmAmUmUmUmUmGmAmUm | NO: 578 | UGAU | NO: 258 |
| ETXS665 | UmsUmsUmGmAmUmGfAmGfAfUfU | SEQ ID | UUUGAUGAGAUUAAUCC | SEQ ID |
| | mAmAmUmCmCmUmGmAmAm | NO: 579 | UGAA | NO: 259 |
| ETXS667 | UmsUmsGmCmCmUmUfCmAfUfCfC | SEQ ID | UUGCCUUCAUCCACAAG | SEQ ID |
| | mAmCmAmAmGmGmAmUmUm | NO: 580 | GAUU | NO: 260 |
| ETXS669 | GmsAmsUmCmUmCmCfAmUfGfAfG | SEQ ID | GAUCUCCAUGAGGCACG | SEQ ID |
| | mGmCmAmCmGmAmUmGmGm | NO: 581 | AUGG | NO: 261 |
| ETXS671 | CmsAmsUmGmCmUmGfGmUfGfGfU | SEQ ID | CAUGCUGGUGGUCCUCA | SEQ ID |
| | mCmCmUmCmAmUmGmGmAm | NO: 582 | UGGA | NO: 262 |
| ETXS673 | UmsUmsUmGmCmCmUfUmCfAfUfC | SEQ ID | UUUGCCUUCAUCCACAA | SEQ ID |
| | mCmAmCmAmAmGmGmAmUm | NO: 583 | GGAU | NO: 263 |
| ETXS675 | GmsCmsCmUmUmCmAfUmCfCfAfC | SEQ ID | GCCUUCAUCCACAAGGA | SEQ ID |
| | mAmAmGmGmAmUmUmUmUm | NO: 584 | UUUU | NO: 264 |
| ETXS677 | UmsAmsCmCmAmAmGfGmAfAfAfU | SEQ ID | UACCAAGGAAAUGCCAC | SEQ ID |
| | mGmCmCmAmCmCmAmUmGm | NO: 585 | CAUG | NO: 265 |
| ETXS679 | UmsUmsGmAmUmGmAfGmAfUfUfA | SEQ ID | UUGAUGAGAUUAAUCCU | SEQ ID |
| | mAmUmCmCmUmGmAmAmAm | NO: 586 | GAAA | NO: 266 |
| ETXS681 | CmsUmsUmCmAmUmCfCmAfCfAfA | SEQ ID | CUUCAUCCACAAGGAUU | SEQ ID |
| | mGmGmAmUmUmUmUmGmAm | NO: 587 | UUGA | NO: 267 |
| ETXS683 | AmsAmsAmGmAmUmCfUmCfCfAfU | SEQ ID | AAAGAUCUCCAUGAGGC | SEQ ID |
| | mGmAmGmGmCmAmCmGmAm | NO: 588 | ACGA | NO: 268 |
| ETXS685 | UmsGmsUmUmUmGmAfUmGfAfGfA | SEQ ID | UGUUUGAUGAGAUUAAU | SEQ ID |
| | mUmUmAmAmUmCmCmUmGm | NO: 589 | CCUG | NO: 269 |
| ETXS687 | AmsUmsGmCmUmGmGfUmGfGfUfC | SEQ ID | AUGCUGGUGGUCCUCAU | SEQ ID |
| | mCmUmCmAmUmGmGmAmGm | NO: 590 | GGAG | NO: 270 |
| ETXS689 | GmsUmsUmUmUmGmCfCmUfUfCfA | SEQ ID | GUUUUGCCUUCAUCCAC | SEQ ID |
| | mUmCmCmAmCmAmAmGmGm | NO: 591 | AAGG | NO: 271 |
| ETXS691 | CmsCmsAmCmCmAmUfGmCfUfGfG | SEQ ID | CCACCAUGCUGGUGGUC | SEQ ID |
| | mUmGmGmUmCmCmUmCmAm | NO: 592 | CUCA | NO: 272 |
| ETXS693 | CmsAmsCmCmAmUmGfCmUfGfGfU | SEQ ID | CACCAUGCUGGUGGUCC | SEQ ID |
| | mGmGmUmCmCmUmCmAmUm | NO: 593 | UCAU | NO: 273 |
| ETXS695 | AmsAmsGmAmUmCmUfCmCfAfUfG | SEQ ID | AAGAUCUCCAUGAGGCA | SEQ ID |
| | mAmGmGmCmAmCmGmAmUm | NO: 594 | CGAU | NO: 274 |
| ETXS697 | AmsGmsUmUmUmUmGfCmCfUfUfC | SEQ ID | AGUUUUGCCUUCAUCCA | SEQ ID |
| | mAmUmCmCmAmCmAmAmGm | NO: 595 | CAAG | NO: 275 |
| ETXS699 | AmsCmsCmAmUmGmCfUmGfGfUfG | SEQ ID | ACCAUGCUGGUGGUCCU | SEQ ID |
| | mGmUmCmCmUmCmAmUmGm | NO: 596 | CAUG | NO: 276 |
| ETXS701 | AmsGmsAmUmCmUmCfCmAfUfGfA | SEQ ID | AGAUCUCCAUGAGGCAC | SEQ ID |
| | mGmGmCmAmCmGmAmUmGm | NO: 597 | GAUG | NO: 277 |
| ETXS703 | CmsCmsAmUmGmCmUfGmGfUfGfG | SEQ ID | CCAUGCUGGUGGUCCUC | SEQ ID |
| | mUmCmCmUmCmAmUmGmGm | NO: 598 | AUGG | NO: 278 |
| ETXS705 | UmsGmsCmCmUmCmCfAmCfCfUfU | SEQ ID | UGCCUCCACCUUUGACA | SEQ ID |
| | mUmGmAmCmAmAmGmAmAm | NO: 599 | AGAA | NO: 279 |
| ETXS707 | CmsCmsUmCmCmAmCfCmUfUfUfG | SEQ ID | CCUCCACCUUUGACAAG | SEQ ID |
| | mAmCmAmAmGmAmAmUmUm | NO: 600 | AAUU | NO: 280 |
| ETXS709 | CmsAmsUmUmAmAmGfGmUfGfCfC | SEQ ID | CAUUAAGGUGCCCAUGA | SEQ ID |
| | mCmAmUmGmAmUmGmUmAm | NO: 601 | UGUA | NO: 281 |
| ETXS711 | CmsUmsGmCmCmCmUfAmCfCfAfA | SEQ ID | CUGCCCUACCAAGGAAA | SEQ ID |
| | mGmGmAmAmAmUmGmCmCm | NO: 602 | UGCC | NO: 282 |
| ETXS713 | CmsAmsAmAmCmUmGfCmCfCfUfA | SEQ ID | CAAACUGCCCUACCAAG | SEQ ID |
| | mCmCmAmAmGmGmAmAmAm | NO: 603 | GAAA | NO: 283 |
| ETXS715 | UmsCmsAmAmAmCmUfGmCfCfCfU | SEQ ID | UCAAACUGCCCUACCAA | SEQ ID |
| | mAmCmCmAmAmGmGmAmAm | NO: 604 | GGAA | NO: 284 |
| ETXS717 | CmsUmsCmCmAmCmCfUmUfUfGfA | SEQ ID | CUCCACCUUUGACAAGA | SEQ ID |
| | mCmAmAmGmAmAmUmUmUm | NO: 605 | AUUU | NO: 285 |
| ETXS719 | CmsGmsAmCmAmCmUfUmUfCfCfA | SEQ ID | CGACACUUUCCACCUGG | SEQ ID |
| | mCmCmUmGmGmAmCmAmAm | NO: 606 | ACAA | NO: 286 |
| ETXS721 | AmsCmsUmUmUmCmCfAmCfCfUfG | SEQ ID | ACUUUCCACCUGGACAA | SEQ ID |
| | mGmAmCmAmAmGmUmAmCm | NO: 607 | GUAC | NO: 287 |
| ETXS723 | AmsAmsAmCmUmGmCfCmCfUfAfC | SEQ ID | AAACUGCCCUACCAAGG | SEQ ID |
| | mCmAmAmGmGmAmAmAmUm | NO: 608 | AAAU | NO: 288 |
| ETXS725 | AmsCmsUmGmCmCmCfUmAfCfCfA | SEQ ID | ACUGCCCUACCAAGGAA | SEQ ID |
| | mAmGmGmAmAmAmUmGmCm | NO: 609 | AUGC | NO: 289 |
| ETXS727 | CmsAmsCmUmUmUmCfCmAfCfCfU | SEQ ID | CACUUUCCACCUGGACA | SEQ ID |
| | mGmGmAmCmAmAmGmUmAm | NO: 610 | AGUA | NO: 290 |
| ETXS729 | CmsCmsAmUmUmAmAfGmGfUfGfC | SEQ ID | CCAUUAAGGUGCCCAUG | SEQ ID |
| | mCmCmAmUmGmAmUmGmUm | NO: 611 | AUGU | NO: 291 |
| ETXS731 | UmsUmsGmCmCmUmCfCmAfCfCfU | SEQ ID | UUGCCUCCACCUUUGAC | SEQ ID |
| | mUmUmGmAmCmAmAmGmAm | NO: 612 | AAGA | NO: 292 |
| ETXS733 | UmsUmsUmCmCmAmCfCmUfGfGfA | SEQ ID | UUUCCACCUGGACAAGU | SEQ ID |
| | mCmAmAmGmUmAmCmAmAm | NO: 613 | ACAA | NO: 293 |
| ETXS735 | UmsCmsCmUmCmAmAfAmCfUfGfC | SEQ ID | UCCUCAAACUGCCCUAC | SEQ ID |
| | mCmCmUmAmCmCmAmAmGm | NO: 614 | CAAG | NO: 294 |
| ETXS737 | AmsAmsCmUmGmCmCfCmUfAfCfC | SEQ ID | AACUGCCCUACCAAGGA | SEQ ID |
| | mAmAmGmGmAmAmAmUmGm | NO: 615 | AAUG | NO: 295 |
| ETXS739 | CmsCmsUmCmAmAmAfCmUfGfCfC | SEQ ID | CCUCAAACUGCCCUACC | SEQ ID |
| | mCmUmAmCmCmAmAmGmGm | NO: 616 | AAGG | NO: 296 |
| ETXS741 | GmsUmsCmCmUmCmAfAmAfCfUfG | SEQ ID | GUCCUCAAACUGCCCUA | SEQ ID |
| | mCmCmCmUmAmCmCmAmAm | NO: 617 | CCAA | NO: 297 |
| ETXS743 | GmsAmsCmAmCmUmUfUmCfCfAfC | SEQ ID | GACACUUUCCACCUGGA | SEQ ID |
| | mCmUmGmGmAmCmAmAmGm | NO: 618 | CAAG | NO: 298 |
| ETXS745 | UmsUmsUmGmCmCmUfCmCfAfCfC | SEQ ID | UUUGCCUCCACCUUUGA | SEQ ID |
| | mUmUmUmGmAmCmAmAmGm | NO: 619 | CAAG | NO: 299 |
| ETXS747 | AmsCmsAmCmUmUmUfCmCfAfCfC | SEQ ID | ACACUUUCCACCUGGAC | SEQ ID |
| | mUmGmGmAmCmAmAmGmUm | NO: 620 | AAGU | NO: 300 |
| ETXS749 | UmsUmsCmCmAmCmCfUmGfGfAfC | SEQ ID | UUCCACCUGGACAAGUA | SEQ ID |
| | mAmAmGmUmAmCmAmAmGm | NO: 621 | CAAG | NO: 301 |
| ETXS751 | CmsUmsUmUmCmCmAfCmCfUfGfG | SEQ ID | CUUUCCACCUGGACAAG | SEQ ID |
| | mAmCmAmAmGmUmAmCmAm | NO: 622 | UACA | NO: 302 |
| ETXS753 | CmsUmsCmAmAmAmCfUmGfCfCfC | SEQ ID | CUCAAACUGCCCUACCA | SEQ ID |
| | mUmAmCmCmAmAmGmGmAm | NO: 623 | AGGA | NO: 303 |
| ETXS755 | UmsUmsAmCmAmUmCfUmUfGfUfU | SEQ ID | UUACAUCUUGUUCAAAG | SEQ ID |
| | mCmAmAmAmGmGmGmAmAm | NO: 624 | GGAA | NO: 304 |
| ETXS757 | UmsGmsCmCmAmCmCfAmUfGfCfU | SEQ ID | UGCCACCAUGCUGGUGG | SEQ ID |
| | mGmGmUmGmGmUmCmCmUm | NO: 625 | UCCU | NO: 305 |
| ETXS759 | UmsAmsUmCmCmAmAfGmAfGfGfU | SEQ ID | UAUCCAAGAGGUAUUUU | SEQ ID |
| | mAmUmUmUmUmGmAmUmAm | NO: 626 | GAUA | NO: 306 |
| ETXS761 | AmsAmsAmUmGmCmCfAmCfCfAfU | SEQ ID | AAAUGCCACCAUGCUGG | SEQ ID |
| | mGmCmUmGmGmUmGmGmUm | NO: 627 | UGGU | NO: 307 |
| ETXS763 | AmsUmsUmAmCmAmUfCmUfUfGfU | SEQ ID | AUUACAUCUUGUUCAAA | SEQ ID |
| | mUmCmAmAmAmGmGmGmAm | NO: 628 | GGGA | NO: 308 |
| ETXS765 | CmsUmsCmCmAmUmGfAmGfGfCfA | SEQ ID | CUCCAUGAGGCACGAUG | SEQ ID |
| | mCmGmAmUmGmGmCmAmAm | NO: 629 | GCAA | NO: 309 |
| ETXS767 | UmsCmsCmAmUmGmCfCmUfCfCfU | SEQ ID | UCCAUGCCUCCUGUCAU | SEQ ID |
| | mGmUmCmAmUmCmAmAmAm | NO: 630 | CAAA | NO: 310 |
| ETXS769 | AmsUmsUmCmCmAmUfGmCfCfUfC | SEQ ID | AUUCCAUGCCUCCUGUC | SEQ ID |
| | mCmUmGmUmCmAmUmCmAm | NO: 631 | AUCA | NO: 311 |
| ETXS771 | CmsAmsAmGmGmAmAfAmUfGfCfC | SEQ ID | CAAGGAAAUGCCACCAU | SEQ ID |
| | mAmCmCmAmUmGmCmUmGm | NO: 632 | GCUG | NO: 312 |
| ETXS773 | UmsGmsGmUmGmGmUfCmCfUfCfA | SEQ ID | UGGUGGUCCUCAUGGAG | SEQ ID |
| | mUmGmGmAmGmAmAmAmAm | NO: 633 | AAAA | NO: 313 |
| ETXS775 | AmsUmsCmUmUmGmUfUmCfAfAfA | SEQ ID | AUCUUGUUCAAAGGGAA | SEQ ID |
| | mGmGmGmAmAmAmUmGmGm | NO: 634 | AUGG | NO: 314 |
| ETXS777 | AmsAmsGmGmAmAmAfUmGfCfCfA | SEQ ID | AAGGAAAUGCCACCAUG | SEQ ID |
| | mCmCmAmUmGmCmUmGmGm | NO: 635 | CUGG | NO: 315 |
| ETXS779 | GmsCmsCmUmCmCmAfCmCfUfUfU | SEQ ID | GCCUCCACCUUUGACAA | SEQ ID |
| | mGmAmCmAmAmGmAmAmUm | NO: 636 | GAAU | NO: 316 |
| ETXS781 | GmsAmsGmUmUmUmUfGmCfCfUfU | SEQ ID | GAGUUUUGCCUUCAUCC | SEQ ID |
| | mCmAmUmCmCmAmCmAmAm | NO: 637 | ACAA | NO: 317 |
| ETXS783 | GmsCmsUmGmCmGmAfAmAfGfAfU | SEQ ID | GCUGCGAAAGAUCUCCA | SEQ ID |
| | mCmUmCmCmAmUmGmAmGm | NO: 638 | UGAG | NO: 318 |
| ETXS785 | AmsGmsUmUmUmGmCfCmUfCfCfA | SEQ ID | AGUUUGCCUCCACCUUU | SEQ ID |
| | mCmCmUmUmUmGmAmCmAm | NO: 639 | GACA | NO: 319 |
| ETXS787 | GmsUmsUmUmGmCmCfUmCfCfAfC | SEQ ID | GUUUGCCUCCACCUUUG | SEQ ID |
| | mCmUmUmUmGmAmCmAmAm | NO: 640 | ACAA | NO: 320 |
| ETXS789 | CmsAmsUmCmUmUmGfUmUfCfAfA | SEQ ID | CAUCUUGUUCAAAGGGA | SEQ ID |
| | mAmGmGmGmAmAmAmUmGm | NO: 641 | AAUG | NO: 321 |
| ETXS791 | GmsGmsAmGmUmUmUfUmGfCfCfU | SEQ ID | GGAGUUUUGCCUUCAUC | SEQ ID |
| | mUmCmAmUmCmCmAmCmAm | NO: 642 | CACA | NO: 322 |
| ETXS793 | CmsUmsGmCmGmAmAfAmGfAfUfC | SEQ ID | CUGCGAAAGAUCUCCAU | SEQ ID |
| | mUmCmCmAmUmGmAmGmGm | NO: 643 | GAGG | NO: 323 |
| ETXS795 | CmsCmsAmUmGmCmCfUmCfCfUfG | SEQ ID | CCAUGCCUCCUGUCAUC | SEQ ID |
| | mUmCmAmUmCmAmAmAmGm | NO: 644 | AAAG | NO: 324 |
| ETXS797 | UmsGmsUmUmUmCmUfGmGfGfCfA | SEQ ID | UGUUUCUGGGCAGGGUG | SEQ ID |
| | mGmGmGmUmGmGmUmGmAm | NO: 645 | GUGA | NO: 325 |
| ETXS799 | GmsCmsCmUmGmCmUfGmCfGfAfA | SEQ ID | GCCUGCUGCGAAAGAUC | SEQ ID |
| | mAmGmAmUmCmUmCmCmAm | NO: 646 | UCCA | NO: 326 |
| ETXS801 | AmsAmsCmUmGmUmUfUmGfAfUfG | SEQ ID | AACUGUUUGAUGAGAUU | SEQ ID |
| | mAmGmAmUmUmAmAmUmCm | NO: 647 | AAUC | NO: 327 |
| ETXS803 | AmsUmsGmCmCmUmCfCmUfGfUfC | SEQ ID | AUGCCUCCUGUCAUCAA | SEQ ID |
| | mAmUmCmAmAmAmGmUmGm | NO: 648 | AGUG | NO: 328 |
| ETXS805 | AmsGmsUmAmUmGmAfGmAfUfGfC | SEQ ID | AGUAUGAGAUGCAUGAG | SEQ ID |
| | mAmUmGmAmGmCmUmGmCm | NO: 649 | CUGC | NO: 329 |
| ETXS807 | AmsAmsGmUmAmUmGfAmGfAfUfG | SEQ ID | AAGUAUGAGAUGCAUGA | SEQ ID |
| | mCmAmUmGmAmGmCmUmGm | NO: 650 | GCUG | NO: 330 |
| ETXS809 | UmsGmsUmCmCmUmCfAmAfAfCfU | SEQ ID | UGUCCUCAAACUGCCCU | SEQ ID |
| | mGmCmCmCmUmAmCmCmAm | NO: 651 | ACCA | NO: 331 |
| ETXS811 | AmsCmsUmGmUmUmUfGmAfUfGfA | SEQ ID | ACUGUUUGAUGAGAUUA | SEQ ID |
| | mGmAmUmUmAmAmUmCmCm | NO: 652 | AUCC | NO: 332 |
| ETXS813 | GmsAmsCmCmAmUmUfAmAfGfGfU | SEQ ID | GACCAUUAAGGUGCCCA | SEQ ID |
| | mGmCmCmCmAmUmGmAmUm | NO: 653 | UGAU | NO: 333 |
| ETXS815 | AmsCmsCmAmUmUmAfAmGfGfUfG | SEQ ID | ACCAUUAAGGUGCCCAU | SEQ ID |
| | mCmCmCmAmUmGmAmUmGm | NO: 654 | GAUG | NO: 334 |
| ETXS817 | GmsUmsUmUmCmUmGfGmGfCfAfG | SEQ ID | GUUUCUGGGCAGGGUGG | SEQ ID |
| | mGmGmUmGmGmUmGmAmAm | NO: 655 | UGAA | NO: 335 |
| ETXS819 | UmsUmsCmUmGmUmUfUmCfUfGfG | SEQ ID | UUCUGUUUCUGGGCAGG | SEQ ID |
| | mGmCmAmGmGmGmUmGmGm | NO: 656 | GUGG | NO: 336 |
| ETXS821 | UmsUmsUmUmCmUmUfUmCfCfGfA | SEQ ID | UUUUCUUUCCGAAGUUC | SEQ ID |
| | mAmGmUmUmCmAmAmGmCm | NO: 657 | AAGC | NO: 337 |
| ETXS823 | UmsUmsUmCmUmUmUfCmCfGfAfA | SEQ ID | UUUCUUUCCGAAGUUCA | SEQ ID |
| | mGmUmUmCmAmAmGmCmUm | NO: 658 | AGCU | NO: 338 |
| ETXS825 | UmsCmsUmGmUmUmUfCmUfGfGfG | SEQ ID | UCUGUUUCUGGGCAGGG | SEQ ID |
| | mCmAmGmGmGmUmGmGmUm | NO: 659 | UGGU | NO: 339 |
| ETXS827 | AmsUmsGmUmCmCmUfCmAfAfAfC | SEQ ID | AUGUCCUCAAACUGCCC | SEQ ID |
| | mUmGmCmCmCmUmAmCmCm | NO: 660 | UACC | NO: 340 |
| ETXS829 | UmsCmsGmAmCmAmCfUmUfUfCfC | SEQ ID | UCGACACUUUCCACCUG | SEQ ID |
| | mAmCmCmUmGmGmAmCmAm | NO: 661 | GACA | NO: 341 |
| ETXS831 | CfsCmsUfUmCfAmUfCmCfAmCfAmA | SEQ ID | CCUUCAUCCACAAGGAU | SEQ ID |
| | fGmGfAmUfUmUf | NO: 662 | UU | NO: 342 |
| ETXS833 | CfsGmsAfAmAfGmAfUmCfUmCfCmA | SEQ ID | CGAAAGAUCUCCAUGAG | SEQ ID |
| | fUmGfAmGfGmCf | NO: 663 | GC | NO: 343 |
| ETXS835 | UfsGmsGfUmGfGmUfCmCfUmCfAm | SEQ ID | UGGUGGUCCUCAUGGAG | SEQ ID |
| | UfGmGfAmGfAmAf | NO: 664 | AA | NO: 344 |
| ETXS837 | GfsAmsAfAmGfAmUfCmUfCmCfAm | SEQ ID | GAAAGAUCUCCAUGAGG | SEQ ID |
| | UfGmAfGmGfCmAf | NO: 665 | CA | NO: 345 |
| ETXS839 | AfsUmsGfAmGfAmUfGmCfAmUfGm | SEQ ID | AUGAGAUGCAUGAGCUG | SEQ ID |
| | AfGmCfUmGfCmUf | NO: 666 | CU | NO: 346 |
| ETXS841 | UfsUmsGfAmUfGmAfGmAfUmUfAm | SEQ ID | UUGAUGAGAUUAAUCCU | SEQ ID |
| | AfUmCfCmUfGmAf | NO: 667 | GA | NO: 347 |
| ETXS843 | AfsGmsAfUmUfAmAfUmCfCmUfGm | SEQ ID | AGAUUAAUCCUGAAACC | SEQ ID |
| | AfAmAfCmCfAmAf | NO: 668 | AA | NO: 348 |
| ETXS845 | AfsUmsGfAmGfAmUfUmAfAmUfCm | SEQ ID | AUGAGAUUAAUCCUGAA | SEQ ID |
| | CfUmGfAmAfAmCf | NO: 669 | AC | NO: 349 |
| ETXS847 | GfsAmsGfAmUfUmAfAmUfCmCfUm | SEQ ID | GAGAUUAAUCCUGAAAC | SEQ ID |
| | GfAmAfAmCfCmAf | NO: 670 | CA | NO: 350 |
| ETXS849 | UfsGmsAfGmAfUmUfAmAfUmCfCm | SEQ ID | UGAGAUUAAUCCUGAAA | SEQ ID |
| | UfGmAfAmAfCmCf | NO: 671 | CC | NO: 351 |
| ETXS851 | GfsUmsUfUmGfAmUfGmAfGmAfUm | SEQ ID | GUUUGAUGAGAUUAAUC | SEQ ID |
| | UfAmAfUmCfCmUf | NO: 672 | CU | NO: 352 |
| ETXS853 | UfsUmsGfCmCfUmUfCmAfUmCfCmA | SEQ ID | UUGCCUUCAUCCACAAG | SEQ ID |
| | fCmAfAmGfGmAf | NO: 673 | GA | NO: 353 |
| ETXS855 | AfsAmsAfGmAfUmCfUmCfCmAfUm | SEQ ID | AAAGAUCUCCAUGAGGC | SEQ ID |
| | GfAmGfGmCfAmCf | NO: 674 | AC | NO: 354 |
| ETXS857 | CfsUmsGfGmUfGmGfUmCfCmUfCmA | SEQ ID | CUGGUGGUCCUCAUGGA | SEQ ID |
| | fUmGfGmAfGmAf | NO: 675 | GA | NO: 355 |
| ETXS859 | UfsUmsCfAmUfCmCfAmCfAmAfGmG | SEQ ID | UUCAUCCACAAGGAUUU | SEQ ID |
| | fAmUfUmUfUmGf | NO: 676 | UG | NO: 356 |
| ETXS861 | AfsAmsGfAmUfCmUfCmCfAmUfGm | SEQ ID | AAGAUCUCCAUGAGGCA | SEQ ID |
| | AfGmGfCmAfCmGf | NO: 677 | CG | NO: 357 |
| ETXS863 | CfsAmsUfCmCfAmCfAmAfGmGfAmU | SEQ ID | CAUCCACAAGGAUUUUG | SEQ ID |
| | fUmUfUmGfAmUf | NO: 678 | AU | NO: 358 |
| ETXS865 | UfsGmsAfUmGfAmGfAmUfUmAfAm | SEQ ID | UGAUGAGAUUAAUCCUG | SEQ ID |
| | UfCmCfUmGfAmAf | NO: 679 | AA | NO: 359 |
| ETXS867 | GfsCmsCfUmUfCmAfUmCfCmAfCmA | SEQ ID | GCCUUCAUCCACAAGGA | SEQ ID |
| | fAmGfGmAfUmUf | NO: 680 | UU | NO: 360 |
| ETXS869 | UfsCmsUfCmCfAmUfGmAfGmGfCmA | SEQ ID | UCUCCAUGAGGCACGAU | SEQ ID |
| | fCmGfAmUfGmGf | NO: 681 | GG | NO: 361 |
| ETXS871 | UmsGmsCmCmUmUmCfAfUfCfCfAm | SEQ ID | UGCCUUCAUCCACAAGG | SEQ ID |
| | CmAmAmGmGmAmUfUmUm | NO: 682 | AUUU | NO: 242 |
| ETXS873 | UmsGmsCmGmAmAmAfGfAfUfCfUm | SEQ ID | UGCGAAAGAUCUCCAUG | SEQ ID |
| | CmCmAmUmGmAmGfGmCm | NO: 683 | AGGC | NO: 243 |
| ETXS875 | GmsCmsUmGmGmUmGfGfUfCfCfUm | SEQ ID | GCUGGUGGUCCUCAUGG | SEQ ID |
| | CmAmUmGmGmAmGfAmAm | NO: 684 | AGAA | NO: 244 |
| ETXS877 | GmsCmsGmAmAmAmGfAfUfCfUfCm | SEQ ID | GCGAAAGAUCUCCAUGA | SEQ ID |
| | CmAmUmGmAmGmGfCmAm | NO: 685 | GGCA | NO: 245 |
| ETXS879 | GmsUmsAmUmGmAmGfAfUfGfCfAm | SEQ ID | GUAUGAGAUGCAUGAGC | SEQ ID |
| | UmGmAmGmCmUmGfCmUm | NO: 686 | UGCU | NO: 246 |
| ETXS881 | GmsUmsUmUmGmAmUfGfAfGfAfUm | SEQ ID | GUUUGAUGAGAUUAAUC | SEQ ID |
| | UmAmAmUmCmCmUfGmAm | NO: 687 | CUGA | NO: 247 |
| ETXS883 | UmsGmsAmGmAmUmUfAfAfUfCfCm | SEQ ID | UGAGAUUAAUCCUGAAA | SEQ ID |
| | UmGmAmAmAmCmCfAmAm | NO: 688 | CCAA | NO: 248 |
| ETXS885 | UmsGmsAmUmGmAmGfAfUfUfAfAm | SEQ ID | UGAUGAGAUUAAUCCUG | SEQ ID |
| | UmCmCmUmGmAmAfAmCm | NO: 689 | AAAC | NO: 249 |
| ETXS887 | AmsUmsGmAmGmAmUfUfAfAfUfCm | SEQ ID | AUGAGAUUAAUCCUGAA | SEQ ID |
| | CmUmGmAmAmAmCfCmAm | NO: 690 | ACCA | NO: 250 |
| ETXS889 | GmsAmsUmGmAmGmAfUfUfAfAfUm | SEQ ID | GAUGAGAUUAAUCCUGA | SEQ ID |
| | CmCmUmGmAmAmAfCmCm | NO: 691 | AACC | NO: 251 |
| ETXS891 | CmsUmsGmUmUmUmGfAfUfGfAfGm | SEQ ID | CUGUUUGAUGAGAUUAA | SEQ ID |
| | AmUmUmAmAmUmCfCmUm | NO: 692 | UCCU | NO: 252 |
| ETXS893 | UmsUmsUmUmGmCmCfUfUfCfAfUm | SEQ ID | UUUUGCCUUCAUCCACA | SEQ ID |
| | CmCmAmCmAmAmGfGmAm | NO: 693 | AGGA | NO: 253 |
| ETXS895 | CmsGmsAmAmAmGmAfUfCfUfCfCm | SEQ ID | CGAAAGAUCUCCAUGAG | SEQ ID |
| | AmUmGmAmGmGmCfAmCm | NO: 694 | GCAC | NO: 254 |
| ETXS897 | UmsGmsCmUmGmGmUfGfGfUfCfCm | SEQ ID | UGCUGGUGGUCCUCAUG | SEQ ID |
| | UmCmAmUmGmGmAfGmAm | NO: 695 | GAGA | NO: 255 |
| ETXS899 | CmsCmsUmUmCmAmUfCfCfAfCfAm | SEQ ID | CCUUCAUCCACAAGGAU | SEQ ID |
| | AmGmGmAmUmUmUfUmGm | NO: 696 | UUUG | NO: 256 |
| ETXS901 | GmsAmsAmAmGmAmUfCfUfCfCfAm | SEQ ID | GAAAGAUCUCCAUGAGG | SEQ ID |
| | UmGmAmGmGmCmAfCmGm | NO: 697 | CACG | NO: 257 |
| ETXS903 | UmsUmsCmAmUmCmCfAfCfAfAfGm | SEQ ID | UUCAUCCACAAGGAUUU | SEQ ID |
| | GmAmUmUmUmUmGfAmUm | NO: 698 | UGAU | NO: 258 |
| ETXS905 | UmsUmsUmGmAmUmGfAfGfAfUfUm | SEQ ID | UUUGAUGAGAUUAAUCC | SEQ ID |
| | AmAmUmCmCmUmGfAmAm | NO: 699 | UGAA | NO: 259 |
| ETXS907 | UmsUmsGmCmCmUmUfCfAfUfCfCm | SEQ ID | UUGCCUUCAUCCACAAG | SEQ ID |
| | AmCmAmAmGmGmAfUmUm | NO: 700 | GAUU | NO: 260 |
| ETXS909 | GmsAmsUmCmUmCmCfAfUfGfAfGm | SEQ ID | GAUCUCCAUGAGGCACG | SEQ ID |
| | GmCmAmCmGmAmUfGmGm | NO: 701 | AUGG | NO: 261 |
| ETXS911 | UmsGmsCmCmUmUfCfAmUfCfCfAfC | SEQ ID | UGCCUUCAUCCACAAGG | SEQ ID |
| | mAmAmGmGmAmUmUmUm | NO: 702 | AUUU | NO: 242 |
| ETXS913 | UmsGmsCmGmAmAfAfGmAfUfCfUf | SEQ ID | UGCGAAAGAUCUCCAUG | SEQ ID |
| | CmCmAmUmGmAmGmGmCm | NO: 703 | AGGC | NO: 243 |
| ETXS915 | GmsCmsUmGmGmUfGfGmUfCfCfUfC | SEQ ID | GCUGGUGGUCCUCAUGG | SEQ ID |
| | mAmUmGmGmAmGmAmAm | NO: 704 | AGAA | NO: 244 |
| ETXS917 | GmsCmsGmAmAmAfGfAmUfCfUfCfC | SEQ ID | GCGAAAGAUCUCCAUGA | SEQ ID |
| | mAmUmGmAmGmGmCmAm | NO: 705 | GGCA | NO: 245 |
| ETXS919 | GmsUmsAmUmGmAfGfAmUfGfCfAf | SEQ ID | GUAUGAGAUGCAUGAGC | SEQ ID |
| | UmGmAmGmCmUmGmCmUm | NO: 706 | UGCU | NO: 246 |
| ETXS921 | GmsUmsUmUmGmAfUfGmAfGfAfUf | SEQ ID | GUUUGAUGAGAUUAAUC | SEQ ID |
| | UmAmAmUmCmCmUmGmAm | NO: 707 | CUGA | NO: 247 |
| ETXS923 | UmsGmsAmGmAmUfUfAmAfUfCfCf | SEQ ID | UGAGAUUAAUCCUGAAA | SEQ ID |
| | UmGmAmAmAmCmCmAmAm | NO: 708 | CCAA | NO: 248 |
| ETXS925 | UmsGmsAmUmGmAfGfAmUfUfAfAf | SEQ ID | UGAUGAGAUUAAUCCUG | SEQ ID |
| | UmCmCmUmGmAmAmAmCm | NO: 709 | AAAC | NO: 249 |
| ETXS927 | AmsUmsGmAmGmAfUfUmAfAfUfCf | SEQ ID | AUGAGAUUAAUCCUGAA | SEQ ID |
| | CmUmGmAmAmAmCmCmAm | NO: 710 | ACCA | NO: 250 |
| ETXS929 | GmsAmsUmGmAmGfAfUmUfAfAfUf | SEQ ID | GAUGAGAUUAAUCCUGA | SEQ ID |
| | CmCmUmGmAmAmAmCmCm | NO: 711 | AACC | NO: 251 |
| ETXS931 | CmsUmsGmUmUmUfGfAmUfGfAfGf | SEQ ID | CUGUUUGAUGAGAUUAA | SEQ ID |
| | AmUmUmAmAmUmCmCmUm | NO: 712 | UCCU | NO: 252 |
| ETXS933 | UmsUmsUmUmGmCfCfUmUfCfAfUfC | SEQ ID | UUUUGCCUUCAUCCACA | SEQ ID |
| | mCmAmCmAmAmGmGmAm | NO: 713 | AGGA | NO: 253 |
| ETXS935 | CmsGmsAmAmAmGfAfUmCfUfCfCfA | SEQ ID | CGAAAGAUCUCCAUGAG | SEQ ID |
| | mUmGmAmGmGmCmAmCm | NO: 714 | GCAC | NO: 254 |
| ETXS937 | UmsGmsCmUmGmGfUfGmGfUfCfCf | SEQ ID | UGCUGGUGGUCCUCAUG | SEQ ID |
| | UmCmAmUmGmGmAmGmAm | NO: 715 | GAGA | NO: 255 |
| ETXS939 | CmsCmsUmUmCmAfUfCmCfAfCfAfA | SEQ ID | CCUUCAUCCACAAGGAU | SEQ ID |
| | mGmGmAmUmUmUmUmGm | NO: 716 | UUUG | NO: 256 |
| ETXS941 | GmsAmsAmAmGmAfUfCmUfCfCfAf | SEQ ID | GAAAGAUCUCCAUGAGG | SEQ ID |
| | UmGmAmGmGmCmAmCmGm | NO: 717 | CACG | NO: 257 |
| ETXS943 | UmsUmsCmAmUmCfCfAmCfAfAfGfG | SEQ ID | UUCAUCCACAAGGAUUU | SEQ ID |
| | mAmUmUmUmUmGmAmUm | NO: 718 | UGAU | NO: 258 |
| ETXS945 | UmsUmsUmGmAmUfGfAmGfAfUfUf | SEQ ID | UUUGAUGAGAUUAAUCC | SEQ ID |
| | AmAmUmCmCmUmGmAmAm | NO: 719 | UGAA | NO: 259 |
| ETXS947 | UmsUmsGmCmCmUfUfCmAfUfCfCfA | SEQ ID | UUGCCUUCAUCCACAAG | SEQ ID |
| | mCmAmAmGmGmAmUmUm | NO: 720 | GAUU | NO: 260 |
| ETXS949 | GmsAmsUmCmUmCfCfAmUfGfAfGf | SEQ ID | GAUCUCCAUGAGGCACG | SEQ ID |
| | GmCmAmCmGmAmUmGmGm | NO: 721 | AUGG | NO: 261 |
| ETXS951 | UmsGmsCmCmUmUmCfAmUfCfCfAf | SEQ ID | UGCCUUCAUCCACAAGG | SEQ ID |
| | CmAmAmGmGmAmUmUmUm | NO: 722 | AUUU | NO: 242 |
| ETXS953 | UmsGmsCmGmAmAmAfGmAfUfCfUf | SEQ ID | UGCGAAAGAUCUCCAUG | SEQ ID |
| | CmCmAmUmGmAmGmGmCm | NO: 723 | AGGC | NO: 243 |
| ETXS955 | GmsCmsUmGmGmUmGfGmUfCfCfUf | SEQ ID | GCUGGUGGUCCUCAUGG | SEQ ID |
| | CmAmUmGmGmAmGmAmAm | NO: 724 | AGAA | NO: 244 |
| ETXS957 | GmsCmsGmAmAmAmGfAmUfCfUfCf | SEQ ID | GCGAAAGAUCUCCAUGA | SEQ ID |
| | CmAmUmGmAmGmGmCmAm | NO: 725 | GGCA | NO: 245 |
| ETXS959 | GmsUmsAmUmGmAmGfAmUfGfCfAf | SEQ ID | GUAUGAGAUGCAUGAGC | SEQ ID |
| | UmGmAmGmCmUmGmCmUm | NO: 726 | UGCU | NO: 246 |
| ETXS961 | GmsUmsUmUmGmAmUfGmAfGfAfUf | SEQ ID | GUUUGAUGAGAUUAAUC | SEQ ID |
| | UmAmAmUmCmCmUmGmAm | NO: 727 | CUGA | NO: 247 |
| ETXS963 | UmsGmsAmGmAmUmUfAmAfUfCfCf | SEQ ID | UGAGAUUAAUCCUGAAA | SEQ ID |
| | UmGmAmAmAmCmCmAmAm | NO: 728 | CCAA | NO: 248 |
| ETXS965 | UmsGmsAmUmGmAmGfAmUfUfAfAf | SEQ ID | UGAUGAGAUUAAUCCUG | SEQ ID |
| | UmCmCmUmGmAmAmAmCm | NO: 729 | AAAC | NO: 249 |
| ETXS967 | AmsUmsGmAmGmAmUfUmAfAfUfCf | SEQ ID | AUGAGAUUAAUCCUGAA | SEQ ID |
| | CmUmGmAmAmAmCmCmAm | NO: 730 | ACCA | NO: 250 |
| ETXS969 | GmsAmsUmGmAmGmAfUmUfAfAfUf | SEQ ID | GAUGAGAUUAAUCCUGA | SEQ ID |
| | CmCmUmGmAmAmAmCmCm | NO: 731 | AACC | NO: 251 |
| ETXS971 | CmsUmsGmUmUmUmGfAmUfGfAfGf | SEQ ID | CUGUUUGAUGAGAUUAA | SEQ ID |
| | AmUmUmAmAmUmCmCmUm | NO: 732 | UCCU | NO: 252 |
| ETXS973 | UmsUmsUmUmGmCmCfUmUfCfAfUf | SEQ ID | UUUUGCCUUCAUCCACA | SEQ ID |
| | CmCmAmCmAmAmGmGmAm | NO: 733 | AGGA | NO: 253 |
| ETXS975 | CmsGmsAmAmAmGmAfUmCfUfCfCf | SEQ ID | CGAAAGAUCUCCAUGAG | SEQ ID |
| | AmUmGmAmGmGmCmAmCm | NO: 734 | GCAC | NO: 254 |
| ETXS977 | UmsGmsCmUmGmGmUfGmGfUfCfCf | SEQ ID | UGCUGGUGGUCCUCAUG | SEQ ID |
| | UmCmAmUmGmGmAmGmAm | NO: 735 | GAGA | NO: 255 |
| ETXS979 | CmsCmsUmUmCmAmUfCmCfAfCfAf | SEQ ID | CCUUCAUCCACAAGGAU | SEQ ID |
| | AmGmGmAmUmUmUmUmGm | NO: 736 | UUUG | NO: 256 |
| ETXS981 | GmsAmsAmAmGmAmUfCmUfCfCfAf | SEQ ID | GAAAGAUCUCCAUGAGG | SEQ ID |
| | UmGmAmGmGmCmAmCmGm | NO: 737 | CACG | NO: 257 |
| ETXS983 | UmsUmsCmAmUmCmCfAmCfAfAfGf | SEQ ID | UUCAUCCACAAGGAUUU | SEQ ID |
| | GmAmUmUmUmUmGmAmUm | NO: 738 | UGAU | NO: 258 |
| ETXS985 | UmsUmsUmGmAmUmGfAmGfAfUfUf | SEQ ID | UUUGAUGAGAUUAAUCC | SEQ ID |
| | AmAmUmCmCmUmGmAmAm | NO: 739 | UGAA | NO: 259 |
| ETXS987 | UmsUmsGmCmCmUmUfCmAfUfCfCf | SEQ ID | UUGCCUUCAUCCACAAG | SEQ ID |
| | AmCmAmAmGmGmAmUmUm | NO: 740 | GAUU | NO: 260 |
| ETXS989 | GmsAmsUmCmUmCmCfAmUfGfAfGf | SEQ ID | GAUCUCCAUGAGGCACG | SEQ ID |
| | GmCmAmCmGmAmUmGmGm | NO: 741 | AUGG | NO: 261 |
| ETXS991 | UmsGmsCmCmUmUmCfAmUfCfCfAf | SEQ ID | UGCCUUCAUCCACAAGG | SEQ ID |
| | CmAmAmGmGmAmUmUmUm | NO: 742 | AUUU | NO: 242 |
| ETXS993 | UmsGmsCmGmAmAmAfGmAfUfCfUf | SEQ ID | UGCGAAAGAUCUCCAUG | SEQ ID |
| | CmCmAmUmGmAmGmGmCm | NO: 743 | AGGC | NO: 243 |
| ETXS995 | GmsCmsUmGmGmUmGfGmUfCfCfUf | SEQ ID | GCUGGUGGUCCUCAUGG | SEQ ID |
| | CmAmUmGmGmAmGmAmAm | NO: 744 | AGAA | NO: 244 |
| ETXS997 | GmsCmsGmAmAmAmGfAmUfCfUfCf | SEQ ID | GCGAAAGAUCUCCAUGA | SEQ ID |
| | CmAmUmGmAmGmGmCmAm | NO: 745 | GGCA | NO: 245 |
| ETXS999 | GmsUmsAmUmGmAmGfAmUfGfCfAf | SEQ ID | GUAUGAGAUGCAUGAGC | SEQ ID |
| | UmGmAmGmCmUmGmCmUm | NO: 746 | UGCU | NO: 246 |
| ETXS1001 | GmsUmsUmUmGmAmUfGmAfGfAfUf | SEQ ID | GUUUGAUGAGAUUAAUC | SEQ ID |
| | UmAmAmUmCmCmUmGmAm | NO: 747 | CUGA | NO: 247 |
| ETXS1003 | UmsGmsAmGmAmUmUfAmAfUfCfCf | SEQ ID | UGAGAUUAAUCCUGAAA | SEQ ID |
| | UmGmAmAmAmCmCmAmAm | NO: 748 | CCAA | NO: 248 |
| ETXS1005 | UmsGmsAmUmGmAmGfAmUfUfAfAf | SEQ ID | UGAUGAGAUUAAUCCUG | SEQ ID |
| | UmCmCmUmGmAmAmAmCm | NO: 749 | AAAC | NO: 249 |
| ETXS1007 | AmsUmsGmAmGmAmUfUmAfAfUfCf | SEQ ID | AUGAGAUUAAUCCUGAA | SEQ ID |
| | CmUmGmAmAmAmCmCmAm | NO: 750 | ACCA | NO: 250 |
| ETXS1009 | GmsAmsUmGmAmGmAfUmUfAfAfUf | SEQ ID | GAUGAGAUUAAUCCUGA | SEQ ID |
| | CmCmUmGmAmAmAmCmCm | NO: 751 | AACC | NO: 251 |
| ETXS1011 | CmsUmsGmUmUmUmGfAmUfGfAfGf | SEQ ID | CUGUUUGAUGAGAUUAA | SEQ ID |
| | AmUmUmAmAmUmCmCmUm | NO: 752 | UCCU | NO: 252 |
| ETXS1013 | UmsUmsUmUmGmCmCfUmUfCfAfUf | SEQ ID | UUUUGCCUUCAUCCACA | SEQ ID |
| | CmCmAmCmAmAmGmGmAm | NO: 753 | AGGA | NO: 253 |
| ETXS1015 | CmsGmsAmAmAmGmAfUmCfUfCfCf | SEQ ID | CGAAAGAUCUCCAUGAG | SEQ ID |
| | AmUmGmAmGmGmCmAmCm | NO: 754 | GCAC | NO: 254 |
| ETXS1017 | UmsGmsCmUmGmGmUfGmGfUfCfCf | SEQ ID | UGCUGGUGGUCCUCAUG | SEQ ID |
| | UmCmAmUmGmGmAmGmAm | NO: 755 | GAGA | NO: 255 |
| ETXS1019 | CmsCmsUmUmCmAmUfCmCfAfCfAf | SEQ ID | CCUUCAUCCACAAGGAU | SEQ ID |
| | AmGmGmAmUmUmUmUmGm | NO: 756 | UUUG | NO: 256 |
| ETXS1021 | GmsAmsAmAmGmAmUfCmUfCfCfAf | SEQ ID | GAAAGAUCUCCAUGAGG | SEQ ID |
| | UmGmAmGmGmCmAmCmGm | NO: 757 | CACG | NO: 257 |
| ETXS1023 | UmsUmsCmAmUmCmCfAmCfAfAfGf | SEQ ID | UUCAUCCACAAGGAUUU | SEQ ID |
| | GmAmUmUmUmUmGmAmUm | NO: 758 | UGAU | NO: 258 |
| ETXS1025 | UmsUmsUmGmAmUmGfAmGfAfUfUf | SEQ ID | UUUGAUGAGAUUAAUCC | SEQ ID |
| | AmAmUmCmCmUmGmAmAm | NO: 759 | UGAA | NO: 259 |
| ETXS1027 | UmsUmsGmCmCmUmUfCmAfUfCfCf | SEQ ID | UUGCCUUCAUCCACAAG | SEQ ID |
| | AmCmAmAmGmGmAmUmUm | NO: 760 | GAUU | NO: 260 |
| ETXS1029 | GmsAmsUmCmUmCmCfAmUfGfAfGf | SEQ ID | GAUCUCCAUGAGGCACG | SEQ ID |
| | GmCmAmCmGmAmUmGmGm | NO: 761 | AUGG | NO: 261 |
| ETXS1031 | iaiaGmsCmsCmUmUmCmAfUmCfCfA | SEQ ID | GCCUUCAUCCACAAGGA | SEQ ID |
| | fCfAmAmGmGmAmUmUmUmUm | NO: 772 | UUUU | NO: 264 |
| ETXS1033 | iaiaGmsCmsCmUmUmCmAmUmCfCf | SEQ ID | GCCUUCAUCCACAAGGA | SEQ ID |
| | AfCmAmAmGmGmAmUmUmUmUm | NO: 773 | UUUU | NO: 264 |
| ETXS1037 | iaiaUmsAmsCmCmAmAmGmGmAfAf | SEQ ID | UACCAAGGAAAUGCCAC | SEQ ID |
| | AfUmGmCmCmAmCmCmAmUmGm | NO: 775 | CAUG | NO: 265 |
| ETXS1041 | iaiaAmsAmsAmGmAmUmCmUmCfCf | SEQ ID | AAAGAUCUCCAUGAGGC | SEQ ID |
| | AfUmGmAmGmGmCmAmCmGmAm | NO: 777 | ACGA | NO: 268 |
| ETXS1043 | iaiaCmsUmsCmCmAmCmCfUmUfUfG | SEQ ID | CUCCACCUUUGACAAGA | SEQ ID |
| | fAfCmAmAmGmAmAmUmUmUm | NO: 778 | AUUU | NO: 285 |
| ETXS1045 | iaiaCmsUmsCmCmAmCmCmUmUfUf | SEQ ID | CUCCACCUUUGACAAGA | SEQ ID |
| | GfAmCmAmAmGmAmAmUmUmUm | NO: 779 | AUUU | NO: 285 |
| ETXS1047 | iaiaGmsGmsAmGmUmUmUfUmGfCfC | SEQ ID | GGAGUUUUGCCUUCAUC | SEQ ID |
| | fUfUmCmAmUmCmCmAmCmAm | NO: 780 | CACA | NO: 322 |
| ETXS1049 | iaiaGmsGmsAmGmUmUmUmUmGfCf | SEQ ID | GGAGUUUUGCCUUCAUC | SEQ ID |
| | CfUmUmCmAmUmCmCmAmCmAm | NO: 781 | CACA | NO: 322 |
| ETXS2127 | GmsCmsCmUmUmCmAfUmCfCfAfC | SEQ ID | GCCUUCAUCCACAAGGA | SEQ ID |
| | mAmAmGmGmAmCmUmUmUm | NO: 798 | CUUU | NO: 791 |
| ETXS2143 | CmsUmsCmCmAmCmCfUmUfUfGfA | SEQ ID | CUCCACCUUUGACAAGA | SEQ ID |
| | mCmAmAmGmAmAmGmUmUm | NO: 799 | AGUU | NO: 792 |
| ETXS2151 | GmsUmsAmGmCmUmUfUmGfCfCfU | SEQ ID | GUAGCUUUGCCUUCAUC | SEQ ID |
| | mUmCmAmUmCmCmAmCmAm | NO: 800 | CACA | NO: 793 |
| ETXS2397 | iaiaUmsAmsCmCmAmAmGmGmAfAf | SEQ ID | UACCAAGGAAAUGCCAC | SEQ ID |
| | AfUmGmCmCmAmCmCmAmUmGm | NO: 819 | CAUG | NO: 265 |
| ETXS2399 | iaiaAmsAmsAmGmAmUmCmUmCfCf | SEQ ID | AAAGAUCUCCAUGAGGC | SEQ ID |
| | AfUmGmAmGmGmCmAmCmGmAm | NO: 820 | ACGA | NO: 268 |

Some of the modified second strand sequences as illustrated above in Table 4 include the preferred 5′ iaia motif. However, it should also be understood that the scope of these modified second strand sequences additionally includes the Me/F modified second strand in the absence of the 5′iaia motif.

Table 5 identifies duplexes with Duplex IDs referencing the modified antisense and sense IDs from previous Tables 3 and 4.

TABLE 5

| Duplex ID | First (Antisense) strand ID | Second (Sense) strand ID |
|---|---|---|
| ETXM1184 | ETXS1036 | ETXS1035 |
| ETXM1157 | ETXS1040 | ETXS1039 |
| ETXM316 | ETXS632 | ETXS631 |
| ETXM317 | ETXS634 | ETXS633 |
| ETXM318 | ETXS636 | ETXS635 |
| ETXM319 | ETXS638 | ETXS637 |
| ETXM320 | ETXS640 | ETXS639 |
| ETXM321 | ETXS642 | ETXS641 |
| ETXM322 | ETXS644 | ETXS643 |
| ETXM323 | ETXS646 | ETXS645 |
| ETXM324 | ETXS648 | ETXS647 |
| ETXM325 | ETXS650 | ETXS649 |
| ETXM326 | ETXS652 | ETXS651 |
| ETXM327 | ETXS654 | ETXS653 |
| ETXM328 | ETXS656 | ETXS655 |
| ETXM329 | ETXS658 | ETXS657 |
| ETXM330 | ETXS660 | ETXS659 |
| ETXM331 | ETXS662 | ETXS661 |
| ETXM332 | ETXS664 | ETXS663 |
| ETXM333 | ETXS666 | ETXS665 |
| ETXM334 | ETXS668 | ETXS667 |
| ETXM335 | ETXS670 | ETXS669 |
| ETXM336 | ETXS672 | ETXS671 |
| ETXM337 | ETXS674 | ETXS673 |
| ETXM338 | ETXS676 | ETXS675 |
| ETXM339 | ETXS678 | ETXS677 |
| ETXM340 | ETXS680 | ETXS679 |
| ETXM341 | ETXS682 | ETXS681 |
| ETXM342 | ETXS684 | ETXS683 |
| ETXM343 | ETXS686 | ETXS685 |
| ETXM344 | ETXS688 | ETXS687 |
| ETXM345 | ETXS690 | ETXS689 |
| ETXM346 | ETXS692 | ETXS691 |
| ETXM347 | ETXS694 | ETXS693 |
| ETXM348 | ETXS696 | ETXS695 |
| ETXM349 | ETXS698 | ETXS697 |
| ETXM350 | ETXS700 | ETXS699 |
| ETXM351 | ETXS702 | ETXS701 |
| ETXM352 | ETXS704 | ETXS703 |
| ETXM353 | ETXS706 | ETXS705 |
| ETXM354 | ETXS708 | ETXS707 |
| ETXM355 | ETXS710 | ETXS709 |
| ETXM356 | ETXS712 | ETXS711 |
| ETXM357 | ETXS714 | ETXS713 |
| ETXM358 | ETXS716 | ETXS715 |
| ETXM359 | ETXS718 | ETXS717 |
| ETXM360 | ETXS720 | ETXS719 |
| ETXM361 | ETXS722 | ETXS721 |
| ETXM362 | ETXS724 | ETXS723 |
| ETXM363 | ETXS726 | ETXS725 |
| ETXM364 | ETXS728 | ETXS727 |
| ETXM365 | ETXS730 | ETXS729 |
| ETXM366 | ETXS732 | ETXS731 |
| ETXM367 | ETXS734 | ETXS733 |
| ETXM368 | ETXS736 | ETXS735 |
| ETXM369 | ETXS738 | ETXS737 |
| ETXM370 | ETXS740 | ETXS739 |
| ETXM371 | ETXS742 | ETXS741 |
| ETXM372 | ETXS744 | ETXS743 |
| ETXM373 | ETXS746 | ETXS745 |
| ETXM374 | ETXS748 | ETXS747 |
| ETXM375 | ETXS750 | ETXS749 |
| ETXM376 | ETXS752 | ETXS751 |
| ETXM377 | ETXS754 | ETXS753 |
| ETXM378 | ETXS756 | ETXS755 |
| ETXM379 | ETXS758 | ETXS757 |
| ETXM380 | ETXS760 | ETXS759 |
| ETXM381 | ETXS762 | ETXS761 |
| ETXM382 | ETXS764 | ETXS763 |
| ETXM383 | ETXS766 | ETXS765 |
| ETXM384 | ETXS768 | ETXS767 |
| ETXM385 | ETXS770 | ETXS769 |
| ETXM386 | ETXS772 | ETXS771 |
| ETXM387 | ETXS774 | ETXS773 |
| ETXM388 | ETXS776 | ETXS775 |
| ETXM389 | ETXS778 | ETXS777 |
| ETXM390 | ETXS780 | ETXS779 |
| ETXM391 | ETXS782 | ETXS781 |
| ETXM392 | ETXS784 | ETXS783 |
| ETXM393 | ETXS786 | ETXS785 |
| ETXM394 | ETXS788 | ETXS787 |
| ETXM395 | ETXS790 | ETXS789 |
| ETXM396 | ETXS792 | ETXS791 |
| ETXM397 | ETXS794 | ETXS793 |
| ETXM398 | ETXS796 | ETXS795 |
| ETXM399 | ETXS798 | ETXS797 |
| ETXM400 | ETXS800 | ETXS799 |
| ETXM401 | ETXS802 | ETXS801 |
| ETXM402 | ETXS804 | ETXS803 |
| ETXM403 | ETXS806 | ETXS805 |
| ETXM404 | ETXS808 | ETXS807 |
| ETXM405 | ETXS810 | ETXS809 |
| ETXM406 | ETXS812 | ETXS811 |
| ETXM407 | ETXS814 | ETXS813 |
| ETXM408 | ETXS816 | ETXS815 |
| ETXM409 | ETXS818 | ETXS817 |
| ETXM410 | ETXS820 | ETXS819 |
| ETXM411 | ETXS822 | ETXS821 |
| ETXM412 | ETXS824 | ETXS823 |
| ETXM413 | ETXS826 | ETXS825 |
| ETXM414 | ETXS828 | ETXS827 |
| ETXM415 | ETXS830 | ETXS829 |
| ETXM416 | ETXS832 | ETXS831 |
| ETXM417 | ETXS834 | ETXS833 |
| ETXM418 | ETXS836 | ETXS835 |
| ETXM419 | ETXS838 | ETXS837 |
| ETXM420 | ETXS840 | ETXS839 |
| ETXM421 | ETXS842 | ETXS841 |
| ETXM422 | ETXS844 | ETXS843 |
| ETXM423 | ETXS846 | ETXS845 |
| ETXM424 | ETXS848 | ETXS847 |
| ETXM425 | ETXS850 | ETXS849 |
| ETXM426 | ETXS852 | ETXS851 |
| ETXM427 | ETXS854 | ETXS853 |
| ETXM428 | ETXS856 | ETXS855 |
| ETXM429 | ETXS858 | ETXS857 |
| ETXM430 | ETXS860 | ETXS859 |
| ETXM431 | ETXS862 | ETXS861 |
| ETXM432 | ETXS864 | ETXS863 |
| ETXM433 | ETXS866 | ETXS865 |
| ETXM434 | ETXS868 | ETXS867 |
| ETXM435 | ETXS870 | ETXS869 |
| ETXM436 | ETXS872 | ETXS871 |
| ETXM437 | ETXS874 | ETXS873 |
| ETXM438 | ETXS876 | ETXS875 |
| ETXM439 | ETXS878 | ETXS877 |
| ETXM440 | ETXS880 | ETXS879 |
| ETXM441 | ETXS882 | ETXS881 |
| ETXM442 | ETXS884 | ETXS883 |
| ETXM443 | ETXS886 | ETXS885 |
| ETXM444 | ETXS888 | ETXS887 |
| ETXM445 | ETXS890 | ETXS889 |
| ETXM446 | ETXS892 | ETXS891 |
| ETXM447 | ETXS894 | ETXS893 |
| ETXM448 | ETXS896 | ETXS895 |
| ETXM449 | ETXS898 | ETXS897 |
| ETXM450 | ETXS900 | ETXS899 |
| ETXM451 | ETXS902 | ETXS901 |
| ETXM452 | ETXS904 | ETXS903 |
| ETXM453 | ETXS906 | ETXS905 |
| ETXM454 | ETXS908 | ETXS907 |
| ETXM455 | ETXS910 | ETXS909 |
| ETXM456 | ETXS912 | ETXS911 |
| ETXM457 | ETXS914 | ETXS913 |
| ETXM458 | ETXS916 | ETXS915 |
| ETXM459 | ETXS918 | ETXS917 |
| ETXM460 | ETXS920 | ETXS919 |
| ETXM461 | ETXS922 | ETXS921 |
| ETXM462 | ETXS924 | ETXS923 |
| ETXM463 | ETXS926 | ETXS925 |
| ETXM464 | ETXS928 | ETXS927 |
| ETXM465 | ETXS930 | ETXS929 |
| ETXM466 | ETXS932 | ETXS931 |
| ETXM467 | ETXS934 | ETXS933 |
| ETXM468 | ETXS936 | ETXS935 |
| ETXM469 | ETXS938 | ETXS937 |
| ETXM470 | ETXS940 | ETXS939 |
| ETXM471 | ETXS942 | ETXS941 |
| ETXM472 | ETXS944 | ETXS943 |
| ETXM473 | ETXS946 | ETXS945 |
| ETXM474 | ETXS948 | ETXS947 |
| ETXM475 | ETXS950 | ETXS949 |
| ETXM476 | ETXS952 | ETXS951 |
| ETXM477 | ETXS954 | ETXS953 |
| ETXM478 | ETXS956 | ETXS955 |
| ETXM479 | ETXS958 | ETXS957 |
| ETXM480 | ETXS960 | ETXS959 |
| ETXM481 | ETXS962 | ETXS961 |
| ETXM482 | ETXS964 | ETXS963 |
| ETXM483 | ETXS966 | ETXS965 |
| ETXM484 | ETXS968 | ETXS967 |
| ETXM485 | ETXS970 | ETXS969 |
| ETXM486 | ETXS972 | ETXS971 |
| ETXM487 | ETXS974 | ETXS973 |
| ETXM488 | ETXS976 | ETXS975 |
| ETXM489 | ETXS978 | ETXS977 |
| ETXM490 | ETXS980 | ETXS979 |
| ETXM491 | ETXS982 | ETXS981 |
| ETXM492 | ETXS984 | ETXS983 |
| ETXM493 | ETXS986 | ETXS985 |
| ETXM494 | ETXS988 | ETXS987 |
| ETXM495 | ETXS990 | ETXS989 |
| ETXM496 | ETXS992 | ETXS991 |
| ETXM497 | ETXS994 | ETXS993 |
| ETXM498 | ETXS996 | ETXS995 |
| ETXM499 | ETXS998 | ETXS997 |
| ETXM500 | ETXS1000 | ETXS999 |
| ETXM501 | ETXS1002 | ETXS1001 |
| ETXM502 | ETXS1004 | ETXS1003 |
| ETXM503 | ETXS1006 | ETXS1005 |
| ETXM504 | ETXS1008 | ETXS1007 |
| ETXM505 | ETXS1010 | ETXS1009 |
| ETXM506 | ETXS1012 | ETXS1011 |
| ETXM507 | ETXS1014 | ETXS1013 |
| ETXM508 | ETXS1016 | ETXS1015 |
| ETXM509 | ETXS1018 | ETXS1017 |
| ETXM510 | ETXS1020 | ETXS1019 |
| ETXM511 | ETXS1022 | ETXS1021 |
| ETXM512 | ETXS1024 | ETXS1023 |
| ETXM513 | ETXS1026 | ETXS1025 |
| ETXM514 | ETXS1028 | ETXS1027 |
| ETXM515 | ETXS1030 | ETXS1029 |
| ETXM1064 | ETXS2128 | ETXS2127 |
| ETXM1072 | ETXS2144 | ETXS2143 |
| ETXM1076 | ETXS2152 | ETXS2151 |
| ETXM1180 | ETXS1032 | ETXS1031 |
| ETXM1181 | ETXS1034 | ETXS1033 |
| ETXM1185 | ETXS1038 | ETXS1037 |
| ETXM1162 | ETXS1042 | ETXS1041 |
| ETXM1188 | ETXS1044 | ETXS1043 |
| ETXM1189 | ETXS1046 | ETXS1045 |
| ETXM1192 | ETXS1048 | ETXS1047 |
| ETXM1193 | ETXS1050 | ETXS1049 |
| ETXM1194 | ETXS1051 | ETXS1033 |
| ETXM1195 | ETXS1052 | ETXS1037 |
| ETXM1196 | ETXS1053 | ETXS1041 |
| ETXM1197 | ETXS1054 | ETXS1045 |
| ETXM1198 | ETXS1055 | ETXS1049 |
| ETXM1199 | ETXS2398 | ETXS2397 |
| ETXM1200 | ETSX2400 | ETXS2397 |
| ETXM1201 | ETXS2402 | ETXS2397 |
| ETXM1202 | ETXS2404 | ETXS2397 |
| ETXM1203 | ETXS2406 | ETXS2397 |
| ETXM1204 | ETXS2408 | ETXS2397 |
| ETXM1205 | ETXS2410 | ETXS2397 |
| ETXM1206 | ETXS2412 | ETXS2397 |
| ETXM1207 | ETXS2414 | ETXS2397 |
| ETXM1208 | ETXS2416 | ETXS2399 |
| ETXM1209 | ETXS2418 | ETXS2399 |
| ETXM1210 | ETXS2420 | ETXS2399 |
| ETXM1211 | ETXS2422 | ETXS2399 |
| ETXM1212 | ETXS2424 | ETXS2399 |
| ETXM1213 | ETXS2426 | ETXS2399 |
| ETXM1214 | ETXS2428 | ETXS2399 |
| ETXM1215 | ETXS2430 | ETXS2399 |
| ETXM1216 | ETXS2432 | ETXS2399 |

For duplexes of Table 5:
ETXM316-ETXM415, ETXM436-ETXM515 and ETXM1180-ETXM1216 have a duplex structure according to FIG. 8A with a 2 nucleoside overhang at the 3′ end of the antisense;
ETXM416-ETXM435: have a duplex structure according to FIG. 8B, namely a 19mer blunt ended construct.

Definitions as provided in the above Tables:
A—adenosine
C—cytidine
G—guanosine
T—thymidine
m—2′-O-methyl
f—2′fluro
s—phosphorothioate bond
o—thermally destabilised nucleoside
ia—inverted abasic nucleoside

### Example 9: Inhibition Screen for ZPI Expression in Human Huh7 Cells

Huh7 cells (human hepatocyte-derived cell line, obtained from JCRB Cell Bank) were maintained in Dulbecco's Modified Eagle Medium (DMEM) supplemented with 10% FBS at 37° C. in at atmosphere of 5% CO₂. Cells were transfected with siRNA duplexes targeting ZPI mRNA or a negative control siRNA (siRNA-control; sense strand 5′-UUCUCCGAACGUGUCACGUTT-3′ (SEQ ID NO: 794), antisense strand 5′-ACGUGACACGUUCGGAGAATT-3′ (SEQ ID NO: 790)) at a final duplex concentration of 5 nM and 0.1 nM. Transfection was carried out by adding 9.7 μL Opti-MEM (ThermoFisher) plus 0.3 μL Lipofectamine RNAiMAX (ThermoFisher) to 10 μL of each siRNA duplex. The mixture was incubated at room temperature for 15 minutes before being added to 100 μL of complete growth medium containing 20,000 Huh7 cells. Cells were incubated for 24 hours at 37° C./5% CO₂ prior to total RNA purification using a RNeasy 96 Kit (Qiagen). Each duplex was tested by transfection in duplicate wells in two independent experiments.

cDNA synthesis was performed using FastQuant RT (with gDNase) Kit (Tiangen). Real-time quantitative PCR (qPCR) was performed on an ABI Prism 7900HT or ABI QuantStudio 7 with primers specific for human ZPI (Hs01547819_m1) and human GAPDH (Hs02786624_g1) using FastStart Universal Probe Master Kit (Roche).

qPCR was performed in duplicate on cDNA derived from each well and the mean Ct calculated. Relative ZPI expression was calculated from mean Ct values using the comparative Ct (ΔΔCt) method, normalised to GAPDH and relative to untreated cells. Based on the results of primary screen, siRNA duplexes displaying good activity were selected for dose-response follow-up. Results are shown in FIG. 9. Sequences of RNAi molecules are depicted in Table 5.

### Example 10: Dose-Response for Inhibition of ZPI Expression in Human Huh7 Cells

Huh7 cells (human hepatocyte-derived cell line, obtained from JCRB Cell Bank) were maintained in Dulbecco's Modified Eagle Medium (DMEM) supplemented with 10% FBS at 37° C. in at atmosphere of 5% CO₂. Cells were transfected with siRNA duplexes targeting ZPI mRNA or a negative control siRNA (siRNA-control; sense strand 5′-UUCUCCGAACGUGUCACGUTT-3′ (SEQ ID NO: 794), antisense strand 5′-ACGUGACACGUUCGGAGAATT-3′ (SEQ ID NO: 790)) using 10×3-fold serial dilutions over a final duplex concentration range of 20 nM to 1 pM. Transfection was carried out by adding 9.7 μL Opti-MEM (ThermoFisher) plus 0.3 μL Lipofectamine RNAiMAX (ThermoFisher) to 10 μL of each siRNA duplex. The mixture was incubated at room temperature for 15 minutes before being added to 100 μL of complete growth medium containing 20,000 Huh7 cells. Cells were incubated for 24 hours at 37° C./5% CO₂ prior to total RNA purification using a RNeasy 96 Kit (Qiagen). Each duplex was tested by transfection in duplicate wells in a single experiment.

cDNA synthesis was performed using FastQuant RT (with gDNase) Kit (Tiangen). Real-time quantitative PCR (qPCR) was performed on an ABI Prism 7900HT or ABI QuantStudio 7 with primers specific for human ZPI (Hs01547819_m1) and human GAPDH (Hs02786624_g1) using FastStart Universal Probe Master Kit (Roche).

qPCR was performed in duplicate on cDNA derived from each well and the mean Ct calculated. Relative ZPI expression was calculated from mean Ct values using the comparative Ct (ΔΔCt) method, normalised to GAPDH and relative to untreated cells. Maximum percent inhibition of ZPI expression and IC50 values were calculated using a four parameter (variable slope) model using GraphPad Prism 9. Results are shown in FIG. 9. Sequences of RNAi molecules are depicted in the relevant Tables herein.

TABLE 6
Relative mRNA Expression

| | | | | | Mean | Mean |
|---|---|---|---|---|---|---|
| | Antisense | | Sense | | Relative | Relative |
| Duplex | strand | SEQ ID NO | strand | SEQ ID NO | Expression/ | Expression/ |
| ID | ID | (AS - mod) | ID | (SS - mod) | 0.1 nM | 5 nM |
| ETXM316 | ETXS632 | SEQ ID NO: 362 | ETXS631 | SEQ ID NO: 562 | 0.72 | 0.38 |
| ETXM317 | ETXS634 | SEQ ID NO: 363 | ETXS633 | SEQ ID NO: 563 | 1.01 | 0.54 |
| ETXM318 | ETXS636 | SEQ ID NO: 364 | ETXS635 | SEQ ID NO: 564 | 0.97 | 0.56 |
| ETXM319 | ETXS638 | SEQ ID NO: 365 | ETXS637 | SEQ ID NO: 565 | 0.91 | 0.43 |
| ETXM320 | ETXS640 | SEQ ID NO: 366 | ETXS639 | SEQ ID NO: 566 | 0.84 | 0.34 |
| ETXM321 | ETXS642 | SEQ ID NO: 367 | ETXS641 | SEQ ID NO: 567 | 0.58 | 0.27 |
| ETXM322 | ETXS644 | SEQ ID NO: 368 | ETXS643 | SEQ ID NO: 568 | 0.5 | 0.27 |
| ETXM323 | ETXS646 | SEQ ID NO: 369 | ETXS645 | SEQ ID NO: 569 | 0.69 | 0.3 |
| ETXM324 | ETXS648 | SEQ ID NO: 370 | ETXS647 | SEQ ID NO: 570 | 0.87 | 0.46 |
| ETXM325 | ETXS650 | SEQ ID NO: 371 | ETXS649 | SEQ ID NO: 571 | 0.74 | 0.31 |
| ETXM326 | ETXS652 | SEQ ID NO: 372 | ETXS651 | SEQ ID NO: 572 | 0.99 | 0.33 |
| ETXM327 | ETXS654 | SEQ ID NO: 373 | ETXS653 | SEQ ID NO: 573 | 0.79 | 0.37 |
| ETXM328 | ETXS656 | SEQ ID NO: 374 | ETXS655 | SEQ ID NO: 574 | 0.9 | 0.49 |
| ETXM329 | ETXS658 | SEQ ID NO: 375 | ETXS657 | SEQ ID NO: 575 | 1.11 | 0.81 |
| ETXM330 | ETXS660 | SEQ ID NO: 376 | ETXS659 | SEQ ID NO: 576 | 1 | 0.83 |
| ETXM331 | ETXS662 | SEQ ID NO: 377 | ETXS661 | SEQ ID NO: 577 | 1.04 | 0.84 |
| ETXM332 | ETXS664 | SEQ ID NO: 378 | ETXS663 | SEQ ID NO: 578 | 0.42 | 0.22 |
| ETXM333 | ETXS666 | SEQ ID NO: 379 | ETXS665 | SEQ ID NO: 579 | 0.58 | 0.28 |
| ETXM334 | ETXS668 | SEQ ID NO: 380 | ETXS667 | SEQ ID NO: 580 | 0.91 | 0.57 |
| ETXM335 | ETXS670 | SEQ ID NO: 381 | ETXS669 | SEQ ID NO: 581 | 1.04 | 0.74 |
| ETXM336 | ETXS672 | SEQ ID NO: 382 | ETXS671 | SEQ ID NO: 582 | 1.07 | 0.85 |
| ETXM337 | ETXS674 | SEQ ID NO: 383 | ETXS673 | SEQ ID NO: 583 | 0.84 | 0.51 |
| ETXM338 | ETXS676 | SEQ ID NO: 384 | ETXS675 | SEQ ID NO: 584 | 0.38 | 0.23 |
| ETXM339 | ETXS678 | SEQ ID NO: 385 | ETXS677 | SEQ ID NO: 585 | 0.85 | 0.4 |
| ETXM340 | ETXS680 | SEQ ID NO: 386 | ETXS679 | SEQ ID NO: 586 | 0.75 | 0.36 |
| ETXM341 | ETXS682 | SEQ ID NO: 387 | ETXS681 | SEQ ID NO: 587 | 0.55 | 0.22 |
| ETXM342 | ETXS684 | SEQ ID NO: 388 | ETXS683 | SEQ ID NO: 588 | 0.55 | 0.42 |
| ETXM343 | ETXS686 | SEQ ID NO: 389 | ETXS685 | SEQ ID NO: 589 | 0.45 | 0.29 |
| ETXM344 | ETXS688 | SEQ ID NO: 390 | ETXS687 | SEQ ID NO: 590 | 0.98 | 1.01 |
| ETXM345 | ETXS690 | SEQ ID NO: 391 | ETXS689 | SEQ ID NO: 591 | 0.78 | 0.57 |
| ETXM346 | ETXS692 | SEQ ID NO: 392 | ETXS691 | SEQ ID NO: 592 | 1.09 | 1.12 |
| ETXM347 | ETXS694 | SEQ ID NO: 393 | ETXS693 | SEQ ID NO: 593 | 0.93 | 0.45 |
| ETXM348 | ETXS696 | SEQ ID NO: 394 | ETXS695 | SEQ ID NO: 594 | 0.91 | 0.65 |
| ETXM349 | ETXS698 | SEQ ID NO: 395 | ETXS697 | SEQ ID NO: 595 | 0.88 | 0.49 |
| ETXM350 | ETXS700 | SEQ ID NO: 396 | ETXS699 | SEQ ID NO: 596 | 0.87 | 0.75 |
| ETXM351 | ETXS702 | SEQ ID NO: 397 | ETXS701 | SEQ ID NO: 597 | 0.96 | 0.96 |
| ETXM352 | ETXS704 | SEQ ID NO: 398 | ETXS703 | SEQ ID NO: 598 | 0.95 | 1.04 |
| ETXM353 | ETXS706 | SEQ ID NO: 399 | ETXS705 | SEQ ID NO: 599 | 0.71 | 0.5 |
| ETXM354 | ETXS708 | SEQ ID NO: 400 | ETXS707 | SEQ ID NO: 600 | 0.7 | 0.43 |
| ETXM355 | ETXS710 | SEQ ID NO: 401 | ETXS709 | SEQ ID NO: 601 | 0.69 | 0.34 |
| ETXM356 | ETXS712 | SEQ ID NO: 402 | ETXS711 | SEQ ID NO: 602 | 0.92 | 0.71 |
| ETXM357 | ETXS714 | SEQ ID NO: 403 | ETXS713 | SEQ ID NO: 603 | 0.88 | 0.49 |
| ETXM358 | ETXS716 | SEQ ID NO: 404 | ETXS715 | SEQ ID NO: 604 | 0.98 | 0.5 |
| ETXM359 | ETXS718 | SEQ ID NO: 405 | ETXS717 | SEQ ID NO: 605 | 0.66 | 0.33 |
| ETXM360 | ETXS720 | SEQ ID NO: 406 | ETXS719 | SEQ ID NO: 606 | 0.75 | 0.54 |
| ETXM361 | ETXS722 | SEQ ID NO: 407 | ETXS721 | SEQ ID NO: 607 | 0.68 | 0.48 |
| ETXM362 | ETXS724 | SEQ ID NO: 408 | ETXS723 | SEQ ID NO: 608 | 0.95 | 0.9 |
| ETXM363 | ETXS726 | SEQ ID NO: 409 | ETXS725 | SEQ ID NO: 609 | 0.96 | 0.75 |
| ETXM364 | ETXS728 | SEQ ID NO: 410 | ETXS727 | SEQ ID NO: 610 | 0.88 | 0.44 |
| ETXM365 | ETXS730 | SEQ ID NO: 411 | ETXS729 | SEQ ID NO: 611 | 0.82 | 0.49 |
| ETXM366 | ETXS732 | SEQ ID NO: 412 | ETXS731 | SEQ ID NO: 612 | 0.95 | 0.6 |
| ETXM367 | ETXS734 | SEQ ID NO: 413 | ETXS733 | SEQ ID NO: 613 | 0.92 | 0.51 |
| ETXM368 | ETXS736 | SEQ ID NO: 414 | ETXS735 | SEQ ID NO: 614 | 1.02 | 0.84 |
| ETXM369 | ETXS738 | SEQ ID NO: 415 | ETXS737 | SEQ ID NO: 615 | 1.02 | 1 |
| ETXM370 | ETXS740 | SEQ ID NO: 416 | ETXS739 | SEQ ID NO: 616 | 1.37 | 0.96 |
| ETXM371 | ETXS742 | SEQ ID NO: 417 | ETXS741 | SEQ ID NO: 617 | 0.94 | 0.65 |
| ETXM372 | ETXS744 | SEQ ID NO: 418 | ETXS743 | SEQ ID NO: 618 | 0.95 | 0.67 |
| ETXM373 | ETXS746 | SEQ ID NO: 419 | ETXS745 | SEQ ID NO: 619 | 1.05 | 0.96 |
| ETXM374 | ETXS748 | SEQ ID NO: 420 | ETXS747 | SEQ ID NO: 620 | 0.97 | 0.91 |
| ETXM375 | ETXS750 | SEQ ID NO: 421 | ETXS749 | SEQ ID NO: 621 | 0.81 | 0.39 |
| ETXM376 | ETXS752 | SEQ ID NO: 422 | ETXS751 | SEQ ID NO: 622 | 0.97 | 0.76 |
| ETXM377 | ETXS754 | SEQ ID NO: 423 | ETXS753 | SEQ ID NO: 623 | 0.93 | 0.59 |
| ETXM378 | ETXS756 | SEQ ID NO: 424 | ETXS755 | SEQ ID NO: 624 | 0.93 | 0.52 |
| ETXM379 | ETXS758 | SEQ ID NO: 425 | ETXS757 | SEQ ID NO: 625 | 0.96 | 0.81 |
| ETXM380 | ETXS760 | SEQ ID NO: 426 | ETXS759 | SEQ ID NO: 626 | 0.61 | 0.31 |
| ETXM381 | ETXS762 | SEQ ID NO: 427 | ETXS761 | SEQ ID NO: 627 | 0.84 | 0.82 |
| ETXM382 | ETXS764 | SEQ ID NO: 428 | ETXS763 | SEQ ID NO: 628 | 0.8 | 0.47 |
| ETXM383 | ETXS766 | SEQ ID NO: 429 | ETXS765 | SEQ ID NO: 629 | 0.82 | 0.37 |
| ETXM384 | ETXS768 | SEQ ID NO: 430 | ETXS767 | SEQ ID NO: 630 | 0.67 | 0.38 |
| ETXM385 | ETXS770 | SEQ ID NO: 431 | ETXS769 | SEQ ID NO: 631 | 0.9 | 0.87 |
| ETXM386 | ETXS772 | SEQ ID NO: 432 | ETXS771 | SEQ ID NO: 632 | 0.91 | 0.73 |
| ETXM387 | ETXS774 | SEQ ID NO: 433 | ETXS773 | SEQ ID NO: 633 | 0.86 | 0.97 |
| ETXM388 | ETXS776 | SEQ ID NO: 434 | ETXS775 | SEQ ID NO: 634 | 0.96 | 0.7 |
| ETXM389 | ETXS778 | SEQ ID NO: 435 | ETXS777 | SEQ ID NO: 635 | 0.95 | 0.68 |
| ETXM390 | ETXS780 | SEQ ID NO: 436 | ETXS779 | SEQ ID NO: 636 | 0.87 | 0.51 |
| ETXM391 | ETXS782 | SEQ ID NO: 437 | ETXS781 | SEQ ID NO: 637 | 0.76 | 0.35 |
| ETXM392 | ETXS784 | SEQ ID NO: 438 | ETXS783 | SEQ ID NO: 638 | 0.99 | 0.76 |
| ETXM393 | ETXS786 | SEQ ID NO: 439 | ETXS785 | SEQ ID NO: 639 | 0.94 | 1.06 |
| ETXM394 | ETXS788 | SEQ ID NO: 440 | ETXS787 | SEQ ID NO: 640 | 0.85 | 0.8 |
| ETXM395 | ETXS790 | SEQ ID NO: 441 | ETXS789 | SEQ ID NO: 641 | 0.95 | 0.53 |
| ETXM396 | ETXS792 | SEQ ID NO: 442 | ETXS791 | SEQ ID NO: 642 | 0.62 | 0.27 |
| ETXM397 | ETXS794 | SEQ ID NO: 443 | ETXS793 | SEQ ID NO: 643 | 0.96 | 0.64 |
| ETXM398 | ETXS796 | SEQ ID NO: 444 | ETXS795 | SEQ ID NO: 644 | 0.93 | 0.55 |
| ETXM399 | ETXS798 | SEQ ID NO: 445 | ETXS797 | SEQ ID NO: 645 | 0.94 | 0.66 |
| ETXM400 | ETXS800 | SEQ ID NO: 446 | ETXS799 | SEQ ID NO: 646 | 0.77 | 0.57 |
| ETXM401 | ETXS802 | SEQ ID NO: 447 | ETXS801 | SEQ ID NO: 647 | 0.69 | 0.25 |
| ETXM402 | ETXS804 | SEQ ID NO: 448 | ETXS803 | SEQ ID NO: 648 | 1.05 | 0.95 |
| ETXM403 | ETXS806 | SEQ ID NO: 449 | ETXS805 | SEQ ID NO: 649 | 0.86 | 0.5 |
| ETXM404 | ETXS808 | SEQ ID NO: 450 | ETXS807 | SEQ ID NO: 650 | 0.83 | 0.35 |
| ETXM405 | ETXS810 | SEQ ID NO: 451 | ETXS809 | SEQ ID NO: 651 | 0.97 | 0.73 |
| ETXM406 | ETXS812 | SEQ ID NO: 452 | ETXS811 | SEQ ID NO: 652 | 0.84 | 0.33 |
| ETXM407 | ETXS814 | SEQ ID NO: 453 | ETXS813 | SEQ ID NO: 653 | 0.77 | 0.51 |
| ETXM408 | ETXS816 | SEQ ID NO: 454 | ETXS815 | SEQ ID NO: 654 | 0.89 | 0.51 |
| ETXM409 | ETXS818 | SEQ ID NO: 455 | ETXS817 | SEQ ID NO: 655 | 1 | 0.59 |
| ETXM410 | ETXS820 | SEQ ID NO: 456 | ETXS819 | SEQ ID NO: 656 | 0.98 | 0.71 |
| ETXM411 | ETXS822 | SEQ ID NO: 457 | ETXS821 | SEQ ID NO: 657 | 0.77 | 0.36 |
| ETXM412 | ETXS824 | SEQ ID NO: 458 | ETXS823 | SEQ ID NO: 658 | 0.97 | 0.42 |
| ETXM413 | ETXS826 | SEQ ID NO: 459 | ETXS825 | SEQ ID NO: 659 | 1 | 0.77 |
| ETXM414 | ETXS828 | SEQ ID NO: 460 | ETXS827 | SEQ ID NO: 660 | 0.98 | 0.79 |
| ETXM415 | ETXS830 | SEQ ID NO: 461 | ETXS829 | SEQ ID NO: 661 | 0.96 | 0.62 |
| ETXM416 | ETXS832 | SEQ ID NO: 462 | ETXS831 | SEQ ID NO: 662 | 0.96 | 0.52 |
| ETXM417 | ETXS834 | SEQ ID NO: 463 | ETXS833 | SEQ ID NO: 663 | 1.03 | 0.75 |
| ETXM418 | ETXS836 | SEQ ID NO: 464 | ETXS835 | SEQ ID NO: 664 | 1.11 | 0.74 |
| ETXM419 | ETXS838 | SEQ ID NO: 465 | ETXS837 | SEQ ID NO: 665 | 0.99 | 0.6 |
| ETXM420 | ETXS840 | SEQ ID NO: 466 | ETXS839 | SEQ ID NO: 666 | 0.91 | 0.54 |
| ETXM421 | ETXS842 | SEQ ID NO: 467 | ETXS841 | SEQ ID NO: 667 | 0.8 | 0.27 |
| ETXM422 | ETXS844 | SEQ ID NO: 468 | ETXS843 | SEQ ID NO: 668 | 0.82 | 0.33 |
| ETXM423 | ETXS846 | SEQ ID NO: 469 | ETXS845 | SEQ ID NO: 669 | 0.91 | 0.36 |
| ETXM424 | ETXS848 | SEQ ID NO: 470 | ETXS847 | SEQ ID NO: 670 | 0.98 | 0.62 |
| ETXM425 | ETXS850 | SEQ ID NO: 471 | ETXS849 | SEQ ID NO: 671 | 0.81 | 0.32 |
| ETXM426 | ETXS852 | SEQ ID NO: 472 | ETXS851 | SEQ ID NO: 672 | 0.91 | 0.49 |
| ETXM427 | ETXS854 | SEQ ID NO: 473 | ETXS853 | SEQ ID NO: 673 | 1.02 | 0.46 |
| ETXM428 | ETXS856 | SEQ ID NO: 474 | ETXS855 | SEQ ID NO: 674 | 1.09 | 0.94 |
| ETXM429 | ETXS858 | SEQ ID NO: 475 | ETXS857 | SEQ ID NO: 675 | 1.14 | 0.7 |
| ETXM430 | ETXS860 | SEQ ID NO: 476 | ETXS859 | SEQ ID NO: 676 | 0.92 | 0.71 |
| ETXM431 | ETXS862 | SEQ ID NO: 477 | ETXS861 | SEQ ID NO: 677 | 1.18 | 0.9 |
| ETXM432 | ETXS864 | SEQ ID NO: 478 | ETXS863 | SEQ ID NO: 678 | 0.7 | 0.26 |
| ETXM433 | ETXS866 | SEQ ID NO: 479 | ETXS865 | SEQ ID NO: 679 | 1.07 | 0.31 |
| ETXM434 | ETXS868 | SEQ ID NO: 480 | ETXS867 | SEQ ID NO: 680 | 1.11 | 0.63 |
| ETXM435 | ETXS870 | SEQ ID NO: 481 | ETXS869 | SEQ ID NO: 681 | 1 | 0.8 |
| ETXM436 | ETXS872 | SEQ ID NO: 482 | ETXS871 | SEQ ID NO: 682 | 0.72 | 0.44 |
| ETXM437 | ETXS874 | SEQ ID NO: 483 | ETXS873 | SEQ ID NO: 683 | 0.98 | 0.47 |
| ETXM438 | ETXS876 | SEQ ID NO: 484 | ETXS875 | SEQ ID NO: 684 | 1.05 | 0.75 |
| ETXM439 | ETXS878 | SEQ ID NO: 485 | ETXS877 | SEQ ID NO: 685 | 0.91 | 0.49 |
| ETXM440 | ETXS880 | SEQ ID NO: 486 | ETXS879 | SEQ ID NO: 686 | 0.91 | 0.46 |
| ETXM441 | ETXS882 | SEQ ID NO: 487 | ETXS881 | SEQ ID NO: 687 | 0.74 | 0.36 |
| ETXM442 | ETXS884 | SEQ ID NO: 488 | ETXS883 | SEQ ID NO: 688 | 0.62 | 0.36 |
| ETXM443 | ETXS886 | SEQ ID NO: 489 | ETXS885 | SEQ ID NO: 689 | 0.73 | 0.3 |
| ETXM444 | ETXS888 | SEQ ID NO: 490 | ETXS887 | SEQ ID NO: 690 | 1 | 0.59 |
| ETXM445 | ETXS890 | SEQ ID NO: 491 | ETXS889 | SEQ ID NO: 691 | 0.71 | 0.37 |
| ETXM446 | ETXS892 | SEQ ID NO: 492 | ETXS891 | SEQ ID NO: 692 | 0.73 | 0.27 |
| ETXM447 | ETXS894 | SEQ ID NO: 493 | ETXS893 | SEQ ID NO: 693 | 0.81 | 0.39 |
| ETXM448 | ETXS896 | SEQ ID NO: 494 | ETXS895 | SEQ ID NO: 694 | 0.81 | 0.61 |
| ETXM449 | ETXS898 | SEQ ID NO: 495 | ETXS897 | SEQ ID NO: 695 | 0.91 | 0.8 |
| ETXM450 | ETXS900 | SEQ ID NO: 496 | ETXS899 | SEQ ID NO: 696 | 0.97 | 0.52 |
| ETXM451 | ETXS902 | SEQ ID NO: 497 | ETXS901 | SEQ ID NO: 697 | 0.96 | 0.61 |
| ETXM452 | ETXS904 | SEQ ID NO: 498 | ETXS903 | SEQ ID NO: 698 | 0.4 | 0.24 |
| ETXM453 | ETXS906 | SEQ ID NO: 499 | ETXS905 | SEQ ID NO: 699 | 0.62 | 0.3 |
| ETXM454 | ETXS908 | SEQ ID NO: 500 | ETXS907 | SEQ ID NO: 700 | 0.81 | 0.46 |
| ETXM455 | ETXS910 | SEQ ID NO: 501 | ETXS909 | SEQ ID NO: 701 | 0.94 | 0.68 |
| ETXM456 | ETXS912 | SEQ ID NO: 502 | ETXS911 | SEQ ID NO: 702 | 0.75 | 0.36 |
| ETXM457 | ETXS914 | SEQ ID NO: 503 | ETXS913 | SEQ ID NO: 703 | 0.98 | 0.52 |
| ETXM458 | ETXS916 | SEQ ID NO: 504 | ETXS915 | SEQ ID NO: 704 | 1 | 0.61 |
| ETXM459 | ETXS918 | SEQ ID NO: 505 | ETXS917 | SEQ ID NO: 705 | 0.92 | 0.44 |
| ETXM460 | ETXS920 | SEQ ID NO: 506 | ETXS919 | SEQ ID NO: 706 | 0.86 | 0.4 |
| ETXM461 | ETXS922 | SEQ ID NO: 507 | ETXS921 | SEQ ID NO: 707 | 0.84 | 0.27 |
| ETXM462 | ETXS924 | SEQ ID NO: 508 | ETXS923 | SEQ ID NO: 708 | 0.72 | 0.33 |
| ETXM463 | ETXS926 | SEQ ID NO: 509 | ETXS925 | SEQ ID NO: 709 | 0.76 | 0.35 |
| ETXM464 | ETXS928 | SEQ ID NO: 510 | ETXS927 | SEQ ID NO: 710 | 0.95 | 0.55 |
| ETXM465 | ETXS930 | SEQ ID NO: 511 | ETXS929 | SEQ ID NO: 711 | 0.77 | 0.36 |
| ETXM466 | ETXS932 | SEQ ID NO: 512 | ETXS931 | SEQ ID NO: 712 | 0.84 | 0.33 |
| ETXM467 | ETXS934 | SEQ ID NO: 513 | ETXS933 | SEQ ID NO: 713 | 0.91 | 0.39 |
| ETXM468 | ETXS936 | SEQ ID NO: 514 | ETXS935 | SEQ ID NO: 714 | 1.14 | 0.8 |
| ETXM469 | ETXS938 | SEQ ID NO: 515 | ETXS937 | SEQ ID NO: 715 | 1.17 | 0.67 |
| ETXM470 | ETXS940 | SEQ ID NO: 516 | ETXS939 | SEQ ID NO: 716 | 1.12 | 0.79 |
| ETXM471 | ETXS942 | SEQ ID NO: 517 | ETXS941 | SEQ ID NO: 717 | 1.16 | 0.86 |
| ETXM472 | ETXS944 | SEQ ID NO: 518 | ETXS943 | SEQ ID NO: 718 | 0.49 | 0.25 |
| ETXM473 | ETXS946 | SEQ ID NO: 519 | ETXS945 | SEQ ID NO: 719 | 0.91 | 0.34 |
| ETXM474 | ETXS948 | SEQ ID NO: 520 | ETXS947 | SEQ ID NO: 720 | 1.12 | 0.68 |
| ETXM475 | ETXS950 | SEQ ID NO: 521 | ETXS949 | SEQ ID NO: 721 | 1.25 | 0.84 |
| ETXM476 | ETXS952 | SEQ ID NO: 522 | ETXS951 | SEQ ID NO: 722 | 0.87 | 0.42 |
| ETXM477 | ETXS954 | SEQ ID NO: 523 | ETXS953 | SEQ ID NO: 723 | 1.12 | 0.52 |
| ETXM478 | ETXS956 | SEQ ID NO: 524 | ETXS955 | SEQ ID NO: 724 | 1.03 | 0.62 |
| ETXM479 | ETXS958 | SEQ ID NO: 525 | ETXS957 | SEQ ID NO: 725 | 1.13 | 0.51 |
| ETXM480 | ETXS960 | SEQ ID NO: 526 | ETXS959 | SEQ ID NO: 726 | 0.93 | 0.56 |
| ETXM481 | ETXS962 | SEQ ID NO: 527 | ETXS961 | SEQ ID NO: 727 | 0.89 | 0.36 |
| ETXM482 | ETXS964 | SEQ ID NO: 528 | ETXS963 | SEQ ID NO: 728 | 0.68 | 0.46 |
| ETXM483 | ETXS966 | SEQ ID NO: 529 | ETXS965 | SEQ ID NO: 729 | 0.82 | 0.5 |
| ETXM484 | ETXS968 | SEQ ID NO: 530 | ETXS967 | SEQ ID NO: 730 | 1.06 | 0.74 |
| ETXM485 | ETXS970 | SEQ ID NO: 531 | ETXS969 | SEQ ID NO: 731 | 0.91 | 0.41 |
| ETXM486 | ETXS972 | SEQ ID NO: 532 | ETXS971 | SEQ ID NO: 732 | 0.68 | 0.23 |
| ETXM487 | ETXS974 | SEQ ID NO: 533 | ETXS973 | SEQ ID NO: 733 | 0.8 | 0.31 |
| ETXM488 | ETXS976 | SEQ ID NO: 534 | ETXS975 | SEQ ID NO: 734 | 0.89 | 0.64 |
| ETXM489 | ETXS978 | SEQ ID NO: 535 | ETXS977 | SEQ ID NO: 735 | 0.89 | 0.67 |
| ETXM490 | ETXS980 | SEQ ID NO: 536 | ETXS979 | SEQ ID NO: 736 | 0.91 | 0.56 |
| ETXM491 | ETXS982 | SEQ ID NO: 537 | ETXS981 | SEQ ID NO: 737 | 1.06 | 0.75 |
| ETXM492 | ETXS984 | SEQ ID NO: 538 | ETXS983 | SEQ ID NO: 738 | 0.43 | 0.22 |
| ETXM493 | ETXS986 | SEQ ID NO: 539 | ETXS985 | SEQ ID NO: 739 | 0.59 | 0.33 |
| ETXM494 | ETXS988 | SEQ ID NO: 540 | ETXS987 | SEQ ID NO: 740 | 0.93 | 0.59 |
| ETXM495 | ETXS990 | SEQ ID NO: 541 | ETXS989 | SEQ ID NO: 741 | 1.08 | 0.74 |
| ETXM496 | ETXS992 | SEQ ID NO: 542 | ETXS991 | SEQ ID NO: 742 | 0.73 | 0.42 |
| ETXM497 | ETXS994 | SEQ ID NO: 543 | ETXS993 | SEQ ID NO: 743 | 1.01 | 0.59 |
| ETXM498 | ETXS996 | SEQ ID NO: 544 | ETXS995 | SEQ ID NO: 744 | 0.95 | 0.59 |
| ETXM499 | ETXS998 | SEQ ID NO: 545 | ETXS997 | SEQ ID NO: 745 | 1.08 | 0.53 |
| ETXM500 | ETXS1000 | SEQ ID NO: 546 | ETXS999 | SEQ ID NO: 746 | 0.87 | 0.46 |
| ETXM501 | ETXS1002 | SEQ ID NO: 547 | ETXS1001 | SEQ ID NO: 747 | 0.6 | 0.27 |
| ETXM502 | ETXS1004 | SEQ ID NO: 548 | ETXS1003 | SEQ ID NO: 748 | 0.6 | 0.28 |
| ETXM503 | ETXS1006 | SEQ ID NO: 549 | ETXS1005 | SEQ ID NO: 749 | 0.73 | 0.36 |
| ETXM504 | ETXS1008 | SEQ ID NO: 550 | ETXS1007 | SEQ ID NO: 750 | 0.9 | 0.68 |
| ETXM505 | ETXS1010 | SEQ ID NO: 551 | ETXS1009 | SEQ ID NO: 751 | 0.72 | 0.36 |
| ETXM506 | ETXS1012 | SEQ ID NO: 552 | ETXS1011 | SEQ ID NO: 752 | 0.62 | 0.25 |
| ETXM507 | ETXS1014 | SEQ ID NO: 553 | ETXS1013 | SEQ ID NO: 753 | 1.21 | 0.33 |
| ETXM508 | ETXS1016 | SEQ ID NO: 554 | ETXS1015 | SEQ ID NO: 754 | 1.08 | 0.83 |
| ETXM509 | ETXS1018 | SEQ ID NO: 555 | ETXS1017 | SEQ ID NO: 755 | 1.09 | 0.85 |
| ETXM510 | ETXS1020 | SEQ ID NO: 556 | ETXS1019 | SEQ ID NO: 756 | 0.98 | 0.62 |
| ETXM511 | ETXS1022 | SEQ ID NO: 557 | ETXS1021 | SEQ ID NO: 757 | 0.81 | 0.87 |
| ETXM512 | ETXS1024 | SEQ ID NO: 558 | ETXS1023 | SEQ ID NO: 758 | 0.34 | 0.17 |
| ETXM513 | ETXS1026 | SEQ ID NO: 559 | ETXS1025 | SEQ ID NO: 759 | 0.49 | 0.22 |
| ETXM514 | ETXS1028 | SEQ ID NO: 560 | ETXS1027 | SEQ ID NO: 760 | 0.84 | 0.56 |
| ETXM515 | ETXS1030 | SEQ ID NO: 561 | ETXS1029 | SEQ ID NO: 761 | 0.93 | 0.74 |

TABLE 7
Dose-Response Data Table

| | Antisense | | Sense | | | % |
|---|---|---|---|---|---|---|
| Duplex | strand | SEQ ID NO | strand | SEQ ID NO | | Max |
| ID | ID | (AS - mod) | ID | (SS - mod) | IC₅₀ [pM] | Inhibition |
| ETXM320 | ETXS640 | SEQ ID NO: 366 | ETXS639 | SEQ ID NO: 566 | 556 | 77 |
| ETXM321 | ETXS642 | SEQ ID NO: 367 | ETXS641 | SEQ ID NO: 567 | 172 | 82 |
| ETXM322 | ETXS644 | SEQ ID NO: 368 | ETXS643 | SEQ ID NO: 568 | 101 | 79 |
| ETXM323 | ETXS646 | SEQ ID NO: 369 | ETXS645 | SEQ ID NO: 569 | 268 | 79 |
| ETXM325 | ETXS650 | SEQ ID NO: 371 | ETXS649 | SEQ ID NO: 571 | 274 | 79 |
| ETXM326 | ETXS652 | SEQ ID NO: 372 | ETXS651 | SEQ ID NO: 572 | 607 | 86 |
| ETXM332 | ETXS664 | SEQ ID NO: 378 | ETXS663 | SEQ ID NO: 578 | 81 | 85 |
| ETXM333 | ETXS666 | SEQ ID NO: 379 | ETXS665 | SEQ ID NO: 579 | 130 | 83 |
| ETXM338 | ETXS676 | SEQ ID NO: 384 | ETXS675 | SEQ ID NO: 584 | 33 | 77 |
| ETXM339 | ETXS678 | SEQ ID NO: 385 | ETXS677 | SEQ ID NO: 585 | 1180 | 78 |
| ETXM341 | ETXS682 | SEQ ID NO: 387 | ETXS681 | SEQ ID NO: 587 | 186 | 83 |
| ETXM342 | ETXS684 | SEQ ID NO: 388 | ETXS683 | SEQ ID NO: 588 | 71 | 63 |
| ETXM343 | ETXS686 | SEQ ID NO: 389 | ETXS685 | SEQ ID NO: 589 | 60 | 79 |
| ETXM420 | ETXS840 | SEQ ID NO: 466 | ETXS839 | SEQ ID NO: 666 | 2450 | 77 |
| ETXM421 | ETXS842 | SEQ ID NO: 467 | ETXS841 | SEQ ID NO: 667 | 305 | 81 |
| ETXM422 | ETXS844 | SEQ ID NO: 468 | ETXS843 | SEQ ID NO: 668 | 502 | 80 |
| ETXM423 | ETXS846 | SEQ ID NO: 469 | ETXS845 | SEQ ID NO: 669 | 1660 | 80 |
| ETXM425 | ETXS850 | SEQ ID NO: 471 | ETXS849 | SEQ ID NO: 671 | 1050 | 89 |
| ETXM426 | ETXS852 | SEQ ID NO: 472 | ETXS851 | SEQ ID NO: 672 | 1570 | 74 |
| ETXM432 | ETXS864 | SEQ ID NO: 478 | ETXS863 | SEQ ID NO: 678 | 239 | 85 |
| ETXM433 | ETXS866 | SEQ ID NO: 479 | ETXS865 | SEQ ID NO: 679 | 703 | 86 |
| ETXM452 | ETXS904 | SEQ ID NO: 498 | ETXS903 | SEQ ID NO: 698 | 82 | 78 |
| ETXM472 | ETXS944 | SEQ ID NO: 518 | ETXS943 | SEQ ID NO: 718 | 130 | 77 |
| ETXM492 | ETXS984 | SEQ ID NO: 538 | ETXS983 | SEQ ID NO: 738 | 47 | 84 |
| ETXM500 | ETXS1000 | SEQ ID NO: 546 | ETXS999 | SEQ ID NO: 746 | 607 | 70 |
| ETXM501 | ETXS1002 | SEQ ID NO: 547 | ETXS1001 | SEQ ID NO: 747 | 190 | 74 |
| ETXM502 | ETXS1004 | SEQ ID NO: 548 | ETXS1003 | SEQ ID NO: 748 | 132 | 76 |
| ETXM503 | ETXS1006 | SEQ ID NO: 549 | ETXS1005 | SEQ ID NO: 749 | 322 | 75 |
| ETXM505 | ETXS1010 | SEQ ID NO: 551 | ETXS1009 | SEQ ID NO: 751 | 199 | 72 |
| ETXM506 | ETXS1012 | SEQ ID NO: 552 | ETXS1011 | SEQ ID NO: 752 | 162 | 82 |
| ETXM512 | ETXS1024 | SEQ ID NO: 558 | ETXS1023 | SEQ ID NO: 758 | 76 | 84 |
| ETXM513 | ETXS1026 | SEQ ID NO: 559 | ETXS1025 | SEQ ID NO: 759 | 146 | 77 |

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

In case of ambiguity between the sequences in this specification and the sequences in the attached sequence listing, the sequences provided herein are considered to be the correct sequences.

### Example 11: Murinisation of ZPI siRNA Sequences

The 5 siRNA sequences identified in a Huh7 cell line transfection screen as being inhibitors of ZPI expression are shown in Table 8.

TABLE 8

| Sequence | Sense | Antisense |
|---|---|---|
| ID | (5′ to 3′) | (5′ to 3′) |
| ETXM342 | AAAGAUCUCCAUGAGG | UCGUGCCUCAUGGAGA |
| | CACGA | UCUUUCG |
| | (SEQ ID NO: 268) | (SEQ ID NO: 148) |
| ETXM339 | UACCAAGGAAAUGCCA | CAUGGUGGCAUUUCCU |
| | CCAUG | UGGUAGG |
| | (SEQ ID NO: 265) | (SEQ ID NO: 145) |
| ETXM338 | GCCUUCAUCCACAAGG | AAAAUCCUUGUGGAUG |
| | AUUUU | AAGGCAA |
| | (SEQ ID NO: 264) | (SEQ ID NO: 144) |
| ETXM359 | CUCCACCUUUGACAAG | AAAUUCUUGUCAAAGG |
| | AAUUU | UGGAGGC |
| | (SEQ ID NO: 285) | (SEQ ID NO: 165) |
| ETXM396 | GGAGUUUUGCCUUCAU | UGUGGAUGAAGGCAAA |
| | CCACA | ACUCCCC |
| | (SEQ ID NO: 322) | (SEQ ID NO: 202) |

ETXM338, ETXM359 and ETXM396 do not cross react with mouse ZPI sequences and will not be used in mouse PoC studies. These sequences were murinised so that they are homologous with mouse ZPI sequence for use in mouse studies. The siRNA sequences were aligned with mouse ZPI transcripts NM_144834.4 and NM_001301404.1 and nucleotides that mismatched the mouse sequence changed to match the mouse sequence.

ETXM338 was changed to a C at position 18 of the sense strand and a G at position 4 of the antisense strand to give ETXM1064.

ETXM359 was changed to a G at position 19 of the sense strand, a C at position 3 of the antisense strand and a C at position 23 of the antisense strand to give ETXM1072.

ETXM396 was changed to a U at position 2 of the sense strand, a C at position 5 of the sense strand, a G at position 17 of the antisense strand and an A at position 20 of the antisense strand to give ETXM1076.

The murinised sequences were checked by BLAST search to ensure that they did not cross-react with other mouse transcripts.

The murinised siRNA sequences are shown in Table 9. Positions changed to mouse sequence are underlined.

TABLE 9

| Sequence | Sense | Antisense |
|---|---|---|
| ID | (5′ to 3′) | (5′ to 3′) |
| ETXM1064 | GCCUUCAUCCACAAGG | AAAGUCCUUGUGGAU |
| | ACUUU | GAAGGCAA |
| | (SEQ ID NO: 791) | (SEQ ID NO: 787) |
| ETXM1072 | CUCCACCUUUGACAAG | AACUUCUUGUCAAAGG |
| | AAGUU | UGGAGGC |
| | (SEQ ID NO: 792) | (SEQ ID NO: 788) |
| ETXM1076 | GUAGCUUUGCCUUCAU | UGUGGAUGAAGGCAAA |
| | CCACA | GCUACCC |
| | (SEQ ID NO: 793) | (SEQ ID NO: 789) |

### Example 12: In Vivo Efficacy Data in a Haemophilia Mouse Model

Haemarthrosis is defined as a bleeding into joint spaces that is a common feature of haemophilia. A long-term consequence of repeated haemarthrosis is the development of permanent joint disease known as haemophilic arthropathy. Around 50% of patients with haemophilia develop severe arthropathy resulting in chronic joint pain, reduced range of motion and function, and reduced quality of life. Haemophilic arthropathy is characterised by synovial hyperplasia, chronic inflammation, fibrosis, and haemosiderosis.

The model of haemarthrosis used was the induction of a knee bleed in haem A mice and the appropriate background wild-type (WT) strain, with progression of the bleed into the joint monitored up to 10 days post-injury. Identical studies were conducted twice to increase number of animals for analysis.

The objective of these repeat studies was to demonstrate that prophylactic administration of ETXM1184 could reduce haemarthrosis in Haemophilia A mice after a joint bleed injury. Fitusiran (siRNA targeting antithrombin (AT)) was used as a reference. Advate (recombinant FVIII) was used as positive control.

For that, a total of 20 Haem A mice (Bi, L., Lawler, A., Antonarakis, S. et al. Targeted disruption of the mouse factor VIII gene produces a model of haemophilia A. Nat Genet 10, 119-121 (1995). https://doi.org/10.1038/ng0595-119) and 10 WT mice were used in this study:

| | | | Dosing time (pre- | Termination time | |
|---|---|---|---|---|---|
| Group | Mouse strain | Treatment | injury; days) | (post injury; days) | N |
| 1 | WT | Vehicle | −8 | 10 | 10 |
| 2 | Haem A | Vehicle | −8 | 10 | 10 |
| 3 | Haem A | Fitusiran - 3 mg/kg s.c | −8 | 10 | 10 |
| 4 | Haem A | Fitusiran - 10 mg/kg s.c | −8 | 10 | 10 |
| 5 | Haem A | ETXM1184 - 3 mg/kg s.c. | −8 | 10 | 10 |
| 6 | Haem A | ETXM1184 - 10 mg/kg s.c. | −8 | 10 | 10 |
| 7 | Haem A | FVIII (Advate) - 300 IU/kg i.v | 15 mins | 10 | 10 |

8 days prior to induction of knee bleed, mice were injected subcutaneously (s.c.) with the GalNAc-siRNA construct ETXM1184, fitusiran or a vehicle (0.9% saline) at a dose volume of 5 ml/kg. Advate was injected intravenously 15 minutes prior to joint bleed induction.

To induce knee bleed, mice were weighed and anaesthetised using isoflurane inhaled anaesthetic. Both legs were shaved to expose the knee joint. Mice were injected s.c. with buprenorphine at 10 ml/kg for analgesia and the diameter of both knees was measured with electronic calipers. Subsequently, both knees were wiped with 70% ethanol.

A 30 G sterile hypodermic needle was inserted into the infrapatellar ligament of one knee. The injected knee was randomised between left and right, and the injected side was recorded. Mice were removed from anaesthetic and allowed to recover in a warmed cage before being returned to the home cage.

Mice were monitored regularly for the first 6 hours and were injected subcutaneously with buprenorphine at 10 ml/kg for analgesia at 6 hours post injury. The visual bleeding score (VBS) of the injured knee was assessed at 72 hours and 10 days post-injury.

All mice were carefully inspected daily for clinical signs of excessive blood loss. Mice showing clinical signs of excessive blood loss, piloerection, withdrawing from cage mates or grimacing were killed for welfare reasons.

Mice were taken off study at 10 days post-injury.

A citrated blood sample was taken by cardiac puncture, under isoflurane anaesthesia, plasma prepared and aliquots frozen on dry ice before storing at −80° C. For that, blood was collected into 3.8% Sodium Citrate at a ratio of 1 to 9 followed by centrifugation at 7000×g for 10 minutes at 4° C. In detail, the following steps were performed:

1. Collect blood by cardiac puncture.
2. Flush the syringe and needle with sodium citrate solution (3.8%), leaving solution in the hub of the syringe (˜30 μl).
3. Following blood collection, expel sample into a 1.5 ml microcentrifuge tube and ensure sufficient sodium citrate solution (3.8%) is added to achieve a 1:9 ratio of sodium citrate:blood. Add the sodium citrate solution to the side of the tube, not directly to the sample. Mix by inverting 4-6 times. If not centrifuging sample immediately, keep in a fridge if available or alternatively on a wrapped ice block and continue to invert the collection tube regularly.
4. Centrifuge the samples as soon as possible at a spin speed of 7000×g for 10 minutes at 4° C.
5. Remove all plasma from the sample and place into a fresh microcentrifuge tube.
6. Aliquot the plasma into pre-labelled tubes (Thermo Scientific: 10775974) as follows:
30 μl for potential TGA assay
100 μl for potential APTT assay
All remaining for potential target protein abundance analysis.
   7. Place all aliquots on wet/dry ice immediately.
   8. Transport samples on wet/dry ice.
   9. Transfer samples to −20° C./−80° C. freezer to be stored until use.

The liver was removed and up to 3 portions of each lobe were placed in RNA later and kept at 4° C. for 24 to 72 hours. Tissue was then blotted dry, weighed and stored at −80° C. In detail, the following steps were performed:

1. Immediately after the cardiac puncture, kill the mouse by cervical dislocation.
2. Make an incision into the abdominal wall and remove the liver as quickly as possible.
3. Place the liver on a petri dish on wet ice, to minimise sample degradation.
4. Cut 3×˜50 mg pieces of liver from each of the following lobes: left lateral lobe, medial lobe, right lateral lobe and caudate lobe. Place these liver pieces immediately into pre-labelled tubes (1.5 ml microcentrifuge tubes) containing 500 μl RNAlater, and place the collection tube on wet ice.
a. Transport on wet ice and transfer to storage at 4° C.
b. After a period of 24-72 hours, blot the liver samples and weigh. Record the weights on the terminal sheet.
c. Transfer to −80° C. for long-term storage.
5. Collect any spare liver and place into separate pre-labelled collection tubes (2 ml microcentrifuge tubes).
a. Freeze on dry ice for potential future analyses.
b. Transport samples on dry ice.
c. Transfer to −80° C. for long-term storage.
6. Clean all dissection tools between animals to prevent any cross contamination.

The skin was removed from the legs and the knee joint measured. Legs were subsequently placed in 10% formalin before decalcification and slide preparation. In detail, the following steps were performed:

1. Following the removal of the liver, measure and record the diameter of both the injured and uninjured knee.
2. Remove the skin from both knees. Carry out a visual bleeding score and measure knee joints.
3. Dissect the legs from the top of the femur to the ankle joint and remove some excess muscle, being careful not to cause any damage to the knee and associated structures. Place the knees in pre-labelled tubes (7 ml bijou tubes) containing 10% neutral buffered formalin to be processed for histological analysis.

Both at day 3 and day 10 after induction of knee bleed, Haem A mice that received the GalNAc-siRNA construct ETXM1184 showed a significantly reduced visual bleeding score in comparison to Haem A mice that received the vehicle (0.9% saline) (see FIGS. 12A-12B). Furthermore, the knee diameter of mice that received the GalNAc-siRNA construct ETXM1184 recovered faster following the induction of knee bleed compared to mice that received the vehicle (FIG. 13A). This observation was confirmed by comparing the differences between the diameter of the injured and non-injured skinned knee diameter (FIG. 13B).

Analysis of the Haem A mice 10 days post injury further revealed less severe bone marrow hyperplasia (FIG. 14A), less severe osteoarthritis (FIG. 14B), less severe chondrocyte degeneration/necrosis (FIG. 14C), less severe haemorrhage (FIG. 14D), less severe haemosiderin deposition (FIG. 14E), less severe haematoma (FIG. 14F), less severe osteoclastogenic bone resorption (FIG. 14G), less severe osteolysis (FIG. 14H), less severe periostitis (FIG. 14I), less severe sub-chondral bone sclerosis (FIG. 14J), less severe tendon degeneration (FIG. 14K), less severe tendonitis (FIG. 14L) and less severe tenosynovitis (FIG. 14M) in mice that received the GalNAc-siRNA construct ETXM1184 in comparison to Haem A mice that received the vehicle (0.9% saline).

Comparative data between ETXM1184 and fitusiran is provided in FIGS. 15-20.

### Example 13: Dose-Response for Inhibition of ZPI Expression in Human Huh7 Cells

Huh7 cells (human hepatocyte-derived cell line, obtained from JCRB Cell Bank) were maintained in Dulbecco's Modified Eagle Medium (DMEM) supplemented with 10% FBS at 37° C. in at atmosphere of 5% CO₂. Cells were transfected with siRNA duplexes designed against the target or a negative control siRNA at 0.1 nM and 1 nM. Transfection was carried out by adding 9.7 μL Opti-MEM (ThermoFisher) plus 0.3 μL Lipofectamine RNAiMAX (ThermoFisher) to 10 μL of each siRNA duplex. The mixture was incubated at room temperature for 15 minutes before being added to 100 μL of complete growth medium containing 20,000 Huh7 cells. Cells were incubated for 24 hours at 37° C./5% CO₂ prior to total RNA purification using a RNeasy 96 Kit (Qiagen). Each duplex was tested by transfection in duplicate wells and the experiment was repeated three time.

cDNA synthesis was performed using FastKing RT kit (with gDNase) Kit (Tiangen). Real-time quantitative PCR (qPCR) was performed on an ABI Prism 7900HT or ABI QuantStudio 7 with primers specific for human ZPI (Hs01547819_m1) and human GAPDH (Hs02786624_g1) using a TaqMan Gene Expression Assay Kit (ThermoFisher Scientific).

qPCR was performed in duplicate on cDNA derived from each well and the mean Ct calculated. Relative target expression was calculated from mean Ct values using the comparative Ct (ΔΔCt) method, normalised to GAPDH and relative to untreated cells.

For the inhibition of ZPI, the siRNA duplexes ETXM1184, ETXM1199, ETXM1200, ETXM1201, ETXM1202, ETXM1203, ETXM1204, ETXM1205, ETXM1206 and ETXM1207 were tested (FIG. 21).

1. A method of preventing or treating a disease related to a disorder of haemostasis, the method comprising administering a nucleic acid to an individual, wherein the nucleic acid inhibits the expression of ZPI, wherein the nucleic acid comprises a duplex region that comprises a first strand and a second strand, wherein the second strand that is at least partially complementary to the first strand, wherein the first strand is:
(i) at least partially complementary to a portion of RNA transcribed from the ZPI gene; and
(ii) comprises at least 17 contiguous nucleosides differing by 0 or 1 nucleosides from any one of the first strand sequences as listed in Table 2,
and wherein the first strand and the second strand comprise a nucleoside sequence differing by 0 or 1 nucleosides from any one of the following first and second sequences, respectively: (1) SEQ ID NO: 145 and SEQ ID NO: 265, (2) SEQ ID NO: 148 and SEQ ID NO: 268, (3) SEQ ID NO: 126 and SEQ ID NO: 246, (4) SEQ ID NO: 127 and SEQ ID NO: 247, (5) SEQ ID NO: 128 and SEQ ID NO: 248, (6) SEQ ID NO: 129 and SEQ ID NO: 249, (7) SEQ ID NO: 131 and SEQ ID NO: 251, (8) SEQ ID NO: 132 and SEQ ID NO: 252, (9) SEQ ID NO: 138 and SEQ ID NO: 258, (10) SEQ ID NO: 139 and SEQ ID NO: 259, (11) SEQ ID NO: 144 and SEQ ID NO: 264, (12) SEQ ID NO: 147 and SEQ ID NO: 267, (13) SEQ ID NO: 149 and SEQ ID NO: 269, (14) SEQ ID NO: 226 and SEQ ID NO: 346, (15) SEQ ID NO: 227 and SEQ ID NO: 347, (16) SEQ ID NO: 228 and SEQ ID NO: 348, (17) SEQ ID NO: 229 and SEQ ID NO: 349, (18) SEQ ID NO: 231 and SEQ ID NO: 351, (19) SEQ ID NO: 232 and SEQ ID NO: 352, (20) SEQ ID NO: 238 and SEQ ID NO: 358, and (21) SEQ ID NO: 239 and SEQ ID NO: 359.

2. The method of claim 1, wherein the first and second strands of the nucleic acid comprise a nucleoside sequence differing by 0 or 1 nucleosides from any one of the following first and second sequences, respectively: (1) SEQ ID NO: 128 and SEQ ID NO: 248, (2) SEQ ID NO: 144 and SEQ ID NO: 264, (3) SEQ ID NO: 148 and SEQ ID NO: 268, (4) SEQ ID NO: 149 and SEQ ID NO: 269, and (5) SEQ ID NO: 138 and SEQ ID NO: 258.

3. The method of claim 1, wherein the first and second strands of the nucleic acid comprise a nucleoside sequence differing by 0 or 1 nucleosides from any one of the following first and second sequences, respectively: (1) SEQ ID NO: 148 and SEQ ID NO: 268, (2) SEQ ID NO: 145 and SEQ ID NO: 265, (3) SEQ ID NO: 144 and SEQ ID NO: 264, and (4) SEQ ID NO: 165 and SEQ ID NO: 285.

4. The method of claim 1, wherein the first and second strands of the nucleic acid comprise a nucleoside sequence differing by 0 or 1 nucleosides from any one of the following first and second sequences, respectively: (1) SEQ ID NO: 145 and SEQ ID NO: 265, and (2) SEQ ID NO: 148 and SEQ ID NO: 268.

5. The method of claim 1, wherein the first strand of the nucleic acid has a length in the range of 17 to 30 nucleosides.

6. The method of claim 5, wherein the first strand of the nucleic acid has a length of 19 or 23 nucleosides.

7. The method of claim 1, wherein the second strand of the nucleic acid has a length in the range of 17 to 30 nucleosides.

8. The method of claim 7, wherein the second strand of the nucleic acid has a length of 19 to 21 nucleosides.

9. The method of claim 1, wherein the duplex region of the nucleic acid is between 17 and 30 nucleosides in length.

10. The method of claim 9, wherein the duplex region of the nucleic acid is 19 or 21 nucleosides in length.

11. The method of claim 1, wherein the region of complementarity between the first strand and the portion of RNA transcribed from the ZPI gene is between 17 and 30 nucleosides in length.

12. The method of claim 1, wherein the nucleic acid comprises one or more single-stranded nucleoside overhangs.

13. The method of claim 1, wherein the nucleic acid is an siRNA oligonucleoside.

14. The method of claim 1, wherein one or more nucleosides on the first strand and/or second strand of the nucleic acid are modified.

15. The method of claim 1, wherein the nucleic acid comprises one or more abasic nucleosides.

16. The method of claim 15, wherein the one or more abasic nucleosides are in a terminal region of the second strand of the nucleic acid, and/or wherein at least one abasic nucleoside is linked to an adjacent basic nucleoside through a reversed internucleoside linkage.

17. The method of claim 1, wherein the nucleic acid comprises one or more phosphorothioate internucleoside linkages.

18. The method of claim 1, wherein the nucleic acid is conjugated directly or indirectly to one or more ligand moieties.

19. The method of claim 18, wherein the one or more ligand moieties is present at a 3′ terminal region of the second strand of the nucleic acid.

20. The method of claim 1, wherein the nucleic acid is in a pharmaceutical composition comprising the nucleic acid and a pharmaceutically acceptable excipient or carrier.
